(19) United States
(12) Patent Application Publication
(10) Pub. No.: US 2026/0015401 A1
(43) Pub. Date: Jan. 15, 2026

(54) COMPOSITIONS AND METHODS COMPRISING EPITOPES AND POLYPEPTIDES

(71) Applicant: Angeles Therapeutics, Inc., Toluca Lake, CA (US)

(72) Inventor: Preet M Chaudhary, Toluca Lake, CA (US)

(21) Appl. No.: 19/265,972

(22) Filed: Jul. 10, 2025

Related U.S. Application Data
(60) Provisional application No. 63/669,238, filed on Jul. 10, 2024.

(51) Int. Cl. C07K 14/705; C07K 14/00; C07K 16/28

(52) U.S. Cl. CPC C07K 14/705; C07K 14/001; C07K 16/2803; C07K 2317/24; C07K 2317/565; C07K 2317/567; C07K 2319/40

(57) ABSTRACT

The application provides novel peptide epitope tags and recombinant polynucleotide, polypeptides, vectors, cells and compositions comprising the tags. The application also provides novel designs for synthetic antigen receptors (SARs), novel antigen binding domains, novel SAR constructs and novel methods for manufacturing of cell therapy products. These novel methods and compositions have broad uses in cellular therapy.

### CROSS REFERENCE TO RELATED APPLICATIONS

The application claims priority to U.S. Provisional Application No. 63/669,238, filed Jul. 10, 2024, the disclosures of which are incorporated entirely herein by reference.

### TECHNICAL FIELD

This invention relates to field of biotechnology, and more specifically, to novel epitope tags and fusion proteins containing these epitope tags.

### INCORPORATION BY REFERENCE OF SEQUENCE LISTING

This application contains a Sequence Listing which has been submitted electronically in XML format and is hereby incorporated by reference in its entirety. The Sequence Listing XML file, was created on Jul. 9, 2025, is named “Compositions and methods comprising epitopes and polypeptides”, and is 15,373 kilobytes in size.

### BACKGROUND OF THE INVENTION

Engineered immune cells such as CAR-T cells have revolutionized immunotherapy, but ensuring their safe and effective use requires improved control over cell selection, tracking, and eradication if needed. Researchers have turned to epitope tags—short peptide sequences inserted into cell-surface proteins—as universal handles for detection and purification. For example, small tags like c-Myc, HA, FLAG, or Strep-tag II peptides have been attached to CAR extracellular domains, allowing flow-cytometric detection with anti-tag antibodies. However, tag placement and immunogenicity are critical: an improperly placed tag can sterically hinder antigen binding or signaling, and foreign immunogenic sequences could provoke immune neutralization of the therapeutic cells. Furthermore, using such tags clinically requires a suitable antibody reagent, raising regulatory hurdles for clinical use.

Several groups have developed tag systems to facilitate selection and safety of gene-modified cells. One approach is to use truncated human surface proteins as “tags” recognized by existing clinical antibodies. A truncated epidermal growth factor receptor (tEGFR) retaining the cetuximab epitope has served as a non-immunogenic marker and elimination switch: transduced T cells expressing EGFRt can be purified and later depleted by administering cetuximab. However, its relatively large size (˜170 aa) is a major limitation. Similarly, the synthetic RQR8 tag (136 amino acids) combines an epitope from human CD34 (for immunomagnetic selection with anti-CD34) and a mimotope of CD20 (for deletion with rituximab). These large protein tags, while effective, require co-expression of a sizeable transgene, adding complexity and potential expression burden. Furthermore, because these tags are not physically linked to the CAR polypeptide, their expression may be selectively lost even when CAR expression is retained, undermining their reliability as markers or control elements.

Thus, there remains a need for a minimal, non-immunogenic epitope tag system with a clinically approved antibody for versatile cell engineering. Ideally, such a tag would be (i) small enough to insert into various proteins without disrupting function, (ii) “hypoimmunogenic” (minimizing new immune epitopes), and (iii) paired with a high-affinity antibody or a binding reagent that is safe for human use. To date, no FDA-approved cell therapy product includes a built-in peptide tag for these purposes, underscoring the novelty and utility of the present invention.

### SUMMARY

In one aspect, the present disclosure relates to methods and compositions comprising one or more novel peptide epitope(s) and variants thereof, and to related polynucleotides, polypeptides, vectors, cells, and compositions comprising or encoding the same. The peptide epitope(s), polynucleotides, polypeptides, vectors, cells, and compositions described herein may be utilized alone or in combination with one or more additional agents for the in vitro and/or in vivo detection, elimination, modulation, or selection of target biological materials, including, but not limited to, peptides, polypeptides, proteins, lipid-associated structures, viral particles, viral like particles, lipid nanoparticles and cells expressing said epitopes tags and tagged proteins. The compositions and methods of the present disclosure are further applicable for the prevention, diagnosis, and/or treatment of a variety of diseases and disorders, including neoplastic, autoimmune, infectious, and degenerative conditions.

In one aspect the present invention relates to a fusion protein comprising: (a) a peptide epitope; and (b) a polypeptide. The present invention also relates to a binding agent (e.g., an antibody) that specifically binds to the peptide comprised in the fusion protein of the invention. The present invention also relates to a fusion protein comprising a peptide that the antibody of the invention binds to. The present invention also relates to a complex comprising a fusion protein of the invention and an antibody of the invention. The present invention also relates to a nucleic acid encoding a fusion protein of the invention or an antibody of the invention. The present invention also relates to a vector comprising the nucleic acid of the invention. The present invention also relates to a host cell comprising a nucleic acid of the invention or a vector of the invention or expressing a fusion protein of the invention or the antibody of the invention. The present invention also relates to a use of an antibody of the invention for the detection, immobilization, isolation, or purification of a fusion protein of the invention. The present invention also relates to a method of detecting and/or isolating a fusion protein of the invention, comprising contacting the fusion protein with an antibody of the invention. Where the fusion protein of the invention comprises an antibody moiety, the present invention also relates to a method of isolation of a specific target of the antibody moiety. The present invention relates to a kit comprising a nucleic acid or a nucleic acid expression construct encoding a peptide as comprised in a fusion protein of the invention and optionally an antibody of the invention. In an embodiment, the fusion protein is a Synthetic Antigen Receptor.

In one embodiment, the invention provides a small (5-50 amino acids) peptide epitope tag, designated Small Hypoimmunogenic Antibody Recognizable Peptide Tag (SHARP-tag or SHARP-tag) and its variants along with methods and tools for its use in protein and cell engineering. In an example embodiment, the SHARP-tag comprises the amino acid sequence RSEDRY (SEQ ID NO:1) or RSEDRYR (SEQ ID NO: 2). In an example embodiment, the SHARP-tags comprise the amino acid sequence represented by SEQ ID NO: 1-123, 150-167, 251-440, 550-579, 631-650, 651-654, 656, 674-676, 686, 689, and 692-720. The SHARP-tags are specifically recognized by a binding moiety referred to as SABR (SHARP Antigen Binding Reagent). In an embodiment, SABR is an antibody, antibody fragment, bispecific antibody, antibody conjugate, a non-immunoglobulin antigen binding scaffold or functional variants thereof. In an example embodiment, SABR is an antibody drug conjugate Polatuzumab vedotin or functional variants thereof. In another example embodiment, SABR is a monoclonal antibody 2F2, SN8, 10D10, H2Mab-250 or functional variants thereof. Polatuzumab vedotin is an FDA-approved antibody with high affinity for specific SHARP-tags (e.g., SEQ ID NO: 1-23). H2Mab-250 is a Her2 specific monoclonal antibody that binds with high affinity to specific SHARP-tags (e.g., SEQ ID NO: 557-566) and their variants. The SHARP-tag/SABR system offers a compact, hypoimmunogenic marker for a wide array of biotechnology and therapeutic applications.

SHARP-tag and Variants: In an embodiment, the invention provides an epitope tag (or tag) peptide of 5 amino acids (SEQ ID NO: 560), 6 amino acids (SEQ ID NO: 1), 7 amino acids (SEQ ID NO: 2) or 8 amino acids (SEQ ID NO: 3) that is derived from sequences with low immunogenicity. In an embodiment, the SHARP-tags (e.g., SEQ ID NOs: 1-123, 150-167, 251-440, 550-579, 631-650, 651-654, 656, 674-676, 686, 689, and 692-720), and variants including those with conservative amino acid substitutions or length modifications, are included as long as they retain binding to a SABR.

The tag's small size and hydrophilic character allow it to be fused into proteins with minimal structural or functional disruption. In an embodiment, the tag is polar. In an embodiment, the tag is not hydrophobic. In an embodiment, the SHARP-tag is designed to lack T-cell epitopes and to minimize antibody responses when used in an appropriate host. In an embodiment, the tag is hypoimmunogenic, minimally immunogenic or nonimmunogenic. In an embodiment, the tag is derived from an endogenous protein. In an embodiment, the tag is identical in sequence to a region of an endogenous protein. In an embodiment, the SHARP-tag is located in and/or derived from the N-terminal, C-terminal, the juxta membrane region, and/or hinge or stalk regions of an endogenous protein. In an embodiment, the SHARP-tag is located in the extracellular region of an endogenous protein. In an embodiment, the endogenous protein is not an intracellular protein. In an embodiment, the endogenous protein is not a human nuclear protein. In an embodiment, the endogenous protein is a target antigen for drug development, optionally wherein the drug is a recombinant polypeptide (e.g., an antibody, antibody conjugate, non-immunoglobulin antigen binding scaffold etc.). In an embodiment, drug(s) targeting the endogenous protein are in pre-clinical and/or clinical development. In an embodiment, the endogenous protein is a cancer antigen, a cell surface marker, a cancer cell-associated antigen, a tumor antigen, an autoimmune-associated antigen, and/or a differentiation antigen. In an embodiment, the epitope tag (i.e., SHARP-tag) has more than 60% (e.g., 70%, 75%, 80%, 85%, 90%, 95%, 99%) sequence identity to a peptide located in the unfolded region of an endogenous protein. In an embodiment, the epitope tag (i.e., SHARP-tag) has more than 60% (e.g., 70%, 75%, 80%, 85%, 90%, 95%, 99%) sequence identity to a peptide located in the random coil or unstructured region of an endogenous protein. In an embodiment, the tag has more than 60% (e.g., 70%, 75%, 80%, 85%, 90%, 95%, 99%) sequence identity to a peptide located in the loops connecting secondary structure elements within an endogenous protein. In an embodiment the tag lacks a cysteine residue. In an embodiment the tag lacks a disulfide bond. In an embodiment, the tag lacks an Asn-X-Ser/Thr motif (where X can be any amino acid residue) that would create an N-linked glycosylation site. In an embodiment, the tag lacks an N-linked glycosylation site. In an embodiment, the tag has an α-helical secondary structure. In one embodiment, the tag is detectable with high sensitivity in vitro, in vivo, or in both contexts, thereby enabling robust identification, tracking, or quantification of the tagged molecule under experimental and/or physiological conditions. In an embodiment, the tag has less than 50% (e.g., 40%, 30%, 20%, 10%, 5%, 1%) sequence identity to a peptide located in a region of an endogenous protein that is a mutational hot-spot and/or is associated with a congenital or acquired disease (e.g., cancer). In an embodiment, the endogenous protein is human CD79b, Her2, Her3, CD19, CD20, CD22, CD30, CD33, CD34, CD123, FLT3, FGFR2, BCMA, CS1, CD38, GPRC5D, EGFR, Nectin-4, ROR1, TROP2, PD1, or PDL1. In an embodiment, the endogenous protein is not human CD79b, Her2, Her3, CD19, CD20, CD22, CD30, CD33, CD34, CD123, FLT3, FGFR2, BCMA, CS1, CD38, GPRC5D, EGFR, Nectin-4, ROR1, TROP2, PD1, or PDL1.

In an embodiment, the epitope tag according to the invention is a small, monomeric epitope tag of 5-50aa, preferably ≤15aa with minimal size. The sequence is preferably uncharged and hydrophilic at physiological pH and most preferably, it is devoid of residues prone to be modified by amine-reactive fixatives and cross-linkers. A further advantage of the epitope tag of the present invention is that it is not restricted in terms of localization (N, C or in between proteins). In addition, the epitope tags of the invention can be placed at the N- or C-terminus of a target protein or even in between two folded protein domains without compromising proper targeting and folding of target proteins. The epitope tag and antibody system presented here is suited for an exceptionally broad range of applications ranging from biotechnology to cell biology. A single tag can therefore simultaneously replace a great variety of traditional epitope tags.

SABR: In an embodiment, the invention provides a binding moiety, designated SABR, that specifically binds the SHARP-tag. In an embodiment, SABR (e.g., an antibody) is in complex with an epitope tag it specifically binds to. In an embodiment, an SABR is a monoclonal antibody that specifically binds a SHARP-tag. In one embodiment, SABR is a fully human or humanized antibody. In an embodiment, SABR has defined heavy (vH) and light (vL) chain variable sequences (e.g., heavy chain variable regions given as SEQ ID NO:899-919, 937-941, 2179-2182 and 2184; and light chain variable regions given as SEQ ID NO: 774-794, 808-818 and 2173-2178, and 2183). In an embodiment, SABR has a vH sequence represented by SEQ ID NO:922 and vL sequence represented by SEQ ID NO:797 and functional variants thereof. In an embodiment, SABR has a vH sequence represented by SEQ ID NO:966-970 and vL sequence represented by SEQ ID NO:846-847 and functional variants thereof. In an embodiment, the antibody may have up to 20 amino acid substitutions in framework regions to optimize properties (affinity, stability, reduced immunogenicity) while retaining the same antigen specificity. In an embodiment, SABR includes antibodies and their functional equivalents (including chimeric or de-immunized variants) that are or will be clinically approved, enabling their use in vivo. In an embodiment, SABR encompasses binding molecules such as Fab fragments, single-chain Fv (scFv), nanobodies, diabodies, and any antigen-binding fragment that recognizes the SHARP-tag. In an embodiment, SABR encompasses conjugated forms of binding reagents (including any fragment, variant, or derivative thereof), including antibody-drug conjugates (ADCs) carrying cytotoxic payloads (e.g., vedotin, calicheamicin), radioactive moiety (e.g., beta-emitters, Auger-emitters, conversion electron-emitters, alpha-emitters, and low photon energy-emitters), or a protein toxin (e.g., ricin or Pseudomonas exotoxin) for imaging or therapy, and binding reagents conjugated to magnetic beads or solid supports for cell isolation. In further embodiments, SABR is a polyclonal antibody. In certain embodiments, the epitope tag (i.e., SHARP-tag) is recognizable by an antibody or antibody fragment, such as a single-chain variable fragment (scFv), Fab, F(ab′)₂, or a derivative thereof, and/or by a non-immunoglobulin antigen-binding scaffold, including but not limited to a DARPIN, CENTYRIN, D-domain, an affibody, an affilin, an adnectin, an affitin, an obody, a repebody, a fynomer, an alphabody, an avimer, an atrimer, a pronectin, an anticalin, a kunitz domain, and an Armadillo repeat protein. In some embodiments, SABR is a monoclonal antibody (mAb) or a functional fragment or variant thereof. In some embodiments, SABR (e.g. antibody or antibody conjugate) is or will be approved by a regulatory authority (e.g., FDA) for administration to a subject, including a human or a non-human subject (e.g., dog, horse, cat, cow, camel, elephant etc.). In some embodiments, SABR (e.g. antibody or antibody conjugate) is or will be in clinical development for use in a human or a non-human subject. In some embodiments, the SABR (e.g. antibody or antibody conjugate) binds to SHARP-tag that is derived from an endogenous protein. In an embodiment, the tag is identical in sequence to a peptide present in an endogenous protein.

In an embodiment, the SABR binds to a SHARP-tag that has more than 60% identity to a peptide located in the unfolded region of an endogenous protein. In an embodiment, the SHARP-tag has more than 60% sequence identity to a peptide located in the random coil or unstructured region of an endogenous protein. In an embodiment, the tag has more than 60% sequence identity to a peptide located in the loops connecting secondary structure elements within an endogenous protein. In an embodiment the tag lacks a cysteine residue. In an embodiment the tag lacks a disulfide bond. In an embodiment, the tag lacks an Asn-X-Ser/Thr motif (where X can be any amino acid residue) that would create an N-linked glycosylation site. In an embodiment, the tag lacks an N-linked glycosylation site. In an embodiment, the tag has an α-helical secondary structure. In one embodiment, the tag is detectable with high sensitivity in vitro, in vivo, or in both contexts, thereby enabling robust identification, tracking, or quantification of the tagged molecule under experimental and/or physiological conditions. In an embodiment, the tag has less than 50% sequence identity to a peptide located in a region of an endogenous protein that is a mutational hot-spot and/or is associated with a congenital or acquired disease (e.g., cancer).

Bispecific and multi-specific antibodies comprising an SABR arm are also included—for example, a bispecific T-cell engager with one arm binding SHARP-tag and the other binding CD3 on T cells, to direct immune clearance of SHARP-tag expressing cells. In other embodiments, the antibody is a bispecific or multi-specific antibody, including but not limited to a bispecific T cell engager (e.g., BiTE), a Tri specific T cell engager, a bispecific NK cell engager (BiKE), or a Tri specific NK cell engager (TriKE).

SABR-based Synthetic Antigen Receptor (SARs) and Chimeric Receptors: In an embodiment, the variable regions of SABR can be incorporated into synthetic or chimeric receptors. The term SAR refers to any non-native antigen binding receptor and includes conventional CARs (e.g., second generation CAR), universal CAR, armored CARs, and next generation CARs (e.g., SIR, STAR, HIT, Ab-TCR, TFP, zSIR, z16SAR, CD16-SAR, uTCR-SAR, Tri-TAC). The term also includes recombinant TCR (T cell receptor). In an embodiment, a SAR comprises a single polypeptide chain. In an embodiment, a SAR comprises more than one polypeptide chains. For instance, SEQ ID NO:2301 presents an anti-SHARP-tag second generation CAR (chimeric antigen receptor) made by fusing the SABR scFv (vH-(G₄S linker)-vL) to signaling domains (e.g., CD3ζ, CD28 or 4-1BB). Examples of SABR based SARs are presented in SEQ ID NO: 2198-2300. Such a SAR enables one engineered cell to recognize and eliminate another cell bearing the SHARP-tag. This forms the basis of tag-directed cell elimination (a “CAR-on-CAR” or anti-tag CAR system for safety), wherein a reserve T cell product expressing an SABR-CAR could be deployed to eradicate SHARP-tagged cells if needed. Likewise, SABR's binding domains can be used in chimeric bispecific receptors or other fusion proteins for targeted delivery.

Tagged Polypeptides (Fusion Proteins): A broad range of polypeptides comprising the peptide tag (i.e., SHARP-tag) are covered. The tag can be fused at the N-terminus, C-terminus, or internally (e.g., in a flexible loop or linker region) of a protein of interest. In an embodiment, the protein of interest (i.e. polypeptide) to which the tag is attached is a globular protein, a membrane protein, a fibrous protein, or natively unfolded protein, or is a subunit of a globular protein, a membrane protein, a fibrous protein, or natively unfolded protein. In an embodiment, the polypeptide to which the tag is attached comprises at least one protein domain. In an embodiment, the peptide tag is fused to the polypeptide at a position that is located outside the at least one protein domain. In an embodiment, fusion protein is in complex with a binding partner that specifically binds to the peptide tag comprised in the fusion protein. In an embodiment, the invention provides a fusion protein comprising a peptide that the SABR binds to. The invention also provides a complex comprising (a) a fusion protein; and (b) an antibody; In an embodiment, fusion protein comprises an antibody. Example categories include the following.

Synthetic Antigen Receptors (SARs): In an embodiment, all generations of CARs (1st generation CAR with CD3ζ only, 2nd generation CAR with one co-stimulatory domain, 3rd generation CAR with multiple costimulatory domain, and armored CARs such as those secreting cytokines, and next generation CARs that provide physiological signaling such as SIR, STAR, HIT, zSIR, z16SAR, TFP, TCR, uTCR-SAR etc.) can incorporate the SHARP-tag. The tag can be placed in the extracellular region—for instance, at the N-terminus before the antigen binding domain (e.g., scFv, vHH, vL, or vH domain etc.), within the scFv linker, in the hinge/spacer, or proximal to the transmembrane domain—to ensure surface exposure. One or more copies of SHARP-tags may be present on one or more chains of a SAR. Examples of polynucleotides encoding TAG-SARs comprising the SHARP-tags described herein are provided in SEQ ID NO (DNA): 7708-8703.

Monoclonal Antibodies and Fragments: Therapeutic or diagnostic antibodies can be engineered to include SHARP-tag, for example, in a flexible loop of the heavy chain constant region or as a C-terminal or N-terminal tag or in the constant regions). This allows such antibodies to be tracked or pulled down with SABR. Moreover, biotherapeutics like antibody-drug conjugates or bispecific antibodies can carry SHARP-tag for quality control or dual-binding functionality.

Cytokines and Chemokines: In an embodiment, immune modulatory proteins (e.g., IL-2, IL-15, interferons) and chemokines can be fused with SHARP-tag. Tagged cytokines can be detected in complex biological fluids using SABR or removed if necessary (for example, a SHARP-tagged IL-12 that can be neutralized or cleared by SABR in case of toxicity).

Receptors and Ligands: Any cell-surface receptor, co-receptor, or ligand protein (native or synthetic) can be modified to include SHARP-tag in an exposed extracellular loop or tail. For instance, a T cell receptor (TCR) or a synthetic Notch (synNotch) receptor could carry the tag for tracking engineered cells expressing these receptors.

Synthetic Fusion Proteins: This includes designer molecules such as “switch receptors” or “suicide proteins” that incorporate the SHARP-tag. For example, a fusion of SHARP-tag to a transmembrane anchor can serve as a stand-alone marker/suicide protein on the cell surface (analogous to RQR8 or truncated EGFR, but much smaller). Such a protein provides no signaling function of its own but permits selection (via SABR-coated beads) and deletion of the cell (via SABR-ADC or SABR-engaging effector cells). Another example is a multipurpose switch protein combining SHARP-tag with a cell-growth or death signal: e.g., a SHARP-tag fused to a co-stimulatory domain that can deliver a proliferative signal when crosslinked by SABR, or a SHARP-tag fused to an inducible caspase domain that triggers apoptosis upon SABR-mediated aggregation. These embodiments illustrate the flexibility of the tag in constructing safety switches.

The invention also provides that naturally occurring protein or an endogenous protein or its isoform can serve as marker/suicide protein on the cell surface. In an embodiment, CD79b (SEQ ID NO (DNA): 3110 and SEQ ID NO (PRT): 660) and CD79b isoform-2 (SEQ ID NO (DNA): 3106 and SEQ ID NO (PRT):656) and variants thereof with up to 30 amino acid substitutions can serve as marker/suicide proteins.

In an embodiment, the invention provides a recombinant polypeptide comprising one or more copies of a tag (e.g., a SHARP-tag) where the tag is derived from an endogenous protein. In an embodiment, the tag is identical in sequence to a region of an endogenous protein. In an embodiment the tag lacks a cysteine residue. In an embodiment the tag lacks a disulfide bond. In an embodiment, the tag lacks an Asn-X-Ser/Thr motif (where X can be any amino acid residue) that would create an N-linked glycosylation site. In an embodiment, the tag lacks an N-linked glycosylation site. In an embodiment, the tag has an α-helical secondary structure. In an embodiment, the tag has less than 50% (e.g., 40%, 30%, 20%, 10%, 5%, 1%) sequence identity to a peptide located in a region of an endogenous protein that is a mutational hot-spot and/or is associated with a congenital or acquired disease (e.g., cancer). In an embodiment, the tag is 5, 6, 7, 8, 9, 10, 11, 12, 13, 14, 15, 16, 17, 18, 19, 20, or 30 amino acids in length. In an embodiment, the tag is recognized by a drug (e.g. antibody or a derivative) that is or will be approved by a regulatory agency for in vivo administration or is in clinical development for administration to a subject.

Genetic Constructs and Vectors: In an embodiment, nucleic acids encoding the SHARP-tag and the various tagged polypeptides are encompassed. This includes DNA and RNA sequences encoding the SHARP-tags (and variants) and vectors for expression. Viral vectors (such as lentiviral, retroviral, adenoviral, or AAV vectors) carrying SARs or other transgenes with SHARP-tag are provided, as well as non-viral vectors like plasmids and mRNA transcripts for transient expression. Packaging systems for viral production can also leverage the SHARP-tag: for instance, a viral envelope protein or a transduction particle can be engineered to display the SHARP-tag epitope, allowing virus-containing cells or virus-like particles (VLPs) to be immunoaffinity-purified or neutralized using SABR. The invention covers production host cells (e.g., retroviral packaging lines) engineered with SHARP-tag markers for lot release testing and safe elimination after vector production. In an embodiment, the invention provides an engineered viral particle, a virus-like particle (VLP) or a lipid nanoparticle that is modified to express a recombinant polypeptide comprising one or more copies of a tag (e.g., a SHARP-tag) where the tag has more than 60% sequence identity to a peptide present in an endogenous protein. In an embodiment the tag lacks a disulfide bond. In an embodiment, the tag lacks an Asn-X-Ser/Thr motif (where X can be any amino acid residue) that would create an N-linked glycosylation site. In an embodiment, the tag lacks an N-linked glycosylation site. In an embodiment, the tag has an α-helical secondary structure. In an embodiment, the tag has less than 50% sequence identity to a peptide located in a region of an endogenous protein that is a mutational hot-spot and/or is associated with a congenital or acquired disease (e.g., cancer). In an embodiment, the tag is 5 to 30 amino acids in length. In an embodiment, the tag is recognized by a drug (e.g. antibody or a derivative) that is or will be approved by a regulatory agency for in vivo administration or is in clinical development for administration to a subject.

Engineered Cells Expressing SHARP-tag Constructs: Any cell that is genetically modified to express a SHARP-tagged polypeptide is within the scope of the invention. Primary human T lymphocytes transduced to express SHARP-tagged SARs are a prime example, but the cell type is not limited to T cells. Natural Killer (NK) cells, NKT cells, macrophages, dendritic cells, or even stem cells (e.g., HSCs or iPSCs engineered to express a therapeutic transgene with SHARP-tag) can be included. The tag provides a universal marker on these cells for the following applications. In an embodiment, the invention provides an engineered cell or a recombinant cell that is genetically modified to express a recombinant polypeptide comprising one or more copies of a tag (e.g., a SHARP-tag) where the tag is derived from an endogenous protein. In an embodiment, the tag is identical in sequence to a region of an endogenous protein. In an embodiment, the epitope tag (i.e., SHARP-tag) is located in the extracellular region of an endogenous protein. In an embodiment, the endogenous protein is not an intracellular protein. In an embodiment, the epitope tag (i.e., SHARP-tag) is located in and/or derived from the N-terminal, C-terminal, the juxta membrane region, and/or hinge or stalk regions of an endogenous protein. In an embodiment, the epitope tag (i.e., SHARP-tag) is located in and/or derived from the unfolded region of an endogenous protein. In an embodiment, the epitope tag (i.e., SHARP-tag) is located in and/or derived from the random coil or unstructured region of an endogenous protein. In an embodiment, the tag is located in and/or derived from the loops connecting secondary structure elements within an endogenous protein. In an embodiment, the tag lacks a cysteine residue, a disulfide bond, an Asn-X-Ser/Thr motif and/or an N-linked glycosylation site. In an embodiment, the tag is 5 to 30 amino acids in length. In an embodiment, the tag is recognized by a drug (e.g. antibody or a derivative) that is or will be approved by a regulatory agency for in vivo administration or is in clinical development for administration to a subject.

Isolation & Enrichment: SHARP-tagged cells can be selectively enriched from a mixed population using SABR-based reagents. For example, magnetic beads coated with SABR will capture tagged cells for separation, analogous to clinical CD34-selection techniques. This allows high-purity recovery of transduced cells prior to patient infusion. In still further aspects, the present disclosure is directed to a method for activating a cell, such as a T cell (e.g., a non-natural T cell), comprising contacting a cell with a binding domain specific for a tag cassette, wherein the cell comprises a nucleic acid molecule encoding a fusion protein (e.g., a TAG-SAR; e.g., SEQ ID NO: 4808) according to this disclosure and the binding domain specific for the tag cassette is attached to a solid surface.

In some aspects, the present disclosure is directed to a method for identifying cell, such as a T cell, comprising contacting a sample comprising a cell, such as a T cell (e.g., a non-natural T cell) with a binding domain specific for a tag cassette, wherein the cell comprises a nucleic acid molecule encoding a fusion protein (e.g., a TAG-SAR) or a recombinant protein (e.g., CD79b; e.g., SEQ ID NO: 3107-3110) according to this disclosure and the binding domain (e.g., 2F2, SN8, huMA79b, or SEQ ID NO:1182-1190, 1198) specific for the tag cassette (i.e., SHARP-tag) comprises a detectable moiety, and detecting the presence of the cell expressing a fusion protein in the sample. In an example embodiment, T cells expressing a TAG-SAR (e.g., SEQ ID NO: 4808) comprising a tag (SEQ ID NO: 3124) could be detected using a Malibu-Glo reagent encoded by SEQ ID NO: 1198 that comprises a huMA79b scFv fused to NanoLuc (Nluc). For example, Jurkat-NFAT-GFP (JNG) cell clone 4827 that co-expresses a double chain SAR (040524-EZdX1; SEQ ID NO: 4938) with an IL15-CD79b isoform2 fusion protein (SEQ ID NO: 3116) was detected using the Malibu-Glo reagent encoded by SEQ ID NO: 1198. Finally, an antibody (e.g., 2F2, SN8 or SEQ ID NO:1182-1190) can substitute for the Malibu-Glo reagent for detection of T cells expressing the TAG-SAR or a recombinant protein comprising the tag.

In certain further aspects, the present disclosure is directed to a method for sorting a T cell, comprising contacting a sample comprising a non-natural T cell with a binding domain specific for a tag cassette, wherein the non-natural T cell comprises a nucleic acid molecule encoding a fusion protein (e.g., a TAG-SAR; e.g., SEQ ID NO: 4808) or a recombinant protein (e.g., CD79b; e.g., SEQ ID NO: 3107-3110) according to this disclosure and the binding domain specific (e.g., 2F2, SN8, huMA79b, or SEQ ID NO:1182-1190, 1198) for the tag cassette comprises a detectable moiety (e.g., FITC, Biotin etc.), and sorting the non-natural T cell expressing a fusion protein from other cells not expressing a fusion protein in the sample.

In certain aspects, the present disclosure is directed to a method for enriching or isolating a T cell, comprising contacting a sample comprising a non-natural T cell with a binding domain specific for a tag cassette, wherein the non-natural T cell comprises a nucleic acid molecule (e.g., a TAG-SAR; e.g., SEQ ID NO: 4808) encoding a fusion protein or a nucleic acid molecule (e.g., CD79b; e.g., SEQ ID NO: 3107-3110) encoding a recombinant protein according to this disclosure and the binding domain (e.g. 2F2 or SN8 antibody) specific for the tag cassette (e.g., SEQ ID NO: 1-11 comprises a detectable moiety. The method involves enriching for or isolating the non-natural T cell expressing a fusion protein away from other cells not expressing a fusion protein in the sample. Example of nucleic acids encoding the tags that can be used in various embodiments of the invention are provided in SEQ ID NO: 3101-3141.

In further aspects, the present disclosure is directed to a method for depleting certain T cells, comprising contacting a non-natural T cell with a binding domain (e.g., Polatuzumab vedotin) specific for a tag cassette (e.g., SEQ ID NO: 1-123), wherein the non-natural T cell comprises a nucleic acid molecule encoding a fusion protein (e.g., TAG-SAR) according to this disclosure and wherein binding of the binding domain (e.g., Polatuzumab vedotin) specific for the tag cassette leads to cell death of the T cells expressing a fusion protein.

In an embodiment, the invention provides a method of detecting, isolating, depleting or enriching a cell, wherein the cell has been modified to express a recombinant polypeptide comprising one or more copies of a tag (e.g., a SHARP-tag) wherein the tag is derived from an endogenous protein. In an embodiment, the epitope tag (i.e., SHARP-tag) is located in the extracellular region of an endogenous protein. In an embodiment, the endogenous protein is not an intracellular protein. In an embodiment, the tag is identical in sequence to a region of an endogenous protein. In an embodiment, the epitope tag (i.e., SHARP-tag) is located in and/or derived from the N-terminal, C-terminal, the juxta membrane region, and/or hinge or stalk regions of an endogenous protein. In an embodiment, the tag lacks a cysteine residue, a disulfide bond, an Asn-X-Ser/Thr motif and/or an N-linked glycosylation site. In an embodiment, the tag is recognized by a drug (e.g. antibody or a derivative) that is or will be approved by a regulatory agency for in vivo administration or is in clinical development for administration to a subject.

In Vitro Expansion & Activation: The SHARP-tag can serve as a target for stimulatory signals. Coating a culture surface or nanoparticle with SABR (or an anti-SHARP-tag Fc fusion) can crosslink SHARP-tagged SARs (e.g., CAR, SIR, zSIR, z16SAR etc.) on T cells, thereby activating them and promoting proliferation independent of native antigen. This provides a universal expansion method for engineered T cells, overcoming limitations of antigen-specific stimulation or non-specific mitogens. Unlike existing methods that require a specific antigen or feeder cells, an SABR-mediated stimulation is universally applicable to any SHARP-tagged SAR-T product.

In further aspects, the present disclosure is directed to a method for promoting cell proliferation or signaling, such as T cell proliferation, comprising contacting a cell (e.g., a non-natural T cell) with a binding domain specific for a tag cassette and optionally a growth factor cytokine for a time sufficient to allow cell growth, wherein the cell comprises a nucleic acid molecule encoding a fusion protein (e.g., a TAG-SAR; e.g., SEQ ID NO: 4808) according to this disclosure. In an embodiment, the cell expresses a SAR that binds to the tag (e.g., SHARP-tag) described herein. In an example embodiment, the SAR has a SEQ ID NO: 2448, or 2407). In an embodiment, the SAR targets more than one antigen. In an embodiment, the tag is derived from an endogenous protein. In an embodiment, the tag is identical in sequence to a region of an endogenous protein. In an embodiment, the binding domain specific for the tag cassette (i.e., SABR) is attached to a solid surface or a nanoparticle. In an alternate embodiment, the method further includes the addition of an agent that binds to SABR, i.e., an anti-SABR antibody.

In an alternate embodiment, the present disclosure further provides a method for providing a stimulatory or a proliferative signal to a cell expressing a SAR directed against the SHARP-tag by exposing the cells to the SHARP-tag or to a polypeptide comprising the SHARP-tag. In an embodiment, the SAR binds to a SHARP-tag represented by SEQ ID NO: 1-123 or a variant thereof. In an embodiment, the SAR binds to a SHARP-tag represented by SEQ ID NO: 1-123, 150-167, 251-440, 550-579, 631-650, 651-654, 656, 674-676, 686, 689, and 692-720. In an embodiment, the SAR binds to CD79b. In an embodiment, the SAR is double chain SAR or a multi-chain SAR. In an embodiment, the SAR is a single chain CAR. In an embodiment, the SAR is SIR, HC-SIR, zSIR, z16-SIR, CD16-SAR, Ab-TCR, TFP or a CAR. In an embodiment, the method is carried out in vitro. In an embodiment, the SAR comprises a vL region of an antibody comprising a sequence represented by SEQ ID NO: 774-782, 790, 792-794, 808-818, 2173-2178 and the complementary vH fragment of the antibody represented by SEQ ID NO:899-907, 915, 917-919, 937-941, 2179-2182 or variants of the forgoing sequences comprising up to 10 amino acid substitutions in the framework region. In an embodiment, the method is carried out in Vivo. In an embodiment, the method results in activation of cell signaling, optionally NFAT signaling, in the cell expressing the SAR. In an embodiment, the cell is an immune cell (e.g., T cell, NK cell, NKT cell, monocyte, macrophage, B cell, neutrophil, dendritic cell etc.). In an embodiment, the invention provides a method of providing a proliferative or activating signal to a cell, wherein the cell has been modified to express a recombinant polypeptide comprising one or more copies of a tag (e.g., a SHARP-tag) wherein the tag is derived from an endogenous protein. In an embodiment, the epitope tag (i.e., SHARP-tag) in the extracellular region of an endogenous protein. In an embodiment, the endogenous protein is not an intracellular protein. In an embodiment, the tag is identical in sequence to a region of an endogenous protein. In an embodiment, the epitope tag (i.e., SHARP-tag) in and/or derived from the N-terminal, C-terminal, the juxta membrane region, and/or hinge or stalk regions of an endogenous protein. In an embodiment, the epitope tag (i.e., SHARP-tag) in and/or derived from the unfolded region of an endogenous protein. In an embodiment, the epitope tag (i.e., SHARP-tag) in and/or derived from the random coil or unstructured region of an endogenous protein. In an embodiment, the tag in and/or derived from the loops connecting secondary structure elements within an endogenous protein. In an embodiment, the tag lacks a cysteine residue, a disulfide bond, an Asn-X-Ser/Thr motif and/or an N-linked glycosylation site.

Tracking & Detection: Once administered, cells expressing a SHARP-tag can be monitored in the patient's blood or tissues by leveraging the SABR. For instance, a fluorescently labeled SABR can be used in flow cytometry to detect and count SAR-T cells during therapy. Likewise, SABR can be used in immunohistochemistry or immuno-PET imaging (after radiolabeling with, e.g., Zirconium-89) to visualize the distribution and persistence of the cells. Having an invariant epitope on the engineered cells means a single detection reagent can be used across different SAR specificities. In an embodiment, the tag is derived from an endogenous protein. In an embodiment, the epitope tag (i.e., SHARP-tag) in the extracellular region of an endogenous protein. In an embodiment, the endogenous protein is not an intracellular protein.

In Vivo Regulation & Safety: A critical feature of the SHARP-tag platform is the ability to modulate or eliminate the engineered cells post-infusion. By administering SABR or its effector-modified variants, clinicians can partially or completely ablate SHARP-tagged cells if adverse events occur. For example, infusion of SABR IgG could opsonize the cells for Fc-mediated clearance (similar to rituximab clearing CD20+ B cells). For more potent elimination, an SABR-ADC carrying a toxin (such as a maytansinoid or saporin) can selectively kill SHARP-tagged cells. Additionally, an SABR×CD3 bispecific T cell engager can recruit the patient's endogenous T cells to attack the SHARP-tagged cells, functioning as a “kill switch” drug. Conversely, the tag can also be used to temporarily dampen cell activity without killing. Administering a non-cytotoxic bivalent SABR (or Fab fragment) in excess can saturate the SHARP-tag on CAR-T cells, blocking their antigen binding sterically or causing checkpoint-like inhibitory signaling if designed appropriately. Once the crisis (e.g., cytokine release syndrome) is managed, the blocking agent can be withdrawn to restore activity. Thus, the SHARP-tag platform enables fine-tuned on-demand control over cell therapies, enhancing safety.

The invention encompasses therapeutic and/or preventive methods where the activation of the engineered immune cells endowed with a TAG-SAR or expressing a SHARP-tagged polypeptide is modulated by depleting the cells by using a SABR (e.g., Polatuzumab vedotin, 2F2, SN8, 10D10, H3Mab-250 or variants thereof) that binds to the tag (i.e., SHARP-tag) of said TAG-SAR. In an embodiment, the method comprises administration of therapeutic effective amounts of the SABR (e.g., antibody) to the subject in need. In an embodiment, the subject has been administered TAG-SAR or T cells expressing a SHARP-tag. In an embodiment, the subject comprises T cells expressing a TAG-SAR (tagged SAR) or a SHAPR-Tag of the disclosure. In one aspect, the subject is diagnosed with one or more complications of immune effector cell therapy. In one aspect, the subject is at risk of developing one or more complications of immune effector cell therapy. Examples of complications of immune effector cell therapy include, but are not limited to, cytokine release syndrome (CRS), immune effector cell associated neurotoxicity syndrome (ICANS), Hemophagocytic lymphohistiocytosis (HLH), non-ICANS neurotoxicities (e.g., Parkinson's disease, cranial nerve palsies, peripheral neuropathies etc.), cytopenia and second T cell cancers. In an embodiment, the tag (i.e., SHAPR-Tag) is derived from an endogenous protein. In an embodiment, the tag is identical in sequence to a region of an endogenous protein. In an embodiment, the epitope tag (i.e., SHARP-tag) in the extracellular region of an endogenous protein. In an embodiment, the endogenous protein is not an intracellular protein. In an embodiment, the epitope tag (i.e., SHARP-tag) in and/or derived from the N-terminal, C-terminal, the juxta membrane region, and/or hinge or stalk regions of an endogenous protein. In an embodiment, the tag lacks a cysteine residue, a disulfide bond, an Asn-X-Ser/Thr motif and/or an N-linked glycosylation site. In an embodiment, the tag is recognized by a drug (i.e., SABR) that is or will be approved by a regulatory agency for in vivo administration or is in clinical development for administration to a subject.

In various embodiments, SABR is administered intravenously at a dose sufficient to occupy all SHARP-tag sites on the target cells. For example, a single dose of about 10 mg/kg SABR IgG may be given to substantially opsonize and deplete the tagged cells, whereas an SABR-drug conjugate can be administered at a lower dose (e.g., ˜0.1 mg/kg) due to higher potency. Dosage and frequency can be adjusted or repeated as needed to achieve partial or complete depletion of the tagged cells, as illustrated in the Examples below.

Multiplex Tagging and Combinatorial Use: The SHARP-tag can be used alone or in combination with other known tags and safety switches. Some embodiments may incorporate multiple copies of SHARP-tag in tandem to increase antibody avidity—for instance, two or three SHARP-tags spaced by linkers on a single protein, allowing bivalent SABR to bind more tightly (avidity effect) or to ensure at least one epitope is accessible if others are masked. In other embodiments, distinct epitope tags are co-expressed: an engineered cell might express both a SHARP-tag and another tag such as FLAG, Myc, His6, RQR8, CD34, or a rituximab/CD20 mimotope. Such combinations can leverage existing selection tools (e.g., clinically approved anti-CD34 reagents) alongside SABR, or provide redundancy (if one tag/antibody system fails or is immunologically neutralized, another can serve as backup). The co-expression of SHARP-tag with an established suicide gene (like inducible caspase-9) is also envisioned, creating layered safety systems. Importantly, incorporating SHARP-tag in various contexts does not interfere with these other tags; it can be added to existing constructs with minimal genetic footprint.

Exemplary Applications: The SHARP-tag platform is broadly applicable across medical domains.

Oncology: SHARP-tagged CAR-T cells targeting leukemia or solid tumor antigens can be controlled to improve safety (mitigating cytokine storm or off-tumor toxicity by partial depletion of cells) and to allow combination therapies. For example, a SHARP-tagged CAR-T for solid tumors can be given along with an SABR-conjugated imaging agent to track tumor infiltration in real time. SHARP-tag can also be used in oncolytic viruses or tumor vaccines to mark infected or modified cells for follow-up elimination.

Viral Infections: CAR-T cells or TCR-T cells targeting viral infections (HIV, HBV, CMV, etc.) can include SHARP-tag for post-therapy elimination once the infection is controlled, reducing long-term risk. Infected cells could potentially be tagged via gene therapy to serve as immunological targets—e.g., delivering SHARP-tag expression selectively to HIV-infected T cells, then using SABR-based effectors to clear those cells.

Autoimmune Diseases: CAR-Tregs (regulatory T cells engineered with CARs to suppress autoimmune reactions) could carry SHARP-tags to assure they can be ablated if they lose regulatory phenotype or cause immunosuppression beyond the therapeutic window. Similarly, cells engineered to express tolerogenic factors (IL-10, TGF-β) in autoimmune disorders might be given SHARP-tag as a safety off-switch.

Inflammatory and Other Disorders: Engineered cell therapies for conditions like graft-versus-host disease (GvHD), metabolic disorders, or regenerative medicine (e.g., mesenchymal stem cells delivered for tissue repair) can benefit from SHARP-tag monitoring and control. In one scenario, donor T cells in a transplant could be modified to express SHARP-tag so that if GvHD occurs, SABR can be administered to selectively deplete the donor T cells while sparing the patient's own cells.

The invention further provides a method for isolating a tag (i.e., SHARP-tag) suitable for biotechnology, genetic engineering and cell and gene therapy applications.

In one aspect the present invention relates to a fusion protein comprising: (a) a peptide epitope (i.e., SHARP-tag); and (b) a polypeptide. In an embodiment, the peptide epitope is derived from an endogenous protein and/or has at least 60% sequence identity to a region of an endogenous protein, optionally from the extracellular domain of the endogenous protein. In an embodiment, the peptide epitope is hydrophilic, polar, linear, 5 to 50 amino acids in length, hypoimmunogenic, located in the N-terminal, C-terminal or juxta membrane region of the extracellular domain of an endogenous protein, non-immunogenic, not an auto-antigen, not a nuclear protein, does not have at least 50% sequence identity to a region of an endogenous protein that is a hot-spot for mutations or has been associated with a congenital or acquired disease. In an embodiment, the peptide epitope has a sequence represented by SEQ ID NO: SEQ ID NO: 1-123, 150-167, 251-440, 550-579, 631-650, 651-654, 656, 674-676, 686, 689, and 692-720 or a functional variant thereof. The present invention also relates to a binding moiety or a SABR (e.g., an antibody) that specifically binds to the peptide comprised in the fusion protein of the invention. The present invention also relates to a fusion protein comprising a peptide that the SABR (e.g., an antibody) of the invention binds to. The present invention also relates to a complex comprising a fusion protein of the invention and the SABR (e.g., an antibody) of the invention. The present invention also relates to a nucleic acid encoding a fusion protein of the invention or SABR (e.g., an antibody) of the invention. The present invention also relates to a vector comprising the nucleic acid of the invention. The present invention also relates to a host cell comprising a nucleic acid of the invention or a vector of the invention or expressing a fusion protein of the invention or the SABR (e.g., an antibody) of the invention. The present invention also relates to a use of a SABR (e.g., an antibody) of the invention for the detection, immobilization, isolation, or purification of a fusion protein of the invention. The present invention also relates to a method of detecting a fusion protein of the invention, comprising contacting the fusion protein with an SABR (e.g., an antibody) of the invention. The present invention also relates to a method of isolating the fusion protein of the invention, comprising contacting the fusion protein with an antibody of the invention. Where the fusion protein of the invention comprises an antibody moiety, the present invention also relates to a method of isolation of a specific target of the antibody moiety. The present invention also relates a method of treatment comprising administration to the subject with a disease a therapeutic effective amount of a polynucleotide, polypeptide, vector, cell, composition and/or a SABR either alone or in combination with other agents. The present invention also relates to a pharmaceutical composition comprising a polynucleotide, polypeptide, vector, cell, and/or SABR and a suitable carrier. The present invention also relates to methods of detecting, tracking, enriching, controlling, regulating and depleting cells comprising the fusion proteins comprising the peptide tags. The present invention also relates to a kit comprising a nucleic acid or a nucleic acid expression construct encoding a peptide as comprised in a fusion protein of the invention and optionally an SABR (e.g., an antibody) of the invention.

Novel Antigen binding domains. The disclosure further provides novel antigen binding domains (e.g., scFv, vL, vH, vHH etc.) targeting different antigens. The novel vL fragments are represented by SEQ ID NO (PRT):722-847, the complementary vH fragments are represented by 848-967 (Tables 2 and 3). The disclosure also provides novel vHH domains, including humanized vHH, targeting different antigens. These vHH domains are represented by SEQ ID NO:580-607, and 1081-1163. The disclosure also provides fully human heavy chain variable domains (FHVH), which are represented by SEQ ID NO: 1164-1172. These novel antigen binding domains (e.g., scFv, vL, vH, vHH, FHVH etc.) can be used in the construction of SAR (e.g., SIR, zSIR, Ab-TCR, CAR, etc.), antibodies, scFv, bispecific antibodies, antibody drug conjugates and radio-labelled antibodies etc. The disclosure also provides novel antigen binding domains with at least 75% (e.g., 75%, 80%, 85%, 90%, 95%, 99%) amino acid sequence identity in the framework region to the novel antigen binding domains (e.g., vL, vH, vHH, FHVH etc.) described herein (Tables 2-4). The disclosure also provides novel antigen binding domains with up to 2 amino acid differences (i.e., 1, 2) in each of the CDRs of the novel antigen binding domains (e.g., vL, vH, vHH, FHVH etc.) provided in Tables 2-4. In an embodiment, the light chain complementary determining regions 1-3 (LC-CDR1-3) for these novel vL domains are as set forth in SEQ ID Nos: 6601-6726, 6970-7095, 7339-7464, respectively. In an embodiment, the heavy chain complementary determining regions 1-3 (HC-CDR1-3) for these novel vH domains are as set forth in SEQ ID Nos: 6727-6849, 7096-7218, 7465-7587, respectively. In an embodiment, the CDR1-3 for the novel vHH and FHVH domains are provided in SEQ ID NO:6850-6969, 7219-7338, 7588-7707, respectively. Example SAR comprising these novel antigen binding domains are provided in SEQ ID NO (DNA): 9039-9045.

Novel Viral Envelopes. The disclosure provides novel viral envelope proteins for pseudotyping of lentiviral vectors. The disclosure also provides tagged modified envelope proteins that express one or more copies of the epitope tags described herein (e.g., SHARP-tag).

Novel SARs including CD79b SARs: In another aspect, the disclosure provides novel SARs comprising novel polynucleotides, polypeptides targeting specific antigens. In one aspect the antigen is human CD79b. In other aspects the antigen is CD19, CD20, CD22, CD33, CD123, CLL1, BCMA, DLL3, IL13Ra2, PSMA, PSCA, STEAP2, CLDN6, CLDN-18.2, MSLN, GPC3, GCC, GPRC5D, Her2, NPM1c, mutant NPM1c, TAG72, MOG, IL23R, HLA-A2, CSF1R, p53, p53-R175H, p53-R248Q, FCRH5, TAJ/TNFRSF19, ROR1, or EGFRviii.

SAR activated by soluble ligand. In another aspect, the disclosure provides a double chain antigen receptor which shows activation by a soluble ligand. In an embodiment, the receptor is not a natural receptor, e.g., a natural TCR. In an embodiment, the receptor is a synthetic or a non-natural receptor. In an embodiment, the receptor is a synthetic antigen receptor (SAR). In an embodiment, the receptor has the design of a SIR, cTCR, Ab-TCR, zSIR, HIT, STAR, z16SAR, z16SAR. In an embodiment, the receptor comprises at least one extracellular domain and at least one hydrophobic domain. In an embodiment, the receptor is a dimer of two polypeptide chains. In an embodiment, the receptor comprises vL and vH fragments as the antigen binding domain. In an embodiment, the vL and vH fragments are present on two polypeptide chains of the double chain receptor and form a Fv fragment that can bind to the cognate ligand. In an embodiment, the ligand is a peptide tag or polypeptide. In an embodiment, the peptide tag is between 5-10, 6-11, 6-13, 5-10, 4-10, 7-16, 8-17 amino acid residues in length. In an embodiment, the peptide tag forms a linear epitope that does not rely on tertiary structure. In an embodiment, the peptide tag is hydrophilic. In an embodiment, the peptide tag is not hydrophobic. In an embodiment the peptide lacks a cysteine residue. In an embodiment the peptide tag lacks a disulfide bond. In an embodiment, the peptide tag lacks an Asn-X-Ser/Thr motif that would create an N-linked glycosylation site. In an embodiment, the peptide tag is a SHARP-tag. In an embodiment, the peptide tag is represented by SEQ ID NO: 1-123, 150-167, 251-440, 550-579, 631-650, 651-654, 656, 674-676, 686, 689, and 692-720 or variants thereof.

### Novel SAR Design.

In an embodiment, the disclosure provides novel multichain SAR (MC-SAR) designs comprising two or more chains. These multichain SAR designs provide an improvement over the current SAR (e.g., CAR) in providing physiological T cell receptor signaling, lack of tonic signaling, high sensitivity, ability to target more than one antigen, safety, and lower cytokine production. Furthermore, these multichain SAR can comprise a SHARP-tag, which allow for their easy detection, enrichment, activation, proliferation, in vivo monitoring, tracking and depletion. Schematic representations are provided in FIG. 9. In an embodiment, the novel SAR comprises:
a) a first polypeptide chain comprising a first antigen-binding domain comprising a vH antibody domain, a first constant antibody domain and a first T cell receptor domain (TCRD) comprising a first transmembrane domain of a first TCR subunit; and
b) an optional second polypeptide chain comprising a second antigen-binding domain comprising a vH antibody domain, a second constant antibody domain and a second T cell receptor domain (TCRD) comprising a second transmembrane domain of a second TCR subunit; and
c) a third polypeptide chain comprising a third antigen-binding domain comprising a vL antibody domain, and a third constant antibody domain;
   wherein the vH antibody domain of the first antigen-binding domain and the vL antibody domain of the third antigen-binding domain form an antigen-binding module that specifically binds to first antigen; and
   wherein the vH antibody domain of the optional second antigen-binding domain and the vL antibody domain of the third antigen-binding domain form an antigen-binding module that specifically binds to second antigen.

In an embodiment, the first antigen and the second antigen are identical or non-identical. In an embodiment, the first antigen-binding domain and the second antigen-binding domain are identical or non-identical. In an embodiment, the first antigen-binding domain and the second antigen-binding domain bind to the same antigen or different antigens. In an embodiment, the first antigen-binding domain and the second antigen-binding domain bind to the same epitope of an antigen or to different epitopes of an antigen. In an embodiment, a hinge region is present between the constant antibody domains and the TCRDs of the first and the optional second polypeptide chains. In an embodiment, a hinge region is present between the first constant antibody domain and the first T cell receptor domain (TCRD). In an embodiment, a hinge region is present between the second constant antibody domain and the second T cell receptor domain (TCRD). In an embodiment, (i) the first TCR subunit is a TCR α chain, and the second TCR subunit is a TCR β chain; or (ii) the first TCR subunit is a TCRβ chain, and the second TCR subunit is a TCR δ chain; (iii) the first TCR subunit is a TCR γ chain, and the second TCR subunit is a TCR δ chain; or (iv) the first TCR subunit is a TCR δ chain, and the second TCR subunit is a TCR γ chain; or (v) the first TCR subunit is a TCR α chain, and the second TCR subunit is a hybrid TCR chain; or (vi) the first TCR subunit is a hybrid TCR chain, and the second TCR subunit is TCR α chain; or vii) the first TCR subunit is a TCR β chain, and the second TCR subunit is a hybrid TCR chain; or (viii) the first TCR subunit is a hybrid TCR chain, and the second TCR subunit is TCR β chain; or (ix) the first TCR subunit is a TCR γ chain, and the second TCR subunit is a hybrid TCR chain; or (x) the first TCR subunit is a hybrid TCR chain, and the second TCR subunit is TCR γ chain; or (xi) the first TCR subunit is a TCR γ chain, and the second TCR subunit is a hybrid TCR chain; or (xii) the first TCR subunit is a hybrid TCR chain, and the second TCR subunit is TCR γ chain.

In an embodiment, the TCR α chain subunit is represented by SEQ ID NO: 1408 or 1409 or a functional variant thereof with at least 75% identity to the above. In an embodiment, the TCR β chain subunit is represented by SEQ ID NO: 1412 or 1413 or a functional variant thereof with at least 75% identity to the above. In an embodiment, the TCR γ chain subunit is represented by SEQ ID NO: 1415 or 1416 or a functional variant thereof with at least 75% identity to the above. In an embodiment, the TCR γ chain subunit is represented by SEQ ID NO: 1418 or 1419 or a functional variant thereof with at least 75% identity to the above.

In an embodiment, the TCRD is a hybrid TCR chain in which the connecting peptide of one TCR chain is substituted by connecting peptide of another TCR chain. In an embodiment, the connecting peptides of TCR constant chains are represented by SEQ ID NO: 1492-1499.

In an embodiment, the TCRD is a hybrid TCR chain in which the transmembrane domain of one TCR chain is substituted by transmembrane domain of another TCR chain. In an embodiment, the transmembrane domains of TCR constant chains are represented by SEQ ID NO: 1502-1509. In an embodiment, the first TCRD and the second TCRD form a T cell receptor module (TCRM) that is capable of recruiting at least one TCR-associated signaling module.

In an embodiment, the first, second and third constant antibody domains are each selected from the group consisting of SEQ ID NO: 1458-1473 or functional variants thereof. In an embodiment, the third constant antibody domain is a CL antibody domain or functional variants thereof. In an embodiment, the third constant antibody domain is represented by SEQ ID NO: 1458 or a functional variant thereof. In an embodiment, the first, second and third constant antibody domains are each selected from the group consisting of a CH1, CH2, CH3, CH4 and CL antibody domain. In an embodiment, the first and optional second constant antibody domains are each selected from the group consisting of a CH1, CH2, CH3, and CH4 antibody domain. In an embodiment, the first and optional second constant antibody domains are represented by a selected from the group consisting of SEQ ID NO: 1459, 1460, 1462, 1463, 1465-1473 or a functional variant thereof. In an embodiment, the first and the optional second constant antibody domains are identical or non-identical. In an embodiment, the first and the optional second constant antibody domains are selected from the group consisting of CH1, CH2, CH3, and CH4 antibody domain. In an embodiment, the first and/or the optional second constant antibody domain is a CH1 antibody domain, and the third constant antibody domain is a CL antibody domain. In an embodiment, the first and/or the optional second constant antibody domain is selected from the group consisting of CH1, CH2, CH3, and CH4 antibody domain, and the third constant antibody domain is a CL antibody domain. In an embodiment, one or more disulfide bonds are formed between the hinge region present between the first and the optional second polypeptide chain. In an embodiment, the hinge region is represented by a SEQ ID NO: 1513-1520, 1780 or a variant thereof. In an embodiment, the hinge region is represented by a SEQ ID NO: 1513-1520, 1780 or a variant thereof in which one or more cysteine residues are mutated to another amino acid residue, optionally wherein the other residue is Ala. In an embodiment, the sequence of the first and the optional second antibody constant domain along with the hinge regions is represented by SEQ ID NO: 1461, 1462, 1501, 1507, and 1512 or a functional variant thereof. In an embodiment, a linker is present between any of the domains of the SAR. In an embodiment, the linker is between 1-25 amino acids in length. In an embodiment, the linker is a SHARP-tag. In an embodiment, one or more non-scFv autonomous antigen binding domains (AABD) are operationally linked via optional linker domains (or linkers) to the N-terminus or near the N-terminus of the vH and vH antibody domains of the first, optional second and/or the third polypeptide chains. In an embodiment, the first, optional second and/or the third polypeptide chains comprise one or more copies of an epitope tag, e.g., SHARP-tag. In an embodiment, the AABD comprises a SHARP-tag. In an embodiment, the linker domain (or linker) comprises and SHARP-tag. In an embodiment, the invention provides a complex comprising the SAR and at least one TCR-associated signaling module selected from the group consisting of CD36F, CD37F, and CD3ζ Q.

In an embodiment, the first polypeptide chain further comprises a first peptide linker between the first antigen-binding domain and the first TCRD and the optional second polypeptide chain further comprises a second peptide linker between the second antigen-binding domain and the second TCRD. In an embodiment, the first TCRD further comprises a first connecting peptide or fragment thereof of a TCR subunit N-terminal to the first transmembrane domain and the optional second TCRD further comprises a second connecting peptide or fragment thereof of a TCR subunit N-terminal to the second transmembrane domain. In an embodiment, the transmembrane domain comprises a sequence selected from the group consisting of SEQ ID NO: 1502-1505 or functional variants with 1, 2, 3 or 4 amino acid substitutions. In an embodiment, the connecting peptide comprises a sequence selected from the group consisting of SEQ ID NO: 1492-1499 or functional variants with 1, 2, 3 or 4 amino acid substitutions.

In an embodiment, the vL and/or vH domains are replaced by vHH domains. In an embodiment, the vL and/or one or both vH domains are replaced by single antibody domains (e.g., single vH domain, or FHVH domain). In an embodiment, the vL and/or vH domains are replaced by scFV domains. In an embodiment, the vL and/or one or both vH domains are replaced by non-immunoglobulin antigen binding scaffolds (e.g., DARPIN). In an embodiment, the vL and/or one or both vH domains are replaced by a single chain TCR domain. In an embodiment, the vL and/or and or both vH domains are replaced by an adaptor, an adaptor binding domain or a tag. In an embodiment, the vL and/or and or both vH domains are replaced by an adaptor, an adaptor binding domain or a tag is a SHARP-tag. In an embodiment, the vL and/or and or both vH domains are replaced by the ligand binding domain of a receptor (e.g., Fc binding region of CD16A or CD16B).

In an embodiment, the disclosure provides novel multichain SAR designs that form non-T cell receptor module (NTCRM). Schematic representations are provided in FIG. 10. In an embodiment, the SAR comprises:
d) a first polypeptide chain comprising a first antigen-binding domain comprising a vH antibody domain, a first constant antibody domain and a first Membrane associated module (MAM); and
e) an optional second polypeptide chain comprising a second antigen-binding domain comprising a vH antibody domain, a second constant antibody domain and a second Membrane associated module (MAM); and
f) a third polypeptide chain comprising a third antigen-binding domain comprising a vL antibody domain, and a third constant antibody domain.
   In an embodiment, the vH antibody domain of the first antigen-binding domain and the vL antibody domain of the third antigen-binding domain form an antigen-binding module that specifically binds to a first antigen; and the vH antibody domain of the optional second antigen-binding domain and the vL antibody domain of the third antigen-binding domain form an antigen-binding module that specifically binds to a second antigen. In an embodiment, the first MAM and the second MAM form a non-T cell receptor module (NTCRM) that activates at least one signaling pathway and/or recruiting at least one signaling adaptor.

In an embodiment, the first, second and third constant antibody domains are each selected from the group consisting of SEQ ID NO: 1458-1473 or functional variants thereof. In an embodiment, the third constant antibody domain is a CL antibody domain or functional variants thereof. In an embodiment, the third constant antibody domain is represented by SEQ ID NO: 1458 or a functional variant thereof. In an embodiment, the first, second and third constant antibody domains are each selected from the group consisting of a CH1, CH2, CH3, CH4 and CL antibody domain. In an embodiment, the first and optional second constant antibody domains are each selected from the group consisting of a CH1, CH2, CH3, and CH4 antibody domain. In an embodiment, the first and optional second constant antibody domains are represented by a selected from the group consisting of SEQ ID NO: 1459, 1460, 1462, 1463, 1465-1473 or a functional variant thereof. In an embodiment, the first and the optional second constant antibody domains are identical or non-identical. In an embodiment, the first and the optional second constant antibody domains are selected from the group consisting of CH1, CH2, CH3, and CH4 antibody domain. In an embodiment, the first and/or the optional second constant antibody domain is a CH1 antibody domain, and the third constant antibody domain is a CL antibody domain. In an embodiment, the first and/or the optional second constant antibody domain is selected from the group consisting of CH1, CH2, CH3, and CH4 antibody domain, and the third constant antibody domain is a CL antibody domain. In an embodiment, one or more disulfide bonds are formed between the hinge region present between the first and the optional polypeptide chain. In an embodiment, the hinge region is represented by a SEQ ID NO: 1513-1520, 1780 or a variant thereof. In an embodiment, the hinge region is represented by a SEQ ID NO: 1513-1520, 1780 or a variant thereof in which one or more cysteine residues are mutated to another amino acid residue, optionally wherein the other residue is Ala. In an embodiment, the sequence of the first and the optional second antibody constant domain along with the hinge regions is represented by SEQ ID NO: 1461, 1462, 1501, 1507, and 1512 or a functional variant thereof. In an embodiment, a linker is present between any of the domains of the SAR. In an embodiment, the linker is between 1-25 amino acids in length. In an embodiment, the linker is a SHARP-tag. In an embodiment, one or more non-scFv autonomous antigen binding domains (AABD) are operationally linked via optional linker domains (or linkers) to the N-terminus or near the N-terminus of the vH and vH antibody domains of the first, optional second and/or the third polypeptide chains. In an embodiment, the first, optional second and/or the third polypeptide chains comprise one or more copies of an epitope tag, e.g., SHARP-tag. In an embodiment, the AABD comprises a SHARP-tag. In an embodiment, the linker domain (or linker) comprises and SHARP-tag.

In an embodiment, the first polypeptide further comprises a first hinge domain or fragment thereof N-terminal to the first MAM; and/or the second polypeptide further comprises a second hinge domain or fragment thereof N-terminal to the second MAM. In an embodiment, the hinge domain comprises a sequence selected from the group consisting of SEQ ID NO: 1729-1743 or functional variants with 1, 2, 3 or 4 amino acid substitutions. In an embodiment, the SAR comprises a disulfide bond between a residue in the first MAM and the second MAM and/or a residue in the first hinge domain and a residue in the second hinge domain.

In an embodiment, first polypeptide further comprises a first cytosolic domain containing an optional activation domain C-terminal to the first transmembrane/membrane-anchoring domain comprising the first MAM; and/or the second polypeptide further comprises a second cytosolic containing an optional activation domain C-terminal to the second transmembrane/membrane anchoring domain comprising the second MAM. In an embodiment, the cytosolic domain comprises a sequence selected from the group consisting of SEQ ID NO: 1744-1766 or functional variants with 1, 2, 3 or 4 amino acid substitutions.

In an embodiment, the first polypeptide chain further comprises a first accessory intracellular domain comprising a co-stimulatory or a co-receptor domain sequence C-terminal to the first transmembrane/membrane anchoring domain of the first MAM; and/or the second polypeptide chain further comprises a second accessory intracellular domain comprising a co-stimulatory, a coreceptor domain sequence or a signaling molecule C-terminal to the second transmembrane/membrane anchoring domain comprising the second MAM. In an embodiment, the co-stimulatory domain comprises a sequence selected from the group consisting of SEQ ID NO: 1759-1766 or functional variants with 1, 2, 3, 4, 5, or 10 amino acid substitutions.

In an embodiment, the a) co-stimulatory domain is selected from the cytosolic domain of CD28, 4-1BB, OX40, 2B4, CD27, CD81, CD2, CD5, BAFF-R, CD30, CD40, HVEM or ICOS, or a variant or a fragment thereof; and b) co-receptor domain is selected from the cytosolic domain of CD8a, CD8b or CD4, or a variant or a fragment thereof and c) signaling molecule is a kinase optionally selected from the group of Lck, FYN, ZAP-70, PLCγ1, SLP-76 and LAT or a functional variant or a fragment thereof. In an embodiment, the first and/or the second MAM and the NTCRM are comprised of the transmembrane/membrane anchored domain, optional cytosolic domain, optional hinge domain and/or optional extracellular domain of a non-T cell receptor and/or a signaling adaptor. In an embodiment, the first and/or the second MAM and the NTCRM are comprised of the transmembrane/membrane anchored domain, optional cytosolic domain, optional hinge domain and/or optional extracellular domain that are all derived from a single or different non-T cell receptor and/or a signaling adaptor or variants thereof.

In an embodiment, the two transmembrane/membrane anchored domains, optional cytosolic domains, optional co-stimulatory domain, optional hinge domains and/or optional extracellular domains are identical or different in sequence and are derived from the same protein.

In an embodiment, a) the non T cell receptor is a naturally occurring receptor and is selected from the group consisting of: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, a variant of any of the foregoing, and fragments thereof; and b) the signaling adaptor is selected from the group consisting of: CD3ζ, FcRγ, DAP10, a variant of any of the foregoing and fragments thereof.

In an embodiment, the a) CD16 lacks a partial or complete cytosolic domain and/or comprise a mutation in the transmembrane domain; and/or b) one or both CD3ζ cytosolic domains comprise a deletion of residue Q101; c) both a) and b).

In an embodiment, one or more autonomous antigen binding domains (AABD) or fragments thereof are operationally linked to the N-terminus or near the N-terminus of the vL, vH, Vα, Vβ, Vγ and/or Vδ domain via one or more optional linkers.

In an embodiment, the transmembrane domain comprises a sequence selected from the group consisting of SEQ ID NO: 1502-1506, 1714-1728 or functional variants with 1, 2, 3 or 4 amino acid substitutions. In an embodiment, the connecting peptide comprises a sequence selected from the group consisting of SEQ ID NO: 1492-1499 or functional variants with 1, 2, 3 or 4 amino acid substitutions. In an embodiment, the connecting peptide comprises a sequence selected from the group consisting of SEQ ID NO: 1492-1499 or functional variants with 1, 2, 3 or 4 amino acid substitutions.

In an embodiment, the first antigen and the second antigen are identical or non-identical. In an embodiment, the first antigen-binding domain and the second antigen-binding domain are identical or non-identical. In an embodiment, the vH domain of the first polypeptide chain and the vH domain of the second polypeptide chain are identical or non-identical. In an embodiment, the first antigen-binding domain and the second antigen-binding domain bind to the same antigen or different antigens. In an embodiment, the first antigen-binding domain and the second antigen-binding domain bind to the same epitope of an antigen or to different epitopes of an antigen.

In an embodiment, the vL and/or vH domains are replaced by vHH domains. In an embodiment, the vL and/or one or both vH domains are replaced by single antibody domains (e.g., single vH domain, or FHVH domain). In an embodiment, the vL and/or vH domains are replaced by scFV domains. In an embodiment, the vL and/or one or both vH domains are replaced by non-immunoglobulin antigen binding scaffolds (e.g., DARPIN). In an embodiment, the vL and/or one or both vH domains are replaced by a single chain TCR domain. In an embodiment, the vL and/or and or both vH domains are replaced by an adaptor, an adaptor binding domain or a tag. In an embodiment, the vL and/or and or both vH domains are replaced by an adaptor, an adaptor binding domain or a tag is a SHARP-tag. In an embodiment, the vL and/or and or both vH domains are replaced by the ligand binding domain of a receptor (e.g., Fc binding region of CD16A or CD16B).

In an embodiment, a hinge region is present between the constant antibody domains and the connecting peptides of the first and the optional second polypeptide chains. In an embodiment, a hinge region is present between the first constant antibody domain and the first MAM and/or between the second constant antibody domain and the second MAM.

In an embodiment, any of the SAR described herein is expressed in a cell, optionally wherein the cell is a T cell, NKT cell, NK cell, or a pluripotent stem cell that can give rise to a T cell, NKT cell or an NK cell. In an embodiment, the cell has impaired or abolished expression of one or more TCR constant chains, wherein optionally the TCR constant chains are selected from the group consisting of TCR α, Rβ1, β2, γ, δ or pre-TCRα. In an embodiment, the SAR is expressed from an endogenous TCR locus, optionally wherein the endogenous TCR locus is TCR α, β1, β2, γ, δ or pre-TCRα gene locus. In an embodiment, the SAR is expressed under the promoter and regulatory element of an endogenous TCR gene, optionally wherein the endogenous TCR gene is TCR α, β1, β2, γ, δ or pre-TCRα gene. In an embodiment, the SAR is co-expressed with an accessory module or a therapeutic control. In an embodiment, the SAR is transduced in a cell using a vector, optionally wherein the vector is a lentiviral vector, retroviral vector, viral like particle, lipid nanoparticle, mRNA vector, DNA vector, transposon, plasmid vector, adenoviral vector or adeno-associated viral vector. In an embodiment, the SAR is transduced in a cell a) in vitro, b) in vivo, c) both a) and b). In embodiments, the SAR is expressed transiently or expressed stably.

The invention provides an effector cell (e.g., a T cell) presenting on its surface the SAR, optionally wherein the effector cell does not express the first TCR subunit and/or the second TCR subunit. The invention provides a method of killing a target cell, comprising contacting the target cell with the effector cell expressing the SAR. The invention provides a pharmaceutical composition comprising the effector cells expressing the SAR and a pharmaceutically acceptable carrier. The invention provides a method of treating a disease in an individual in need thereof comprising administering to the individual an effective amount of the pharmaceutical composition comprising the effector cells expressing the SAR.

Provided herein are pharmaceutical compositions that include any of the nucleic acids, polypeptides, envelopes, vectors, cells and compositions described herein that encode any of the epitope tags, single chain, double chain and multi-chain SARs and/or accessory modules described herein, or any of the sets of nucleic acids described herein that together encode any of the single chain, double chain and multi chain SARs and/or accessory modules described herein, and a pharmaceutically acceptable carrier. Also provided are kits that include any of the any of the nucleic acids, polypeptides, envelopes, vectors, cells and pharmaceutical compositions described herein.

Also provided are methods of treatment and/or prevention of disease by using any of the polynucleotides, polypeptides, vectors, cells and compositions described herein. In one aspect, the SAR (e.g., CD79b SAR) of the disclosures are used for the treatment of patients with leukemia and lymphoma, autoimmune disorders (e.g., Lupus, idiopathic myositis, myasthenia gravis, rheumatoid arthritis etc.) and/or allergic disorders (e.g., asthma).

The invention provides a cell-based method to determine the potency and/or titer of a vector encoding a SAR (e.g., a SIR, a HC-SIR, Ab-TCR etc.) by infecting T cells or a T cell line with impaired or abolished expression of one or more endogenous TCR constant chain, optionally wherein the TCR constant chain is selected from the group consisting of TCRα, β1, β2, γ, δ or preTCRα constant chain. In an embodiment, the SAR is a double chain SAR. In an embodiment, the SAR comprises a TCR constant chain, optionally wherein the TCR constant chain is selected from the group consisting of TCRα, β1, β2, γ, δ or preTCRα constant chain. In an embodiment the method offers greater sensitivity and accuracy in determining the titer of the vector as compared to the assay performed in wild-type T cell or T cell lines. In an embodiment, the T cell or a T cell line with impaired or abolished expression of one or more endogenous TCR constant chains can be obtained by methods known in the art, such as CRISP/Cas9, siRNA or Zn finger nucleases.

The invention also provides a method to detect the expression of a SAR based on staining with an antibody, antibody fragment or a non-immunoglobulin antigen binding domain raised or directed against the antigen binding domain(s) of the SAR. In an embodiment, the antibody is directed to an immunoglobulin or an antibody or an antibody fragment (e.g., Fab, Fab2 etc.). In an embodiment, the antibody is not an anti-ideotype antibody.

The invention also provides a method to measure the expression of SAR constructs comprising TCR constant chains. The expression of the SIR can be difficult to detect with the conventional methods, e.g., staining with Protein L due to poor sensitivity and high background. The invention provides a novel approach to measure the expression of a SAR comprising TCR constant chains. Examples of such SAR constructs include SIR, HIT, STAR, HC-SAR, multi-chain SAR etc. In an embodiment, to detect the expression of such SAR with greater sensitivity, the cells are stained with antibodies against human TCRα, TCRβ1 and/or TCRβ2 constant chains. In an embodiment, the antibodies or the antibody is conjugated to one or more fluorochromes and the analysis is done using flow cytometry. In an embodiment, the T cell is a T cell line, optionally Jurkat cell line or a clone thereof. In an embodiment, the expression of the TCRβ2 constant chain on JNG cells is used to determine the expression of the SAR. In an embodiment, the T cell or the T cell line has impaired or abolished expression of one or more endogenous TCR constant chains, optionally wherein the TCR constant chain is selected from the group consisting of TCRα, β1, β2, γ, δ or preTCRα constant chain. In an embodiment, the cell expressing the SAR is a primary T cell. In an embodiment, the T cell(s) are stained with antibodies against TCRβ1 and TCRβ2 constant chains. In an embodiment, the presence of T cells that show staining with both TCRβ1 and TCRβ2 constant chain specific antibodies (i.e., double positive cells) is used as a measure of the expression of the SAR. In an embodiment, the method is used to measure the potency of the vector encoding the SAR and as a product release assay.

In an embodiment, the invention provides a short method for manufacturing of a SIR, HC-SIR, zSIR, z16-SIR, CD16-SIR comprising the following steps. 1. An optional step of giving a mobilizing agent to the donor to mobilize immune effector cells, optionally wherein the mobilizing agent is selected from the group consisting of 1) CXCR4 antagonist, 2) cytokine, 3) Dasatinib, 4) exercise, 5) chemotherapy, 6) combination of one or more of 1-5; 2; collecting immune effector cells via apheresis; 3) Isolating T cells using CD3 selection or CD4/CD8 enrichment and/or depletion of non-T cells; 4) Activating the enriched T cells, optionally in the presence of IL2 or IL15/IL7, and optionally using CD3/CD28 beads or antibodies for 12-18 hours; 5) Transducing 300-400 million isolated T cells with a nucleic acid encoding the SAR, optionally wherein the transduction is done using a vector, optionally wherein the vector is a lentiviral vector, retroviral vector, viral like particle or lipid nanoparticle. In an embodiment, the transduction is done using spin-infection. In an embodiment, the transduction is done in the presence of vectofuscin; 6) expanding the cells for 12 hours to 7 days in the presence of IL2 or IL15/IL7 and CD3/CD28 beads or antibodies; 7) harvesting the cells. In an embodiment, the manufacturing is done in close automated system. In an embodiment, the system is Prodigy® CliniMACS. In an embodiment, the process is performed using the T Cell Transduction Large Scale (TCT-LS) Process from Prodigy. In an embodiment, the optional steps of cryopreservation of the apheresed product and its subsequent thawing prior to T cell isolation is included.

In an embodiment, the invention provides a method of improving the efficacy of an immune effector cell composition comprising a SAR targeting PSMA (e.g., a PSMA-targeted CAR, SIR, zSIR, Ab-TCR etc.) by administration of an anti-androgen agent. The anti-androgen agent may be selected from the group consisting of bicalutamide, flutamide, nilutamide, enzalutamide, apalutamide, darolutamide, abiraterone acetate, or a pharmaceutically acceptable salt, ester, prodrug, or formulation thereof. In some embodiments, the anti-androgen agent is a non-steroidal androgen receptor antagonist or an androgen biosynthesis inhibitor. In various embodiments, the anti-androgen drug is administered systemically, including but not limited to oral, intravenous, subcutaneous, or intramuscular routes. In preferred embodiments, the drug is administered orally.

In some embodiments, the anti-androgen drug is administered at a dosage that is therapeutically effective for treating prostate cancer in a human subject. In certain embodiments, the dose is selected from one of the following: Bicalutamide: from about 10 mg to about 150 mg per day; preferably about 50 mg per day; Flutamide: from about 250 mg to about 1000 mg per day, administered in two or more divided doses; Nilutamide: from about 150 mg to about 300 mg per day; Enzalutamide: from about 40 mg to about 200 mg per day; preferably about 160 mg per day; Apalutamide: from about 60 mg to about 300 mg per day; preferably about 240 mg per day; Darolutamide: from about 300 mg to about 1800 mg per day, administered in one or more divided doses; preferably about 1200 mg per day; Abiraterone acetate: from about 250 mg to about 1000 mg per day; preferably about 1000 mg per day in combination with a corticosteroid (e.g., prednisone 5 mg twice daily). In certain embodiments, the anti-androgen drug is administered daily, once daily, or in divided doses two or more times per day. In some embodiments, the anti-androgen drug is administered in combination with other agents, including but not limited to corticosteroids (e.g., prednisone), GnRH agonists or antagonists, chemotherapeutic agents, or immune checkpoint inhibitors. In various embodiments, the anti-androgen drug is formulated in a composition comprising one or more pharmaceutically acceptable excipients, carriers, diluents, or stabilizers, suitable for systemic administration.

The following detailed description and examples illustrate various embodiments of this platform and methods for its use, without limiting the scope of the invention.

### BRIEF DESCRIPTION OF THE DRAWINGS

FIG. 1. Malibu Glo assay showing specific binding of the Malibu Glo reagent (SEQ ID NO:1199) to T cells expressing the two TAG-SAR constructs (SEQ ID NO:5045 and 5046) as compared to untagged SAR construct (SEQ ID NO: 5044).

FIG. 2. Flow cytometry showing specific binding of the Malibu Glo reagent (SEQ ID NO: 1199) to T cells expressing the two TAG-SAR constructs (SEQ ID NO: 5045 and 5046) as compared to the untagged SAR construct (SEQ ID NO: 5044).

FIG. 3. Flow cytometry showing specific binding of the FLAG-tagged SN8 antibody to T cells expressing the two TAG-SAR constructs (SEQ ID NO: 5045 and 5046) as compared to the untagged SAR construct (SEQ ID NO: 5044).

FIG. 4. Flow cytometry showing specific binding of the HuMa79b (SEQ ID NO: 1182) and 2F2 (SEQ ID NO: 1189) to T cells expressing the indicated TAG-SAR constructs (SEQ ID NO: 4818, 4824, 4819, 4995) as compared to the untagged SAR construct (SEQ ID NO: 5044).

FIG. 5. Matador Cytotoxicity Assay on LucPPe-expressing LNCaP cells confirms selective depletion of T cells expressing the TAG-SAR constructs (SEQ ID NO: 5045 and 5046) as compared to the untagged SAR construct (SEQ ID NO: 5044) following treatment with Polatuzumab vedotin as reflected by loss of cytotoxicity on LNCaP cells.

FIG. 6. Bioluminescence Imaging (BLI) showing that in vivo treatment with Polatuzumab vedotin protects mice against toxicity of T cells expressing STEAP2 targeted second-generation CAR (SEQ ID NO: 5057) comprising a SHARP-tag.

FIG. 7. Bioluminescence imaging of NSG mice xenografted with JEKO-1 cells and administered either control T cells or T cells expressing the indicated SAR constructs.

FIG. 8. Bioluminescence imaging of NSG mice xenografted with LNCaP cells and administered either control T cells or T cells expressing the indicated SAR constructs.

FIG. 9. Schematic representations of multi-chain unispecific and bispecific Synthetic Antigen Receptors (MC-SAR) comprising TCR signaling chains. vH1 and vH2 reflect two different vH domains that share a common light chain variable region (i.e., vL domain). Cα, Cβ, Cγ and Cδ represent modules comprising the connecting peptide, transmembrane and/or cytosolic domains of TCRα, β, γ and δ constant chains. FIGS. 7C, D, G and H show hybrid TCR constant chains.

FIG. 10. Schematic representations of multi-chain unispecific and bispecific Synthetic Antigen Receptors (MC-SAR) comprising non-TCR signaling domains. Abbreviations used are: CD3z, a module comprising the transmembrane and cytosolic domain of CD3z with the optional hinge domain of CD3z; CD16, a module comprising the transmembrane and optional cytosolic domain of CD16 with the optional hinge domain of CD16; FcRγ, a module comprising the transmembrane and cytosolic domain of FcRγ with the optional hinge domain of FcRγ; BB, cytosolic domain of 4-1BB, CD3z-BB-CD3z, a module comprising the transmembrane domain of CD3z, cytosolic domain of 4-1BB and cytosolic domain of CD3z. vH1 and vH2 reflect two different vH domains that share a common light chain variable region (i.e., vL domain).

### DETAILED DESCRIPTION

In one aspect the present disclosure provides an isolated epitope tag that comprises or consists of 5, 6, 7, 8, 9, 10, 11, 12, 13, 14, 15, 16, 17, 18, 19, 20 or more than 20 amino acid residues. In one aspect the present disclosure provides an isolated epitope tag that comprises or consists of 5, 6, 7, 8, 9, 10, 11, 12 or 13 amino acid residues. In one aspect, the present disclosure provides an epitope tag comprising 5 amino acid residues. In one aspect, the present disclosure provides an epitope tag comprising 6 amino acid residues. In another aspect the present disclosure provides an epitope tag comprising 7 amino acid residues. In another aspect the present disclosure provides an epitope tag comprising 8 amino acid residues. In one aspect, the present disclosure provides an isolated epitope tag comprising from about 6 to about 30 amino acid residues. In other embodiments, the epitope tag comprises at least 6, 7, or 8 amino acids. In yet other embodiments, the epitope tag ranges from 6 to 11, 6 to 13, 6 to 20, 6 to 25, 6 to 50, 6 to 60, 7 to 30, 7 to 20, 7 to 15, 8 to 30, 8 to 20, 8 to 15, or 9 to 11 amino acids in length. In various embodiments, the epitope tag may have about 6, 7, 8, 9, 10, 11, 13, 14, 16, or 18 amino acid residues. The epitope tag may be linear and optionally designed to minimize immunogenicity while enabling reliable detection, isolation, or modulation of tagged polypeptides.

In one aspect, the invention provides an epitope tag that is 5, 6, 7, 8, 9, 10, 11, 12, 13, 14, 15, 16, 17, 18, 19, 20, 21, 22, 23, 25, 26, 30, 35, 40, or 50 amino acids in length.

In one aspect the epitope tag is recognized by a drug that is or will be approved by a regulatory agency. In one aspect the epitope tag is recognized by a drug that is approved or will be approved by a regulatory agency in USA, Europe, UK, Canada, China, Japan, India, Brazil, Israel, South Korea, Australia, New Zealand, Malaysia, Indonesia, Singapore, Thailand, Colombia, Chile, South Africa, Saudia Arabia, UAE and/or Mexico. In one aspect the regulatory agency is the US Food and Drug Administration (FDA) or an equivalent agency in other countries or jurisdictions. In one aspect, the regulatory agency is EMA (European Medical Agency), Medicines and Healthcare Products Regulatory Agency (MHRA) of UK, Health Canada, Pharmaceutical and Medical Devices Agency (PMDA) of Japan, National Medical Products Administration (NMPA) of China, or Central drug Standard Control Organization (CDSCO) of India.

In an embodiment, the drug is an antibody, antibody fragment, or a non-immunoglobulin antigen binding scaffold.

In an embodiment the epitope tag is recognized by a drug (e.g., an antibody or an antibody drug conjugate or an ADC) that is approved or would be approved by a regulatory agency (e.g., FDA or EMA) for in vivo administration to a subject. In an embodiment, the subject is a human subject. In one aspect the epitope tag is recognized by an antibody, a bispecific antibody and/or an antibody drug conjugate. In one aspect, the drug (e.g., an antibody or an antibody drug conjugate) is Polatuzumab or Polatuzumab vedotin or Polatuzumab vedotin-piiq (sold as Polivy®) or a generic version of the forgoing. In one aspect, the epitope tag is bound by Polatuzumab or Polatuzumab vedotin (Polivy®) or a generic version of the forgoing. In an embodiment, the drug (e.g., an antibody, a bispecific antibody or an antibody drug conjugate) binds to the same epitope as Polatuzumab or Polatuzumab vedotin or Polatuzumab vedotin-piiq (sold as Polivy®) or a generic version of the forgoing. In an embodiment, the drug binds to an overlapping epitope as Polatuzumab or Polatuzumab vedotin or Polatuzumab vedotin-piiq (sold as Polivy®) or a generic version of the forgoing. In an embodiment, the drug competes for binding with Polatuzumab or Polatuzumab vedotin-piiq (Polivy®) or a generic version of the forgoing. In an embodiment, the drug (e.g., antibody, bispecific antibody or ADC) binds to CD79b. In an embodiment, the drug (e.g., antibody, bispecific antibody or ADC) binds to human CD79b. In an embodiment, the drug binds to CD79b from a non-human species (e.g., mouse, rabbit, dog, rat, monkey, cat, elephant etc.). In an embodiment, the antibody has light chain variable regions represented by SEQ ID NO: 774-794, 808-818 and 2173-2178, and 2183 and heavy chain variable regions represented by SEQ ID NO: 899-919, 937-941, 2179-2182 and 2184 or variants thereof with up to 20 amino acid substitutions.

In an embodiment, the epitope tag, polypeptide and cells comprising the same are recognized by monoclonal antibody SN8 (Thermofisher Scientific Catalog #Catalog #604-490), 3A2-2E7 (BD Bioscience; Catalog 557592), CD79b-2F2, CB3-1 (BD Biscience), AT105 (Abcam), huMA79b, Polatuzumab or Polatuzumab vedotin or variants thereof. In an embodiment, the epitope tag, polypeptides (e.g., CAR, next generation CAR, antibodies, cytokines, chemokines etc.) and cells comprising the same are recognized by an antibody or antibody fragment, antibody conjugates (e.g., scFv, Fab, bispecific antibody, antibody drug conjugate, scFv etc.) described in PCT/US2024/10592, US20070207142, WO2009012268, WO2009012256 and EP2176295B1, which are incorporated in their entirety by reference herein, or the functional variants or derivatives thereof.

In an embodiment, the epitope tag, polypeptides (e.g., CAR, next generation CAR, antibodies, cytokines, chemokines etc.), vectors, cells and compositions comprising the epitope tag described herein are recognized by an antibody, an antibody fragment or an antibody derivative (e.g., scFv, Fab, bispecific antibody, antibody drug conjugate etc.) that comprises the CDRs (complementary determining regions) of antibodies, antibody fragments and antibody conjugates described in US20070207142, PCT/US2024/10592, WO2009012268, WO2009012256 and EP2176295B1, or variants with 1, 2 or 3 amino acids substitutions in one or more of the CDRs. In an embodiment, the epitope tag, polypeptides (e.g., CAR, next generation CAR, antibodies, cytokines, chemokines etc.), vectors, cells and compositions comprising the epitope tag described herein are recognized by an antibody comprising the heavy chain and light chain regions represented by SEQ ID NO:1194 and 1195, respectively, or functional variants with 70%, 75%, 80%, 85%, 90%, 95%, 97%, 98% or 99% sequence identity thereto or variants with up to 10, 20, 30, or 40 amino acids substitutions in the framework regions. In an embodiment, the epitope tag, recombinant polypeptides (e.g., CAR, next generation CAR, antibodies, cytokines, chemokines etc.), vectors, cells and compositions comprising the epitope tag described herein are recognized by an antibody, an antibody fragment or an antibody conjugate (e.g., scFv, Fab, bispecific antibody, antibody drug conjugate etc.) that comprises the CDRs (complementary determining regions) of an antibody with heavy chain and light chain regions represented by SEQ ID NO:1194 and 1195, respectively, or functional variants with up to 1, 2 or 3 amino acid substitutions in one or more CDR regions. In an embodiment, the epitope tag, polypeptides (e.g., CAR, next generation CAR, antibodies, cytokines, chemokines etc.), vectors, cells and compositions comprising the one or more epitope tags described herein are recognized by an antibody (e.g., monoclonal antibody, polyclonal antibody, bispecific antibody etc.), an antibody fragment (e.g., scFv, vL, vH, Fv etc.), or an antibody conjugates (e.g., antibody drug conjugate etc.) that comprise the CDRs (complementary determining regions) of an antibody, an antibody fragment (e.g., scFv, vL, vH) and/or an antibody conjugate described herein or functional variants with 1 or 2 amino acids substitutions in the CDRs. In an embodiment, the epitope tag, polypeptides (e.g., CAR, next generation CAR, antibodies, cytokines, chemokines etc.) and cells comprising the epitope tag(s) are recognized by an antibody, antibody fragment, antibody drug conjugate comprising the vL fragment of an antibody represented by SEQ ID NO: 774-782, 790, 792-794, 808-818, 2173-2178 and the complementary vH fragment of the antibody represented by SEQ ID NO:899-907, 915, 917-919, 937-941, 2179-2182 or variants of the forgoing sequences comprising up to 20 amino acid substitutions in the framework region. In an embodiment, the antibody or its functional variant requires the residue E located at position 3 of SEQ ID NO: 1-11 for binding to the SHARP-tag or the polypeptide, cell, vector, and/or composition comprising the SHARP-tag. The CDRs of the above vL and vH are provided in Tables 2-3.

In an embodiment, the epitope tag, polypeptides (e.g., CAR, next generation CAR, antibodies, cytokines, chemokines etc.) and cells comprising the same are recognized by an antibody, an antibody fragment, or an antibody drug conjugate comprising the vL fragment of an antibody (2F2) represented by SEQ ID NO: 783-789 and 791 and the complementary vH fragment of the antibody represented by SEQ ID NO: 908-914 and 916 or functional variants of the forgoing sequences comprising up to 20 amino acid substitutions in the framework region. In an embodiment, the epitope tag, polypeptides (e.g., CAR, next generation CAR, antibodies, cytokines, chemokines etc.) and cells comprising the epitope tag(s) are recognized by an antibody, antibody fragment, antibody drug conjugate comprising the vL fragment of an antibody (2F2) represented by SEQ ID NO: 783-789 and 791 and the complementary vH fragment of the antibody represented by SEQ ID NO: 908-914 and 916 or functional variants of the forgoing comprising up to 20 amino acid substitutions in the framework region. In an embodiment, the SABR (i.e., antibody, antibody derivatives, antibody fragments or functional variant) requires the residue E located at position 3 of SEQ ID NO: 1-11 for binding to the SHARP-tag or the polypeptide, cell, vector, and/or composition comprising the SHARP-tag.

In an embodiment, the epitope tag, polypeptides (e.g., CAR, next generation CAR, antibodies, cytokines, chemokines etc.) and cells comprising the epitope tag(s) are recognized by an antibody, antibody fragment, antibody drug conjugate or a bispecific antibody comprising the vL fragment of an antibody (10D10) represented by SEQ ID NO: 797 and the complementary vH fragment of the antibody represented by SEQ ID NO: 922 or functional variants of the forgoing comprising up to 20 amino acid substitutions in the framework region.

In an embodiment, the epitope tag, polypeptides (e.g., CAR, next generation CAR, antibodies, cytokines, chemokines etc.) and cells comprising the epitope tag(s) are recognized by an antibody, antibody fragment, antibody drug conjugate, bispecific antibody and/or radiolabeled antibody comprising the vL fragment of an antibody (H2Mab-250) represented by SEQ ID NO: 846-847 and the complementary vH fragment of the antibody represented by SEQ ID NO: 966-970 or functional variants of the forgoing comprising up to 10 amino acid substitutions in the framework region. In an embodiment, the functional variant of H2Mab-250 is a fully human, humanized or chimeric antibody. In an embodiment, the functional variant of H2Mab-250 comprises 1, 2, 3 amino acid substitution in one or more of the CDRs provided it retains binding to the epitope represented by SEQ ID NO: 557-566 or a variant thereof. In an embodiment, the functional variant of H2Mab-250 comprises 1, 2, 3 amino acid substitution in one or more of the CDRs provided interacts with the Trp (W) residue for binding to the epitope represented by SEQ ID NO: 557-566 or a variant thereof. The Trp (W) is located at position 4 in SEQ ID NO: 557 and is located between residues I and K. In an embodiment, the SABR is any antibody, antibody fragment, variant thereof that requires the Trp (W) located at position 4 of the SHARP-tag with SEQ ID NO: 557 or a functional variant thereof. The present disclosure further provides recombinant polypeptides (e.g., synthetic antigen receptors, or SARs), cells, vectors, and compositions comprising a SHARP-tag represented by SEQ ID NO: 557-566 or a variant thereof that is specifically recognized by an antibody, antibody fragment, antibody conjugate, bispecific antibody, antibody derivative, or a variant thereof. In certain embodiments, the binding moiety (i.e., a SABR) comprises a variable light (vL) chain comprising a sequence represented by SEQ ID NOs: 846-847 and a complementary variable heavy (vH) chain comprising a sequence represented by SEQ ID NOs: 966-970, or functional variants thereof containing up to 20 amino acid substitutions within the framework regions and/or or 1-3 substitutions in one or more CDRs, provided such variants retain specific binding to the SHARP-tag represented by SEQ ID NO: 557-566 or a variant thereof.

As used herein, the term “CDR” or “complementarity determining region” is intended to mean the non-contiguous antigen combining sites found within the variable region of both heavy and light chain polypeptides. These particular regions have been described by Kabat et al., J. Bio. Chem. 252:6609-6616 (1977); Kabat et al., U.S. Dept. of Health and Human Services, “Sequences of proteins of immunological interest” (1991); Chothia et al., J. Mol. Bio. 196:901-917 (1987); and MacCallum et al., J. Mol. Bio. 25 262:732-745 (1996), where the definitions include overlapping or subsets of amino acid residues when compared against each other. CDRs sequences of the disclosure may follow the definition by AbM used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Kabat, Chothia, MacCallum, Contact, IGMT and AbM are within the scope of the present disclosure. Nevertheless, application of either definition to refer to a CDR of an antibody or grafted antibodies or variants thereof is intended to be within the scope of the term as defined and used herein. As used herein, the different CDRs of an antibody could be also defined by a combination of the different definitions. For example, vHCDR1 could be defined based on Kabat and VHCDR2 could be defined based on Chothia, IGMT or Contact. The amino acid residues which encompass the CDRs as defined by Kabat, Chothia, MacCallum of the above cited references are as follows:

CDR DEFINITIONS

| | Kabat | Chothia | MacCallum |
|---|---|---|---|
| VHCDR1 | 31-35 | 26-32 | 30-35 |
| VHCDR2 | 50-65 | 53-55 | 47-58 |
| VHCDR3 | 95-102 | 96-10 | 193-101 |
| VLCDR1 | 24-34 | 26-32 | 30-36 |
| VLCDR2 | 50-56 | 50-52 | 46-55 |
| VLCDR3 | 89-97 | 91-96 | 89-96 |

(Residue Numbers correspond to the identified reference).

In some embodiments, an antibody or antibody fragment described herein (e.g., SABR) may comprise one or more mutations (e.g., amino acid insertions, deletions, or substitutions) relative to a CDR, vH, vL, heavy chain, or light chain sequence provided in Tables 2 and Table 3. In some embodiments, an antibody or antibody fragment described herein may comprise 1, 2, 3, 4, 5, 6, 7, 8, 9, 10, or more mutations (e.g., amino acid insertions, deletions, or substitutions) relative to a CDR, VH, VL, heavy chain, or light chain sequence provided in Table 2 and Table 3. In some embodiments, an antibody or antibody fragment described herein may be at least 60%, at least 70%, at least 80%, at least 90%, at least 95%, or at least 99% identical to a CDR, VH, VL, heavy chain, or light chain sequence provided in Table 2 and Table 3.

In some embodiments, the antibody or antibody fragment of the present disclosure comprises a heavy chain complementarity determining region 1 (CDR-H1) (according to the IMGT definition system), a heavy chain complementarity determining region 2 (CDR-H2) (according to the Kabat definition system), a heavy chain complementarity determining region 3 (CDR-H3) (according to the Kabat definition system), a light chain complementarity determining region 1 (CDR-L1) (according to the Kabat definition system), a light chain complementarity determining region 2 (CDR-L2) of (according to the Kabat definition system), and a light chain complementarity determining region 3 (CDR-L3) (according to the Kabat definition system).

In some embodiments, the antibody, the antibody fragment or the SAR of the present disclosure comprises a heavy chain variable region (vH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the vH comprising the amino acid sequence of SEQ ID NO: 848-967. Alternatively or in addition (e.g., in addition), the antibody, the antibody fragment or the SAR of the present disclosure comprises a light chain variable region (vL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the vL comprising the amino acid sequence of SEQ ID NO: 722-847. In some embodiments, the single domain antibody (e.g., vHH, FHVH), the antibody fragment or the SAR of the present disclosure comprises a vHH or FHVH domain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the vHH or FHVH comprising the amino acid sequence of SEQ ID NO: 580-607, 1081-1163 and 1164-1172.

In some embodiments, the antibody, the antibody fragment or the SAR of the present disclosure comprises a heavy chain comprising an amino acid sequence least 75% (e.g., 75%, 80%, 85%, 90%, 95%, 98%, or 99%) identical to the amino acid sequence of SEQ ID NO: 848-967. In some embodiments, the antibody, the antibody fragment or the SAR of the present disclosure comprises a light chain comprising an amino acid sequence least 75% (e.g., 75%, 80%, 85%, 90%, 95%, 98%, or 99%) identical to the amino acid sequence of SEQ ID NO: 722-847. In some embodiments, the antibody, the antibody fragment or the SAR of the present disclosure comprises a vHH or FHVH comprising an amino acid sequence least 75% (e.g., 75%, 80%, 85%, 90%, 95%, 98%, or 99%) identical to the amino acid sequence of SEQ ID NO: 580-607, 1081-1163 and 1164-1172.

In an embodiment, the drug (e.g., an antibody, antibody drug conjugate etc.) binds to an epitope (i.e., SHARP-tag) derived from an endogenous protein. In an embodiment, the drug binds to the extracellular domain of an endogenous protein. In an embodiment, the drug binds to an epitope located in the N-terminal 6, 7, 8, 9, 10, 11, 12, 13, 14, 15, 16, 17, 18, 19, 20, 21, 22, 23, 24, 25 or 30 amino acid residues of the extracellular domain of an endogenous protein. In an example embodiment, an epitope tag (i.e., SHARP-tag) located in the N-terminal region of the extracellular domain of an endogenous protein is represented by SEQ ID NO: 550-579, 692-707, 712-720 or a variant thereof with 1, 2, 3, 4 or 5 amino acid substitutions. In an embodiment, the drug binds to an epitope located in the C-terminal 6, 7, 8, 9, 10, 11, 12, 13, 14, 15, 16, 17, 18, 19, 20, 21, 22, 25, or 30 amino acid residues of the extracellular domain of an endogenous protein. In an example embodiment, an epitope tag (i.e., SHARP-tag) located in the C-terminal region of the extracellular domain of an endogenous protein is represented by SEQ ID NO: 708-711 or a variant thereof with 1, 2, 3, 4 or 5 amino acid substitutions. In an embodiment, the amino acid residues are numbered based on the mature proteins that lack the signal peptide. In an embodiment, the drug binds to an epitope that is in the juxta membrane 6, 7, 8, 9, 10, 11, 12, 13, 14, 15, 16, 17, 18, 19, 20, 21, 22, 23, 24, 25, 30, 35, 40, 50 or 60 amino acid residues of the extracellular domain of an endogenous protein. In an embodiment, the juxta membrane region is the region located on the extracellular side of the transmembrane region of a protein. In an example embodiment, an epitope tag (i.e., SHARP-tag) located in the juxta membrane region of the extracellular domain of an endogenous protein is represented by SEQ ID NO: 557-579 or a variant thereof with 1, 2, 3, 4 or 5 amino acid substitutions. In an embodiment, an epitope tag (i.e., SHARP-tag) is in or derived from the unfolded region of an endogenous protein. In an embodiment, an epitope tag (i.e., SHARP-tag) is in or derived from the random coil or unstructured region of an endogenous protein. In an embodiment, the tag is in or derived from the loops connecting secondary structure elements within an endogenous protein. In one embodiment, the epitope (i.e., SHARP-tag) comprises, consists of, or overlaps with an amino acid sequence located at or near the N-terminus, C-terminus, juxta membrane, hinge, stalk, or linker regions of an endogenous protein, wherein the sequence corresponds to a contiguous stretch of amino acids selected from the group consisting of residues 1-6, 1-7, 1-8, 1-9, 1-10, 1-11, 1-12, 1-15, 1-20, 1-25, 1-30, 1-50; 2-8, 2-9, 2-10, 2-11, 2-12, 2-15, 2-20, 2-25, 2-30, 2-50; 3-9, 3-10, 3-11, 3-12, 3-15, 3-20, 3-25, 3-50; 4-10, 4-15, 4-20, 4-25, 4-30, 4-50; 5-11, 5-15, 5-20, 5-25, 5-30, 5-50; 6-12, 6-15, 6-20, 6-25, 6-30, 6-50; and 7-13, 7-15, 7-20, 7-20, 7-50, 8-14, 8-20, 8-25, 8-50, 9-15, 9-20, 9-25, 9-30, 9-50, 10-16, 10-20, 10-25, 10-30, 10-50, 11-17, 11-25, 11-30, 11-50, 12-18, 12-25, 12-30, 13-19, 13-25, 13-30, 14-20, 14-25, 14-30, 15-21, 15-25, 15-30, 15-50, 20-50, 25-50, 30-50, 35-50, or 40-50. In an embodiment, the endogenous protein is listed in Table 1. The disclosure also provides recombinant polynucleotides encoding recombinant polypeptide comprising the epitope tags (i.e., SHARP-tag) described herein. Examples of TAG-SAR comprising the SHARP-tags described herein are provided in SEQ ID NO: 7708-8703.

In one aspect, the epitope tag is identical in composition to a sequence present in an endogenous human protein. In one aspect, the epitope tag is identical in composition to a sequence present in the extracellular domain of an endogenous protein. In an embodiment, the epitope tag (i.e., SHARP-tag) is in the extracellular region of an endogenous protein. In an embodiment, the endogenous protein is not an intracellular protein. In an embodiment, the endogenous protein is a human protein. In an embodiment, the human protein is human CD79b. In an embodiment, the endogenous protein is a monkey protein. In an embodiment, the monkey protein is CD79b. In one aspect, the epitope tag is identical in composition to a sequence present at the N-terminus or near the N-terminus of the extracellular domain of an endogenous mature protein or polypeptide. A mature protein or polypeptide refers to a protein or polypeptide that lacks the signal peptide. In one aspect, the epitope tag is identical in composition to a sequence present at the C-terminus or near the C-terminus of the extracellular domain of an endogenous protein. In one aspect, the epitope tag is identical in composition to a sequence present in the juxta membrane region of the extracellular domain of an endogenous protein. In one aspect, the endogenous protein is a Type I or a Type II transmembrane protein. In one aspect, the epitope tag is identical in composition to a sequence present within the N-terminal 5, 6, 7, 8, 9, 10, 11, 12, 13, 14, 15, 16, 17, 18, 19, 20, 21, 22, 23, 24, 25, 26, 27, 28, 29 or 30 residues of the extracellular domain of a mature endogenous human protein, optionally wherein the protein is a Type I transmembrane protein. In one aspect, the epitope tag is identical in composition to a sequence present within the C-terminal 5, 6, 7, 8, 9, 10, 11, 12, 13, 14, 15, 16, 17, 18, 19, 20, 21, 22, 23, 24, 25, 26, 27, 28, 29 or 30 residues of the extracellular domain of an endogenous protein, optionally wherein the endogenous protein is a Type II transmembrane protein. In one aspect, the epitope tag is identical in composition to a sequence present within the juxta membrane 5, 6, 7, 8, 9, 10, 11, 12, 13, 14, 15, 16, 17, 18, 19, 20, 21, 22, 23, 24, 25, 26, 27, 28, 29 or 30 residues of the extracellular domain of an endogenous protein, optionally wherein the endogenous protein is a human protein.

In an embodiment, the endogenous protein is not a nuclear protein. A nuclear protein is a protein found in the cell nucleus. In an embodiment, the endogenous protein is not human La protein. In an embodiment, the epitope tag (i.e., SHARP-tag) has less than 50% (e.g., 40%, 30%, 20%, 10%, 5%, 1%) sequence identity to a peptide present in the human La protein. In an embodiment, the epitope tag is not a human La protein epitope (e.g., E5B9 or E7B6). In an embodiment, the endogenous protein is not an autoantigen. As used herein, the term “autoantigen” refers to an endogenous (self-derived) antigen that is recognized by the immune system of the host, leading to an autoimmune response. In an embodiment, antibodies against the endogenous protein or the epitope tag are not associated with any autoimmune disease. In an embodiment, the endogenous protein and/or the epitope tag does not bear homology to a viral protein. In an embodiment, the endogenous protein is not expressed in both epithelial and mesenchymal cells. In an embodiment, the endogenous protein is not expressed in both the cytoplasm and cell membrane. In an embodiment, the endogenous protein is not known to be localized in the nucleus. In an embodiment, the endogenous protein is not known to and/or believed to act as a transcription factor. In an embodiment, the endogenous protein is not involved in the normal development of organs and tissues, optionally wherein the tissue is kidney or breast. In an embodiment, mutations in the endogenous protein are not linked to a congenital disease, optionally wherein the disease is lacrimo-auriculo-dento-digital (LADD) syndrome, Alpert syndrome or syndromic craniosynostosis. In an embodiment, the endogenous protein is not human FGFR2 protein. In an embodiment, the epitope tag is not a wild type or a mutant FGFR2 epitope. In an embodiment, the epitope is less than 10 amin acids in length. In an embodiment, the epitope tag (i.e., SHARP-tag) lacks two consecutive Val residues. In an embodiment, mutations in the epitope or the sequence comprising the epitope tag (i.e., SHARP-tag) have not been linked to development of any human disease. In an embodiment, mutations in the epitope or in the sequence comprising the epitope tag (i.e., SHARP-tag) have not been linked to increased risk of the development of a cancer, optionally wherein the cancer is endometrial, breast, melanoma, and/or cholangiocarcinoma. In an embodiment, mutations in the epitope or in the sequence comprising the epitope tag (i.e., SHARP-tag) have not been linked to increased risk of a congenital disease, optionally wherein the disease is lacrimo-auriculo-dento-digital (LADD) syndrome, Alpert syndrome and/or syndromic craniosynostosis. In an embodiment, the sequence comprising the epitope tag (i.e., SHARP-tag) is not found to be mutated in patient who is at an increased risk of a congenital disease and/or a cancer, optionally wherein the disease is Alpert syndrome or syndromic craniosynostosis and cancer is endometrial, breast, melanoma, and/or cholangiocarcinoma. In an embodiment, the sequence comprising the epitope tag (i.e., SHARP-tag) or a variant thereof is not a mutation hot-spot for any known somatic or a germline mutation. In an embodiment, the sequence comprising the epitope tag (i.e., SHARP-tag) or a variant thereof is not derived from the region of an endogenous protein that is a mutation hot-spot for any known somatic or a germline mutation. In an embodiment, the sequence comprising the epitope tag (i.e., SHARP-tag) or a variant thereof is not derived from the region of an endogenous protein that is a mutation hot-spot for any known mutation linked to a human disease, optionally wherein the disease is a congenital disease or a cancer. “As used herein, a ‘mutation hot spot’ refers to one or more amino acid residues or nucleotide positions within a gene or protein that are recurrently mutated across independent biological samples. In an embodiment, epitope tag does not comprise, consist of, or contain any sequence represented by SEQ ID NOs: 450-455 or a variant with at least 80% sequence identity thereto. In an embodiment, the tag sequence exhibits less than 80% sequence identity to any one of SEQ ID NOs: 450-455, as determined by BLAST using default parameters.

In an embodiment, the endogenous protein is expressed on hematopoietic cells. In an embodiment, the endogenous protein is expressed in lymphoid cells. In an embodiment, the endogenous protein is expressed on B-lymphoid cells. In an embodiment, the endogenous protein is expressed on T-lymphoid cells. In an embodiment, the endogenous protein is expressed on plasma cells. In an embodiment, the endogenous protein is expressed preferentially and/or selectively on hematopoietic cells. In an embodiment, the endogenous protein is expressed preferentially and/or selectively on lymphoid cells. In an embodiment, the endogenous protein is expressed preferentially and/or selectively on B lymphoid cells. In an embodiment, the endogenous protein is expressed preferentially and/or selectively on T lymphoid cells. In an embodiment, the endogenous protein is expressed on B lymphocytes and/or plasma cells at a level that is at least 2-fold greater than its expression on T lymphocytes. In an embodiment, the endogenous protein is expressed preferentially and/or selectively on plasma cells at a level that is at least 2-fold greater than its expression on T lymphocytes. In an embodiment, the endogenous protein is not expressed on lung, gastrointestinal, liver, kidney, heart, brain, prostate, ovarian, uterine, nerve, muscle, stomach, esophageal and/or skin cells. In an embodiment, the endogenous protein is expressed on lung, gastrointestinal, liver, kidney, heart, brain, prostate, ovarian, uterine, nerve, muscle, stomach, esophageal and/or skin cells at a level that is less than 20%, (e.g., 19% 15%, 10%, 7%, 5%, 2%, 1% or 0.5% etc.) of its expression on peripheral blood derived B lymphocytes.

In an embodiment, the expression of the endogenous protein is measured at the transcript (i.e., mRNA) level. In an embodiment, the expression of the endogenous protein is measured using quantitative PCR. In an embodiment, the expression of the endogenous protein is measured using next generation sequencing or microarrays. In an embodiment, the expression of the endogenous protein is measured at the protein level. In an embodiment, the expression of the endogenous protein is measured using flow cytometry or immunohistochemistry. In an embodiment, the endogenous protein is selected from the group consisting of CD8a, CD8b, CD4, CD19, CD20, CD22, CD23, CD30, CD33, CD38, CD45, CD56, CD70, CD79a, CD79b, CD123, CD138, CD157, CD179b, CD200R, CD229, ICAM1, CD276, CD324, FcRH5, MPL (or TPO-R), FLT3, Lym1, Lym2, CS1, BCMA, TAC1, GPRC5D, CLL1, CSF2RA, LAMP1, TSHR, TnAg, Her2, EGFR, TROP2, Nectin 4, STEAP1, STEAP2, PSMA, PSCA, NKG2D, CXCR4, TCRβ1, TCRβ2, BST1, IL1RAP, ALK, Folate Receptor 1, TAJ, ROR1, CEA, DLL3, FAP, CSFR1, PD1, PDL1, MUC16, c-MET, EpCAM and TCRgd.

In an embodiment, the endogenous protein is selected from a protein shown in TABLE 1.

TABLE 1
Example of endogenous proteins and antigens

CD19; CD5; CD123; CD22; CD30; CD171; CS-1 (CRACC, SLAMF7, CD319, and 19A24);
CD45, C-type lectin-like molecule-1 (CLL-1 or CLECL1); CD33; epidermal growth factor
receptor variant III (EGFRviii); ganglioside G2 (GD2); TNF receptor family member B cell
maturation (BCMA); Tn antigen ((Tn Ag); prostate-specific membrane antigen (PSMA);
Receptor tyrosine kinase-like orphan receptor 1 (ROR1); Fms Like Tyrosine Kinase 3 (FLT3);
Tumor-associated glycoprotein 72 (TAG72); CD38; CD44v6; a glycosylated CD43 epitope
expressed on acute leukemia or lymphoma but not on hematopoietic progenitors; a
glycosylated CD43 epitope expressed on non-hematopoietic cancers; Carcinoembryonic
antigen (CEA); Epithelial cell adhesion molecule (EPCAM); B7H3 (CD276); KIT (CD117);
Interleukin-13 receptor subunit alpha-2 (IL-13Ra2 or CD213A2); Mesothelin; Interleukin 11
receptor alpha (IL-11Ra); prostate stem cell antigen (PSCA); vascular endothelial growth
factor receptor 2 (VEGFR2); Lewis(Y) antigen; CD24; Platelet-derived growth factor receptor
beta (PDGFR-beta); Stage-specific embryonic antigen-4 (SSEA-4); CD20; Folate receptor
alpha; Receptor tyrosine-protein kinase ERBB2 (Her2/neu); Mucin 1, cell surface associated
(MUC1); epidermal growth factor receptor (EGFR); neural cell adhesion molecule (NCAM);
carbonic anhydrase IX (CA1X); tyrosinase; Fucosyl GM1; sialyl Lewis adhesion molecule
(sLe); ganglioside GM3; transglutaminase 5 (TGS5); high molecular weight-melanoma
associated antigen (HMWMAA); claudin 6 (CLDN6); thyroid stimulating hormone receptor
(TSHR); G protein coupled receptor class C group 5, member D (GPRC5D); chromosome X
open reading frame 61 (CXORF61); CD97; CD179a; anaplastic lymphoma kinase (ALK);
mammary gland differentiation antigen (NY-BR-1); Wilms tumor protein (WT1); Cancer/testis
antigen 1 (NY-ESO-1); Melanoma-associated antigen 1 (MAGE-A1); melanoma antigen
recognized by T cells 1 (MelanA or MARTI); Rat sarcoma (Ras) mutant; human Telomerase
reverse transcriptase (hTERT); human papilloma virus E6 (HPV E6); human papilloma virus
E7 (HPV E7); CD79a; CD79b; CD72; Leukocyte-associated immunoglobulin-like receptor 1
(LAIR1); C-type lectin domain family 12 member A (CLEC12A); EGF-like module-
containing mucin-like hormone receptor-like 2 (EMR2); lymphocyte antigen 75 (LY75);
Glypican-3 (GPC3); Fc receptor-like 5 (FCRL5); immunoglobulin lambda-like polypeptide 1
(IGLL1); Biotin; c-MYC epitope Tag; CD34; LAMP1 TROP2; GFRalpha4; CDH17; CDH6;
CDH19; CD200R; Slea (CA19.9; Sialyl Lewis Antigen) Fucosyl-GM1; PTK7; CDH1-CD324;
DLL3; CD276/B7H3; IL11Ra; IL13Ra2; CD179b-IGLl1; ALK, TCR-gamma-delta; NKG2D;
CD32 (FCGR2A); CSPG4-HMW-MAA; Tim1-/HVCR1; CSF2RA (GM-CSFR-alpha);
TGFbetaR2; VEGFR2/KDR; Lewis Ag; TCR-alpha chain, TCR-beta1 chain; TCR-beta2
chain; TCR-gamma chain; TCR-delta chain; FITC; Luteinizing hormone receptor (LHR);
Follicle stimulating hormone receptor (FSHR); Chorionic Gonadotropin Hormone receptor
(CGHR); CCR4; SLAMF6; SLAMF4; HIV1 envelope glycoprotein; HTLV1-Tax; CMV pp65;
EBV-EBNA3c; influenza A hemagglutinin (HA); GAD; PDL1; Guanylyl cyclase C (GCC);
KSHV-K8.1 protein; KSHV-gH protein; auto antibody to desmoglein 3 (Dsg3); autoantibody
to desmoglein 1 (Dsg1); HLA-A2; HLA-A2:01, HLA-B; HLA-C; HLA-DP; HLA-DM; HLA-
DOA; HLA-DOB; HLA-DQ; HLA-DR; HLA-G; IGE; CD99; Lym1; Lym2; RAS G12V;
Tissue Factor 1 (TF1); AFP; claudin18.2 (CLD18A2 OR CLDN18A.2); STEAP1; STEAP2,
LIV1; NECTIN-4; CRIPTO; GPA33; BST1/CD157; low conductance chloride channel;
TAJ/TNFRSF19, MPL (TPO-R), KIR3DL2, CD32b, CD229, Toso, BAFF-R, OR2H1, p95-
Her2, huTAG2, immunoglobulin kappa light chain, immunoglobulin gamma light chain,
SARS-cov2 spike glycoprotein, SARS-cov2 Receptor binding domain, CSF1R, mutant p53,
p53-R175H mutant, p53-R248Q mutant, NPM1c, mutant NPM1c, PRAME1, Melanoma-
associated antigen 4 (MAGE-A4), gp100, IL23R, MYCN, PD1, PDL1, and Myelin
Oligodendrocyte Glycoprotein (MOG).

In an example embodiment, the epitope tags derived from an endogenous protein has a sequence represented by SEQ ID NO:692-720, 550-579 or variants thereof that differ in length by 1, 2, 3, 4 or 5 amino acids and/or have conservative substitutions of 1, 2, 3, 4 or 5 amino acids. In an embodiment, the invention provides antibodies, antibody fragments, antibody drug conjugate, radio-labelled antibodies and non-immunoglobulin binding domains that bind to SEQ ID NO:692-720, 550-579 or variants thereof that differ in length by 1, 2, 3, 4 or 5 amino acids and/or have conservative substitutions of 1, 2, 3, 4 or 5 amino acids. In an embodiment, the invention provides recombinant polynucleotides, recombinant polypeptides (e.g. SAR, e.g., CAR, SIR, zSIR etc.), cells, vectors, and compositions comprising epitope tags derived from an endogenous protein with sequence represented by SEQ ID NO:692-720, 550-579 or variants thereof that differ in length by 1, 2, 3, 4 or 5 amino acids and/or have conservative substitutions of 1, 2, 3, 4 or 5 amino acids. Also provided are methods for detection, isolation, separation, enrichment, depletion, tracking and control of cells and compositions comprising recombinant proteins (e.g. SAR, e.g., CAR, SIR, zSIR etc.) comprising epitope tags derived from an endogenous protein with sequence represented by SEQ ID NO:692-720, 550-579 or variants thereof that differ in length by 1, 2, 3, 4 or 5 amino acids and/or have conservative substitutions of 1, 2, 3, 4 or 5 amino acids.

In one embodiment, the SHARP-tag does not interfere with one or more biological functions of the recombinant protein to which it is attached. Such recombinant proteins may include, for example, synthetic antigen receptors (SARs), antibodies, antibody fragments, cytokines, chemokines, or combinations thereof. The biological functions preserved in the presence of the pe SHARP-tag may include, but are not limited to, protein expression, proper folding, in vivo half-life, target binding activity, signaling activity, and/or immunogenicity.

The SHARP-tag is engineered to minimize immune recognition by the patient's immune system. It may be derived from human self-proteins but from regions typically not exposed to the immune system. Computational immunogenicity prediction tools can be used to verify that the peptide has low predicted binding affinity to common HLA class II alleles (reducing risk of helper T cell-driven antibody responses against the tag). In some embodiments, the tag sequence has been screened against known allergen or pathogen sequences to ensure it does not mimic any common viral or bacterial epitope that could lead to pre-existing immunity. The small size also contributes to low immunogenicity. By keeping SHARP-tag usage typically as a single copy, the chances for a strong anti-tag immune response are further mitigated. Nonetheless, as a precaution, patients can be monitored for anti-drug antibodies (ADAs) against SHARP-tag or SABR, and if necessary, a switch to an alternate tag variant (one of the many sequences provided) and corresponding antibody variant can be used for subsequent treatment cycles.

In one aspect, the epitope tag is non-immunogenic, hypoimmunogenic, or minimally immunogenic. In certain embodiments, the epitope tag does not induce, or induces only a minimal or reduced immune response when administered to a subject. The immune response may be humoral and/or cellular in nature. Methods for evaluating immunogenicity are well known in the art and include, without limitation, measurement of antibody production using ELISA, Western blotting, or ELISPOT, and assessment of cytokine production. The cytotoxic activity of T cells specific for cells expressing the tagged protein may be measured using cytotoxicity assays.

In various embodiments, the immune response is assessed by detecting antibodies specific to the epitope tag, or to polypeptides, vectors, cells, or compositions comprising the epitope tag. Alternatively, or additionally, the immune response may be measured by assessing the presence or expansion of T cell clones reactive against the epitope tag.

In certain embodiments, epitope tags described herein (e.g., SEQ ID NOs: 1-123, 150-167, 251-440, 631-650, 651-654, 656, 674-676, 686, and 689) elicit no immune response, or a minimal or reduced immune response, when compared to conventional epitope tags such as FLAG (e.g., SEQ ID NO: 1527) or Strep-tag (e.g., SEQ ID NO: 1532). In one aspect, a recombinant protein (e.g., a TAG-SAR) comprising an epitope tag described herein (e.g., SEQ ID NOs: 1-123) elicits no immune response or a reduced immune response compared to the same protein bearing a FLAG tag or a Strep-tag. Similarly, recombinant cells expressing such tagged proteins demonstrate no greater immunogenicity, or reduced immunogenicity, relative to cells expressing proteins with conventional epitope tags. In one aspect, the epitope tag does not induce, or induces only a minimal immune response (e.g., humoral and/or cellular) when administered by any of various routes, including oral, subcutaneous, intradermal, intravenous, or intraperitoneal administration.

In an embodiment, the presence of an epitope tag (e.g., a tagged cassette) does not significantly stimulate an immune response against cells (e.g., T cells) that are engineered to express TAG-SAR. In an embodiment, the cells comprising the TAG-SAR demonstrate in vivo persistence of at least 30%, 40%, 50%, 60%, 70%, 80%, 90%, 95%, or 99% compared to cells expressing the SAR without the tagged cassette. In vivo persistence can be measured using techniques known in the art, such as flow cytometry and/or quantitative polymerase chain reaction (qPCR). In an embodiment, the cells expressing the TAG-SAR with the tagged cassette show a humoral or cellular antibody response that is no greater than 30%, 40%, 50%, 60%, 70%, 80%, 90%, 95%, or 99% greater than the humoral or cellular immune response against cells expressing SAR without an epitope tag or tagged cassette. The humoral and cellular immune responses can be measured using techniques known in the art, such as ELISA, ELISPOT, mass spectrometry, or next-generation sequencing. In another embodiment, the presence of the tag does not significantly interfere with generating a viral vector that incorporates the nucleic acid with the tag. The titer of a viral vector with nucleic acid that incorporates the tag (e.g., TAG-SAR) is at least 30%, 40%, 50%, 60%, 70%, 80%, 90%, 95%, or 99% of a comparable viral vector with nucleic acid (e.g., SAR) that does not have a tagged cassette. The viral titer can be measured using techniques known in the art, such as qPCR or p24 ELISA.

In an embodiment, incorporation of an epitope tag (e.g., a tag cassette) does not substantially impair the expression and/or functional activity of the synthetic antigen receptor (SAR) or the cells engineered to express the SAR. In certain embodiments, a SAR comprising the tag cassette (i.e., TAG-SAR) demonstrates expression levels, when expressed in a suitable host cell, that are at least about 25%, 30%, 40%, 50%, 60%, 70%, 80%, 90%, 95%, or 99% of the expression level observed for an otherwise identical SAR lacking the tag cassette. Expression levels may be determined using any suitable method known in the art, including but not limited to quantitative reverse transcription PCR (qRT-PCR), Protein L staining, or the Topanga assay.

In an embodiment, the functional activity of the TAG-SAR is at least about 30%, 40%, 50%, 60%, 70%, 80%, 90%, 95%, or 99% of the activity observed for an untagged SAR. Activity may be assessed using any suitable assay, including but not limited to the Jurkat NFAT-GFP reporter assay, measurement of cytokine production (e.g., IFNγ, TNFα, IL-2 via ELISA), and/or cytotoxicity assays (e.g., Matador cytotoxicity assay).

In a further embodiment, the TAG-SAR retains binding to its cognate target antigen at a level that is at least about 30%, 40%, 50%, 60%, 70%, 80%, 90%, 95%, or 99% of the antigen-binding activity of an otherwise identical SAR lacking the tag cassette, Binding activity may be measured using methods known in the art, such as the Topanga binding assay. In one aspect, the inclusion of an epitope tag (e.g., a tag cassette) does not substantially elicit an immune response against the engineered cells (e.g., T cells) that express the tag. In certain embodiments, cells comprising the epitope tag or tag cassette exhibit in vivo persistence that is at least about 30%, 40%, 50%, 60%, 70%, 80%, 90%, 95%, or 99% of the persistence observed in otherwise identical cells lacking the tag or tag cassette. In vivo persistence may be assessed using techniques known in the art, including but not limited to flow cytometry or quantitative PCR (qPCR). In another aspect, the presence of the epitope tag or tag cassette does not induce a humoral or cellular immune response that exceeds the response elicited by untagged cells by more than about 30%, 40%, 50%, 60%, 70%, 80%, 90%, 95%, or 99%. The humoral and/or cellular immune response may be evaluated using methods known in the art.

In one aspect the presence of the SHARP-tag (e.g., epitope tag or the tagged cassette) does not significantly interfere with the expression and/or activity of the cells (e.g., T cells) engineered to express the tag. In one aspect the cells expressing the SHARP-tag, or the tagged cassette show activity that is at least 30%, 40%, 50%, 60%, 70%, 80%, 90%, 95% or 99% of the activity of the cells without the tagged cassette. Activity of the cells can be measured using techniques known in the art, such as Jurkat NFAT-GFP assay, cytokine production (e.g., ELISA for IFNγ, TNFα, IL2 etc) and/or measurement of cytotoxicity (e.g., Matador cytotoxicity assay).

In one aspect, the SHARP-tag (or epitope tag) is encoded by a recombinant polynucleotide. In one aspect, the epitope tag is part of a non-natural (i.e., synthetic or recombinant) protein or non-natural polypeptide. In one aspect, the epitope tag is part of a recombinant polypeptide. In one aspect, the epitope tag is part of a recombinant polypeptide that encodes for a receptor, cytokine, chemokine, ligand, antibody, cytoskeleton protein, secreted protein, membrane glycoprotein or an adhesion molecule. In one aspect, the receptor is a synthetic receptor. In one aspect, the epitope tag is part of a chimeric antigen receptor (CAR), a next generation chimeric antigen receptor (CAR), a synthetic antigen receptor (e.g., SIR, zSIR, z16SAR, uTCR-SAR, Ab-TCR, HIT, STAR, TFP, TRI-TAC, KIR-CAR or a recombinant TCR etc.). In one aspect, the epitope tag is part of a recombinant polypeptide or a recombinant protein that is expressed on a recombinant cell, i.e., a cell that is genetically engineered. In one aspect, the epitope tag is part of a recombinant polypeptide or a recombinant protein that is secreted from a recombinant cell, i.e., a cell that is genetically engineered. In an embodiment, the recombinant cell expresses a synthetic receptor (e.g., a CAR, a next generation CAR, a SAR etc.). In an embodiment, the epitope tag is a part of a recombinant protein that is co-expressed with a synthetic receptor. In an embodiment, the epitope tag is a part of a recombinant protein that is expressed on a vector, e.g., a viral vector, viral like particle, a lipid nanoparticle etc. In an embodiment, the epitope tag is a part of a recombinant protein that is expressed on packaging cells used to produce a viral vector.

In one aspect, the epitope tag comprises, contains, or consists of a linear epitope (also referred to as a sequential epitope). A linear epitope is a continuous sequence of amino acids (i.e., primary structure) that is recognized by an antibody based on its amino acid sequence, rather than its three-dimensional conformation. In one aspect, the epitope tag is not a conformational epitope, i.e., it does not rely on a specific tertiary structure for antibody recognition. In certain embodiments, the epitope tag lacks disulfide bonds, including intramolecular and intermolecular disulfide bonds, and may further lack cysteine residues. In one aspect, the epitope tag is not a mimotope.

In certain embodiments, the linear epitope is derived from the extracellular domain of an endogenous human protein or a variant thereof. In a specific embodiment, the source protein is preferentially expressed on hematopoietic cells, B lymphocytes, or plasma cells.

In an embodiment, the SAR is a single chain SAR, a double chain SAR or a multichain SAR. In an embodiment, the SAR is selected from the group consisting of CAR, SIR, HIT, STAR, Ab-TCR, zSIR, z16SAR, uTCR-SAR and/or recombinant TCR. In one embodiment, the disclosure provides a recombinant polynucleotide, the encoded recombinant polypeptide, and cells, vector and composition comprising and/or encoding such as recombinant polypeptide, wherein the recombinant polynucleotide encodes an epitope tag (or tag) comprising one or more features selected from the group consisting of the following: a) the tag is less than 50, 30, 29, 28, 27, 26, 25, 24, 23, 22, 21, 20, 19, 18, 17, 16, 15, 14, 13, 12, 11, 10, 9, 8, 7, 6 or 5 amino acids in length; b) the tag is non-immunogenic or hypoimmunogenic or minimally immunogenic, c) tag is hydrophilic and/or polar; optionally wherein the tag is non-hydrophobic; d) tag is recognized by an drug that is approved or will be approved by a regulatory agency, optionally wherein the drug is antibody or antibody fragment or antibody drug conjugate or radiolabeled antibody; e) the tag does not interfere with the binding of the SAR to its antigen; f) the treatment of the tag-expressing SAR cells results in their killing; g) treatment of the tag expressing SAR cells results in killing of the bystander cells; and/or h) the tag is derived from an endogenous protein. In an embodiment, the recombinant polynucleotide encodes a SAR. In an embodiment, the SAR is a single chain SAR (e.g., a CAR) or double chain SAR. In an embodiment, the SAR is selected from the group consisting of SIR, HIT, STAR, Ab-TCR, TFP, zSIR, z16SAR, CD16-SAR, uTCR-CAR, Link-SAR, zSAR, and/or recombinant TCR.

In some embodiments, the epitope tag comprises a peptide selected from SEQ ID NOs: 692-720, 550-579 or a variant thereof. Variants may include polypeptides having one, two, or three amino acid substitutions, deletions, or additions relative to SEQ ID NOs: 692-720, 550-579, provided the variant retains antibody recognition and/or desired properties. The disclosure further provides recombinant polynucleotides, polypeptides, expression vectors, host cells, and pharmaceutical compositions comprising one or more of the epitope tags represented by SEQ ID NOs: 692-720, 550-579 or variants thereof.

In certain embodiments, the invention provides synthetic antigen receptors (e.g., SARs or CARs) in which one or more epitopes, such as those represented by SEQ ID NOs: 674-676, are replaced with the epitope tags represented by SEQ ID NOs: 692-720, 550-579 or variants thereof. In an embodiment, the epitope tags described herein (e.g., SEQ ID NOs: 692-720, 550-579 or variants) are minimally immunogenic or hypoimmunogenic and do not substantially interfere with the expression and/or activity of the recombinant polypeptide to which they are fused. In a further embodiment, recombinant polynucleotides, polypeptides, vectors, cells, and compositions comprising the disclosed epitope tags are minimally immunogenic or hypoimmunogenic when administered to a human subject.

In an embodiment, the invention provides a SAR (e.g., CAR) comprising an epitope tag with the sequence represented by SEQ ID NO:721 or variants thereof. In an embodiment, the invention provides a cell expressing a SAR (e.g., CAR) comprising an epitope tag with the sequence represented by SEQ ID NO:721 or variants thereof. In an embodiment, the SAR is a double chain SAR. In an embodiment, the SAR is selected from the group consisting of SIR, HIT, STAR, Ab-TCR, zSIR, z16SAR, uTCR-SAR and/or recombinant TCR. In an embodiment, the SAR comprising the epitope tag with the sequence represented by SEQ ID NO:721 or variants thereof can be detected in vitro and/or in vivo using luminescence by the addition of a furimazine and LgBiT.

In one embodiment, the epitope tag comprises natural or naturally occurring amino acid residues. In another embodiment, the epitope tag comprises non-natural, non-naturally occurring, or synthetic amino acid residues. Another embodiment includes a mixture of natural and non-natural amino acid residues.

In one aspect, the present invention provides novel epitope tags, designated SHARP-tags. and functional variants thereof, as well as polynucleotides, polypeptides, expression vectors, host cells, and pharmaceutical compositions comprising such epitope tags. In certain embodiments, the epitope tag comprises a sequence selected from SEQ ID NOs: 1-123, 150-167, 251-440, 550-579, 631-650, 651-654, 656, 674-676, 686, 689, and 692-720, or a functional variant thereof. In other embodiments, the SHARP-tags consists of a sequence selected from SEQ ID NOs: 1-123, 150-167, 251-440, 550-579, 631-650, 651-654, 656, 674-676, 686, 689, and 692-720, or a functional variant thereof.

As used herein, the term “functional variant” refers to a polypeptide sequence that differs from a reference sequence (e.g., SEQ ID NO: 1-123, 150-167, 251-440, 550-579, 631-650, 651-654, 656, 674-676, 686, 689, and 692-720, or a functional variant thereof) by one or more amino acid substitutions, deletions, or insertions, but retains at least one functional property of the reference epitope tag. Such functional properties may include, but are not limited to, (i) recognition by an antibody that specifically binds the reference epitope, (ii) ability to be detected or enriched using an antibody or ligand, (iii) absence or minimization of interference with the biological function of the protein to which the tag is fused, and/or (iv) low or minimal immunogenicity. In certain embodiments, functional variants exhibit at least 70%, 80%, 85%, 90%, 95%, 98%, or 99% sequence identity to a reference sequence, and retain binding reactivity to an antibody that specifically recognizes the corresponding unmodified epitope tag. In example embodiments, the epitope tag comprises or consists of the sequence RSEDRY (SEQ ID NO: 1), RSEDRYR (SEQ ID NO: 2), RSEDRYRN (SEQ ID NO: 3), RSEDRYRNP (SEQ ID NO: 4), RSEDRYRNPK (SEQ ID NO: 5), RSEDRYRNPKG (SEQ ID NO: 6), RSEDRYRNPKGS (SEQ ID NO: 7), RSEDRYRNPKGS (SEQ ID NO: 8), ARSEDRY (SEQ ID: 46), ARSEDRYR (SEQ ID NO:47), ARSEDRYRNPK (SEQ ID NO: 5073), ARPAKSEDLYPNPK (SEQ ID NO: 689), AKSEDLY (SEQ ID NO: 645) or functional variants (including deletion, point and length mutants) thereof with 1-5 amino acid substitutions. In an embodiment, the epitope tag (SHARP-tag) comprises additional 1 to 50 amino acids (e.g., 1, 5, 10, 25 or 50) that are present at the N-terminal and/or C-terminal of any of the sequences described in this disclosure.

The invention covers SHARP-tag variants, which may be created for various reasons (e.g., to fine-tune immunogenicity, adjust affinity for SABR variants, or to create a panel of tags distinguishable by specific antibodies). In an embodiment, the variant may be a conservative substitution, for example, replacing one basic residue (lysine) with another (arginine) or one acidic residue (glutamic acid) with aspartic acid, etc., in a way that preserves overall charge pattern and hydrophilicity. Such minor changes often do not abolish antibody binding. In another embodiment, the variant may be a length variant, for example, adding one or two residues at either end (perhaps to create a unique protease site or to add a spacing residue). An N- or C-terminal glycine or serine could be appended without fundamentally changing the epitope. In another aspect, the variant may involve order shuffling or structurally similar motifs. In some cases, the core epitope might accommodate a permutation of residues or insertion of a small spacer. Any peptide sequence that retains binding by a SABR (or functional equivalent thereof) is considered a functional SHARP-tag. This can be empirically tested by ELISA or surface plasmon resonance with SABR.

As used herein the term “binds” or “recognized by” means that the SHARP-tag peptide has a T_{1/2} of at least about 2 min for dissociation from a SABR represented (e.g., Polatuzumab vedotin, a generic variant of Polatuzumab vedotin, huMA79b, 2F2, or SN8, H2Mab-250 or antibodies with SEQ ID NO: 1182-1189). In an embodiment, a SHARP-tag peptide described herein has a Kd of about 30 nM or less for the binding to its SABR (e.g., Polatuzumab vedotin, a generic variant of Polatuzumab vedotin, huMA79b, or SN8).

An antibody or antibody molecule/fragment is said to “specifically” bind to an antigen when it recognizes its target antigen within a complex mixture of proteins and/or macromolecules. Typically, the antibody is capable of specifically interacting with and/or binding to its target but does not essentially bind to another epitope or antigen. Antibodies are said to “bind to the same epitope” if the antibodies cross-compete so that only one antibody can bind to the epitope at a given point of time, i.e. one antibody prevents the binding or modulating effect of the other.

Typically, binding that is considered specific may also have a high affinity, e.g. when the binding affinity is higher than 10⁻⁶ M (in terms of K_{d}). In particular. the binding affinity may be about 10⁻⁸ to 10⁻¹¹ M (Kt), or of about 10⁻⁹ to 10⁻¹¹ M or even higher. Thus, antibody molecules with an affinity in the picomolar range (with a K_{d} of 9.9×10⁻¹⁰ M to 10⁻¹² M) are also encompassed in the present invention. If necessary, nonspecific binding of a binding site can be reduced without substantially affecting specific binding by varying the binding conditions.

An antibody according to the invention may be an isolated antibody molecule. The term “isolated antibody molecule” as used herein refers to an antibody molecule that has been identified and separated and/or recovered from a component of its natural environment. Contaminant components of its natural environment are matter that would interfere with uses for the antibody, and may include enzymes, hormones, and other proteinaceous or non-proteinaceous solutes. In some embodiments the antibody molecule is purified to greater than 95% by weight of antibody as determined by the Lowry method, such as more than 99% by weight. An isolated antibody molecule may in some embodiments be present within foreign host cells with one or more component(s) of the antibody's natural environment not being present. Typically, an isolated antibody is prepared by at least one purification step.

The disclosure presents an extensive collection of possible SHARP-tag sequences and their analogs. These may include single amino acid mutants at each position, alanine scans, or other libraries from which SABR cross-reactive sequences were identified. The flanking N- and C-terminal residues) can be altered or omitted as long as the SABR can still bind.

In an example embodiments, the SHARP-tag comprises or consists of the sequence RSEDRY (SEQ ID NO: 1) or RSEDRYR (SEQ ID NO: 2) or a variant thereof wherein the amino acid residue R (Arg) at position 1 is mutated to E, S, N, D, F or H; and/or the amino acid residue S (Ser) at position 2 is mutated to T, A, L, M, F, G, H, I, L, or T; and/or the amino acid residue D (Asp) at position 4 is mutated to S, N, G, A, T, K, L, Y, W, R, or V; and/or the amino acid residue R (Arg) at position 5 is mutated to H, P, L, T or C; and/or the amino acid residue Y (Tyr) at position 6 is mutated to M, F, H, D, A, G, I or V; and/or the amino acid residue R (Arg) at position 7 in case of SEQ ID NO: 2 is mutated to K, E, D, G, H, L, M, Q, V, W, S, F, I, Y, P, N, T or A. In an example embodiment, the SHARP-tag comprises or consists of the sequence represented by SEQ ID NO: 2 or a variant thereof wherein the residue R (Arg) at position 7 is any naturally occurring amino acid. In an embodiment, the residue R (Arg) at position 7 is not Cys (C). In an embodiment, the SHARP-tag comprises additional 1 to 50 amino acids that are present at the N-terminal and/or C-terminal of the above sequences. In an embodiment the SHARP-tag lacks a Cys (C). In an embodiment the SHARP-tag lacks a disulfide bond. In an embodiment, the SHARP-tag lacks an Asn-X-Ser/Thr motif that would create an N-linked glycosylation site. In an embodiment, the epitope is recognized by Polatuzumab vedotin, a generic variant of Polatuzumab vedotin, 2F2, SN8 antibody, or a functional variant thereof. In an embodiment, the SHARP-tag is recognized by an antibody comprising the light chain CDR1-3 represented by SEQ ID NO: 6653-6673, 7022-7042, 7391-7411, respectively, and heavy chain CDR1-3 represented by SEQ ID NO: 6778-6798, 7147-7167, and 7516-7536, respectively. In an embodiment, the antibody binds with at least 5-fold lower affinity to a mutant of SEQ ID NO: 1-45, 93-94, 97-98, 102-103, 108,110, 112-116, 118-123, 282-320, 354-377, 410-432 in which the residue Glu (E) at position 3 is mutated to any other amino acid. In an embodiment, the SHARP-tag represents any variant of SEQ ID NO: 1-45, 93-94, 97-98, 102-103, 108,110, 112-116, 118-123, 282-320, 354-377, 410-432 which binds with at least 5-fold higher affinity to the Polatuzumab vedotin, a generic variant of Polatuzumab vedotin, 2F2, or SN8 antibody as compared to a mutant of the above sequences in which the residue Glu (E) at position 3 is mutated to any other amino acid. In an embodiment, a SHARP-tag peptide (e.g., SEQ ID NO: 1-123, 150-167) may have a T_{1/2} of at least about 2 min for dissociation from a SABR represented by Polatuzumab vedotin, a generic variant of Polatuzumab vedotin, huMA79b, 2F2, or SN8, or antibodies with SEQ ID NO: 1182-1189. In an embodiment, a SHARP-tag peptide (e.g., SEQ ID NO: 1-123, 150-167) may have a Kd of about 30 nM or less for the binding to Polatuzumab vedotin, a generic variant of Polatuzumab vedotin, huMA79b, 2F2, or SN8, or antibodies with SEQ ID NO: 1182-1189.

In an embodiment, the epitope tag (SHARP-tag) comprises or consists of the sequence X₁X₂EX₃X₄X₅X₆ wherein X₁ is selected form R, E, or H; X₂ is selected form S, T or A; X₃ is selected form D, S, N, G, A, T, K, L; X₄ is selected form R or H; X₅ is selected form Y or M; and X₆ is any amino acid or no amino acid. In an embodiment, the SHARP-tag comprises or consists of the sequence X₁X₂EX₃X₄X₅X₆ wherein X₁ is selected form R, E, or H; X₂ is selected form S, T or A; X₃ is selected form D, S, N, G, A, T, K, Q, L; X₄ is R or H; X₅ is selected form Y or M; and X₅ is any naturally occurring amino acid. In an embodiment, the SHARP-tag comprises or consists of the sequence X₁X₂EX₃X₄X₅X₆ wherein X₁ is selected form R, E, or H; X₂ is selected form S, T or A; X₃ is selected form D, S, N, G, A, T, K, L; X₄ is selected form R or H; X₅ is selected form Y or M; and X₆ is selected form R, K, E, D, G, H, L, M, Q, V, W, S, F, I, Y, P, N, T or A. In an embodiment, X₆ is any amino acid except Cys (C). In an embodiment, the SHARP-tag comprises additional 1 to 50 amino acids that are present at the N-terminal and/or C-terminal of the above sequences. In an embodiment, the SHARP-tag is recognized by Polatuzumab vedotin, a generic variant of Polatuzumab vedotin, SN8 antibody, or a functional variant thereof. In an embodiment, the SHARP-tag is recognized by an antibody comprising the light chain CDR1, CDR2 and CDR3 represented by SEQ ID NO: 6653-6661; 7022-7030 and 7391-7398, respectively, and heavy chain CDR1, CDR2 and CDR3 represented by SEQ ID NO: 6778-6786, 7147-7155, and 7516-7524, respectively. In an embodiment, a SHARP-tag peptide described herein may have a T_{1/2} of at least about 2 min for dissociation from a SABR represented by Polatuzumab vedotin, a generic variant of Polatuzumab vedotin, huMA79b, or SN8, or antibodies with SEQ ID NO: 1182-1184. In an embodiment, a SHARP-tag peptide described herein may have a Kd of about 30 nM or less for the binding to Polatuzumab vedotin, a generic variant of Polatuzumab vedotin, huMA79b, or SN8, or antibodies with SEQ ID NO: 1182-1184.

In an embodiment, the SHARP-tag comprises or consists of the sequence X₁X₂EX₃X₄X₅X₆ wherein X₁ is selected form R, S, N, D, F or H; X₂ is selected form S, T, I, L, M, V, F, H, G, or A; X₃ is selected form D, G, A, Y, W, R, or V; X₄ is selected form R, H, P, L, T or C; X₅ is selected form Y, M, F, H, D, A, G, I or V; and X₆ is any amino acid or no amino acid. In an embodiment, the SHARP-tag comprises or consists of the sequence X₁X₂EX₃X₄X₅X₆ wherein X₁ is selected form R, S, N, D, F or H; X₂ is selected form S, T, I, L, M, V, F, H, G, or A; X₃ is selected form D, G, A, Y, W, R, or V; X₄ is selected form R, H, P, L, T or C; X₅ is selected form Y, M, F, H, D, A, G, I or V; and X₆ is any naturally occurring amino acid. In an embodiment, X₆ is any amino acid except Cys (C). In an embodiment, the SHARP-tag comprises or consists of the sequence X₁X₂EX₃X₄X₅X₆ wherein X₁ is selected form R, S, N, D, F or H; X₂ is selected form S, T, I, L, M, V, F, H, G, or A; X₃ is selected form D, G, A, Y, W, R, or V; X₄ is selected form R, H, P, L, T or C; X₅ is selected form Y, M, F, H, D, A, G, I or V; and X₆ is selected form R, K, E, D, G, H, L, M, Q, V, W, S, F, I, Y, P, N, T or A. In an embodiment the SHARP-tag lacks a Cys (C). In an embodiment the SHARP-tag lacks a disulfide bond. In an embodiment, the SHARP-tag lacks an Asn-X-Ser/Thr motif that would create an N-linked glycosylation site. In an embodiment, the SHARP-tag comprises additional 1 to 50 amino acids that are present at the N-terminal and/or C-terminal of the above sequences. In an embodiment, the epitope is recognized by 2F2 antibody or a functional variant thereof. In an embodiment, a SHARP-tag peptide described herein may have a T_{1/2} of at least about 2 min for dissociation from a SABR represented by 2F2 or an antibody with SEQ ID NO: 1189. In an embodiment, a SHARP-tag peptide described herein may have a Kd of about 30 nM or less for the binding to Polatuzumab vedotin, a generic variant of Polatuzumab vedotin, huMA79b, or SN8, or antibodies with SEQ ID NO: 1182-1189.

In an embodiment, the SHARP-tag comprises or consists of the sequence X₁X₂EX₃X₄X₅X₆ wherein X₁ is selected from R, E, S, N, D, F or H; X₂ is S, T, I, L, M, V, F, H, G, or A; X₃ is D, S, N, G, A, T, K, Q, L, Y, W, R, or V; X₄ is R, H, P, L, T or C; X₅ is Y, M, F, H, D, A, G, I or V; and X₆ is any amino acid or no amino acid. In an embodiment, the SHARP-tag comprises or consists of the sequence X₁X₂EX₃X₄X₅X₆ wherein X₁ is selected from R, E, S, N, D, F or H; X₂ is selected from S, T, I, L, M, V, F, H, G, or A; X₃ is selected from D, S, N, G, A, T, K, Q, L, Y, W, R, or V; X₄ is selected from R, H, P, L, T or C; X₅ is selected from Y, M, F, H, D, A, G, I or V; and X₆ is any naturally occurring amino acid. In an embodiment, the SHARP-tag comprises or consists of the sequence X₁X₂EX₃X₄X₅X₆ wherein X₁ is selected from R, E, S, N, D, F or H; X₂ is selected from S, T, I, L, M, V, F, H, G, or A; X₃ is selected from D, S, N, G, A, T, K, Q, L, Y, W, R, or V; X₄ is R, H, P, L, T or C; X₅ is selected from Y, M, F, H, D, A, G, I or V; and X₆ is selected from R, K, E, D, G, H, L, M, Q, V, W, S, F, I, Y, P, N, T or A. In an embodiment, X₆ is any amino acid except Cys (C). In an embodiment the SHARP-tag lacks a Cys (C). In an embodiment the SHARP-tag lacks a disulfide bond. In an embodiment, the SHARP-tag lacks an Asn-X-Ser/Thr motif that would create an N-linked glycosylation site. In an embodiment, the SHARP-tag comprises additional 1 to 50 amino acids that are present at the N-terminal and/or C-terminal of the above sequences. In an embodiment, the epitope is recognized by Polatuzumab vedotin, a generic variant of Polatuzumab vedotin, 2F2, SN8 antibody, or a functional variant thereof. In an embodiment, a SHARP-tag peptide described herein may have a T_{1/2} of at least about 2 min for dissociation from a SABR represented by Polatuzumab vedotin, a generic variant of Polatuzumab vedotin, huMA79b, 2F2, or SN8, or antibodies with SEQ ID NO: 1182-1189. In an embodiment, a SHARP-tag peptide described herein may have a Kd of about 30 nM or less for the binding to Polatuzumab vedotin, a generic variant of Polatuzumab vedotin, huMA79b, 2F2, or SN8, or antibodies with SEQ ID NO: 1182-1189.

In an embodiment, the SHARP-tag comprises or consists of the sequence X₁X₂EX₃X₄X₅X₆ wherein X₁ is selected from K or R; X₂ is selected from S, T, I, L, M, V, F, H, G, or A; X₃ is selected from D, S, N, G, A, T, K, Q, L, Y, W, R, or V; X₄ is selected from L, R, H, P, T or C; X₅ is selected from Y, M, F, H, D, A, G, I or V; and X₆ is any amino acid or no amino acid. In an embodiment, X₆ is any amino acid except Cys (C). In an embodiment, the SHARP-tag comprises or consists of the sequence X₁EX₂ X₃X₄X₅X₆ wherein X₁ is selected from S, T, I, L, M, V, F, H, G, or A; X₂ is selected from D, S, N, G, A, T, K, Q, L, Y, W, R, or V; X₃ is selected from L, R, H, P, T or C; X₄ is selected from Y, M, F, H, D, A, G, I or V; X₅ is P; and X₆ is any amino acid. In an embodiment, the SHARP-tag comprises or consists of the sequence X₁X₂X₃EX₄X₅X₆ wherein X₁ is selected from A, G or S; X₂ is selected from K or R; X₃ is selected from S, T, I, L, M, V, F, H, G, or A; X₄ is selected from D, S, N, G, A, T, K, Q, L, Y, W, R, or V; X₅ is selected from L, R, H, P, T or C; and X₆ is selected from Y, M, F, H, D, A, G, I or V. In an embodiment, X₆ is any amino acid except Cys (C). In an embodiment the SHARP-tag lacks a Cys (C). In an embodiment the SHARP-tag lacks a disulfide bond. In an embodiment, the SHARP-tag lacks an Asn-X-Ser/Thr motif that would create an N-linked glycosylation site. In an embodiment, the SHARP-tag comprises additional 1 to 50 amino acids that are present at the N-terminal and/or C-terminal of the above sequences. In an embodiment, the epitope is recognized by 10D10 antibody or functional variants. In an embodiment, the epitope is recognized by an antibody or antibody fragment comprising the vL region represented by SEQ ID NO: 797 and vH region represented by SEQ ID NO:922 or functional variants comprising up to 10 substitutions in the framework region.

In an example embodiment, the SHARP-tag comprises or consists of the sequence represented by SEQ ID NO: 557-566 or a variant thereof with substation, deletion or addition of 1, 2, 3, 4 or 5 amino acids. In an embodiment, the epitope is recognized by an antibody or antibody fragment comprising the vL region represented by SEQ ID NO: 846-847 and vH region represented by SEQ ID NO: 966-970 or functional variants comprising up to 10 substitutions in the framework region. In an embodiment, the SHARP-tag is recognized by an antibody or its variants (H2Mab-250) comprising the light chain CDR1, CDR2 and CDR3 represented by SEQ ID NO: 6725-6; 7094-5 and 7463-7464, respectively, and heavy chain CDR1, CDR2 and CDR3 represented by SEQ ID NO: 6845-8, 7214-8, and 7583-7587, respectively. In an embodiment, the SHARP-tag comprises a variant of SEQ ID NO: 557-566 which binds with at least 5-fold greater affinity to the antibody, or the antibody fragment as compared to a mutant in which the amino acid residue Trp (W) is replaced by Ala (A). In an embodiment, the epitope is recognized by an antibody or antibody fragment described in U.S. Ser. No. 11/981,747B1 and/or WO2022114163 or a variant with up to 10 substitutions in the framework region. In an embodiment, the antibody is H2Mab-250. In an embodiment the SHARP-tag lacks a Cys (C). In an embodiment the SHARP-tag lacks a disulfide bond. In an embodiment, the SHARP-tag lacks an Asn-X-Ser/Thr motif that would create an N-linked glycosylation site. In an embodiment, a SHARP-tag peptide described herein may have a T_{1/2} of at least about 2 min for dissociation from a SABR represented by H2Mab-250, or an antibody with vL region represented by SEQ ID NO: 846-847 and vH region represented by SEQ ID NO: 966-970. In an embodiment, a SHARP-tag peptide described herein may have a Kd of about 30 nM or less for the binding to Polatuzumab vedotin, a generic variant of Polatuzumab vedotin, huMA79b, 2F2, or SN8, or antibodies with SEQ ID NO: 1182-1189.

The sequences described herein (e.g., SEQ ID NO: SEQ ID NO: 1-123, 150-167, 251-440, 550-579, 631-650, 651-654, 656, 674-676, 686, 689, and 692-720) define the core structure of the SHARP-tag peptide and may further comprise up to two additional amino acids at the N terminus and up to two additional amino acids at the C terminus. Such additional amino acids at the ends of the core structure of the peptide usually do not necessarily influence the secondary structure of the of the peptide or specific binding of the peptide to an antibody specific for the peptide, but may serve as linker structures in the fusion protein. Accordingly, type and number of the additional amino acids may depend on the location of the peptide in the fusion protein and may vary depending on whether the peptide is located N-terminal or C-terminal or somewhere in between of the polypeptide.

The invention also provides that naturally occurring protein or an endogenous protein or its isoform can serve as marker/suicide protein on the cell surface. In an embodiment, CD79b (SEQ ID NO (DNA): 3110 and SEQ ID NO (PRT): 660) and CD79b isoform-2 (SEQ ID NO (DNA): 3106 and SEQ ID NO (PRT):656) and variants thereof with up to 30 amino acid substitutions can serve as marker/suicide proteins. The SHARP-tag is present in these naturally occurring proteins and they can be recognized by SABR (e.g., SN8, huMA79b, 2F2, Polatuzumab etc.) described herein. In an embodiment, the extracellular domain of CD79b and CD79b-isoform-2 (SEQ ID NO: 659 and 656) or their functional variants serves as SHARP-tags. In an embodiment, the extracellular domain of CD79b or a functional variant thereof is fused to another protein (e.g., a cytokine) to serve as a marker/suicide protein or a multipurpose switch. An example embodiment is provided in SEQ ID NO: 666. The CD79b protein, CD79b isoform, and CD79b-fusion proteins can be expressed in recombinant cells using either alone or co-expressed with proteins of interest (e.g., SAR) using from a single vector or separate vectors. In an embodiment, the CD79b protein, CD79b isoform, and CD79b-fusion proteins and the proteins of interest are expressed from a single polynucleotide or separate polynucleotides. In an embodiment, they are expressed from a single polypeptide or separate polypeptides. In an embodiment, the CD79b protein, CD79b isoform, and CD79b-fusion proteins serve as an accessory module and/or therapeutic control that is used to isolate, enrich, deplete, track or control the expression and/or activity of the recombinant cell. Examples of constructs encoding a SAR along with CD79b protein, CD79b isoform, and CD79b-fusion proteins are presented in SEQ ID NO:4797, 4911 and 4988.

In one aspect, the invention provides an epitope tag that is 5, 6, 7, or 8 amino acids in length. In another aspect, the invention provides an epitope tag that is at least 6 amino acids in length and, in certain embodiments, up to 16 amino acids in length. In various embodiments, the epitope tag is at least 5, 6, 7, 8, 9, 10, 11, 12, 13, or 14 amino acids in length, and at most 16, 17, 18, 19, 20, 21, 25, or 30 amino acids in length. The SHARP-tag may comprise a linear sequence of amino acids that is accessible for antibody recognition and may be designed to minimize interference with the function, expression, or stability of the fusion protein to which it is attached. In an embodiment the tag is recognized by an antibody that is approved by a regulatory agency (e.g., FDA) for in vivo administration. In an embodiment the tag is recognized by a drug (e.g., an antibody) that is in pre-clinical or clinical development, optionally for in vivo administration to a subject. Optionally, the subject is a human subject. In one aspect, the invention provides a tag that is recognized by an antibody drug conjugate. In an embodiment, the antibody drug conjugate is Polatuzumab or Polatuzumab vedotin (CAS Registry Number 1313206-42-6). In an embodiment, Polatuzumab vedotin has an International Nonproprietary Names number (INN) of 9714 and PubChem SID 252166614. In an embodiment, the antibody drug conjugate has the formula C₆₆₇₀H₁₀₃₁₇N₁₇₄₅O₂₀₈₇S₄₀. In an embodiment, the variant is recognized by Polatuzumab or Polatuzumab vedotin, monoclonal antibody SN8 (Thermofisher Scientific Catalog #Catalog #604-490), CD79b-2F2, huMA79b, Polatuzumab or Polatuzumab vedotin or a variant thereof.

In an embodiment, the drug is administered at a dose of between 0.1 mg/kg to 10 mg/kg. In an embodiment, the drug (e.g., antibody, antibody drug conjugate) is administered at a dose of 1.8 mg/kg intravenously. In an embodiment, the drug (e.g., antibody, antibody drug conjugate) is administered at a dose of between 0.1 mg/kg to 10 mg/kg intravenously. The drug can be administered via intravenous, subcutaneous, intradermal, intraperitoneal, intrathecal, intrapleural, oral, nasal, or intraventricular routes. In another embodiment, the drug is administered using an auto-injector. In another embodiment, the subject receives a single dose of the drug. In another embodiment, the subject receives multiple doses of the drug. The drug is administered at a dose ranging from 0.1 to 10 mg/kg. In another embodiment, the tag is non-immunogenic. In another embodiment, the tag is non-immunogenic to a human subject. The tag comprises a sequence of 5, 6, 7, 8, 9, 10, 11, 12, 13, 14, 15, or 16 amino acid residues. The tag may comprise a sequence identical to one present in an endogenous protein. Another embodiment includes a tag with a sequence that has 80-99% identity to a sequence in an endogenous protein (e.g., 80%, 85%, 90%, 92%, 95%, 96%, 97%, 98%, 99%, or 99.5%). In one embodiment, the tag comprises a sequence that is 80-99% identical to a sequence present in an endogenous protein (e.g., 80%, 85%, 90%, 92%, 95%, 96%, 97%, 98%, 99%, or 99.5%). In one embodiment, the endogenous protein is a human protein. The tag may be derived from a sequence located at or near the N-terminus of an endogenous protein. In another embodiment, the tag is derived from a sequence located within the N- or C-terminal 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues of an endogenous protein. In another embodiment, the endogenous protein is a transmembrane protein. In one aspect, the epitope (or tag) comprises the sequence RSEDRY (SEQ ID NO: 1) or a variant thereof and has additional 1 or more amino acid residues at the N and/or C-termini. In one aspect, the epitope comprises or consist of the sequence RSEDRYR (SEQ ID NO: 2) or a variant thereof and has additional 1 or more amino acid residues at the N and/or C-termini. In one aspect, the epitope comprises or consists of the sequence KSEDLY (SEQ ID NO: 650) or a variant thereof and has additional 1 or more amino acid residues at the N and/or C-termini. In an embodiment, the SHARP-tag comprises or consists of the sequence RSEX₁X₂Y (SEQ ID NO: 93) and has additional 1 more amino acid residues at the N and/or C-termini; wherein X₁ is D, S, N, G, A, T, K, L and X₂ is R or H. In an embodiment, the SHARP-tag comprises the sequence with SEQ ID NO:1-123, 251-440, 631-650, 674-676, 686 or 689 and has additional 1 more amino acid residues at the N and/or C-termini.

In one embodiment, the invention provides a recombinant construct comprising a polynucleotide encoding a polypeptide that includes one or more SHARP-tags described herein. The recombinant construct may be contained within an expression vector, introduced into a host cell, or formulated in a pharmaceutical composition. In certain embodiments, the polypeptide comprises a single SHARP-tag; in other embodiments, the polypeptide comprises multiple SHARP-tags, such as two or more copies of a SHARP-tag.

In one aspect, the multiple SHARP-tags are identical in amino acid sequence. In another aspect, the multiple tags are non-identical, each comprising a distinct amino acid sequence. In some embodiments, the recombinant construct further comprises one or more additional tags or functional domains known in the art, including, but not limited to: FLAG tag (e.g., SEQ ID NO: 1527); Strep-tag (e.g., SEQ ID NO: 1532); Poly histidine tag (His-tag); Myc tag; Rituximab tag; RQR8; CD34 tag; Masking peptide (e.g., SEQ ID NO: 1203); Cleavable linkers such as MMP-cleavable (SEQ ID NO: 1204), PSA-cleavable (SEQ ID NO: 1205), or TEV-cleavable (SEQ ID NO: 1206); or Masking domain (e.g., SEQ ID NO: 1202). Such tags may be used individually or in combination, depending on the desired function (e.g., detection, enrichment, in vivo modulation, or regulated activation).

In one embodiment, when a polypeptide comprises two or more SHARP-tags, the tags are separated by a spacer region comprising one or more amino acid residues. The spacer may be a naturally occurring sequence or an artificial linker. In various embodiments, the inter-tag spacer comprises at least 1, 2, 3, 4, 5, 6, 7, 8, 10, 12, 15, 20, 25, 50, 75, 100, 200, 300, 400, 500, 600, 700, 800, 900, or 1000 amino acids. In another aspect, the spacer comprises a peptide linker, which may be flexible, rigid, cleavable, or masking in nature. In specific embodiments, the linker is: a flexible linker, such as SEQ ID NO: 1207; a non-flexible linker, such as SEQ ID NO: 1208; a cleavable linker, such as SEQ ID NOs: 1204-1206; a masking peptide, such as SEQ ID NO: 1203. In one aspect, the linker is selected from SEQ ID NOs: 1203-1208. The linker may be of any suitable length, including ranges of 1-500 or 1-1000 amino acids. In certain embodiments, the SHARP-tag itself may function as a modular linker to connect two or more functional protein domains or may serve as an adaptor domain that facilitates interaction with other molecular components (e.g., antibodies, ligands, or regulatory domains). These embodiments support methods for expressing, purifying, tracking, or regulating the activity of engineered polypeptides in research, diagnostic, or therapeutic applications.

The present invention provides recombinant polynucleotides encoding one or more of the SHARP-tags described herein, as well as recombinant polypeptides comprising one or more such tags. In certain embodiments, the recombinant polypeptide is expressed in a host cell, and the invention further provides cells that express polypeptides bearing one or more SHARP-tags. The invention also encompasses soluble proteins or polypeptides comprising the disclosed SHARP-tags.

In various embodiments, the invention provides methods for the detection, isolation, purification, enrichment, depletion, or regulation of polypeptides, proteins, or cells that comprise one or more of the disclosed SHARP-tags. In certain embodiments, the invention provides methods for modulating or controlling the activity of tagged polypeptides or proteins (e.g., synthetic antigen receptors), and/or of cells expressing such tagged proteins. In additional aspects, the invention provides methods of treatment comprising administration of nucleic acids, polypeptides, vectors, viral vectors, envelopes, cells, and pharmaceutical compositions that comprise one or more of the disclosed SHARP-tags.

In some embodiments, the recombinant polynucleotides encode polypeptides comprising a single epitope tag. In other embodiments, the polynucleotides encode polypeptides comprising two or more epitope tags, which may be identical or distinct in sequence. The one or more epitope tags may be positioned at the N-terminus, C-terminus, internal regions, or within the extracellular or intracellular domains of the polypeptide. The tagged polypeptides may be naturally occurring proteins that have been engineered to include the tag(s), or fully recombinant proteins designed to include the tag(s) for functional or regulatory purposes.

The epitope tags described herein, including their variants and tag cassettes, may be incorporated into chimeric antigen receptors (CARs), synthetic antigen receptors (SARs), or next-generation CAR platforms such as SIR, zSIR, uTCR-SAR, Ab-TCR, and KIR-CAR. In such constructs, the epitope tags may function as detection elements, regulatory modules, linkers, or adaptors. These tags may be used to detect, isolate, purify, eliminate, or regulate the activity of antigen-binding domains (e.g., antibodies, scFVs), fusion proteins, viral vectors, or engineered immune receptors including CARs, SARs, and TCRs.

The use of such tags for detection, purification, or regulation of recombinant molecules is further supported by the technologies and teachings of international patent applications PCT/US2017/024843, PCT/US2017/052344, PCT/US2017/025602, PCT/US2017/042248, PCT/US2017/064379, PCT/US2018/053247, PCT/US2019/035096, PCT/US2019/044255, PCT/US2020/014237, PCT/US21/22643, PCT/US2021/022641, PCT/US22/17177, and PCT/US24/10592, each of which is incorporated herein by reference in its entirety.

In certain embodiments, the epitope-tagged polypeptide or protein described herein corresponds to or encodes a receptor, ligand, cytokine, chemokine, transmembrane protein, secreted protein, cytosolic protein, or other recombinant protein, including but not limited to antigen-binding receptors. In specific embodiments, the antigen-binding receptor is a Synthetic Antigen Receptor (SAR). As used herein, the term “synthetic antigen receptor” or SAR refers to both conventional chimeric antigen receptors (CARs) and next-generation engineered receptor platforms. In each case, the epitope tag may be incorporated without substantially affecting expression, structure, activity, or immunogenicity, as described in prior sections.

In one aspect, the invention provides tagged synthetic antigen receptors (TAG-SARs) comprising one or more epitope tags described herein. In certain embodiments, the extracellular antigen-binding domain of the SAR—such as a variable light chain (vL), variable heavy chain (vH), single-domain antibody (vHH), or single-chain variable fragment (scFv)—is modified by insertion or fusion of an epitope tag. The epitope tag enables tracking, detection, sorting, depletion, and/or regulation of TAG-SAR-expressing cells in vitro and in vivo, without impairing SAR functionality.

The present disclosure further provides recombinant cells, including but not limited to immune cells and stem cells, that comprise or express one or more TAG-SARs. Such recombinant cells may be used for therapeutic, diagnostic, or manufacturing purposes. In various embodiments, the recombinant cells include immune cells (e.g., T cells, NK cells, NKT cells, monocytes/macrophages, B cells, or dendritic cells) or stem cells (e.g., hematopoietic stem cells, embryonic stem cells, induced pluripotent stem cells). The recombinant cells may also include genetically engineered variants thereof and other cell types suitable for cell-based therapy, including muscle, nerve, heart, liver, skin, and epithelial cells.

In specific embodiments, the SAR comprises a single-chain receptor construct (e.g., a conventional CAR or TFP), or a multichain synthetic receptor system (e.g., SIR, zSIR, Ab-TCR, cTCR, HIT, or STAR). The epitope tags described herein can be incorporated into any of these formats to enable improved detection, selection, modulation, and safety control of engineered immune or stem cell products for use in immunotherapy or regenerative medicine.

In certain embodiments, the synthetic antigen receptor (SAR) is configured as a single-chain construct. A single-chain SAR typically comprises an extracellular antigen-binding domain operably linked via an optional hinge or connector region to a transmembrane domain or membrane-associated domain. The construct may further include an intracellular signaling domain comprising one or more effector or costimulatory domains (e.g., CD3ζ, CD28, 4-1BB). One or more epitope tags, as described in preceding sections (e.g., SEQ ID NOs: 692-698), may be incorporated into the extracellular portion to generate a TAG-SAR, enabling enhanced detection, sorting, or depletion.

In alternative embodiments, the SAR is configured as a double-chain receptor comprising two distinct polypeptide chains. Each chain includes an extracellular domain operably linked, directly or via a hinge domain, to a transmembrane domain and optionally to an intracellular domain. One or both chains may contain an antigen-binding domain. For example, one chain may encode a variable light (vL) region and the other a variable heavy (vH) region of an antibody; alternatively, the chains may comprise complementary variable domains of a T cell receptor, such as Vα/Vβ or Vγ/Vδ pairs. The intracellular domains may include effector functions (e.g., CD3ζ) or may serve as scaffolds for recruitment of endogenous signaling adaptors (e.g., CD3z, DAP10, DAP12). Representative double-chain SARs include SIR, Ab-TCR, zSIR, zCD16-SAR-uTCR-SAR, Hybrid-chain SAR (HC-SAR), and recombinant TCR constructs. As with single-chain formats, one or more epitope tags may be incorporated into the extracellular region of one or both chains to facilitate functional control and tracking of TAG-SAR-expressing cells.

In some embodiments, SAR constructs—whether single or double chain—further comprise one or more Autonomous Antigen Binding Domains (AABDs), which are non-scFv-based binding domains. AABDs may include single-domain antibodies, designed ankyrin repeat proteins (DARPins), fibronectin domains, or other modular targeting moieties.

In additional aspects, the SARs described herein comprise at least one extracellular component and optionally at least one intracellular component, connected via a hydrophobic transmembrane domain. In certain embodiments, the SAR is configured as a fusion protein, wherein one or more epitope tag cassettes are positioned (a) at the N-terminus of the extracellular binding domain, (b) within the binding domain itself, or (c) between the extracellular domain and the transmembrane domain. The modular design allows for flexibility in detection, tracking, regulation, or depletion of engineered cells.

Further embodiments of the TAG-SARs provide flexible, multi-tagged fusion proteins. For example: In one configuration, the fusion protein comprises (from N- to C-terminus): an extracellular binding domain, a single tag cassette, a hinge-containing connector region, a transmembrane segment, and an optional intracellular domain. In another configuration, the fusion protein comprises: an extracellular binding domain, a first connector, a tag cassette, a second hinge-containing connector, a transmembrane domain, and an intracellular component. In additional embodiments, multiple tag cassettes are interleaved with connector domains, e.g., an extracellular domain followed by tag cassette 1→connector 1→tag cassette 2→connector 2 (hinge)→transmembrane→intracellular signaling. In extended embodiments, three or more tag cassettes are incorporated in sequence, each flanked by connector domains, to enable precise detection or control of the SAR construct. In double-chain TAG-SARs, one or both polypeptide chains may independently follow any of the above-described domain designs.

In certain other TAG-SAR embodiments, the fusion protein comprises from amino-terminus to carboxy-terminus: a tag cassette, an extracellular binding domain, a connector region comprising a hinge, a hydrophobic portion, and an optional intracellular component comprising an effector domain and/or a costimulatory domain. In still other TAG-SAR embodiments, the fusion protein comprises from amino-terminus to carboxy-terminus: an extracellular scFv or scTCR binding domain comprising a variable region linker containing a tag cassette disposed between the variable regions (e.g., at or closer to the N-terminal end of the variable region linker, at or closer to the C-terminal end of the variable region linker, or imbedded closer to the middle of the variable region linker), a connector region comprising a hinge, a hydrophobic portion, and an optional intracellular component comprising an effector domain and/or a costimulatory domain.

Examples of different configurations of SAR are provided in Tables A1-27 of provisional application. The SHARP-tag can replace the Linker region or AABD.

The tagged fusion protein may be cell-bound (e.g., expressed on a cell surface) or in soluble form. In certain embodiments, nucleic acid molecules encoding TAG-SAR fusion proteins may be codon optimized (e.g., human codon optimized) to enhance or maximize expression in certain types of cells, such as T cells. In still further embodiments, TAG-SAR may further comprise a functional component (e.g. an immunostimulatory moiety, cytokine, immune modulator, immunoglobulin protein, or the like).

A TAG-SAR may have at least two different tag cassettes. In some embodiments, a first tag cassette can provide a stimulation signal and a distinct second tag cassette might be used to associate with a detection reagent or associate with an antibody-toxin conjugate or with an antibody-imaging agent conjugate. In further embodiments, the two or more first tag cassettes may be in different areas of a TAG-SAR. In certain embodiments, a first tag cassette in the connector region and a second tag cassette is located at the amino-terminus or internal or both of a TAG-SAR. In certain embodiments, a tag cassette comprises from about five to about 500 amino acids, or from about six to about 100 amino acids, or from about seven to about 50 amino acids, or from about six to about 20 amino acids. In some embodiments, a tag cassette has seven to eleven amino acids. Preferably, a tag cassette is non-immunogenic, hypoimmunogenic or minimally immunogenic. Essentially, a tag cassette can function as a handle or beacon to allow for the identification, enrichment, isolation, promotion of proliferation, activation, tracking, or elimination of cells expressing a TAG-SAR.

In certain embodiments, a tag cassette is located within a connector region of a fusion protein of this disclosure. For example, a connector region may further comprise a linker module adjacent to a tag cassette, wherein the linker module comprises a flexible linker. Exemplary flexible linkers are provided in SEQ ID NO: 1330-1331 and 1453. Additional flexible linkers are known in the art.

A single chain fusion protein comprising one or more tag cassettes as described herein will be capable of associating with a cognate binding partner, wherein the cognate binding partner is heterologous to the host or cell expressing a fusion protein comprising a tag cassette as described herein. In certain embodiments, a tag or a tag cassette present in a TAG-SAR of this disclosure has Polatuzumab vedotin, 2F2, SN8 or huMA79b as a cognate binding partner, or is recognized by antibodies specific for a CD79b. In certain embodiments, a tag or a tag cassette present in a TAG-SAR of this disclosure has H2Mab-250 as a cognate binding partner or is recognized by antibodies specific for a Her2. In certain embodiments, the cognate binding partner (e.g., receptor, protein, antibody) may be soluble, part of a matrix composition, or conjugated to a solid surface (e.g., plate, bead). Exemplary solid surfaces include beads and particles (e.g., micro and nano), such as magnetic beads and particles. In single chain TAG-SAR fusion protein embodiments, a protein complex can form between a fusion protein and a cognate tag cassette binding partner, which is a result of binding between the tag cassette and the binding partner. In certain embodiments, a TAG-SAR comprises a scFv or scTCR binding domain where the tag cassette is located within the variable region linker (between the binding domain subunits). In other embodiments, a TAG-SAR has a tag cassette located at the amino-terminus of the binding domain. In such protein complexes or fusion protein structures, a TAG-SAR binding domain will retain its target specificity or its specific target binding affinity.

A connector (or linker) region comprising a hinge in a fusion protein according to the present disclosure may be located (a) immediately amino-terminal to a hydrophobic portion, (b) interposed between and connecting a tag cassette (e.g., SHARP-tag) and an effector domain, (c) immediately carboxy-terminal to a binding domain, or (d) interposed between and connecting a linker module and an effector domain.

A connector (or linker) region can be comprised of a hinge only, linker modules only, a hinge and linker modules, or a hinge, one or more linker modules and one or more tag cassettes. Examples of different locations where a linker region containing a tag of the disclosure can be located in a SAR are provided in Tables A1-27 of PCT/US24/10592.

Hydrophobic Portion. A hydrophobic portion contained in a fusion protein of the present disclosure (e.g., TAG-SAR) will allow a fusion protein of this disclosure to associate with a cellular membrane such that a portion of the fusion protein will be located extracellularly (e.g., tag cassette, connector domain, binding domain) and a portion will be located intracellularly (e.g., an optional effector domain).

Effector Domain. An optional effector domain contained in a fusion protein of the present disclosure (e.g., TAG-SAR) will be an intracellular component and capable of transmitting functional signals to a cell. In some embodiments, an effector domain of a TAG-SAR of the instant disclosure is CD3z and CD28, is CD3z and 4-1BB, or is CD3z, CD28 and 4-1BB.

Binding Domain. As described herein, a TAG-SAR fusion protein of the present disclosure comprises a binding domain that specifically binds a target. A binding domain may be any peptide that specifically binds a target of interest. Sources of binding domains include antibody variable regions from various species (which can be in the form of antibodies, scFVs, scFVs, Fabs, scFv-based grababody, or soluble vH domain or domain antibodies), including human, rodent, avian, or ovine. Additional sources of binding domains include variable regions of antibodies from other species, such as camelid, nurse sharks, spotted ratfish, or lamprey. These antibodies can form antigen-binding regions using only a heavy chain variable region, i.e., these functional antibodies are homodimers of heavy chains only (referred to as “heavy chain antibodies”).

An alternative source of binding domains of this disclosure includes sequences that encode random peptide libraries or sequences that encode an engineered diversity of amino acids in loop regions of alternative non-antibody scaffolds, such as scTCR, designed ankyrin repeat proteins (DARPins), fibronectin binding domains (adnectins or monobodies), cysteine-knot miniproteins, tetratricopeptide repeat domains, leucine-rich repeat domains, lipocalin domains, V-like domains, C-type lectin domains, mAb2 or Fcab™, armadillo repeat proteins, affilin, affibody, avimers, knottins, fynomers, atrimers, cytotoxic T-lymphocyte associated protein-4 or the like. Binding domains of this disclosure can be generated as described herein or by a variety of methods known in the art. In some embodiments, a binding domain is a single chain Fv fragment (scFv) that comprises vH and vL regions specific for a target of interest. In certain embodiments, the VH and vL regions are human. Exemplary vH and vL regions are provided in Tables 2 and 3. In certain other embodiments, a tag cassette is a part of or is located within a (GlyₙSer)-based linker used to link the vH and vL domains of a binding domain. In still further embodiments, a (GlyₙSer)-based linker may be used to connect one or more tag cassettes to the N-terminal end of a TAG-SAR binding domain.

### Host Cells and Nucleic Acids

In certain aspects, the present disclosure provides nucleic acid molecules that encode any one or more of the TAG-SAR described herein. Such nucleic acid molecules can be inserted into an appropriate vector (e.g., viral vector or non-viral plasmid vector) for introduction in a host cell of interest (e.g., hematopoietic progenitor cell, T cell).

As used herein, the term “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering.

Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a gene or operon. In certain embodiments, a cell, such as a T cell, obtained from a subject may be converted into a non-natural or recombinant cell (e.g., a non-natural or recombinant T cell) by introducing a nucleic acid that encodes a TAG-SAR as described herein and whereby the cell expresses a cell surface located TAG-SAR.

A vector that encodes a core virus is referred to herein as a “viral vector.”

In certain embodiments, a viral vector is used to introduce a non-endogenous nucleic acid sequence encoding a TAG-SAR specific for a target.

Other vectors also can be used for polynucleotide delivery including DNA viral vectors, including, for example adenovirus-based vectors and adeno-associated virus (AAV)-based vectors; and vectors derived from herpes simplex viruses (HSVs).

In certain embodiments, hematopoietic progenitor cells or embryonic stem cells are modified to comprise a non-endogenous nucleic acid molecule that encodes a TAG-SAR of this disclosure. In certain embodiments, the host T cell transfected to express a TAG-SAR of this disclosure is a functional T cell, such as a virus-specific T cell, a tumor antigen specific cytotoxic T cell, a naive T cell, a memory stem T cell, a central or effector memory T cell, or a CD4+ CD25+ regulatory T cell.

Diseases that may be treated with cells expressing TAG-SAR as described in the present disclosure include cancer, infectious diseases (viral, bacterial, protozoan infections), immune diseases (e.g., autoimmune), or aging-related diseases (e.g., senescence). Adoptive immune and gene therapy are promising treatments for various types of cancer. A TAG-SAR of this disclosure may be administered to a subject in cell-bound form {e.g., gene therapy of target cell population (mature T cells {e.g., CD8+ or CD4+ T cells) or other cells of T cell lineage)). In a particular embodiment, cells of T cell lineage expressing TAG-SAR administered to a subject are syngeneic, allogeneic, or autologous cells. In other embodiments, TAG-SAR may be administered to a subject in soluble form.

Pharmaceutical compositions including TAG-SAR of this disclosure may be administered in a manner appropriate to the disease or condition to be treated (or prevented) as determined by persons skilled in the medical art. An appropriate dose, suitable duration, and frequency of administration of the compositions will be determined by such factors as the condition of the patient, size, type and severity of the disease, particular form of the active ingredient, and the method of administration. The present disclosure provides pharmaceutical compositions comprising cells expressing a TAG-SAR as disclosed herein and a pharmaceutically acceptable carrier, diluents, or excipient. Suitable excipients include water, saline, dextrose, glycerol, or the like and combinations thereof.

An advantage of the instant disclosure is that TAG-SAR expressing cells administered to a patient can be depleted using the cognate binding partner to a tag cassette. In certain embodiments, the present disclosure provides a method for depleting a T cell expressing a TAG-SAR by using an antibody specific for the tag cassette, using a cognate binding partner specific for the tag cassette, or by using a second T cell expressing a CAR and having specificity for the tag cassette. In certain embodiments, a tag cassette allows for immunodepletion of a T cell expressing a TAG-SAR of this disclosure. Elimination of engineered T cells may be accomplished using depletion agents specific for a tag cassette. For example, if a P11 tag (SEQ ID NO: 674) is used, then Polatuzumab vedotin, SN8 or 2F2 antibody may be used. Similarly, if a Tag with SEQ ID NO: 557-566 is used, then H2Mab-250 or its variants may be used.

In certain other embodiments, cells expressing a TAG-SAR of this disclosure can be identified, sorted, enriched or isolated by binding to antibodies having specificity to a tag cassette (e.g., anti-tag antibodies), or by other proteins that specifically bind a tag cassette (e.g., SN8, 2F2, huMA79b binding to the P7, P8, P9, P10 or P11), which are conjugated to beads, a cell culture plate, agarose, or any other solid surface matrix. In certain embodiments, such cells are sorted, enriched or isolated by using an affinity column.

In certain embodiments, the present disclosure provides a method for selectively activating a T cell by contacting a non-natural or recombinant T cell expressing a TAG-SAR with a binding domain specific for a tag cassette. In an embodiment, the binding domain is attached to a solid surface or as part of a biocompatible matrix (e.g., alginate, basement membrane matrix (Matrigel®), biopolymer). The recombinant T cell comprises an exogenous nucleic acid molecule encoding a TAG-SAR fusion protein of this disclosure. For example, a T cell expressing a TAG-SAR may be activated with beads coated or conjugated with a cognate binding partner (e.g., antibody) specific for the tag cassette. For example, if the tag cassette is a P11-16 (SEQ ID NO: 1-123, 674-676) then appropriate antibodies (e.g., SN8, 2F2, huMA79b) or antibody-coated beads can be used to induce T cell activation. Similarly, if the tag cassette has a sequence of SEQ ID NO:557-566 or a variant thereof, then HuMab-250 or HuMab-250-coated beads can be used to induce T cell activation. In certain embodiments, the method comprises activating ex vivo recombinant T cells expressing a TAG-SAR of this disclosure and is optionally further expressing a chimeric antigen receptor (CAR). Such activated T cells are useful in the disease treatment methods described herein. In an embodiment, the activation occurs in vitro. In an

In another aspect, the present disclosure provides a method for selectively promoting proliferation of a recombinant T cell expressing a TAG-SAR of this disclosure. In certain embodiments, the method comprises selective ex vivo proliferation of T cells expressing a TAG-SAR using a tag binding partner, such as an antibody. In further embodiments, the method comprises expanding functional T cells (e.g., virus-specific, TAA (tumor-associated antigen) specific CTL, or specific T cell subsets, such as naive T cells, memory stem T cells, central or effector memory T cells, CD4+ CD25+ regulatory T cells) with a tag binding partner, such as an antibody, which may optionally be done in the presence of a costimulatory molecule binding partner (such as an anti-CD27 or antiCD28 antibody). In certain embodiments, anti-tag binding partners may be used to activate a TAG-SAR transduced hematopoietic stem cell, embryonic stem cell, or tissue stem cell (e.g., neural stem cell) to self-renew, proliferate or differentiate into one or more desired phenotype for therapeutic use.

In still further embodiments, a TAG-SAR allows for selective promotion of T cell proliferation in vivo when expressing a TAG-SAR of this disclosure. In certain embodiments, a T cell expressing a SAR comprising a tag cassette allows for expansion of the SAR T cells in vivo when contacting cells expressing a ligand {e.g., including T cell suppressor cell ligands PD-L1, PD-L2). Such expanded T cells are useful in the disease treatment methods described herein. In certain embodiments, proliferation or expansion of cells expressing TAG-SAR as disclosed herein is induced in vivo, which may be induced with a tag cassette binding partner (such as an anti-tag antibody) and optionally a costimulatory molecule binding partner (such as an anti-CD27 or antiCD28 antibody).

In certain further embodiments, cells expressing TAG-SAR as disclosed herein are activated in vivo, such as at the site of a tumor. For example, a composition (e.g., alginate, basement membrane matrix (Matrigel®), biopolymer, or other matrix) or a carrier {e.g., microbead, nanoparticle, or other solid surface) comprising a tag cassette binding partner (such as an anti-tag antibody or SABR) and a costimulatory molecule binding partner (such as an anti-CD27 or antiCD28 antibody) may be used to locally activate at the site of a tumor (e.g., a solid tumor) a T cell expressing a TAG-SAR as disclosed herein.

In certain embodiments, recombinant cells expressing a TAG-SAR may be detected or tracked in vivo by using antibodies that bind with specificity to a tag cassette (e.g., anti-Tag antibodies), or by other cognate binding proteins that specifically bind the tag cassette sequence (e.g., SN8, 2F2 or huMA79b binding to tags with SEQ ID NO: 1-123, 674-676 etc), which binding partners for the tag cassette are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu et al., Theranostics 2:3, 2012).

In further embodiments, cells expressing TAG-SAR of the instant disclosure may be used in diagnostic methods or imaging methods, including methods used in relation to the indications or conditions identified herein.

In another aspect, the disclosure provides novel design of uni-specific, bispecific, and multi-specific SARs (e.g., SIR, Ab-TCR, etc.) comprising one or more hybrid TCR constant chains or functional variants thereof, including variants from non-human species (e.g., mouse, cat, dog, monkey, etc.). In some embodiments, the HC-SAR are TAG-SAR.

The disclosure provides novel viral envelope proteins for pseudotyping of lentiviral vectors. Examples of viral envelope proteins include modified baboon envelope (mBaEV) and modified HERV-W1 envelope proteins and are provided in SEQ ID NO (DNA): 3680-3706, 3708, 3725-3732, 3749, 3756 and SEQ ID NO (PRT): 1230-1256, 1258, 1275-1282, 1299 and 1306 of the present disclosure. In an embodiment, the modified BaEV envelope glycoprotein of the present disclosure is not the BaEV/TR envelope glycoprotein, which is represented by SEQ ID NO: (PRT): 1218. In an embodiment, the modified BaEV envelope glycoprotein of the present disclosure lacks one or more (e.g., 1, 2, 3, 4, 5, 6, 7, 8, 9, 10, 11, 12, 13 etc.) carboxy-terminal amino acid residues of BaEV envelope glycoprotein represented by SEQ ID NO: (PRT): 1218.

In an embodiment, the modified BaEV envelope glycoprotein of the present disclosure adds at least one (e.g., 1, 2, 3, 4, 5 6, 7, 8, 9, 10, 11, 12, 13, 14, 15, 20, 25 etc.) amino acid to the carboxy terminus of the BaEVRless envelope described in WO2013045639 and which is represented by SEQ ID NO (PRT): 1211. In an embodiment, the modified BaEV envelope glycoprotein of the present disclosure adds at least two amino acids to the carboxy terminus of the BaEVRless envelope (SEQ ID NO (PRT): 1211). In an embodiment, the modified BaEV envelope glycoprotein of the present disclosure adds more than one amino acid residues (e.g., 2, 5, 10, 50, 99 amino acid residues) to the carboxy terminus of the BaEVRless envelope glycoprotein (SEQ ID NO (PRT): 1211). In an embodiment, the modified BaEV envelope glycoprotein of the present disclosure is at least one amino acid longer than the BaEVRless envelope glycoprotein (SEQ ID NO (PRT):1211. In an embodiment, the modified BaEV envelope glycoprotein of the present disclosure is more than one amino acid (e.g., 2, 3, 4, 5, 6, 7, 8, 9, 10, 15, 20, or 99 amino acid residues) longer than the BaEVRless envelope glycoprotein (SEQ ID NO (PRT): 1211. In an embodiment, the modified envelope glycoprotein of the present disclosure does not comprise the TR domain, which is represented by SEQ ID NO (PRT): 1219.

In an embodiment, the modified BaEV envelope glycoprotein of the present disclosure has a cytoplasmic domain that has a non-aromatic amino acid, a basic, an acid, a neutral, an aromatic, a polar, a negative charged, or a positively charged residue at position 23 of the cytoplasmic domain. In an embodiment, the modified BaEV envelope glycoprotein of the present disclosure has a cytoplasmic domain that has any amino acid residue at position 23 of the cytoplasmic domain. In an embodiment, the modified BaEV envelope glycoprotein of the present disclosure has a cytoplasmic domain wherein the amino acid residue at position 23 is any amino acid other than Val (V). In an embodiment, the modified BaEV envelope glycoprotein of the present disclosure has a cytoplasmic domain wherein the residue at position 23 is any amino acid other than Val (V), the residue at position 24 is any amino acid other than Leu (L) or Ser (S), residue at position 25 is any amino acid other than Thr (T), residue at position 26 is any amino acid other than Q, residue at position 27 is any amino acid other than Q, residue at position 28 is any amino acid other than Y, residue at position 29 is any amino acid other than Q residue at position 30 is any amino acid other than V, residue at position 31 is any amino acid other than L, and/or residue at position 32 is any amino acid other than R. In an embodiment, the first amino acid of the cytoplasmic domain is calculated from Glycine at residue 525 of SEQ ID NO (PRT): 1210 or 1211.

In an embodiment, the modified BaEV envelope glycoprotein of the present disclosure has a cytoplasmic domain that is at least 23 amino acid residues in length and does not comprise the TR domain, which is represented by SEQ ID NO (PR):1274.

In another aspect, the disclosure provides SARs comprising novel polynucleotides, polypeptides targeting specific antigens. In one aspect the antigen is human CD79b. Examples of novel antigen binding domains (e.g., vL and vH) targeting human CD79b and SAR incorporating such antigen binding domains are provided in Tables 2 and 3. In one aspect, the CD79b SAR comprises a vL domain represented by SEQ ID NO (DNA):4623-4628 and SEQ ID NO (PRT): 2173-2178 or a functional variant thereof and a complementary vH domain represented by SEQ ID NO (DNA): 4623-4632 and SEQ ID NO (PRT): 2179-2182 or a functional variant thereof. In one aspect, the vL domain of a double chain SAR targeting human CD79b is operably linked via an optional linker to a first signaling chain comprising an Ig-like linker domain derived from human TCRβ constant chain that is fused in frame to a module comprising the hinge domain, transmembrane domain and cytosolic domain of human CD3z or a functional variant thereof. In one aspect, the first signaling chain is represented by SEQ ID NO (DNA): 4635 or 4636 and SEQ ID NO (PRT): 2185 or 2186 or a functional variant thereof. In one aspect, the first signaling chain is represented by SEQ ID NO (DNA): 4643 or 4644 and SEQ ID NO (PRT): 2193 or 2194 or a functional variant thereof. In one aspect, the vH domain of a double chain SAR targeting human CD79b is operably linked via an optional linker to a second signaling chain comprising an Ig-like linker domain derived from human TCRα constant chain that is fused in frame to a module comprising the hinge domain, transmembrane domain and cytosolic domain of human CD16a or a functional variant thereof. In one aspect, the second signaling chain is represented by SEQ ID NO (DNA): 4637 or 4638 and SEQ ID NO (PRT): 2187 or 2188 or a functional variant thereof. In one aspect, both chains of the double chain SAR targeting human CD79b comprise an N-terminal signal peptide (e.g., SEQ ID NO (PRT): 3170 and 3171). In one aspect, the two chains of a double chain SAR targeting human CD79b are joined via a sequence encoding a furine cleavage site (SEQ ID NO (DNA):3906 and SEQ ID NO (PRT): 1456, a Ser-Gly linker (e.g., SEQ ID NO (DNA): 3903 and SEQ ID NO: (PRT):1453) and a cleavable linker (e.g., P2A; SEQ ID NO (DNA):3900 and SEQ ID NO (PRT): 1450) or a functional variant thereof. In one aspect, the double chain SAR targeting human CD79b is represented by SEQ ID NO (DNA):4648-4671 and SEQ ID NO (PRT): 2198-2221. In one aspect, the double chain SAR targeting human CD79b is represented by SEQ ID NO (DNA):4672-4695 and SEQ ID NO (PRT): 2222-2245 or a functional variant thereof.

In one aspect, the CD79b SAR comprises a vL domain represented by SEQ ID NO (DNA):4623-4628 and SEQ ID NO (PRT): 2173-2178 and a complementary vH domain represented by SEQ ID NO (DNA): 4623-4632 and SEQ ID NO (PRT): 2179-2182. In one aspect, the vL domain of a double chain SAR targeting human CD79b is operably linked via an optional linker to a first signaling chain comprising an Ig-like linker domain derived from human TCRβ constant chain that is fused in frame to a module comprising the hinge domain, transmembrane domain and cytosolic domain of human CD3z. In one aspect, the first signaling chain is represented by SEQ ID NO (DNA): 4635 or 4636 and SEQ ID NO (PRT): 2185 or 2186. In one aspect, the first signaling chain is represented by SEQ ID NO (DNA): 4643 or 4644 and SEQ ID NO (PRT): 2193 or 2194. In one aspect, the vH domain of a double chain SAR targeting human CD79b is operably linked via an optional linker to a second signaling chain comprising an Ig-like linker domain derived from human TCRα constant chain that is fused in frame to a module comprising the hinge domain, transmembrane domain and cytosolic domain of CD3z. In one aspect, the second signaling chain is represented by SEQ ID NO (DNA): 4645 or 4646 and SEQ ID NO (PRT): 2195 or 2196 or a functional variant thereof. In one aspect, both chains of the double chain SAR targeting human CD79b comprise an N-terminal signal peptide (e.g., SEQ ID NO (PRT): 3170 and 3171). In one aspect, the two chains of a double chain SAR targeting human CD79b are joined via a sequence encoding a furine cleavage site (SEQ ID NO (DNA):3906 and SEQ ID NO (PRT): 1456, a Ser-Gly linker (e.g., SEQ ID NO (DNA): 3903 and SEQ ID NO: (PRT):1453) and a cleavable linker (e.g., P2A; SEQ ID NO (DNA):3900 and SEQ ID NO (PRT): 1450) or a functional variant thereof. In one aspect, the double chain SAR targeting human CD79b is represented by SEQ ID NO (DNA):4696-4719 and SEQ ID NO (PRT): 2246-2269 or a functional variant thereof.

In one aspect, the CD79b SAR comprises a vL domain represented by SEQ ID NO (DNA):4623-4628 and SEQ ID NO (PRT): 2173-2178 and a complementary vH domain represented by SEQ ID NO (DNA): 4623-4632 and SEQ ID NO (PRT): 2179-2182. In one aspect, the vL domain of a double chain SAR targeting human CD79b is operably linked via an optional linker to a first signaling chain comprising a human TCRβ constant chain. In one aspect the human TCRβ constant chain is human codon optimized and comprises a S57C mutation. In one aspect, the first signaling chain comprising human TCRβ constant chain is represented by SEQ ID NO (DNA): 4639 or 4640 and SEQ ID NO (PRT): 2189 or 2190 or a functional variant thereof that can dimerize with the complementary TCRα constant chain. In one aspect, the vH domain of a double chain SAR targeting human CD79b is operably linked via an optional linker to a second signaling chain comprising a human TCRα constant chain. In one aspect the human TCRα constant chain is human codon optimized and comprises a T48C mutation. In one aspect, the second signaling chain comprising human TCRα constant chain is represented by SEQ ID NO (DNA): 4641 or 4642 and SEQ ID NO (PRT): 2191 or 2192 or a functional variant thereof that can dimerize with the complementary TCRβ constant chain. In one aspect, the both chains of the double chain SAR targeting human CD79b comprise an N-terminal signal peptide (e.g., SEQ ID NO (PRT): 3170 and 3171). In one aspect, the two chains of a double chain SAR targeting human CD79b are joined via a sequence encoding a furine cleavage site (SEQ ID NO (DNA):3906 and SEQ ID NO (PRT): 1456, a Ser-Gly linker (e.g., SEQ ID NO (DNA): 3903 and SEQ ID NO: (PRT):1453) and a cleavable linker (e.g., P2A; SEQ ID NO (DNA):3900 and SEQ ID NO (PRT): 1450) or a functional variant of the forgoing. In one aspect, the double chain SAR targeting human CD79b is represented by SEQ ID NO (DNA):4720-4743 and SEQ ID NO (PRT): 2270-2293 or a functional variant thereof. In one aspect, the TCRβ constant chain of the double chain SAR targeting human CD79b is replaced by a hybrid chain comprising the Ig like linker domain and connecting peptide of TCRβ fused in frame to the transmembrane and cytosolic domain of human TCRγ chain. An example of a hybrid TCRβ and TCRγ chain is represented by SEQ ID NO (DNA):4622 and SEQ ID NO (PRT):2172 or a functional variant thereof that can dimerize with the complementary human TCRα chain.

In another aspect, the SAR targeting human CD79b is represented by SEQ ID NO (DNA):4744-4750, 4752, 4898, 4857, 5060-5067, 5069-5071, 5073 and SEQ ID NO (PRT): 2294-2300, 2302, 2448, 2407, 2610-2617, 2619-2621, 2623, or a functional variant thereof. In another aspect, the SAR targeting human CD79b is represented by SEQ ID NO (DNA): 5068 and SEQ ID NO (PRT): 2618 or a functional variant thereof.

In one aspect, the SAR of the disclosure e.g., a SAR targeting human CD79b, is co-expressed with an accessory module. In one aspect, the accessory module encodes for human IL2, IL12 or IL15. In one aspect, the human IL2, IL12 or IL15 are membrane anchored. Examples of accessory modules are provided in SEQ ID NO (PRT): 665-673, 679-684, 1781-1805, 2622 and SEQ ID NO (DNA):3115-3123, 3129-3134, 4231-4255 and 5072.

Also provided herein are polypeptides encoding any of the epitopes and SAR (e.g., CD79b SAR) described herein. Also provided are one or more vectors comprising the polynucleotides described herein. Also provided herein are cells (e.g., T, NK, NKT, iPSC, hematopoietic stem cells) transformed with one or more vectors comprising one or more polynucleotide that encode any of the epitope tags and SAR (e.g., CD79b SAR) described herein. In one aspect, the cells are autologous. In one aspect, the cells are allogeneic. In one aspect, the cells are obtained from umbilical cord. In one aspect, the cells are obtained from peripheral blood or bone marrow. In one aspect, the cells are obtained from a donor who has been administered a mobilizing agent (e.g., a CXCR antagonist, e.g., Mavorixafor, Plerixafor, and/or a cytokine, e.g., G-CSF, GM-CSF).

In another aspect, the disclosure provides a double chain antigen receptor which shows activation by a soluble ligand. In an embodiment, the receptor is not a natural receptor, e.g., a natural TCR. In an embodiment, the receptor is a synthetic or a non-natural receptor. In an embodiment, the receptor is a synthetic antigen receptor (SAR). In an embodiment, the receptor has the design of a SIR, cTCR, Ab-TCR, zSIR, HIT, STAR, z16SAR, z16SAR. In an embodiment, the receptor comprises at least one extracellular domain and at least one hydrophobic domain. In an embodiment, the receptor is a dimer of two polypeptide chains. In an embodiment, the receptor comprises vL and vH fragments as the antigen binding domain. In an embodiment, the vL and vH fragments are present on two polypeptide chains of the double chain receptor and form a Fv fragment that can bind to the cognate ligand. In an embodiment, the ligand is a peptide or polypeptide. In an embodiment, the peptide is between 6-11, 6-13, 5-10, 4-10, 7-16, 8-17 amino acid residues in length. In an embodiment the peptide forms a linear epitope. In an embodiment the peptide lacks a cysteine residue.

Also provided herein are pharmaceutical compositions that include any of the mammalian cells described herein and a pharmaceutically acceptable carrier. Also provided herein are kits that include any of the pharmaceutical compositions described herein.

Also provided herein are pharmaceutical compositions that include any of the nucleic acids described herein that encode any of the epitope tags, single chain, double chain and multi-chain SARs and/or accessory modules described herein, or any of the sets of nucleic acids described herein that together encode any of the single chain, double chain and multi chain SARs and/or accessory modules described herein, and a pharmaceutically acceptable carrier. Also provided herein are kits that include any of the pharmaceutical compositions described herein.

Also provided are methods of treatment and/or prevention of disease by using any of the polynucleotides, polypeptides, vectors and compositions described herein. In one aspect, the CD79b SAR of the disclosures are used for the treatment of patients with leukemia and lymphoma. In one aspect, the CD79b SAR of the disclosures are used for the treatment of patients with autoimmune disorders (e.g., Lupus, idiopathic myositis, myasthenia gravis, rheumatoid arthritis etc.) and/or allergic disorders (e.g., asthma).

The disclosure also provides a method of generating a cell therapy product comprising the steps of administering a mobilizing agent (e.g., a CXCR4 antagonist, e.g., Plerixafor and/or a cytokine, e.g., G-CSF or GM-CSF), collecting blood/bone marrow, isolating immune effector cells, transforming them with a polynucleotide comprising an immune receptor and a safety switch comprising a tag (e.g., a tag described herein), optionally expanding the cells, administering them to a subject with dose of 1 million to 1000 million cells/kg in single or multiple doses. In an embodiment, the cell dose is 10 to 1000 million cells/kg in single or multiple doses. In an embodiment, the cell dose is 50 to 5000 million cells/kg in single or multiple doses. In an embodiment, the cell dose is 50 to 10000 million cells/kg in single or multiple doses.

The invention provides a cell-based method to determine the potency and/or titer of a vector encoding a SAR (e.g., a SIR, a HC-SIR, Ab-TCR etc) by infecting T cells or a T cell line with impaired or abolished expression of one or more TCR constant chain. In an embodiment, the TCR constant chain is selected from the group consisting of constant chain of TCR α, β1, β2, γ, or δ. In an embodiment, the vector is a viral vector, a viral like particle, or a lipid nanoparticle. In an embodiment, the T cell line is a Jurkat cell line. In an embodiment, the Jurkat cell line has impaired and/or abolished expression of a TCR constant chain selected from the group consisting of constant chain of TCR α, β1, β2, γ, or δ. In an embodiment, the Jurkat cells are engineered to express a reporter (e.g., EGFP), optionally wherein the reporter is expressed under an NFAT driven promoter. In an embodiment, the method comprises exposing a T cell or T cell line that has impaired and/or abolished expression of a TCR constant chain selected from the group consisting of constant chain of TCR α, β1, β2, γ, or δ with a vector under conditions that lead to transduction of the cell with the vector, optionally culturing the cells for a time interval ranging from 1 h to 7 days, measuring the expression and/or activity of the SAR in the transduced cells. The expression and/or activity of the SAR can be measured using methods known in the art, including but not limited to Protein L staining, antibody staining, Topanga assay, JNG-NFAT-GFP assay, cytokine production, cytotoxicity assay and qPCR etc.

The invention also provides a broadly applicable method to detect the expression of a fusion protein, (e.g., a SAR) based on staining with an antibody, antibody fragment or a non-immunoglobulin antigen binding domain raised or directed against the antigen binding domain(s) of the SAR. In an embodiment, the SAR is a CAR (e.g., a second-generation CAR, a third generation CAR, an armored CAR etc.), a next generation CAR (e.g., a SIR, HIT, STAR, Ab-TCR, TFP, zSIR, z16SAR, CD16-SAR, HC-SAR etc.). In an embodiment, the antigen binding domain of the SAR comprises of an scFv, a vL/vH fragment, Fab, (Fab′)2, camelid vHH domain or a non-immunoglobulin antigen binding scaffold (e.g., D-domain, DARPIN, Centyrin etc). In an embodiment, the antibody is directed to an immunoglobulin or an antibody or an antibody fragment (e.g., Fab, (Fab′)2 etc.). In an embodiment, the antibody is not an anti-ideotype antibody.

In one aspect, the present disclosure provides methods and compositions for detecting a target antigen (e.g., an epitope tag and/or a SAR). In various embodiments, the detection is mediated by an antibody or antibody-derived binding molecule that specifically binds to the target antigen. Antibodies suitable for flow cytometric applications include monoclonal antibodies, polyclonal antibodies, and antibody fragments or derivatives thereof.

In one embodiment, the antibody is a monoclonal antibody that recognizes a single epitope on the antigen. In another embodiment, the antibody is a polyclonal antibody, which recognizes multiple epitopes on the target antigen. Monoclonal antibodies may be produced using hybridoma technology, phage display, or recombinant expression systems.

In certain embodiments, the antibody is a recombinant antibody, including chimeric antibodies, humanized antibodies, or fully human antibodies. In yet another embodiment, the antibody is a bispecific antibody or multi-specific antibody capable of recognizing two or more epitopes or antigens.

In some embodiments, antibody fragments are used instead of full-length antibodies. These include: (i) Fab fragments (antigen-binding fragment); (ii) F(ab′)2 fragments (bivalent antigen-binding); (iii) scFv fragments (single-chain variable fragments); (iv) nanobodies (VHH) derived from camelid species; and (v) other engineered fragments such as dsFv, diabodies, triabodies, and minibodies. Such fragments may retain antigen specificity while offering advantages such as improved tissue penetration or reduced background.

Target Regions of Antibodies include Anti-H+L: Antibodies directed against both the heavy and light chains of target immunoglobulins, often used for secondary detection. Anti-Fab: Antibodies specific to the Fab region of immunoglobulins, useful in detecting antigen-binding regions without recognizing Fc. Anti-F(ab′)2: Antibodies directed against F(ab′)2 fragments, offering selective recognition while avoiding Fc-mediated effects.

Antibodies used in flow cytometry may be derived from or raised in various species, including but not limited to mouse, rat, hamster, rabbit, goat, sheep, human, and camelid (e.g., llama, alpaca). Species selection may be based on compatibility with detection reagents (e.g., anti-species secondary antibodies) or reduction of cross-reactivity.

Antibodies may be of various isotypes depending on the species of origin. For example: (i) Mouse: IgG1, IgG2a, IgG2b, IgG3, IgM, IgA; (ii) Rat: IgG1, IgG2a, IgG2b, IgG2c, IgM; (iii) Rabbit: IgG; and (iv) Human: IgG1, IgG2, IgG3, IgG4, IgM, IgA. Isotype information is relevant for appropriate secondary antibody selection and understanding Fc-mediated effects.

Antibodies may be supplied or purified as whole serum (unpurified polyclonal antibody), affinity-purified (e.g., protein A/G or antigen-specific), IgG fraction (partially purified), or ascites fluid (hybridoma-derived). Highly purified and low-endotoxin formulations are preferred for certain in vivo or clinical applications.

To minimize background staining and improve specificity, antibodies may be: (i) isotype control-matched; (ii) species-adsorbed (e.g., mouse anti-human antibody adsorbed against mouse proteins); and/or (iii) Fc receptor-blocked or used in conjunction with Fc-blocking agents. Such adsorption improves signal-to-noise ratio in flow cytometry, particularly in multi-color or cross-species panels.

For detection, antibodies may be conjugated to: (i) fluorophores, such as FITC, PE, APC, PerCP, Alexa Fluor dyes (e.g., Alexa Fluor 488, 647), Brilliant Violet dyes (e.g., BV421), etc.; (ii) metal isotopes (for CyTOF/mass cytometry); (iii) enzymes (e.g., HRP or alkaline phosphatase for rare flow formats); (iv) biotin, with subsequent detection using fluorophore-labeled streptavidin; or (v) DNA barcodes (for multiplexed detection). Antibodies may be directly conjugated to the label or used with secondary reagents specific to the primary antibody species and isotype. The antibodies or fragments described herein may be used to: (i) detect surface, intracellular, or secreted antigens; (ii) quantify antigen expression; (iii) enrich or deplete cell populations by FACS or MACS; (iv) analyze immune cell phenotypes; and (v) assess functional states (e.g., activation, exhaustion). Flow cytometry may be performed on live or fixed cells, and intracellular staining may involve permeabilization protocols.

The present invention also relates to a method of isolating the fusion protein of the invention. Such a method comprises contacting the fusion protein with an antibody of the invention, preferably under conditions allowing formation of a complex between the antibody and the peptide comprised in the fusion protein. Thereby, binding of the fusion protein and the antibody is enabled. This contacting step, also referred to as capture step, may be conducted by contacting a sample, for example a solution, comprising the fusion protein with the antibody.

The sample to be contacted with the tag specific antibody can be any type of sample comprising a fusion protein of the invention and can be processed to separate the polypeptide. Preferably the sample is a solution, for example a lysate of a host cell or a body fluid, comprising the fusion protein of the invention, or a supernatant, such as a supernatant obtainable by centrifugation of a liquid comprising a host cell comprising or capable of expressing fusion protein of the invention, wherein the host cell is capable of secreting or otherwise transporting the fusion protein of the invention to the liquid.

The antibody used in the method of the present invention for isolation and/or purification can be used in solution or immobilized. To immobilize the antibody, the antibody can be bound to a sample carrier, solid support, or matrix. This immobilization step can occur prior to or after the binding of the antibody to the peptide comprised in the fusion protein. Methods for immobilizing antibodies and parts thereof are well-known to the person skilled in the art and any method that allows immobilization without impairing binding properties can be used.

If the antibody of the present invention is not immobilized to a solid support, then the method may comprise a further step of isolating the complex, for example by using a specific binding partner for the complex, such as a secondary antibody that is specific for example for the complex or for the antibody or for a detectable label, such as an affinity tag, that is conjugated to the antibody. The secondary binding partner can be in solution or can be immobilized or immobilizable to a solid support.

In an optional further step following the capture step, the solid support comprising the immobilized antibody bound to the fusion protein is washed to remove unbound and unspecifically bound constituents. Optionally, in a further step, the fusion protein can be eluted to obtain the isolated fusion protein. Elution of the fusion protein bound to the immobilized antibody can be achieved by methods known in the art. For example, the fusion protein can be eluted by competitive elution with an epitope peptide as described herein in isolated form. This isolated epitope peptide will then be in competition with the fusion protein to bind the immobilized tag-specific antibody. If the isolated peptide is added in surplus concentration, the reaction balance of the binding will be shifted to the binding of the immobilized antibody with the isolated epitope tag. This results in the release of the fusion protein. The epitope peptide used for elution may be the same epitope peptide that is comprised in the fusion protein. The epitope peptide used for elution may also be a different peptide than the epitope peptide comprised in the fusion protein. If the epitope peptide used for elution is a different one, it is preferred that the epitope peptide used for elution has a higher binding affinity to the antibody than the epitope peptide comprised in the fusion protein. Additional steps for further purifying the released polypeptide can optionally be added, such as method steps well-known to the skilled person.

The fusion protein may also remain immobilized to the solid support, such as (magnetic) beads, and processed further in downstream application such as mass spectrometry, without the elution step. The fusion protein may comprise a linker with a cleavage site that can be cleaved with an appropriate means, for example a protease, to remove the peptide. Thereby the polypeptide of the fusion protein may be released from the immobilized antibody, and the polypeptide can be obtained in its native form. For this embodiment, the nucleic acid sequence encoding the fusion protein should not only comprise a sequence encoding the epitope tag but also a sequence encoding a linker with a breakable site, for example a cleavage site recognized by a protease. The release step by enzymatic cleave can replace or follow the elution step.

Where the fusion protein of the invention comprises an antibody moiety, the present invention also envisions a method of isolating the target of the antibody moiety of the invention. In principle, this method can be carried out as described above for the isolation of a fusion protein. The method may comprise the additional step of contacting the fusion protein with a specific target of the antibody moiety comprised in the fusion protein. This contacting step may be conducted prior to or after contacting the fusion protein with the antibody that binds to the peptide tag comprised in the fusion protein, with the latter alternative being preferred. In a preferred method, first the antibody specific for the peptide tag is immobilized on the solid support, and then the fusion protein is immobilized via binding to the antibody that is specific to the peptide tag, followed by binding the target of the antibody moiety of the fusion protein to the fusion protein. Elution can be carried out as described above. The specific target may be a cell. For example, a cell surface receptor on the cell, such as CD62L The antibody moiety of the fusion protein may be specific to a structure on the cell, such as CD62L, The antibody moiety of the fusion protein may be a single domain antibody.

The present invention also envisions that detection and isolation of a fusion protein of the invention can be combined. Accordingly, the present invention envisions a method of detection and isolation of a fusion protein of the invention comprising a method of detection of the invention and a method of isolation and/or purification of the invention.

Combination of both methods may thus be carried out by using one antibody conjugated to a detectable label for detection, and another antibody conjugated to a solid support for isolation of the same fusion protein. Both antibodies may be any antibody of the invention. This combination may have the advantage that only one tagged fusion protein has to be generated and detection and isolation carried out with the same transgenic construct/cell. Sometimes, it may be desired that both antibodies have an identical sequence or at least an identical antigen-binding site.

Combination of both methods can also be carried out by using the same antibody and two different peptides for detection and purification. This has the advantage, that only one antibody has to be produced which, depending on the application, can be conjugated to a detectable label or a solid support. Combination of both methods can also be carried out by using two peptides and two antibodies. The fusion protein of interest may comprise a peptide having a high affinity to a given antibody. The present invention further relates to a system comprising one peptide tag and two antibodies or two peptide tags and one antibody or two peptide tags and two antibodies as described herein.

The present invention also relates to a complex comprising (a) fusion protein and (b) an antibody, wherein the fusion protein is a fusion protein of the invention and/or wherein the antibody is an antibody of the invention.

### Additional Description and Exemplary Embodiments of the Invention

In some embodiments, the epitope present on the extracellular protein is referred to as adaptor while the epitope present on the SAR is referred as adaptor binding domain. However, the two domains can be switched. Thus, in some embodiments, the extracellular polypeptide (i.e., SAR adaptor) may comprise a tag (e.g., SEQ ID NO: 1-11) that interacts with the scFv (i.e., adaptor binding domain) present on the signaling chain of the SAR. In other embodiments, the extracellular polypeptide (i.e., SAR adaptor) may comprise an antigen binding domain (e.g., scFv domain) that interacts with the epitope tag (i.e., adaptor binding domain) present on the signaling chain of the SAR.

In some aspects, the SAR of the disclosure includes an adaptor binding domain (e.g., P6, P7, P8, P9, P10, P11, P14, P16 etc.) that allows it to bind to an extracellular polypeptide or SAR adaptor. The immune cells expressing such SAR constructs can be redirected to different target cells via the use of different SAR adaptors (i.e., different antigen binding domains (e.g., antibodies. antibody fragments, vHH, FHVH etc.) fused to the adaptors). Exemplary adaptors and adaptor binding domains are provided herein.

The disclosure provides useful configuration and location for incorporation of adaptors (e.g., a tag or an epitope tag, e.g., Tag1-11 e.g., SEQ ID NO: 1-11 etc.) in a SAR. The disclosure provides useful configurations and locations for incorporation of multiple adaptors. The disclosure provides SAR constructs comprising 1, 2, 3 or more adaptors, e.g., adaptors comprising P7-16 or other protein interaction domains described herein. In an embodiment, the SARs comprise two or more adaptors of the same type (e.g., two P11 domains, two P13 domains etc.). In other embodiments, the SARs comprise two or more adaptors of different types (e.g., one P11 and one P13, or one P11 and one Q domain or one P11 and one RZIP domain or one P11 and one Strep-tag domain etc.).

The disclosure provides several configurations and designs for construction of SARs with AABDs (autonomous antigen binding domains) comprising one or more adaptor binding domain (e.g., a tag or an epitope tag, e.g., P8, P9, P11, P13, e.g., SEQ ID NO: 1-11 etc.). In an embodiment, the disclosure provides that a useful location for attachment of a tag to a SAR is at or near the N-terminus of one or more fragments comprising its antigen binding domain (e.g., vL, vH, scFv, AABD, vHH, FHVH, SVH, SVL, non-immunoglobulin antigen binding scaffold, Va, Vb, Vg, Vd, svd-TCR etc.) via an optional linker. Examples of such SAR are provided in SEQ ID NO: 4757-4759, 4761-4762. Tables A1-27 of PCT/US24/10592 provide schematic representation of various single chain and double chain SAR. One or more tags can be located in any of the linker regions (L) shown in these schematics. Furthermore, due to their relatively small size, the tags of the disclosure can be positioned between the different fragments comprising a SAR. In an embodiment, the tag is located C-terminus to the signal peptide; i.e., between the signal peptide and the antigen binding domain (e.g., scFv, vHH domain). In another embodiment, the tag in the linker region between the vL and vH (or vH and vL) fragments comprising an scFv of a SAR (e.g., SEQ ID NO:4754). In another embodiment, the tag is located between the scFv region and the hinge domain of a SAR (e.g., a CAR). In yet another embodiment, the tag is located between the hinge domain and the transmembrane domain of a SAR.

In an embodiment, the SAR is a double chain SAR (e.g., a SIR, Ab-TCR, zSIR, z16-SAR, TCR etc.). In an embodiment, one or more tags are located C-terminal to one or both signal peptides of a double chain SAR; i.e., between the signal peptide and the antigen binding domain (e.g., vL, vH, scFv, AABD, vHH, FHVH, SVH, SVL, non-immunoglobulin antigen binding scaffold, Va, Vb, Vg, Vd, svd-TCR etc.) (see SEQ ID NO: 4818-4827), In an embodiment, one or more tags are located N-terminal to one or more signaling chains of a double chain SAR. In an exemplary embodiment, the one or more tags are located between the vL fragment and TCRb constant chain and/or vH domain and TCRa constant domain of a double chain SIR (e.g., SEQ ID NO: 4892-4894). In an exemplary embodiment, the one or more tags are located between the different fragments comprising a hybrid chain of a hybrid chain SAR, e.g., between the TCRa and TCRb chains or between TCRb and TCRg chains or between TCRb and IgCL domains or between TCRa and IgG-CH1 domain etc. Similarly, a tag can be located between the TCR linker domains and the CD3z chain of an SIR or between Ig-like linker domain (e.g., IgCL and IgG-CH1) and CD3z domain of a zSIR or z16 SIR, In an embodiment, one or more tags can be located at any location (e.g., N-terminus, C-terminus or internal) of a polypeptide.

In an embodiment, the SAR is a single chain SAR. In another embodiment, the SAR is a multi-chain (e.g., double chain or triple chain) SAR. In an embodiment, an optional linker is present between the adaptor binding domain (tagged cassette) of the SAR and one or more fragments comprising its antigen binding domain. In an embodiment, the epitope tag serves as the linker between one or more fragments of a SAR. In an embodiment, the disclosure provides that a useful location for attachment of an adaptor binding domain (e.g., tag) to a SAR is at the N-terminus or near the N-terminus of an Ig linker, an Ig like linker or a long linker (e.g. linker more than 25 amino acids in length) which, in turn, is attached to a signaling chain (e.g., connecting peptide of a TCRα, TCR β, TCRγ, TCRδ etc.), The disclosure provides useful configuration and location for incorporation of adaptors (tags) in a SAR. The disclosure also provides useful configurations and locations for incorporation of multiple adaptors binding domains (tags). The disclosure provides SAR constructs comprising 1, 2, 3 or more adaptor binding domains, e.g., adaptor binding domains comprising SHARP-tag (e.g., P11, P13 or P16) or other protein interaction domains described herein. In an embodiment, the SARs comprise two or more adaptor binding domains of the same type. In other embodiments, the SARs comprise two or more adaptor binding domains of different types. In an embodiment, the two or more adaptor binding domains are located on the same polypeptide chain of the SAR. In an embodiment, the two or more adaptor binding domains are located on the same polypeptide chain of the SAR and are separated from each other by linkers. In exemplary embodiments, the linker can be a flexible linker or protease cleavable linker. In an embodiment, the two or more adaptor binding domains are located on different polypeptide chains of a multi-chain SAR (e.g., a double chain SIR).

Examples of SAR encoding nucleic acids targeting P11 and CD19 is represented by SEQ ID NO (DNA):8715-8716. Examples of SAR encoding nucleic acids targeting P13, CD19 and CD22 are represented by SEQ ID NO (DNA):8847 and 8849. The immune cells expressing these SARs can be used to target CD19 and/or BCMA expressing cells but can be redirected to target other antigens in combination with suitable antibody, antibody fragments and AABD (e.g., non-immunoglobulin antigen binding scaffolds) comprising a P11 domain.

Epitopes and recombinant polypeptides comprising one or more epitopes can be incorporated in antibodies, antibody fragments, non-immunoglobulin antigen binding scaffolds, cytokines, chemokines, biologicals, recombinant proteins, viral envelopes as epitope tags, linkers and adaptors and can be an be used to detect, isolate, purify, eliminate and/or regulate the activity of their fusion partners. Methods and compositions comprising Epitopes and recombinant polypeptides comprising one or more epitopes are described in WO2021055349 which is incorporated in its entirety by reference herein.

Conditionally active therapeutic proteins comprising epitope tags are presented in WO2012033953A1, which is incorporated by reference herein. In an embodiment, the epitope is inserted into an endogenous protein using gene targeting approaches (e.g., CRISPR/Cas9).

The present invention provides methods and compositions useful for treatment of a disease (e.g., cancer, autoimmune, infectious, degenerative disease etc.) and/or for initiating or modulating immune responses. In some embodiments, the present invention provides cellular therapeutics (e.g., immune cells) comprising a constitutive expression construct, which comprises a promoter operably linked to a gene of interest. In some embodiments, the present invention provides cellular therapeutics (e.g., immune cells) comprising (i) an antigen binding receptor, wherein the antigen binding receptor comprises an antigen-binding domain, a transmembrane domain, and a cytosolic signaling domain, and (ii) a constitutive or inducible expression construct, which comprises a promoter operably linked to a gene of interest. Among other things, the present invention encompasses the recognition that a combination of a cellular therapeutic described herein and one or more additional therapies (e.g., one or more additional cellular therapeutics (e.g., CAR-T cell, CAR-NK cell, TCR-T cell, TIL cell, allogenic NK cell, and autologous NK cell), antibody-drug conjugate, an antibody, and/or a polypeptide described herein), can lead to improved induction of beneficial immune responses, for example a cellular response (e.g., T-cell activation).

In general, a cellular therapeutic described herein can be produced from an immune cell, e.g., a cell useful in or capable of use in adoptive cell therapy. The article of manufacture can comprise a container and a label or package insert on or associated with the container. Kits are also provided that are useful for various purposes, e.g., for treatment of a target antigen-positive disease or disorder described herein, optionally in combination with the articles of manufacture.

In some embodiments, provided are methods for regulating and/or depleting SAR-expressing engineered immune cells from a subject administered with said cells. Depletion can be by inhibition or elimination. Regulation can be positive or negative control.

In one aspect, a method for depleting engineered immune cells expressing a SAR comprising an epitope tag specific for a monoclonal antibody (e.g., SN8, 2F2, Polatuzumab-vedotin, Polivy etc.) comprises contacting said engineered immune cell with a monoclonal antibody specific for the epitope tag. In some embodiments, a method for depleting from a subject administered with engineered immune cells expressing a TAG-SAR comprising an epitope tag specific for a monoclonal antibody comprises administering to the subject a monoclonal antibody specific for the epitope tag. In these embodiments, administration of the monoclonal antibody specific for the epitope tag present in the extracellular domain of the TAG-SAR to the subject eliminates, regulates or inhibits the activity of engineered TAG-SAR-expressing immune cells from the subject. In one aspect, depletion of engineered TAG-SAR expressing immune cells allows for recovery of an endogenous population of TAG-SAR-expressing cells.

In one aspect, the disclosure relates to a method for promoting recovery of endogenous antigen-expressing cells in a subject administered with engineered immune cells expressing at cell surface a TAG-SAR that target one or more antigens expressed on endogenously expressing cells comprising an epitope tag specific for an antigen binding agent (e.g., monoclonal antibody, vHH domain, antibody fragment, non-immunoglobulin antigen binding scaffold etc.), the method comprising administering an antigen binding agent (e.g., monoclonal antibody, vHH domain, antibody fragment, non-immunoglobulin antigen binding scaffold etc.) specific for the epitope tag to the subject.

In some embodiments, the antigen binding agent used in the method for depleting TAG-SAR-expressing engineered immune cells are selected from SN8, CD79b-2F2, Polatuzumab-vedotin, Polivy, a P11-specific CAR or a P11-specific next generation CAR and combinations thereof (e.g., SEQ ID NO: 646, 915). In some embodiments, said epitope specific for a monoclonal antibody (SHARP-tag) is Tag-1-123 (SEQ ID NO: 1-123; 3101-3114; 3124-3128, 3136-3141) or a mutant or a variant thereof and the antigen binding agent specific for the epitope is SN8, 2F2, Polatuzumab-vedotin, 10D10 or a SAR (e.g., SEQ ID NO: 646, 915). In some embodiments, the amount of epitope-specific mAb administered to the subject is sufficient to eliminate at least 20%, 30%, 40%, 50%, 60%, 70%, 80% or 90% of the SAR-expressing immune cell in the subject. In an embodiment, the monoclonal antibody is administered at a dose of 0.1-10 mg/kg intravenously. In an embodiment, the monoclonal antibody is administered at a dose of 1-2 mg/kg intravenously. In an embodiment, the monoclonal antibody is administered by any route including but not limited to subcutaneous, intradermal, intraperitoneal, intraventricular, intrapleural, intracerebral and intrathecal route.

In some embodiments, the antibody is conjugated to an agent.

In some embodiments, the agent is a drug or a radioligand. In some embodiments, the drug is selected from the group consisting of: auristatin E, auristatin F, monomethyl auristatin D (MMAD), monomethyl auristatin F (MMAF), monomethyl auristatin E (MMAE), or venetoclax.

In some embodiments, the antibody and the drug are conjugated via a linker.

In some embodiments, the linker is a cleavable linker. In some embodiments, the linker is a pH-sensitive linker, a glutathione-sensitive linker, or a protease-cleavable linker. In some embodiments, the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, CL2A, maleimidocaproyl (MC), and Mal-EBE-Mal.

In some embodiments, the linker is a non-cleavable linker. In some embodiments, the non-cleavable linker is selected from the group consisting of: N-succinimidyl 4-(N-mal eimidomethyl)cyclohexane-1-carboxylate (SMCC) and maleimidom ethyl cyclohexane-1-carboxylate (MCC), MC-VC-PAB.

In some embodiments, the ratio of the antibody to the drug is between 1:1 and 1:10. In some embodiments, the ratio of the antibody to the agent is 1:4.

In some embodiments, the agent is a radioisotope. In some embodiments, the radioisotope is selected from the group consisting of: Iodine-131, Rhenium-188, Yttrium-90, Bismuth-213, and Actinium-225.

The present disclosure further provides molecular conjugates that comprise an antibody described herein (e.g., SABR) which is linked through a chemical linker to an agent. Any of the conjugates described herein may be synthesized using methods known in the art. See, e.g., Yao et al., Int J Mol Sci. 2016 Feb. 2; 17(2).

The ADCs comprising an antibody conjugated to a drug are advantageous to use therapeutically, in part because the drugs (e.g., chemotherapeutic drugs) are toxic and can be targeted to particular cell types expressing cell surface antigen (e.g., SHARP-tag-expressing cancer cells). By conjugating the drug or radio-isotope to the antibody, the toxicity of the antibody may be reduced by at least 20%, at least 30%, at least 40%, at least 50%, at least 60%, at least 70%, at least 80%, at least 90%, at least 99%, compared to the drug or radio-isotope in its free from.

The present disclosure further provides compositions comprising an antibody that binds to an antigen, or a tag provided herein (e.g., an antibody that binds to a SHARP-tag). In some embodiments, the pharmaceutical composition further comprises a pharmaceutically acceptable carrier. The components of the pharmaceutical compositions also are capable of being co-mingled with the molecules of the present disclosure, and with each other, in a manner such that there is no interaction which would substantially impair the desired pharmaceutical efficacy.

The present disclosure further provides for the administration of an antibody described herein, a conjugate comprising an antibody described herein, or a composition thereof (e.g., a pharmaceutical composition) to a subject.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or a non-human animal. In some embodiments, the subject is a companion animal (e.g., a pet or service animal). “A companion animal,” as used herein, refers to pets and other domestic animals. In some embodiments, the administration occurs more than once.

The disclosure also provides novel accessory modules that can be co-expressed with the recombinant polypeptides (e.g., SARs) of the disclosure. The disclosure provides vectors comprising nucleic acids encoding polypeptides for a) membrane anchored low-affinity variants of cytokines with epitope tags (e.g., IL-2 and/or IL-15); b) membrane anchored cytokines with epitope tags; and c) multi-purpose gene switches with epitope tags that serve suicide, survival, and marker functions.

A “multipurpose switch” or “multipurpose gene” encodes for a protein that provide suicide, survival, and marker functions. In an embodiment, all the above functions are provided by a single polypeptide chain. In example embodiments, nucleic acids encoding multipurpose switches include CD8SP2-P11QP11-IL2-SythCD28TM (SEQ ID NO: 3115) and IL15-CD79b-Iso2-TM-CP4 (SEQ ID NO: 3116) etc.

“Suicide gene”, “suicide switch” or a “Kill-switch” encodes for a protein which possesses an inducible capacity to lead to cellular death. Examples of suicide genes include HSV-TK, iCaspase 9, tEGFR, CD20, tCD19, tHer2, tBCMA, RQR8, CD79b-Iso2 (SEQ ID NO: 3106), CD8SP2-CD79b-Iso2 (SEQ ID NO: 3107), CD8SP2-CD79b-FL (SEQ ID NO: 3108), CD8SP2-P16QP16-8 (SEQ ID NO: 3112), CD8SP2-P13QP13-8 (SEQ ID NO: 3114) etc. In an embodiment, any gene that encodes for a SHARP-tag described herein can serve as a suicide gene or a kill-switch.

A “survival gene”, “survival switch” or a “Life-switch” encodes for a protein that provides a pro-survival signal to a cell. Examples of survival genes include membrane anchored form of IL2 and membrane anchored form of IL15.

The disclosure also provides a therapeutic controls/accessory module comprising epitope tags described herein that serves as “suicide genes”, “Kill switch”, or a “multi-purpose switch”. Examples of nucleic acids encoding “Kill switch” are CD79b-Iso2 (SEQ ID NO: 3106), CD8SP2-CD79b-Iso2 (SEQ ID NO: 3107), CD8SP2-CD79b-FL (SEQ ID NO: 3108), CD8SP2-P16QP16-8 (SEQ ID NO: 3112), CD8SP2-P13QP13-8 (SEQ ID NO: 3114) etc.

Multiple switches, suicide switches and survival switches are described in PCT/US22/17177, which is incorporated in its entirety by reference herein.

The disclosure provides multi-purpose gene switches that serve suicide, survival, and marker functions. In an exemplary embodiment, a multipurpose switch serves as a life-death (or survival-suicide) switch for the purpose of adoptive cell therapy when ectopically expressed in a cell. In an embodiment, the multipurpose switch has the following formula: SP-D1-L1-D2-L2-D3-L3-D4 or SP-D2-L1-D1-L2-D3-L3-D4; where SP is an optional signal peptide that allows cell surface transport of the multipurpose switch and is cleaved to yield the mature peptide, D1 is receptor binding domain which binds to a receptor that promotes cell survival, D2 is a marker/suicide domain that comprise one or more epitope tags described herein, D3 is a hinge domain/stalk domain that allows the D1 and D2 domains to be projected away from the surface of the target cell, D4 is a membrane associating domain (e.g., a transmembrane domain or a membrane anchoring domain) that anchors the molecular switch to the cell membrane and L1, L2 and L3 are optional linker domains.

In an embodiment, the multipurpose switch comprises an in-frame fusion of a first module (D1) comprising a receptor-binding domain to a second module (D2) that serves as a marker/suicide-switch and a third module (D3) that serves as a hinge/stalk domain and a fourth module (D4) that serves as a membrane associating domain. In an embodiment, the D2, D3 and D4 modules are derived from the same endogenous protein. In an embodiment, the D2, D3 and D4 module are derived from different endogenous proteins. In an embodiment, D3 and D4 are derived from the same endogenous protein. In an embodiment, D3 and D4 are derived from the different endogenous proteins.

In an embodiment, the first module (D1) binds to a receptor that is expressed on cell surface, i.e., it binds to the extracellular domain of a receptor. In an embodiment, the first module (D1) binds to a receptor which when bound transmits a pro-survival and/or proliferative signal to the cell. In an embodiment, the first module binds to the receptor in cis (i.e., bind to the receptor expressed on the same cell as the cell expressing the molecular switch). In an embodiment, the first module binds to the receptor in trans (i.e., bind to receptor expressed on a cell other than the cell expressing the molecular switch). In an embodiment, the first module binds to the receptor in cis and in trans. In an embodiment, the first module (D1) comprises the receptor binding domain of a cytokine, a chemokine, a ligand, or a variant or a fragment thereof. Exemplary cytokines, chemokines and ligands include, but are not limited to, one of the following: IL-1α, IL-1β, IL-2, IL-3, IL-4, IL-5, IL-6, IL-7, IL-8, IL-9, IL-10, IL-11, IL-12, IL-13, IL-14, IL-15, IL-16, IL18, IL-19, IL20, IL-21, IL-22, IL-23, IL27, IL-28, CD40L, 4-1BBL, CD30L, OX40L, FLT3-L, APRIL, BAFF, RANTES, MIP, Erythropoietin, Thrombopoietin, SCF (stem cell factor), G-CSF, GM-CSF and M-CSF etc. In an embodiment, the first module is an antibody, an antibody fragment (e.g., scFv, vL, vH, Fab etc.), a single domain antibody (e.g., vHH, FHVH etc.) or a non-immunoglobulin antigen binding module that can bind to a receptor. In exemplary embodiments, the receptor is selected from one of the following: IL-1R, IL2R, IL-3R, IL-4R, IL-5R, IL-6R, IL-7R, IL-8R, IL-9R, IL-10R, IL-11R, IL-12R, IL-13R, IL-15R, IL-18R, IL-19R, IL-20R, IL-21R, IL-22R, IL-23R, IL-27R, IL-28R, CCR1, CCR3, CCR5, MIP-1R, PF4 receptor, Erythropoeitin-Receptor (Epo-R), TPO-R/MPL, GSF-R, c-Kit, and M-CSF receptor.

In an embodiment, a multipurpose switch serves as a life-death switch for the purpose of adoptive cell therapy when ectopically expressed in a cell. In an embodiment, the multipurpose switch comprises an in-frame fusion of a first module comprising a receptor-binding domain to a second module that serves as a kill-switch and a third module that serves as a membrane anchoring module. In an embodiment, the first module binds to a receptor that is expressed on cell surface, i.e., it binds to the extracellular domain of a receptor. In an embodiment, the first module binds to a receptor which when bound transmits a pro-survival and/or proliferative signal to the cell. In an embodiment, the first module binds to the receptor in cis (i.e., bind to the receptor expressed on the same cell as the cell expressing the molecular switch). In an embodiment, the first module binds to the receptor in trans (i.e., bind to receptor expressed on a cell other than the cell expressing the molecular switch). In an embodiment, the first module binds to the receptor in cis and in trans. In an embodiment, the second and the third modules are derived from the same endogenous protein. In an embodiment, the second and the third module are derived from different endogenous proteins. In an embodiment, the second module comprises of the extracellular domain of an endogenous protein (e.g., CD79b) or an epitope tag or a SHARP-tag (e.g., P11, P13, P16 etc.) thereof. In an embodiment, the extracellular domain of an endogenous protein (e.g., CD79b) or a tag (e.g., P11, P13, P16 etc.) thereof comprise the epitopes described herein. In an embodiment, there are multiple copies of the epitope tag present in the multipurpose switch. In an embodiment, the second module can be used to induce death of the cells expressing the molecular switch. In an embodiment, the second module can be used to induce death of the cells expressing the molecular switch when bound by an agent. In an exemplary embodiment, the agent that induces death of cells expressing the molecular switch when bound to the second module is a SABR. In an embodiment, the SABR is an antibody, a single domain antibody, a non-immunoglobulin antigen binding domain, an antibody drug conjugate, a bispecific antibody, or a fragment thereof. In an embodiment, the second module can be used to selectively enrich or deplete cells expressing the molecular switch. In an embodiment, the second module can be used to selectively detect, enrich and/or deplete cells expressing the molecular switch when bound by an agent. In an exemplary embodiment, the agent that can be used to selectively detect, enrich and/or deplete the cells expressing the molecular switch when bound to the second module is a SABR, i.e., an antibody, a single domain antibody, a non-immunoglobulin antigen binding domain or a fragment thereof. In an embodiment, the molecular switch is used to selectively detect, enrich and/or deplete cells ex vivo. In an embodiment, the molecular switch is used to selectively deplete cells in vivo. In an embodiment, the SABR (i.e., an antibody, antibody drug conjugate, bispecific antibody, a non-immunoglobulin antigen binding domain or a fragment thereof) that is used to detect, deplete, or enrich cells expressing the molecular switch has been approved for human administration by the US FDA or an equivalent regulatory agency in another country. Agents that have been approved by the US FDA for human administration are known in the art and include, but are not limited to, Polatuzumab (Polivy) etc. In an embodiment, the agent that is used to detect, deplete, or enrich cells expressing the molecular switch is approved by the FDA for ex vivo clinical use. An example of such agent is an antibody against CD34 that has been approved by the FDA to be used in conjunction with the clinically approved CliniMACS CD34 system (Miltenyi). In an embodiment, multipurpose switches are encoded by nucleic acids represented by CD8SP2-P13QP13-IL2-SythCD8TM (SEQ ID NO: 3115) and IL15-CD79b-Iso2-TM-CP4 (SEQ ID NO: 3116) etc. These multipurpose switches when expressed in immune cells (e.g., T cells or NK cells etc.) provides them with a survival signal by binding to the IL2 or IL15 receptor through the N-terminal module comprising IL2 or IL15. The second module of this multipurpose switch comprises the extracellular domain of CD79b or P11QP11 which is recognized by P11-binding agents (e.g., SN8 or Polatuzumab) and can be used for the detection, selective depletion and/or enrichment of transgene (e.g., SAR) expressing cells. The extracellular domain of CD79b or CD79b isoform 2 comprising the second module can be also used for selective suicide of transgene (e.g., SAR) expressing cells by the use of the epitope-targeted agents (e.g., SABR), such as an antibody or an antibody drug-conjugates targeting the SHARP-tag (e.g., SEQ ID NO: 674, 557 etc.). The third module in this molecular switch comprises of the hinge and/or transmembrane domain of CD79b-Isoform 2 and serves to anchor the switch to the cell membrane. In an embodiment, the second module is a synthetic module comprising one or more copies of a SHARP-tag described here. In an embodiment, the epitope is present in the extracellular domain of an endogenous protein. An example of a synthetic module comprising two or more copies of an epitope is P11QP11, a module harboring a CD34 epitope and two P11 epitopes. The P11QP11 module allows selection with the clinically approved CliniMACS® CD34 system (Miltenyi Biotec, Germany). As the multipurpose switches are modular in format, one module can be replaced with a different module. Thus, the IL2 module can be replaced by a different cytokine (e.g., IL15, IL18, IL21 etc.). These multipurpose proteins provide a pro-survival signal through their cytokine moiety (e.g., IL2, IL15, IL18, IL21 etc.) but can be used to kill-off the cells by the use of an agent (e.g., an antibody) that binds to the second module, thereby acting as a suicide gene that allow selective deletion of administered T cells in the face of toxicity. The second module (e.g., P11QP11 etc.) can be also used as a marker for measurement of transduction and to allow selection of transduced cells. The second module can be replaced by any of the SHARP-tags described herein (e.g., SEQ ID NO: 1-123, 150-167, 251-440, 550-579, 631-650, 651-654, 656, 674-676, 686, 689, and 692-720).

The multipurpose switch expression cassettes are compact in size and can be easily packaged in viral vectors. They are much more manageable size than the expression cassettes encoding individual marker, suicide, and survival genes, which would require separate promoters. They have the added advantage of comprising survival, suicide, and marker gene elements with sensitivity at least equal to that demonstrated by the individual genes.

In an embodiment, the second module (D2) is a synthetic module comprising one or more copies of an epitope or a mimotope. In an embodiment, the epitope is present in the extracellular domain of an endogenous protein. The polynucleotide encoding the multipurpose switches of the disclosure may comprise or consist of a variant of the sequence shown as SEQ ID No. 3115-3116, which has at least 70%, 80% or 90% identity with the sequence shown as SEQ ID No. 3115-3116, as long as it encodes for a polypeptide that retains the functional activity of the polypeptide encoded by SEQ ID No. 3115-16.

The multipurpose molecular switches of the disclosure can be in the form of a fusion protein, in which the polypeptide is fused to a protein of interest (POI). The fusion protein may comprise a self-cleaving peptide (e.g., P2A or F2A) between the polypeptide encoding the multipurpose switch and the protein of interest. The protein of interest is a molecule for expression at the surface of a target cell. The POI may exert a therapeutic or prophylactic effect when the target cell is in vivo. The POI may be a SAR (e.g., a CAR, SIR, zSIR, HIT, STAR, cTCR, Ab-TCR, TFP, TAC, KIR-CAR, recombinant TCR etc.) or an endogenous TCR.

The disclosure also provides a nucleic acid sequence capable of encoding a multipurpose switch encoding polypeptide or fusion protein of the disclosure.

The nucleic acid, when expressed by a target cell, causes the encoded multipurpose switch polypeptide to be expressed at the cell-surface of the target cell. Where the nucleic acid encodes both the multipurpose switch polypeptide and POI (for example as a fusion protein), it should cause both the polypeptide of the disclosure and the POI to be expressed at the surface of the target cell. The nucleic acid sequence may be RNA or DNA, such as cDNA. The disclosure also provides a vector which comprises a nucleic acid sequence of the multipurpose molecular switch. The vector may also comprise a transgene of interest, i.e., a gene encoding a POI (e.g., a SAR).

The vector should be capable of transfecting or transducing a target cell, such that they express the polypeptide encoding the multipurpose switch and optionally a protein of interest.

The vector may be a non-viral vector such as a plasmid. The vector may be a viral vector, such as a retroviral or lentiviral vector. The vector may comprise a nucleic acid encoding the polypeptide and a nucleic acid comprising the POI as separate entities, or as a single nucleotide sequence. If they are present as a single nucleotide sequence, they may comprise one or more internal ribosome entry site (IRES) sequences between the two encoding portions to enable the downstream sequence to be translated. In an embodiment, the multipurpose molecular switch and the POI may be expressed from a single vector using separate promoters. In another embodiment, the multipurpose switch and the POI may be expressed from separate vectors.

The disclosure also provides a cell which expresses a multipurpose switch polypeptide of the disclosure. The cell may co-express the multipurpose switch polypeptide and a POI (e.g., a SAR) at the cell surface. The disclosure also comprises a cell population which comprises a cell according to the disclosure. The disclosure provides a method for measuring transduction with a transgene of interest (which encodes a protein of interest POI, e.g., a SAR), which comprises the step of transducing a population of cells with a vector which co-expresses the multipurpose switch polypeptide of the disclosure and the protein of interest (e.g., a SAR) and detecting expression of the multipurpose switch on the surface of cells, wherein the proportion of cells expressing the multipurpose switch polypeptide of the disclosure corresponds to the proportion of cells transduced with the transgene of interest.

The disclosure also provides a method for selecting cells expressing a POI (e.g., a SAR) which comprises the following steps:
(i) detecting expression of the multipurpose switch on the surface of cells transfected or transduced with a vector of the disclosure which comprises a nucleotide sequence encoding the POI (e.g., a SAR); and
(ii) selecting cells which are identified as expressing the multipurpose switch.

Cells may be identified and/or sorted by methods known in the art such as FACS or Miltenyi cliniMACS® system.

The disclosure also provides a method for preparing a purified population of cells enriched for cells expressing a POI (e.g., SAR) which comprises the step of selecting cells expressing a POI (e.g., a SAR) from a population of cells using the method described above.

The disclosure also provides a method for tracking transduced cells in vivo which comprises the step of detection of expression of the polypeptide of the disclosure at the cell surface. Cells may be tracked in vivo by methods known in the art such as bioluminescence imaging. For such applications, the polypeptide of the disclosure may be engineered to be co-expressed with a detectable protein, such as luciferase or a luciferase fragment, such as HiBIT (e.g., SEQ ID NO: 721). The HiBIT tag can be used to detect the cells using complementation assay with LgBIT that is known in the art.

The disclosure also provides a method for deleting cells transduced by a vector according to the disclosure, which comprises the step of exposing the cells to an agent that binds to the multipurpose switch polypeptide. In an embodiment, the agent binds to the D2 domain of the multipurpose switch polypeptide. In an embodiment, the agent is an antibody (e.g., SN8, 2F2, Polatuzumab, huMA79b or H2Mab-250) and cells are exposed to the antibody in the presence of complement. In an embodiment, the agent is an antibody drug conjugate (e.g., Polatuzumab-vedotin or Polivy etc.). In an embodiment, the multipurpose switch comprises CD79b or the variants or fragments thereof and the agent is SN8, 2F2, Polatuzumab-vedotin (Polivy), or huMA79b. In an embodiment, the multipurpose switch comprises a SHARP-tag represented by SEQ ID NO: 557-566 or a functional variant thereof and the agent is H2Mab-250 or a functional variant or a derivative thereof.

When the multipurpose switch polypeptide of the disclosure is expressed at the surface of a cell, binding of the SABR (e.g., SN8, 2F2, huMA79b, Polatuzumab-vedotin or H2Mab-250 etc.) to the D2 domain of the polypeptide causes lysis of the cell. More than one molecule of agent (e.g., Polatuzumab-vedotin) may bind per multipurpose switch polypeptide expressed at the cell surface. Deletion of cells may occur in vivo, for example by administering the agent (e.g., Polatuzumab-vedotin, SN8 etc.) to a patient.

A technical challenge with the next generation SAR constructs (e.g., SIR, zSIR, Ab-TCR, HIT, STAR etc.) is the lack of an easy method for their detection, isolation, purification, or depletion. The addition of the kill switches on the CAR constructs have been described but suffer from the problems of interfering with the SAR binding to its target antigen and/or off-target signaling. The applicant has discovered that cytokines (e.g., IL2 and 1L15 etc.) can be fused in frame to the membrane anchored form of a molecule (e.g., CD79b) or a fragment thereof (e.g., P11 etc.) without interfering with their binding and signaling activity. The disclosure provides cytokines (e.g., IL2 and IL15 etc.) and their low affinity variants fused to a membrane anchored molecule (e.g., CD79b, P11QP11). The fusion construct may also comprise one or more additional epitope tags (e.g., SEQ ID NO: 1-123, 150-167, 251-440, 550-579, 631-650, 651-654, 656, 674-676, 686, 689, and 692-720 etc.) that can be used for detection, isolation, purification and or depletion of the cells expressing them, including SAR expressing cells. These epitope tags can be used for detection, isolation, purification and or depletion of the immune cells (e.g., SAR-expressing NK or T cells) using the methods described in PCT/US2021/022641.

Adoptive transfer of genetically modified T cells is an attractive approach for generating desirable immune responses, such as an anti-tumor immune response. The disclosure provides a method for treating and/or preventing a disease in a subject, which comprises the step of administering a cell according to the disclosure to the subject. The method may comprise the step of administering a population of cells to a subject. The population of cells may be enriched for cells expressing a transgene of interest using a method described above. The method may involve the following steps:
(i) taking a sample of cells, such as a blood sample from a patient,
(ii) extracting the T-cells,
(iii) transducing or transfecting the T cells with a vector of the disclosure which comprises a nucleic acid sequence encoding the multipurpose switch and a transgene (e.g., SAR) of interest,
(iv) expanding the transduced cells ex-vivo
(v) returning the cells to the patient.
   The transduced cells may possess a desired therapeutic property such as enhanced tumor specific targeting and killing.

The invention further provides a method for isolating a tag suitable for biotechnology, genetic engineering and cell and gene therapy applications. The method involves the steps comprising of one or more of the following a) obtaining the sequence of an endogenous protein(s) and its isoforms; b) optionally analyzing the sequence(s) to determine the type of the protein; c) optionally determine the location of the signal peptide and transmembrane domain; d) determine the sequence of the N-terminal, C-terminal, juxta membrane, hinge, stalk, linker regions of the protein; e) optionally analyzing the secondary structure of the protein; f) select the regions of the protein that are located i) C-terminal to the signal peptide in the case of type I transmembrane protein or a membrane anchored protein, ii) near the C-terminal of a type II protein, iii) in the juxta membrane region of a protein facing the extracellular side, iv) unfolded region of the protein, v) random coil or unstructured region of a protein, vi) in the loops connecting secondary structure elements within a protein; vii) hinge, stalk, connecting peptide or linker regions of a protein; viii) extracellular region of a protein; ix) various combination of i) to viii); g) optionally check the hydrophilicity and polarity of the regions; h) optionally check that the region is not a mutational hot spot and/or is not associated with a congenital or acquired disease; i) optionally check whether the region identified above is recognized by a drug that is approved by a regulatory agency and/or is in clinical development; j) optionally generate recombinant polypeptides (e.g., SAR) incorporating the one or more copies of the regions identified above as tags; k) optionally check the expression and/or activity of the tagged polypeptide using appropriate assays; 1) optionally develop an antibody or a binding reagent against the tag; m) optionally check the ability of the antibody or the binding reagent to recognize the tag and/or the tagged polypeptide.

In some embodiments, the invention relates to a recombinant DNA construct comprising sequences encoding a SAR as defined above. In some embodiments, the SAR comprises an extracellular domain, wherein said extracellular binding domain comprises at least one SABR-specific epitope (SHARP-tag) to be bound by an epitope-specific mAb or SABR for in vitro cell sorting and/or in vivo cell depletion of T cells expressing said SAR.

According to one aspect, the invention relates to a method for in vitro sorting the recombinant fusion protein (e.g., TAG-SAR)-expressing immune cell, comprising contacting a population of said engineered immune with a SABR (preferably monoclonal Antibodies or mAbs) to collect only cells expressing the recombinant fusion protein (e.g., TAG-SAR).

In some embodiments, the invention relates to a method for in vitro sorting SAR-expressing immune cell, wherein said TAG-SAR comprises at least one extracellular binding domain comprising at least one SHARP-specific epitope as described above, comprising contacting a population of said immune cells with a monoclonal antibody specific for said SHARP-specific epitope to collect only said TAG-SAR-expressing immune cell.

In some embodiments, the methods described herein apply to all proteins comprising the SHARP-tag. The methods also apply to all host cells expressing a protein comprising a SHARP-tag

In some embodiments, the invention relates to a method for in vitro sorting TAG-SAR-expressing immune cells, wherein said TAG-SAR comprises at least one extracellular binding domain comprising at least one SHARP-tag, comprising contacting a population of said immune cells with an epitope-specific SABR specific for said SHARP-tag, selecting the cells that bind to the SABR to obtain a population of cells enriched in TAG-SAR-expressing immune cell.

In some embodiments, said SABR (e.g., monoclonal antibody) specific for said SHARP-tag epitope is conjugated to a fluorophore and the step of selecting the cells that bind to the monoclonal antibody is done by Fluorescence Activated Cell Sorting (FACS).

In some embodiments, said SABR (e.g., monoclonal antibody) specific for said SHARP-tag is conjugated to a magnetic particle and the step of selecting the cells that bind to the monoclonal antibody is done by Magnetic Activated Cell Sorting (MACS).

In some embodiments, the extracellular binding domain of the SAR comprises a SHARP-tag of SEQ ID NO 1-123, 150-167, 251-440, 550-579, 631-650, 651-654, 656, 674-676, 686, 689, and 692-720.

In some embodiments, the extracellular binding domain of the SAR comprises a SHARP-tag of SEQ ID NO 1-123, 252-252, 557-566 and the antibody used to contact the population of immune cells is Polatuzumab vedotin, 2F2, SN8 or hu.MA79b or H2Mab-250 or functional variant thereof.

In some embodiments, the population TAG-SAR-expressing immune cells obtained when using the method for in vitro sorting TAG-SAR-expressing immune cells described above, comprises at least 70%, 75%, 80%, 85%, 90%, 95% of SAR-expressing immune cells. In some embodiments, the population TAG-SAR-expressing immune cells obtained when using the method for in vitro sorting TAG-SAR-expressing immune cells described above, comprises at least 85% TAG-SAR-expressing immune cells.

In some embodiments, the population of TAG-SAR-expressing immune cells obtained when using the method for in vitro sorting SAR-expressing immune cells described above shows increased cytotoxic activity in vitro compared with the initial (non-sorted) cell population using the protocol described here. In a preferred embodiment, said cytotoxic activity in vitro is increased by 10%, 20%, 30% or 50%.

Preferably, the SABR (e.g., mAbs) are previously bound onto a support such as a column or on beads such as routinely realized by the skilled in the art.

According to a favored embodiment, immune cells are T-cells, NK cells, NKT cells, monocytes, or macrophages.

According to the invention, cells to be administered to the recipient may be enriched in vitro from the source population.

Methods of expanding source populations are well known in the art, and may include selecting cells that express an antigen such as SHARP-tag, using combinations of density centrifugation, immuno-magnetic bead purification, affinity chromatography, and fluorescent activated cell sorting, known to those skilled in the art.

In an embodiment, the method used for sorting cells expressing a SHARP-tagged protein (e.g., TAG-SAR) is the Magnetic-Activated Cell Sorting (MACS).

Amongst other technique, FACS is a technique of choice to purify cell populations of known phenotype as very high purity of the desired population can be achieved, or when the target cell population expresses a very low level of the identifying marker, or when cell populations require separation based on differential marker density.

In an embodiment of the invention, the mAb used in the method for sorting T cells expressing the SAR is chosen 2F2, SN8, huMA79b, 10D10 or H2Mab-250 or a variant.

### Method for Depleting SHARP-Tagged-Expressing Immune Cells

By “in vivo depletion” is meant in the present invention the administration of a treatment to a mammalian organism aiming to stop the proliferation of SHARP-tag-expressing immune cells (e.g., TAG-SAR) by inhibition or elimination.

One aspect of the invention is related to a method for in vivo depleting an engineered immune cell expressing a TAG-SAR comprising an SHARP-tag as previously described, comprising contacting said engineered immune cell or said TAG-SAR-expressing immune cell with at least one epitope-specific SABR or mAbs. Another aspect of the invention relates to a method for in vivo depleting immune TAG-SAR-expressing immune cell which comprises a SHARP-tag by contacting said engineered immune cell with epitope-specific antibodies. The method, however, can apply to a cell that expresses any protein that comprises a SHARP-tag on its extracellular domain.

Preferably, said immune cells are T-cells and/or the SABR are monoclonal antibodies.

According to one embodiment, the in vivo depletion of immune engineered cell is performed on engineered immune cell which has been previously sorted using the in vitro method of the present invention. In this case, this will be the same infused mAb used.

According to an embodiment, the SHARP-tag is a CD79b peptide antigen (e.g., SEQ ID NO: 1-123, 150-167, 251-440) and the epitope-specific mAb is Polatuzumab, SN8, 2F2, H2Mab-250 or a functional variant thereof.

According to an embodiment, the SHARP-tag is a Her2 peptide antigen (e.g., SEQ ID NO: 557-566) and the epitope-specific mAb is H2Mab-250 or a functional variant thereof.

In some embodiments, the invention relates to a method for in vivo depleting an engineered immune cell expressing a TAG-SAR comprising an SHARP-tag (SAR-expressing immune cell) as previously described, in a patient comprising contacting said TAG=SAR-expressing immune cell with at least one epitope-specific SABR (e.g., mAb).

In an embodiment of the invention, the mAb used in the method for depleting an engineered immune cell expressing a TAG-SAR is chosen amongst Polatuzumab vedotin, 2F2, huMA79b, SN8 and H2Mab-250.

In some embodiments, the step of contacting said engineered immune cell or said SAR-expressing immune cell with at least one epitope-specific mAb comprises infusing the patient with epitope-specific SABR (e.g., mAb), preferably Polatuzumab vedotin, 2F2, huMA79b, SN8 or H2Mab-250 or their variants. In some embodiments, the amount of SABR (mAb) administered to the patient is sufficient to eliminate at least 20%, 30%, 40%, 50%, 60%, 70%, 80% or 90% of the TAG-SAR-expressing immune cell in the patient.

In some embodiments, the step of contacting said engineered immune cell or said TAG-SAR-expressing immune cell with at least one epitope-specific mAb or SABR comprises infusing the patient with 1-2 mg/Kg of Polatuzumab vedotin, once or several times, preferably once weekly.

In some embodiments, when immune cells expressing a SAR comprising an SHARP-tag (CAR-expressing immune cells) are depleted in a CDC assay using epitope-specific mAb, the amount of viable SAR-expressing immune cells decreases, preferably by at least 10%, 20%, 30%, 40%, 50%, 60%, 70%, 80% or 90%. Preferably the CDC assay is the assay disclosed in Example 3, Example 4 or Example 7.4. In some embodiments, said SHARP-tag is a SEQ ID NO: 1-123, preferably SEQ ID NO 1, 2, 3, 50-59 and the epitope-specific mAbs is Polatuzumab vedotin.

To one particular embodiment, the in vivo depletion of SAR-engineered immune cells is performed by infusing bi-specific antibodies or bispecific T cell engager.

According to another particular embodiment, the infused bi-specific mAb is able to bind both the SHARP-tag (e.g., tag, e.g., P7, P8, P9, P10, P11 etc.) borne on engineered immune cells expressing and to a surface antigen on an effector and cytotoxic cell.

According to a particular embodiment, a cytotoxic drug is coupled to the epitope-specific mAbs or SABR which are used in to deplete TAG-SAR-expressing immune cells, By combining targeting capabilities of monoclonal antibodies with the cancer-killing ability of cytotoxic drugs, antibody-drug conjugate (ADC) allows a sensitive discrimination between healthy and diseased tissue when compared to the use of the drug alone.

According to another particular embodiment, the epitope-specific mAb or SABR to be infused is conjugated beforehand with a molecule able to promote complement dependent cytotoxicity (CDC). Therefore, the complement system helps or complements the ability of antibodies to clear pathogens from the organism. When stimulated by one of several, is triggered an activation cascade as a massive amplification of the response and activation of the cell-killing membrane attack complex.

In some embodiments of the invention, the epitope-specific mAb or SABR used in the method for sorting and depleting an engineered immune cell expressing a SAR is the same and is chosen amongst Polatuzumab vedotin, 2F2, SN8, huMA79b and H2Mab-250.

In some embodiments of the invention, different antibodies are used for sorting and depleting the cells. SARs and immune cells comprising them have been extensively disclosed and can be prepared by the skilled person according to known methods.

The different methods described above involve expressing SAR at the surface of a cell. As a non-limiting example, said SAR can be expressed by introducing the latter into a cell. SARs can be introduced as transgene encoded by one plasmid vector. Said plasmid vector can also contain a selection marker which provides for identification and/or selection of cells which received said vector.

In another embodiment, isolated cell or immune cell expressing a SAR as described herein obtained by the different methods or cell line derived from said isolated cell as previously described can be used as a medicament. In another embodiment, said medicament can be used for treating pathologies such as cancer in a patient in need thereof.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efaliztimab treatment for psoriasis patients or other treatments for PML patients. In further embodiments, the T cells of the invention may be used in combination with chemotherapy, anti-androgen agents, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation.

The article of manufacture can comprise a container and a label or package insert on or associated with the container. Suitable containers include, for example, bottles, vials, syringes, etc. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a target antigen-positive cancer or a target antigen-positive viral infection. Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a target antigen-positive disease or disorder described herein, optionally in combination with the articles of manufacture. Kits of the disclosure include one or more containers comprising a tag and/or SAR effector cell composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. A kit for detection or purification of a fusion protein may comprise a nucleic acid or a nucleic acid expression construct encoding a peptide/epitope tag as defined herein, which may be present in the fusion protein. The nucleic acid may comprise a site, such as a cleavage or recombination site, that facilitates genetically fusing a polypeptide to the peptide/epitope tag. A nucleic acid sequence encoding the peptide/epitope tag may be operably linked to sequence elements that contain information regarding to transcriptional and/or translational regulation. The kit may also comprise an antibody of the invention, optionally conjugated to a detectable label described herein, preferably an optically detectable label or an affinity tag. Alternatively or additionally, the kit may comprise a detectable moiety that can be conjugated to the antibody of the invention. The kit may also comprise buffers and reagents necessary for the isolation/purification and/or detection methods of the present invention. The kit may also comprise buffers and reagents necessary to introduce the nucleic acid or the nucleic acid expression construct comprised in the kit into a host cell. The kit may also comprise at least one (secondary) specific binding partner as described herein or a (further) specific binding partner that specifically binds the (secondary) specific binding partner as described herein. The kit may also comprise a solid support comprising the antibody of the invention immobilized or attached to the solid support. The kit may also comprise an isolated peptide as described herein suitable for competitive elution of a fusion protein bound to an antibody of the invention, or other means for elution of the fusion protein, such as a proteinase. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The instructions relating to the use of the SAR effector cell compositions generally include information as to dosage, dosing schedule, and route of administration for the intended treatment. The containers may be unit doses, bulk packages (e.g., multi-dose packages) or sub-unit doses. Kits may also include multiple unit doses of the SAR and pharmaceutical compositions and instructions for use and packaged in quantities sufficient for storage and use in pharmacies, for example, hospital pharmacies and compounding pharmacies.

The invention is further described by reference to the following experimental examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### Examples

Cells were cultured at 37° C., in a 5% CO₂ humidified incubator. The cell lines were obtained from ATCC, NIH AIDS reagent program or were available in the laboratory. Jurkat cell line (clone E6-1) engineered with a NFAT-dependent EGFP (or GFP) reporter gene and named JNG was a gift from Dr. Arthur Weiss at University of California San Francisco and have been described to study CAR-signaling ((Wu, C Y et al., Science 350:293-302, 2015). Jurkat cells were maintained in RPMI-1640 medium supplemented with 10% FBS. NK92MI cells were obtained from ATCC and were maintained as per the instructions provided. NK92 cells were also obtained from ATCC and maintained in RPMI medium with 20% FBS and 200 U/mL of hIL2. T2 cells were from ATCC.

### Generation of Lentiviral Vectors Encoding SARs

The SAR constructs were cloned in the lentiviral, retroviral or sleeping beauty transposon vectors. Exemplary vectors are provided in SEQ ID NO: 1-6. Other vectors that can be used for generating SARs of the disclosure are known in the art. The psPAX2 vector was a gift from Didier Trono (Addgene plasmid #12260). The pLP/VSVG envelope plasmid and 293FT cells were obtained from Invitrogen (Thermofisher Scientific). The retroviral transfer vector MSCVneo, MSCVhygro, and MSCVpac and the packaging vector pKAT have been described previously (PCT/US2018/53247). The methods for generation of SAR (e.g., 2^{nd} generation CARs, SIRs, Ab-TCR and TFP etc.), the generation and use of GGS-NLuc fusion proteins, and the generation and use of luciferase (e.g., GLuc and Luc146-1H2) reporter cell lines for measurement of cellular cytotoxicity using the Matador assays have been described (PCT/US2017/024843, PCT/US2017/025602, PCT/US2017/052344, PCT/US2017/064379 and PCT/US2018/53247), which are incorporated in their entirety by reference herein.

The sequences comprising the antigen binding domains of SAR are codon optimized and synthesized artificially using publicly available software (e.g. Thermofisher or IDT) and commercial vendors (e.g. IDT). The resulting fragments are PCR amplified and cloned in different vectors containing the different SAR backbones using standard Molecular Biology techniques. In general SAR constructs are typically cloned in a lentiviral vector. The sequences of the SIR constructs are confirmed using automated sequencing.

### Lentivirus and Retrovirus Vectors

Lentiviruses were generated in 293FT cells by transfection of transfer plasmids encoding the different SAR constructs and 2^{nd} or 3^{rd} generation packaging plasmids using polyethylene amine (PEI) essentially as described previously (Natarajan et al, Scientific Reports, 10:2318) and PCT/US2018/53247.

### Infection of T Cells, NK Cells and PBMC

Buffy coat cells were obtained from healthy de-identified adult donors from the Blood Bank at Children Hospital of Los Angeles and used to isolate peripheral blood mononuclear cells (PBMC) by Ficoll-Hypaque gradient centrifugation. PBMC were either used as such or used to isolate T cells or NK cells using magnetic microbeads (Miltenyi Biotech) and following the manufacturer's instructions. PBMC or isolated T cells were re-suspended in XVIVO medium (Lonza) supplanted with 10 ng/ml CD3 antibody, 10 ng/ml CD28 antibody and 100 IU recombinant human-IL2. Cells were cultured at 37° C., in a 5% CO2 humidified incubator. Cells were activated in the above medium for 1 day prior to infection with lentiviral vectors. In general, primary cells (e.g., T cells) were infected in the morning using spin-infection (1800 rpm for 90 minutes at 37° C. with 300 μl of concentrated virus that had been re-suspended in XVIVO medium in the presence of 8 g/ml of Polybrene® (Sigma, Catalog no. H9268). The media was changed in the evening and the infection was repeated for two more days for a total of 3 infections. After the 3rd infection, the cells were pelleted and resuspended in fresh XVIVO media containing 10 ng/ml CD3 antibody, 10 ng/ml CD28 antibody and 100 IU recombinant human-IL2 and supplemented with respective antibiotics (if indicated) and place in the cell culture flask for selection, unless indicated otherwise. Cells were cultured in the above medium for 10-15 days in case no drug selection was used and for 20-30 days in case drug-selection was used. For infection of JNG and cancer cell lines, approximately 500,000 cells were infected with 2 ml of the un-concentrated viral supernatant in a total volume of 3 ml with Polybrene® (Sigma, Catalog no. H9268). Then next morning, the cells were pelleted and resuspended in the media with respective antibiotics, where appropriate, and place in the cell culture flask for selection.

Antibodies, peptides and drugs: Digitonin was purchased from Sigma (Cat. no D141) and a stock solution of 100 mg/ml was made in DMSO. A diluted stock of 1 mg/ml was made in PBS. Final concentration of digitonin used for cell lysis was 30 g/ml unless indicated otherwise. Polatuzumab vedotin (Polivy) was from Genentech/Roche.

### FACS Analysis for Detecting Expression of SAR.

Biotinylated protein L was purchased from GeneScript (Piscataway, NJ), reconstituted in phosphate buffered saline (PBS) at 1 mg/ml and stored at 4° C. Streptavidin-APC (SA1005) was purchased from Thermofisher Scientific.

FACS was done using FACSVerse analyzer from BD Biosciences.

### Cell Death Assay

To measure cell death, Matador assay based on ectopic cytosolic expression of Glue, NLuc or thermostable beetle luciferase (LucPPe or Luc146-1H2; SEQ ID NO: 3528)) was utilized as described in PCT/US2017/052344 “A Non-Radioactive Cytotoxicity Assay”.

### Assay to Detect the Expression of Antigens on Target Cells and to Determine the Antigen Binding Activity of Various of Antigen Binding Moieties Used in the Construction of the SARs.

The expression of antigens on target cells was determined by bioinformatics in combination with immunostaining with antibodies or a highly sensitive antigen detection assay (Malibu Glo assays) as described in PCT/US2017/025602 and incorporated herein in its entirety by reference. This assay involves the fusion of a GLuc or NLuc reporter fragment to the antigen binding domain of an antibody, a scFv, a vHH or any other antigen binding fragment or any receptor and ligand. The fusion construct also carried several epitope tags for detection using flow cytometry. This assay was also used to detect the presence of tags on the TAG-Cells expressing the different TAG-polypeptides, including TAG-SAR constructs.

ELISA to Identify the Sequence Recognized by Polatuzumab, huMA79b, and 2F2.

A large panel of tagged fusion constructs were generated in which different tags recognized by Polatuzumab vedotin (SHARP-tags or Pola tags) (e.g., SEQ ID NO: 1-11) were fused in frame between an scFv domain (e.g., hu-mROO5-1) and a 4×FLAG-×2STREP-8×His cassette. Examples of such constructs are provided in Table 5 (SEQ ID NO (DNA): 5075-5457 and SEQ ID NO (PRT): 2625-3007). Expression constructs for different antibodies were also constructed. SHARP-tags (or Pola tag) supernatants and antibody supernatants were generated by transfecting 293FT cells with the SHARP-tags/antibody constructs. Forty-eight hours post-transfection supernatants were collected, filtered and stored at −20° C. for storage. The day before ELISA, SHARP-tags (PolSHARP-tag) containing supernatants were diluted 1:1 in carbonate bicarbonate buffer pH 9.6 to coat a 384-well clear microplate (Cat No. 464718, Thermofisher) with 50 μL/well of diluted supernatant. The plate was sealed with an adhesive strip and incubated at 4° C. for overnight. Next morning, each well was aspirated and washed with wash buffer (0.05% Tween20 in PBS). The process was repeated 2 times for a total of 3 washes. The plate was blocked by adding 50 μL block buffer (2% BSA in PBS) to each well. The plate was covered and incubated at 37° C. for 1 hour. The aspiration/wash step was repeated, and 50 μL/well of antibody containing supernatants were added in triplicate. The plate was covered and incubated at 37° C. for 1 hour. The aspiration/wash step was repeated followed by addition of 50 L/well anti-human IgG-HRP (Cat No. sc-2453, SCBT) at 1:5000 dilution in reagent diluent (1% BSA in PBS). The plate was covered and incubated at 37° C. for 1 hour. The aspiration/wash step was repeated. TMB (3,3′,5,5′ tetramethyl-benzidine) substrate was prepared by mixing hydrogen peroxide and TMB in 1:1 ratio and added 25 μL to each well. The plate was incubated at room temperature in dark until blue color was developed in the wells followed by addition of 25 μL/well of stop solution (2 N H₂SO₄) to each well. The absorbance was measured at 450 nm using BioTek synergy H4 plate reader. As shown in Table 5, SHARP-tags showed differential binding to supernatants containing huMA79b antibody (SEQ ID NO: 1182 and 1183) and CD79b-2F2-CY antibody (SEQ ID NO: 1189). In general, SHARP-tags (e.g., P8, P9, P11, P13, P14, P16 etc.) showed significantly higher binding with huMA79b antibodies as compared to CD79b-2F2-CY antibody. The sequences RSEDRY (SEQ ID NO: 1) RSEDRYR (SEQ ID NO: 2) were determined to be sufficient for binding by huMA79b antibody (SEQ ID NO: 1182 and 1183) and CD79b-2F2-CY antibody (SEQ ID NO: 1189). We also generated mutations of SHARP-tags (e.g., RSEDRYR, ARSEDRYR, ARSEDRYRNPK) and tested several point mutants for binding with huMA79b (SEQ ID NO: 1182 and 1183) and CD79b-2F2-CY (SEQ ID NO: 1189) antibodies. The following mutants based on SEQ ID NO: 1 or 2 were recognized by huMA79b and/or (SEQ ID NO: 1182 and 1183) and CD79b-2F2-CY (SEQ ID NO: 1189) in ELISA: R1E, R1S, R1N, R1D, R1F, R1H, S2T, S2A, S2L, S2V, S2M, S2F, S2G, S2H, S2I, D4S, D4N, D4G, D4A, D4T, D4K, D4L, D4W, D4R, D4V, D4Y, R5H, R5P, R5L, R5T, R5C, Y6M, Y6F, Y6H, Y6D, Y6A, Y6G, Y6I, and Y6V. In addition, mutants in which R (Arg) at position 7 of SEQ ID NO: 2 is mutated to a number of different amino acids (e.g., K, E, D, G, H, L, M, Q, V, W, S, F, I, Y, P, N, T or A) were recognized by huMA79b and CD79b-2F2-CY antibodies. Therefore, R (Arg) at position 7 can be mutated to any amino acid or any naturally occurring amino acid to be recognized by huMA79b and CD79b-2F2-CY antibodies or their variants. The following mutants based on SEQ ID NO: 1 or 2 were recognized by huMA79b antibody (SEQ ID NO: 1182 and 1183) or its variants: R1E, R1H, S2T, S2A, D4S, D4N, D4G, D4A, D4T, D4K, D4L, R5H and Y6M. In addition, mutants in which R (Arg) at position 7 in case of SEQ ID NO: 2 is mutated to several different amino acids (e.g., K, E, D, G, H, L, M, Q, V, W, S, F, I, Y, P, N, T or A) were recognized by huMA79b antibody and its variants. The following mutants based on SEQ ID NO: 1 or 2 were recognized by 2F2 antibody (SEQ ID NO: 1189) or its variants: R1E, R1S, R1N, R1D, R1F, R1H, S2T, S2A, S2L, S2M, S2F, S2G, S2H, S2V, S2I, D4G, D4A, D4W, D4R, D4V, R5H, R5P, R5L, R5T, R5C, Y6M, Y6F, Y6H, Y6D, Y6A, Y6G, Y6I, and Y6V. In addition, mutants in which R (Arg) at position 7 in case of SEQ ID NO: 2 is mutated to several different amino acids (e.g., K, E, D, G, H, L, M, Q, V, W, S, F, I, Y, P, N, T or A) were recognized by 2F2. The single point mutants of R7K, R7S, R7F, R7Y, R7A, R7I, R7P, R7N, and R5H showed nearly equivalent binding to huMA79b and CD79b-2F2-CY antibodies, but mutants D4S, D4N, D4G, D4K, D4T, D4Q, D4A showed higher binding affinity with huMA79b antibodies. In contrast, mutants Y6D, Y6A, Y6G, Y6F, Y6H, D4W, D4Y, S2A, S2F, S3H, S3I, S3G, S3L, S3M, S3T, R1S, R1H, R1D, R1S, R5P, R5L, R5T, R5C showed higher binding affinity with CD79b-2F2-CY antibody.

The above experiment further demonstrated the ability of huMA79b antibody (SEQ ID NO (PRT): 1182 and 1183 and SEQ ID NO (DNA): 3632-3633) and CD79b-2F2-CY antibody (SEQ ID NO (PRT): 1189 and SEQ ID NO (DNA): 3639) to recognize the SHARP-tags (or SHARP-tags). A number of variants of huMA79b and CD79b-2F2-CY antibody are generated and are represented by SEQ ID NO:1184 and SEQ ID NO: 1185-1188, respectfully. Additional variants of will be generated by replacing the variable regions (vL and vH) of huMA79b antibody with vL fragments represented by SEQ ID NO: 774-782, 790, 792-794, 808-818, 2173-2178 and the complementary vH fragment represented by SEQ ID NO:899-907, 915, 917-919, 937-941, 2179-2182 or variants of the forgoing sequences comprising up to 10 amino acid substitutions in the framework region. Similarly, additional variants of 2F2 will be generated by replacing the variable regions (vL and vH) of 2F2 antibody with vL fragments represented by SEQ ID NO: 783-789 and 791 and the complementary vH fragment represented by SEQ ID NO: 908-914 and 916 or variants of the forgoing sequences comprising up to 10 amino acid substitutions in the framework region. These antibody variants will be tested in the ELISA and found to bind to the recombinant polypeptides comprising SHARP-tags (e.g., SEQ ID NO: 1-10). Finally, ELISA was repeated using the Antibody (SN8) (Cat No. sc-18902, Santa Cruz). 384 well plates were coated overnight with the SN8 antibody at working concentration 5 μg/mL in carbonate bicarbonate buffer pH 9.6. Plates were washed, blocked with blocking buffer and after washes, 50 μl of supernatants containing the recombinant polypeptides (e.g., SEQ ID NO (PRT): 2643, 2663 and 2664) comprising the different SHARP-tags were added. After washes, the bound recombinant polypeptides were detected by the addition of Mouse anti DDDDK-Tag mAb (Cat No. AE024, ABclonal Technology). The absorbance (OD 450 nm) value for control supernatant was 0.084, while it increased to 0.330, 0.441 and 0.134 for supernatants comprising SHARP-tag comprising polypeptides with SEQ ID NO (PRT): 2643, 2663 and 2664, respectively, demonstrating specific binding.

### ELISA

Peptides (e.g., P8, P9, P10, P11) were synthesized by GenScript. Peptides were diluted to the working concentration 2 μg/mL in sodium carbonate buffer pH 9.6. A 384-well white plate (Cat No. 460372; Thermofisher) was coated at 4° C. overnight with 50 μL/well of diluted antigen. Plates were washed, blocked and then 50 μl of control medium or supernatant containing Malibu-Glo reagent (110323-BBeT2) was added. After washes, 50 μL of 1:400 CTZ in PBS was added and luminescence was read for 10 sec. Results showed increase in light production in peptide-coated wells in which supernatant containing the Malibu-Glo reagent was added as compared to control wells with the P11 peptide showing the highest nearly a 30-fold increase.

The peptides (e.g., P8, P9, P10, P11 etc.) were coated as above and ELISA was done using huMA79b (SEQ ID NO: 1182) and 2F2 (SEQ ID NO: 1189) antibody supernatants. The OD values using huMA79b (SEQ ID NO: 1182) were 0.103 for control peptide (SEQ ID NO: 1209) and increased to 0.125, 0.892, 0.271, 2.851, and 3.116 for peptides with SEQ ID NO; 47, 48, 49, 5, 11, respectively. The OD values using 2F2 (SEQ ID NO: 1189) antibody were 0.069 for buffer alone, 0.074 for control peptide and 0.074, 0.094, 0.132, 0.108 and 1.865 for peptides with SEQ ID NO: 47, 48, 49, 5, 11, respectively. These results confirm that isolated SHARP-tags can be recognized by SABRs.

### Induction of NFAT Promoter Driven GFP Expression.

A number of different single chain and multi chain SAR constructs with different structure (e.g., 2^{nd} generation CAR, double chain SIR, one and half chain SIR, zSIR, Ab-TCR, z16SAR, MC-SIR, MC-zSIR, MC-z16SAR etc.) targeting different antigens were constructed. While some constructs targeted a single antigen, others targeted more than one antigen or two different epitopes of a single antigen.

The different SARs were stably expressed in Jurkat NFAT-GFP cells by lentiviral mediated gene transfer, followed by selection with puromycin or blasticidin. No drug selection was used in case the construct lacked an antibiotic resistance gene. The Jurkat-NFAT-GFP cells are engineered in such a way that the IL-2 promoter, which carries NFAT binding sites, is cloned upstream of the GFP gene. These cells have been used to study signaling via TCR and CAR. The SAR-expressing Jurkat-NFAT-GFP are cocultured for 4-24 hours with the target antigen-expressing cell line (described above) and their ability to bind to the target antigen is assayed by measuring induction of GFP expression using Flow Cytometry. Cellular supernatant is collected and assayed for the induction of cytokines (e.g., IL2). The Jurkat-NFAT-GFP (parental) cells were used as control.

The results with different SARs are summarized in the summary Table 6. A SAR is considered positive in the assay in case the SAR expressing Jurkat-NFAT-GFP cells show greater % GFP positive cells when cultured with the target cell line as compared to parental Jurkat-NFAT-GFP cells. The signs +/−, +, 2+ etc. after the name of the cell lines indicate the relative degree of positivity on the Jurkat-NFAT-GFP assay as measured by the % GFP positive cells after culture of the SAR expressing Jurkat-NFAT-GFP cells with that cell line above the % GFP cells observed in parental Jurkat NFAT-GFP cells serving as controls. In general, the number +/−, +, 2+, 3+, 4+, 5+ refer to approximately 5%, 10%, 20%, 30%, 40% and 50% GFP positive cells. It is to be noted that the drug resistance gene (e.g., PAC) in the SAR constructs is optional and in vivo studies were conducted with SAR constructs lacking the PAC gene.

The results show that several single chain and double chain TAG-SAR constructs show strong positivity in the JNG-NFAT assay. These constructs vary in the composition of the SHARP-tags or the tag cassettes (e.g., P11, P13, P14, P16 etc.), the location of the tag cassette, the copies of tag cassettes, the antigen binding domains, and the signaling chains. For example, the construct with SEQ ID NO: 4754 and 4794 are single chain second-generation CARs targeting CD19 that contain a Pola-11 (P11) tag (SEQ ID NO: 674) inserted in the linker between the vL and vH fragments derived from hu-mROO5-1 antibody. The JNG cells expressing these constructs showed NFAT driven GFP on culture with RAJI and JEKO cells. The construct with SEQ ID NO: 4757 is a double chain SIR targeting CD19 that comprises a Pola-14 (P14) tag (SEQ ID NO: 53) inserted downstream of the signal peptide comprising the second polypeptide chain. The constructs with SEQ ID NO: 4758 and 4761 are also double chain SIR targeting CD19 that comprise a tag (Pola-11) inserted downstream of the signal peptide comprising the second polypeptide chain. The JNG cells expressing these constructs showed strong NFAT driven GFP on culture with RAJI (2-2.5+), JEKO (1-1.5+) and Daudi (2+) cells. The construct with SEQ ID NO: 4762 is a double chain SIR targeting CD19 and BCMA that comprises a tag (Pola-11) inserted downstream of the signal peptide comprising the first polypeptide chain. The JNG cells expressing this construct showed strong NFAT driven GFP on culture with RAJI (2.5+), and JEKO (1+) and Daudi (2+) cells. The constructs with SEQ ID NO: 4765, 4768, 4770 are double chain SIRs targeting CD19 that comprises a tag (Pola-11) inserted between the vL fragment and the TCRβ constant chain of first polypeptide chain. The construct with SEQ ID NO: 4774 is a double chain z16 SAR (CD3z-CD16 SAR) targeting CD19 that comprises a tag (Pola-11) inserted between the vL fragment and the signaling module of the first polypeptide chain. The construct with SEQ ID NO: 4777 is a double chain SAR targeting BCMA that comprises a tag (Pola-11) inserted between the vL fragment and the signaling module of the first polypeptide chain.

The construct with SEQ ID NO: 4787 is a double chain SAR targeting BCMA that comprises a tag (Pola-11) inserted between the vH fragment and the signaling module of the second polypeptide chain. The construct with SEQ ID NO: 4779 is a double chain SIR targeting CD19 that comprises a tag (Pola-11) inserted between the vH fragment and the TCRα constant chain of second polypeptide chain. The construct with SEQ ID NO: 4787 is a double chain SIR targeting CD19 that comprises a tag (Pola-11) inserted between the vH fragment and the signaling chain of the second polypeptide chain. The construct with SEQ ID NO: 4788 is a double chain SAR targeting CD19 that comprises a tag (Pola-11) inserted between the vH fragment and the CD8 hinge module of the second polypeptide chain. The construct with SEQ ID NO: 4795 is a double chain SIR targeting CD19 that comprises a tag (Pola-11) inserted downstream of the signal peptide comprising the second polypeptide chain. The constructs with SEQ ID NO: 4797-4799 are double chain SAR targeting CD19 that co-express a SHARP-tagged-polypeptide (e.g., CD79b-Iso2, CD79b-FL or CD8SP2-P16QP16-8). The constructs that co-express a SHARP-tagged polypeptide are indicated by C(+) in the Table 6. The construct with SEQ ID NO: 4801 is a double chain SAR targeting BCMA that comprises a tag (Pola-11) inserted between the vL fragment and the signaling module of the first polypeptide chain and a second tag (Pola-11) between the vH fragment and the signaling module of the second polypeptide chain. The construct with SEQ ID NO: 4802 is a double chain SAR targeting CD19 that comprises a tag (Pola-11) inserted between the vL fragment and the TCRβ constant chain of the first polypeptide chain and a second tag (Pola-11) between the vH fragment and the TCRα constant chain of the second polypeptide chain. The construct with SEQ ID NO: 4804 is also double chain SIR targeting CD19 that comprise a tag (P16QP16) comprising two P16 tags (SEQ ID NO: 55) separated by a “Q linker inserted downstream of the signal peptide comprising the second polypeptide chain. The Q linker is recognized by QBEND antibody.

The constructs with SEQ ID NO: 4807-4812 are hybrid chain SAR targeting CD19 that comprise a tag (Pola-11) inserted downstream of the signal peptide of the second polypeptide chain. The constructs with SEQ ID NO: 4818-4819 are double chain SAR targeting CD19 that comprises a tag (P8 or P9) inserted downstream of the signal peptide of the second polypeptide chain. The constructs with SEQ ID NO: 4832 and 4860 are double chain SIR targeting STEAP2 that comprise a tag (Pola-11) inserted downstream of the signal peptide comprising the second polypeptide chain. The construct with SEQ ID NO: 4838 is a double chain SIR targeting STEAP2 that comprises a tag (Pola-11) inserted downstream of the signal peptide comprising the first polypeptide chain and a second tag (Pola-11) inserted downstream of the signal peptide comprising the second polypeptide chain. The constructs with SEQ ID NO: 4833-4837, 4839 are double chain SAR targeting CLDN18.2 that comprises a tag (Pola-11) inserted downstream of the signal peptide of the second polypeptide chain. The construct with SEQ ID NO: 4840 is a double chain SAR targeting CLDN6 that comprises a tag (Pola-11) inserted downstream of the signal peptide of the second polypeptide chain. The construct with SEQ ID NO: 4859 is a double chain SAR targeting CD19 that comprises a tag (Pola-14) inserted between the vL fragment and the TCRβ constant chain of the first polypeptide chain and a second tag (Pola-11) between the vH fragment and the TCRα constant chain of the second polypeptide chain. The construct with SEQ ID NO: 4871 is a double chain SAR targeting CLDN6 that comprises a tag (Pola-11) inserted downstream of the signal peptide of the first polypeptide chain. The construct with SEQ ID NO: 4872 is a double chain SAR targeting CLDN18.2 that comprises a tag (Pola-11) inserted downstream of the signal peptide of the first polypeptide chain. The construct with SEQ ID NO: 4873 is a double chain SAR targeting CLDN18.2 that comprises a tag (Pola-14) inserted downstream of the signal peptide of the second polypeptide chain. The construct with SEQ ID NO: 4875 is a double chain SAR targeting PSMA that comprises a tag (Pola-11) inserted downstream of the signal peptide of the first polypeptide chain. The construct with SEQ ID NO: 4876 is a double chain SIR targeting CD19 and BCMA that comprises a tag (CYD11) represented by SEQ ID NO: 689 inserted downstream of the signal-peptide comprising the first polypeptide chain. The constructs with SEQ ID NO: 4892-94 are double chain SIR targeting STEAP2, CLDN18.2 and MSLN respectively that comprises a tag (Pola-11) inserted downstream of the signal peptide comprising the first polypeptide chain and a second tag (Pola-11) inserted upstream of the TCRα constant chain of the second polypeptide chain.

The constructs with SEQ ID NO: 4901-4907, 4967-8 are single or double chain SARs targeting different antigens that co-express a SHARP-tagged polypeptide (e.g., SEQ ID NO: 685, 658, and 657). The constructs with SEQ ID NO: 4912, 4925, 4929-31, 4933, 4935, 4937, 4938, 4939, 4980, 4981 are single or double chain SARs targeting different antigens that co-express a SHARP-tagged-polypeptide comprising a multipurpose switch (e.g., SEQ ID NO: 666, 682-684). The constructs with SEQ ID NO: 4793-96, 5005 are also double chain SIR targeting CD19 or CD19/BCMA that comprise a tag (SEQ ID NO: 2, 47) inserted downstream of the signal peptide comprising the first or the second polypeptide chain. The construct with SEQ ID NO: 5006 is a double chain zSIR targeting PSMA that comprises a tag (Pola-11) inserted downstream of the signal peptide of the second polypeptide chain. The constructs with SEQ ID NO: 5007-8 are double chain SIR targeting PSMA that comprises a tag (SEQ ID NO: 50 and 2) inserted downstream of the signal peptide of the first polypeptide chain. Several additional SAR constructs with a variety of SHARP-tags (e.g., SEQ ID NO: 1-11) and their variants are expressed in JNG cells and shown to be functional. Their expression is detected by flow cytometry using staining with the appropriate antibodies (e.g., SN8, 2F2, huMa79b).

Similarly, JNG cells will be transduced with SAR constructs comprising a variety of SHARP-tags. These SAR constructs are represented by SEQ ID NO: 7708-8703. The expression of the SAR is confirmed by staining with Protein L, using the Topanga Assay and or using the appropriate SABR (e.g., SN8, 2F2, huMA79b or H2Mab-250). The functional activity of the SAR is confirmed by JNG assay after overnight co-culture with target cell lines (e.g., RAJI and LNCaP). These results will demonstrate that the presence of SHARP-tags (e.g., SEQ ID NO: 1-123, 150-167, 251-440, 550-579, 631-650, 651-654, 656, 674-676, 686, 689, and 692-720) do not interfere with the expression and/or activity of the fusion proteins that incorporate them (e.g., SAR).

Taken collectively the above results with a wide range of unispecific, bispecific and multispecific TAG-SAR comprising single chain, double chain and hybrid signaling chains of different compositions and comprising a large number of antigen binding domains (e.g., vL, vH, vHH etc.) targeting a variety of antigens (e.g., CD19, BCMA, PSMA, STEAP2, CLDN6, CLDN18.2, Her2, MSLN etc.) demonstrate that tags can be inserted at a variety of locations (e.g., in the linker between vL and vH or vH and vL domains, upstream of transmembrane domain of a single chain SAR, between the hinge and scFv domain of a single chain SAR, at the N-terminus or near N-terminus of antigen binding domains of single or double chain SAR, between the linker between an AABD and a vL/vH fragment, downstream of vL/vH fragment etc.) without interference with the expression and activity of the SAR construct. The results further demonstrate that more than one tag can be inserted in a SAR construct. The results further demonstrate that the two or more tags can be identical or non-identical in composition.

In addition to TAG-SAR, the JNG assay was also used to test several non-tagged single chain (e.g., CAR), double chain (e.g., SIR), hybrid chain (e.g., HC-SIR) and multi-chain (e.g., MC-SAR) constructs targeting antigens such as CD79b, CLDN6, CLDN18.2, PSMA, PSCA, BCMA, CD19, GPC3 etc. The results with these constructs are summarized in Table 6.

Table 6 also shows results with several unispecific and bispecific multi-chain SAR constructs (e.g., SEQ ID NO: 8740-8803, 8931-8977) on different designs (e.g., MC-SAR, MC-zSAR, and MC-16SAR). Examples of multichain SAR constructs targeting CD79b are SEQ ID NO: 8931-8933 and 8951-8959. Examples of multichain SAR constructs targeting CD19, CD20 or CD22 are SEQ ID NO: 8788, 8778, 8752 and 8785). Examples of bispecific multichain SAR constructs are SEQ ID NO:8741, 8744, 8745, 8750, 8751, 8753-8755, 8757-8759 and 8761-8784. Examples of multichain SAR with a CD3z signaling chain (MC-zSAR) are SEQ ID NO: 8802-8803. Examples of multichain SAR with a CD16 signaling chains (MC-16SAR) are SEQ ID NO:8970-8971.

The JNG cells expressing the different TAG-SAR constructs are next stained with the appropriate antibodies (e.g., SN8, 2F2, huMa79b, 10D10 etc.) and/or Malibu Glo reagents directed against the tag and the presence of the TAG-SAR is detected using Flow cytometry and/or luminometer. In parallel the JNG cells are stained with Protein L. It is determined that staining with anti-tag antibodies and/or Malibu Glo reagents result in detection of the TAG-SAR with greater sensitivity and specificity as compared to staining with Protein L.

### Detection of TAG-SAR Constructs Using Malibu-Glo Reagents

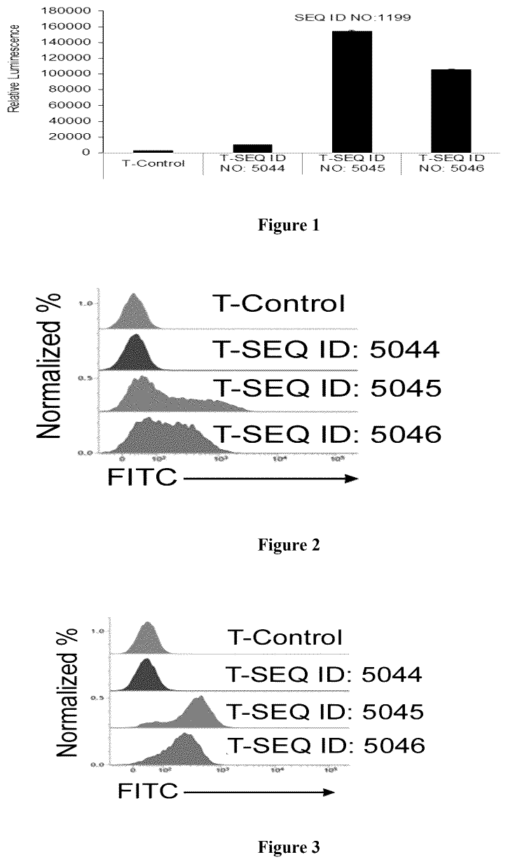

To detect the expression of TAG-SAR constructs, an Nluc fusion protein (Malibu Glo reagent) was used that comprises an scFv targeting human CD79b fused in frame to NLuc (NanoLuc) via a Gly-Ser linker. The fusion protein also carries a 4×FLAG-×2STREP-8×His cassette at its C-terminus for detection by flow cytometry. The fusion protein construct is represented by SEQ ID NO (PRT): 1199 and SEQ ID NO (DNA): 3649. The assay was performed essentially as described previously (see PCT/US2017/025602). Using this assay, JNG cells expressing the SAR construct with SEQ ID NO: 4754 showed a 386-fold increase in luciferase activity over the control JNG cells. This SAR construct comprises a SHARP-tag (P11; SEQ ID NO:674) in the linker between the vL and vH fragment. Essentially similar results were obtained with JNG cells expressing a variety of SAR constructs of different structure and comprising different SHARP-tags of different lengths and composition (e.g., SEQ ID NO:1-12), including variants. For example, JNG cells expressing SAR constructs containing tags with SEQ ID NOs: 160, 167, and 168 exhibited a 47.5-fold, 85.6-fold, and 83.4-fold increase in luciferase activity, respectively, compared to control JNG cells. Malibu-Glo reagent also successfully detected the presence of TAG-SAR on primary T cells (FIG. 1.) These results demonstrate that the tag present in these TAG-SAR constructs can be detected by the Malibu-Glo reagent comprising the CD79b targeted-scFv as the antigen binding domain. Essentially similar results are obtained when the CD79b-targeted scFv in the Malibu-Glo reagent is replaced by a different CD79b-targeted scFv. This Malibu-Glo reagent is represented by SEQ ID NO (PRT): 1198 and SEQ ID NO (DNA): 3648. Finally, as the Malibu-Glo reagents also comprise the FLAG, Strep-tag and Poly-His tags, the expression of TAR-SAR in JNG cells was also detected by primary labeling with the Malibu-Glo reagents followed by secondary labelling with FITC-conjugated FLAG or Strep-tag antibodies and analysis by flow cytometry (see FIG. 2).

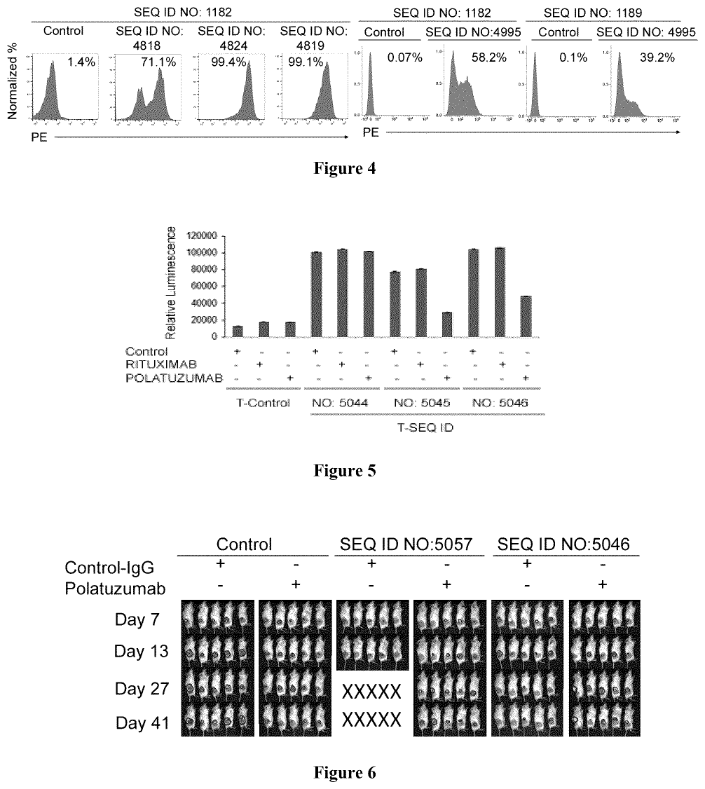

Detection of TAG-SAR Constructs Using SN8 Antibody JNG cells expressing the SHARP-tagged SAR construct with SEQ ID NO:4832 and 4852 were stained with FITC-conjugated antibody targeting CD79b (SN8 clone) on ice for 1 hour in dark. Post-incubation, Cells were washed with 1 mL of ice-cold wash buffer (1% FBS in PBS). After 2 washes, cells were re-suspended in 300 μL of buffer and analyzed by Flow Cytometry. Approximately 83% and 69% of JNG cells expressing the SAR construct with SEQ ID NO:4832 and 4852 showed positive staining as compared to the parental JNG cells or those expressing a control SAR lacking the SHARP-tag. The mean log fluorescence value increased from 191 in control JNG cells to 564 and 370 in JNG cells expressing SHARP-tagged SAR construct with SEQ ID NO:4832 and 4852. Essentially similar results were obtained when the experiment is repeated with JNG and T cells expressing different SAR constructs on a variety of backbones (e.g., CAR, SIR, Ab-TCR, zSIR, z16SAR, CD16-SAR, TFP, TCR etc.) and comprising SHARP-tags of different lengths and compositions (e.g., SEQ ID NO: 1-92). A representative example is provided in FIG. 3. The staining with SN8 antibody and/or SN8 coated beads are also used to isolate SHARP-tagged SAR expressing cells using a variety of techniques, FACS (fluorescence assisted cell sorting), MACS (Magnet assisted cell sorting), immune-affinity separation etc. Similarly, huMA79b and 2F2 antibodies and antibody-coated beads are used to detect, enrich, isolate and separate SHARP-tagged SAR expressing cells using techniques such as FACS, MACS and affinity purifications. Examples are provided in FIG. 4.
Detection of TAG-SAR Constructs Using huMA79b and 2F2 Antibodies

JNG cells transduced with lentiviral vectors encoding a variety of TAG-SAR constructs of different structure and comprising SHARP-tags of different lengths and composition (e.g., SEQ ID NO:150-168) were stained with supernatant containing the huMA79b antibody (Clone Code: 041924-BBhJ2)(SEQ ID NO: 1182) followed by Goat-anti-Human-PE secondary antibody. Representative TAG-SAR constructs are represented by SEQ ID NO: 4819-4820, 4824-4827. The cells were analyzed by flow cytometry. JNG cells expressing the TAG-SAR constructs stained positive with the huMA79b antibody (SEQ ID NO: 1182). For example, 97.6%, 98.9%, 99.5% and 98.5% of JNG cells expressing the TAG-SAR constructs with SEQ ID NO: 4819, 4820, 4824 and 4825, respectively, showed positive staining with the huMA79b antibody (FIG. 4). These results compare favorably with the staining observed with Protein-L and demonstrate that cells expressing the recombinant proteins (e.g., SIR, Ab-TCR, zSIR, HC-SIR etc.) comprising the SHARP-tags can be detected with very high sensitivity and specificity using the SABR (e.g., huMA79b antibody or its variants). Essentially similar results are obtained when the cells are stained with the 2F2 antibody (clone 041924-BBhO1; SEQ ID NO:1189) and its variants (FIG. 4). For example, JNG cells transduced with lentiviral vectors comprising the TAG-SAR constructs with SEQ ID NO (DNA): 4993, 4994 and 4995 showed 27.89%, 2.61% and 39.27% of cells, respectively, showing positive staining with the 2F2 antibody represented by SEQ ID NO:1189. The corresponding staining with the huMA79b antibody (SEQ ID NO: 1182) was seen in 38.48%, 51.90% and 58.28% of cells, respectively. JNG, T and NK cells transduced with a large panel of additional TAG-SAR constructs of different structure (e.g., 2^{nd} generation CAR, SIR, Ab-TCR, zSIR, z16-SAR, CD16-SAR, TFP etc.) and comprising SHARP-tags of different lengths and composition are tested and will show positive staining with huMA79b and 2F2 antibodies and their variants. Finally, JNG cells expressing other recombinant fusion proteins (e.g., membrane bound cytokines, multi-purpose switches etc.) comprising SHARP-tags show positive staining with SABR, including huMA79b, 2F2, SN8 and 10D10 and their variants. The staining with these antibodies and antibody coated beads will be also used to isolate SHARP-tagged proteins-expressing cells using a variety of techniques, FACS (fluorescence assisted cell sorting), MACS (Magnet assisted cell sorting), immune-affinity separation etc. A large number of TAG-SAR constructs with wild-type and mutant SHARP-tags were generated and were recognized by huMA79b and 2F2 antibodies by flow cytometry.

In summary, despite variability in TAG-SAR surface expression resulting from differences in transduction efficiency, viral titer, or cell type, SHARP-tag expression is consistently detected across a broad spectrum of experimental conditions and SAR formats. Specifically, more than 100 SHARP-tagged SAR constructs incorporating diverse structural configurations-including single-chain, dual-chain, and hybrid receptor formats—and differing tag insertion sites (e.g., N-terminal, juxta membrane, or extracellular linker regions)—are robustly and reproducibly detected in both transformed cell lines (e.g., JNG cells) and primary human T cells.

Detection of the SHARP-tag is achieved using multiple orthogonal methods, including flow cytometry, Malibu-Glo assay and enzyme-linked immunosorbent assay (ELISA), demonstrating high sensitivity, specificity, and reproducibility across assay platforms. These findings are particularly notable given the inventor's prior testing of alternative hypoimmunogenic tags based on FDA-approved antibodies—such as rituximab, Obinutuzumab, brentuximab vedotin, and belantamab mafodotin-blmf—which yielded poor detection sensitivity and significant variability in performance, especially in primary cells or low-expression settings.

The ability to detect SHARP-tagged SAR constructs reliably and with high fidelity across a range of formats and conditions provides a substantial advantage for clinical translation. Specifically, the SHARP-tag facilitates critical release testing, lot-to-lot consistency assessments, and in-process monitoring of gene-modified cell products-key parameters required by regulatory authorities such as the FDA and EMA for Good Manufacturing Practice (GMP) compliance. Moreover, the tag's robust detectability supports in vivo tracking and pharmacokinetic analysis of therapeutic cells, which are essential components of clinical development, product characterization, and regulatory approval of cellular immunotherapies.

SHARP-tagged proteins with protein degradation motifs: A panel of SHARP-tagged constructs (SEQ ID NO: 4997-5003) encoding fusion proteins comprising IKZF degradation motifs (SEQ ID NO: 2547-2553) are constructed and expressed in JNG cells. Treatment with IMiDs (e.g., Lenalidomide, Pomalidomide, Mezigdomide CC-92480 100 nM, Eragidomide CC-90009, 100 nM, Avadomide CC-122, 100 nM etc.) is shown to lead to rapid downregulation of expression of the SAR-TAG constructs as measured by Malibu-Glo assay and staining with antibodies (e.g., huMA79b, 2F2 and SN8). These results demonstrate that expression of SHARP-tagged fusion proteins can be controlled, and they can be expressed in an inducible manner. In alternate embodiment, expression of SHARP-tagged fusion proteins (e.g., TAG-SAR) is controlled by administration of doxycycline when they are expressed from a tetracycline-inducible promoter.

Treatment with Polatuzumab Vedotin (Pola) Results in Selective Depletion of TAG-SAR Expressing Jurkat (JNG) Cells.

JNG-P and JNG-4667 and JNG-4668 which express SHARP-tagged SAR constructs with SEQ ID NO:4838 and 4839) were plated at 0.5 million cells/well/ml in a 24-well plate. The cells were either left untreated or treated with Rituximab (10 g/ml) or Polatuzumab vedotin (10 g/ml) for 72 h. An aliquot of Cells was stained with the Malibu-Glo reagent (SEQ ID NO (PRT):1199) as described above. Treatment with Polatuzumab vedotin was shown to result in significant reduction in luciferase activity in JNG-4667 and JNG-4668 cells as compared to the control JNG-P cells. Treatment with Rituximab was used as a control and had no significant effect. These results demonstrate that treatment with Polatuzumab vedotin can be used to selectively deplete TAG-cells, i.e., cells that express a SHARP-tag described herein.

The depletion of TAG-SAR cells following treatment with Polatuzumab vedotin was confirmed using staining with the Malibu-Glo reagent followed by staining with a FITC-conjugated antibody against the Strep-tag present in the Malibu-Glo reagent. Post-treatment, the cells were centrifuged and resuspended in fresh RPMI 10% FBS medium to stop antibody treatment. The cells were cultured in 24-well suspension plates and later in 6-well plates without any drug treatment to expand these cells to see if TAG-SAR expression gets lost in Polatuzumab vedotin-treated cells. Approximately 2 weeks after Polatuzumab vedotin treatment, 1 ml of cells from each well were collected and stained with trypan blue and live cells were counted using hemocytometer. The results showed significant death of JNG-4667 and JNG-4668 cells expressing the TAG-SAR following treatment with Polatuzumab vedotin.

Treatment with Polatuzumab Vedotin (Pola) Results in Selective Depletion of TAG-SAR Expressing T Cells.

Human peripheral blood T cells isolated using CD3 magnetic beads are infected with lentiviruses expressing the SAR constructs 032223-VVB (SEQ ID NO: 5044), 022124-BBdC1 (SEQ ID NO: 5045) and 022824-EZyO1 (SEQ ID NO:5046) targeting STEAP2. The constructs with SEQ ID NO: 5045 and 5046 are TAG-SAR that comprise the Pola-11 tag inserted downstream of signal peptides of the second or the first polypeptide chains. Staining with Protein-L showed expression of SAR on 36%, 38% and 41% of T cells. A Malibu Glo assay with 110323-BBeT2 supernatant showed significant increase in luciferase activity (RLU) in the T cells expressing the two TAG-SAR constructs (SEQ ID NO: 5045 and 5046) as compared to the control T cells or those expressing the untagged SAR construct (SEQ ID NO: 5044) (FIG. 1).

The expression of the TAG-SAR is confirmed by flow-cytometry based Malibu-Glo assay. FIG. 2 shows binding of the Malibu-Glo reagent to 42% and 50% of T cells expressing the TAG-SAR constructs with SEQ ID NO: 5045 and SEQ ID NO:5046), respectively. Staining with the SN8 antibody detected expression of the TAG-SAR constructs with SEQ ID NO: 5045 and SEQ ID NO:5046 on 83% and 68% of T cells, respectively (FIG. 3). No significant staining was observed in control T cells or those expressing the untagged SAR construct (FIG. 3).

Treatment with Polatuzumab Vedotin Results in Killing of TAG-SAR Cells.

Control T cells and TAG-SAR-expressing T cells were seeded at a density of 0.5 million cells per well per mL in 24-well plates. Cells were either left untreated or treated with Rituximab (RTX) (10 μg/mL) or Polatuzumab (10 μg/mL) for 72 hours. Following treatment, 100 μL of cells were collected from each well and stained using the BD Pharmingen™ FITC Annexin V Apoptosis Detection Kit I (Cat. No. 556547) to assess cell death. T cells expressing TAG-SAR constructs corresponding to SEQ ID NOs: 5045 and 5046 showed an increase in cell death from 26.4% and 25.9% (untreated controls) to 90.42% and 81.3%, respectively, following Polatuzumab exposure. Polatuzumab had no significant effect on induction of cell death among the T cells expressing the untagged SAR (SEQ ID NO: 5044). Rituximab failed to induce cell death in any cell. Staining with Trypan blue showed a decrease in live-cell count of T cells expressing the TAG-SARs from 1.5 million cells/ml in untreated cells to approximately 0.25 million cells/ml following treatment with Polatuzumab, reflecting a more than 80% induction of cell killing.

Matador Assay Confirms Depletion of TAG-SAR Cells Following Treatment with Polatuzumab Vedotin.

LNCaP cells expressing Luc-PPE were plated at 100,000/well/400 mL in a 24-well plate in XVIVO medium. The LNCaP cells were co-cultured with T cells (T-control or T-STEAP2 TAG-SAR) that had been either left untreated or treated with Rituximab 10 μg/ml or Polatuzumab vedotin 10 μg/ml for 72 hours. LNCaP and T cells were co-cultured in 24-well plate and incubated at 37° C. for 24 hours. Post-incubation, cells were collected, centrifuged at 1500 rpm for 5 min at 4° C. Luciferase activity was measured in the supernatant by adding 15 μL of D-Luciferin, DTT and ATP. FIG. 5 demonstrates a significant reduction in the killing of LNCaP cells by T cells expressing the TAG-SAR constructs with SEQ ID NO: 5045 and 5046 following treatment with Polatuzumab vedotin, indicating depletion of TAG-SAR expressing T cells. Treatment with Rituximab had no effect.

The in vivo activity of the SHARP-tagged SARs was tested using a LNCaP xenograft model in NSG mice. Mice (n=5) were injected subcutaneously with 1 million Luc-PPE-expressing LNCaP cells in Matrigel. Five days later, each animal was injected with 10 million control T cells or SAR-T cells. Animals were followed using bioluminescence imaging. Mice administered T cells expressing the untagged SAR and SHARP-tagged-SAR constructs with SEQ ID NO: 5044, 5045 and 5046 showed effective and near complete tumor control as compared to mice given control T cells. Thus, SHARP-tag does not interfere with the in vivo activity of TAG-SAR-T cells.

In Vivo Treatment with Polatuzumab Vedotin Protects Mice Against STEAP2 Directed SHARP-Tagged CAR-T-Induced Toxicity.

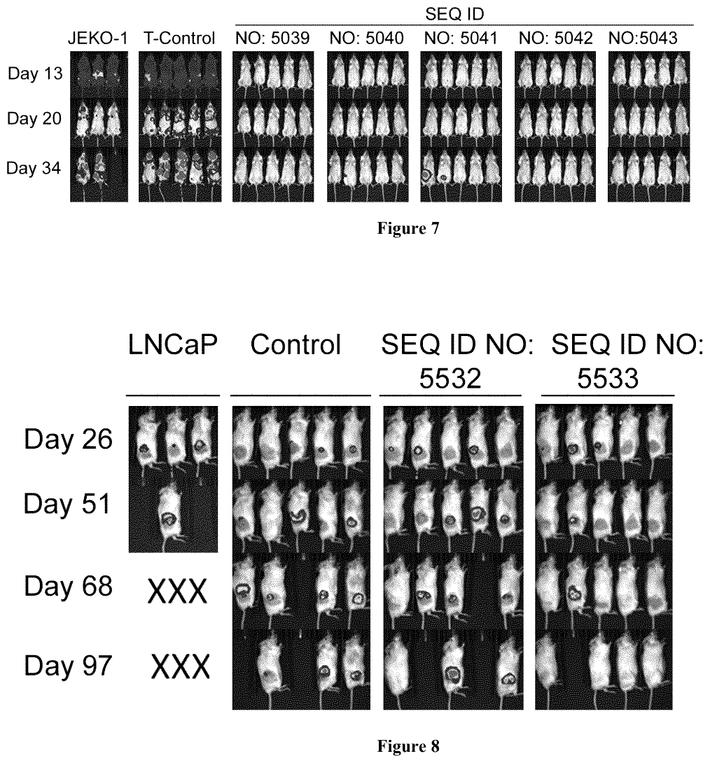

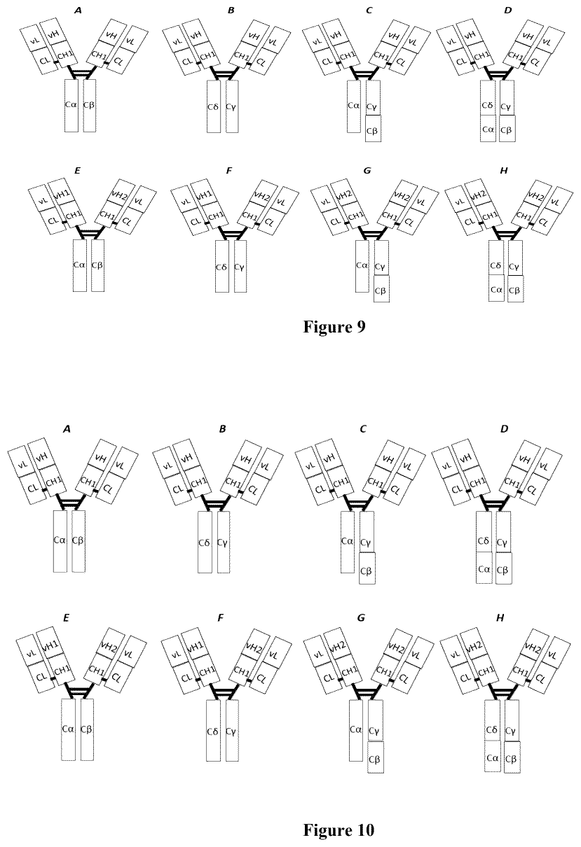

To demonstrate the in vivo efficacy of Polatuzumab vedotin in protecting against CAR-T-induced toxicity, we took advantage of the observation that T cells expressing a STEAP2 directed second generation CAR induce early lethality in mice. This is not seen with T cells expressing a STEAP2-SIR. T cells were transduced with lentiviral constructs comprising SHARP-tagged SAR constructs with SEQ ID NO: 5057 and 5046, respectively. The construct with SEQ ID NO: 5057 has the design of a second-generation CAR that comprise a P11 SHARP-tag (SEQ ID NO: 674) in the linker between the vL and vH fragments of a scFv targeting STEAP2. In contrast, the SAR construct with SEQ ID NO:5046 has the design of a SIR that comprises a P11 tag (SEQ ID NO: 674) inserted at the N-terminus of the vH fragment. The expression of the two SAR constructs was confirmed by staining with huMA79b antibody (SEQ ID NO: 1182) and flow cytometry, which showed the expression of SAR on approximately 77% and 24% of T cells transduced with constructs with SEQ ID NO: 5057 and 5046 respectively. Staining with 2F2 antibody (SEQ ID NO: 1189) confirmed the expression of the SAR with SEQ ID NO: 5057 on approximately 80% of T cells. Matador cytotoxicity assay on LNCaP-LucPPe cells confirmed significant cytotoxicity induction by T cells transduced with both SAR constructs. For in vivo studies, 10⁶ LNCaP-LucPPE cells were injected subcutaneously in NSG mice in Matrigel (50:50). One week later, 5×10⁶ SAR-T cells were given per mouse through tail vein injection. Next day, mice were administered control IgG1 or Polatuzumab vedotin at a dose of 1 mg/kg by tail vein injection. Animals were followed by bioluminescence imaging. All mice given T cells expressing the SAR with SEQ ID NO: 5057 followed by control IgG1 died on day 26, indicating toxicity of the STEAP2 CAR-T in mice (FIG. 6). In contrast, all mice given T cells expressing the SAR with SEQ ID NO: 5046 followed by Polatuzumab vedotin survived until day 41, although with progressive tumor (FIG. 6). These results demonstrate that in vivo treatment with Polatuzumab vedotin can deplete SHARP-tagged STEAP2-directed CAR-T cells and protect against their toxicity.

### Use of SHARP-Tags to Prevent GVHD.

Donor T cells engineered with SHARP-tagged SARs or membrane proteins (e.g., SEQ ID NO: 662 are infused into recipient mice following irradiation. At signs of GvHD, mice are treated with an Polatuzumab. SHARP-tagged T cells are selectively eliminated, reducing GvHD severity without affecting host hematopoiesis. In a similar experiment, donor T cells are transduced with a vector comprising a SHARP-tagged construct (e.g., SEQ ID NO: 8350-51) and T cells are selectively eliminated by treatment with H2Mab-250.

### Use of SHARP-Tag for Selective Depletion of Recombinant Cells.

SHARP-tagged CAR-T cells are incubated with appropriate SABR fused to or comprising a human IgG Fc domain. Peripheral blood mononuclear cells (PBMCs) are added to the cultures to mediate ADCC. Depletion is assessed by flow cytometry. SHARP-tagged CAR-T cells are selectively depleted in the presence of the SABR-Fc fusion and PBMCs, demonstrating that the tag can be used as a safety switch. In some experiments, the SHARP-tag comprises the sequence represented by SEQ ID NO: 1-123, 674-676, and the SABR is 2F2, SN8 or huMA79b (SEQ ID NO: 1182) antibodies. In other experiments, SHARP-tag comprises the sequence represented by SEQ ID NO: 557-559 and the SABR is H2Mab-250 or an antibody comprising the vL and vH fragments comprising the SEQ ID NO: 846 and 966, respectively.

Expression of TAG Constructs in Cells Other than T Cells.

293FT cells were transfected with lentiviral vectors comprising the SAR which comprise SHARP-tags P11 (SEQ ID NO: 674) or P14 (SEQ ID NO: 53) in the linker regions between the vL and vH fragments. SAR constructs BBaE1 and BBaG1 lack the epitope tags and served as negative controls. 48 h post-transfection, cells were stained with Malibu-Glo reagent represented by SEQ ID NO (DNA): 3649 and SEQ ID NO (PRT):1199. 293FT cells transfected with the TAG-SAR constructs showed increase in Nluc activity when stained with the Malibu-glo reagent with SEQ ID NO:1199 as measured by Malibu-glo assay as compared to control 293FT cells. The results were confirmed using Flow cytometry using Strep-tagII antibody. These results demonstrated that SAR constructs comprising SHARP-tags (e.g., P11 or P14) epitope tags can be identified using huMA79b antibody, scFv or fragment thereof. In addition, expression of tags could be detected in NK92 cells that had been transduced with lentiviral vectors encoding TAG-SAR constructs (e.g., SEQ ID NO: 4794).

Expression of TAG-polypeptides (e.g., TAG-SAR, e.g., SEQ ID NO: 4794) is also detected in other cell types (e.g., breast, lung, kidney, liver, skin, gastrointestinal, prostate, ovarian, muscle cells and neurons) that are transduced with the appropriate expression constructs.

Double Chain CD79b SAR Constructs Respond to Treatment with Soluble Peptides and Polypeptides.

Human CD79b-targeted SAR constructs huMA79b-WT-SIR (SEQ ID NO: 4830), huMA79b-mutant-SIR-AT5 (SEQ ID NO: 4895), huMA79b-WT-HC-SIR (SEQ ID NO: 4898) were transduced into JNG cells. The resulting cells and JNG parental cells were exposed to 3 different doses of CD79b-derived peptides at 37° C. for overnight. A p53 peptide was used as a negative control. Strong GFP induction was seen in JNG cells expressing the three SAR constructs when exposed to 10 mM dose of p11 peptide (SEQ ID NO: 50) with 10%, 6% and 41.5% of cells expressing the constructs with SEQ ID NO: 4830, 4895 and 4898, respectively, showing GFP induction. GFP induction was also seen in JNG cells expressing the SAR construct with SEQ ID NO: 4898 when exposed to a p10 peptide (SEQ ID NO: 49) and a p9 peptides (SEQ ID NO: 48). No GFP induction was seen with the p53 peptide (SEQ ID NO: 1209).

JNG cells expressing the different CD79b SAR constructs {(e.g., (SEQ ID NO: 2448 and 4898), (SEQ ID NO: 2407 and 4857) and (SEQ ID NO: 2448 4898)} were exposed to supernatants containing different SHARP-tagged scFv fusion proteins. The results are shown in Table 5 and demonstrate robust GFP induction by SHARP-tagged scFV supernatants comprising SHARP-tags with SEQ ID NO: 1-11 (e.g., Clone 031124-BBeV2), P8 (Clone 041024-EZeJ10), P9 (Clone 031124-BBeT1), P10 (Clone 031124-BBeS1), P11 (clone 031124-BBeR1) as well as several mutants of the above SHARP-tags. SHARP-tagged fusion proteins supernatants represented by SEQ ID NO: 5075-5116, 5120-5121, 5134, 5136, 5138, 5140, 5150, 5199, 5223, 5250, 5266-7, 5269-5270, 5312, 5315-6, 5357, 5377, 5368, 5417 and 5451 showed GFP induction when co-cultured with JNG cells expressing the CD79b SAR constructs with SEQ ID NO: 2448, 4898, 2407, 4857, 2448 and 4898. GFP induction was also seen upon co-culture of JNG clones expressing CD79b SAR constructs with SEQ ID NO:4828, 4829, 5065 with the supernatants containing different SHARP-tags (e.g., SEQ ID NO: 1-6, 50-52.). However, JNG cells expressing the CD79b SAR constructs represented by SEQ ID NO: 4895 showed low or no GFP induction when incubated with the supernatants containing the SHARP-tagged fusion proteins. Similarly, JNG cells expressing the single-chain second-generation CAR represented by SEQ ID NO: 4899 showed low or no GFP induction when incubated with the supernatants containing the tagged-fusion proteins.

Taken collectively, the above results demonstrate that immune effector cells (e.g., T cells, e.g., JNG cells) expressing double-chain SAR constructs targeting CD79b and comprising specific antigen binding domains can be activated by soluble peptides (e.g., SEQ ID NO: 49, 50 etc.) and soluble polypeptides (e.g., SEQ ID NO: 5075-5116 and 5120-5121 etc.) comprising SHARP-tags (e.g. SEQ ID NO: 1-6, 50-52) that are recognized by these SAR constructs. In example embodiments, the SARs constructs targeting CD79b comprise vL fragments with SEQ ID NO: 774-782 and complementary vH fragments with SEQ ID NO:899-907 and functional variants thereof with up to 20 amino acids substitutions in the framework regions and up to 2 amino acids substitutions in the CDRs that bind to CD79b.

### Binding of 10D10 Antibody and 10D10 Malibu Reagent to Cyano-CD79b and to SHARP-Tags Represented by CY11 and CY13.

JNG cells were transduced with a lentivirus vector construct with SEQ ID NO: 4968 encoding a CD19 SAR that co-expressed cyno-CD79b. The expression of Cyno-CD79b on the SAR-expressing JNG cells was detected by staining with 10D10 antibody supernatant (051824-EZyJ1; SEQ ID NO (PRT):1193) followed by flow cytometry. Robust staining was observed following staining with 10D10 with approximately 58% cells showing positive staining. The results were confirmed using Malibu-Glo assay using a 10D10-Nluc fusion protein encoded by construct with SEQ ID NO: 5539. These results indicate that cyno-CD79b can be used as a multipurpose switch for detection of SAR expressing cells.

In a complementary experiment, JNG cells were transduced with lentiviral constructs 041624-EZjB2 (SEQ ID NO: 4958) and 041624-EZjC2 (SEQ ID NO: 4959) that encode for double chain SARs comprising 10D10 vL (SEQ ID NO: 797) and 10D10 vH (SEQ ID NO: 797) domains targeting cyno-CD79b. The resulting JNG clones showed specific binding to supernatants containing Malibu-Glo reagents encoded by SEQ ID NO (DNA): 5536 and 5537 that encode for CYD11QCYD11 (SEQ ID NO: 689) and CYD11 (SEQ ID NO: 690) tags, respectively. The binding of fusion protein to the JNG cells was determined by staining with Strep-tag-FITC and flow cytometry. These results confirm specific recognition of the CYD11 tag by the 10D10 antibody, 10D10 scFV and T cells expressing a SAR comprising a 10D10-based antigen binding domains. Thus, CYD11 tag can be used to mark cells for detection, selection, enrichment, depletion by 10D10 based antibody, scFv and/or SAR-T cells.

In a similar experiment, JNG cells, NK92 cells and primary T cells will be generated that express the TAG-SARs (e.g., a second-generation CAR) containing the SHARP-tags represented by SEQ ID NO:557-566. The expression of the TAG-SAR is examined by staining with H2Mab-250 antibody and/or by using a Malibu-Glo reagent comprising an scFv comprising vL regions comprising SEQ ID NO: 846 or 847 and complementary vH regions comprising SEQ ID NO: 966 or 967. Expression of the SHARP-tag on the cell surface is evaluated by flow cytometry using fluorophore-conjugated H2Mab-250. A FITC conjugated antibody against the FLAG or the StrepTag is used to detect the binding by the Malibu-Glo reagent. Cells expressing SHARP-tagged SARs are specifically stained by H2Mab-250 or the Malibu-Glo reagent but not by isotype control, confirming the presence of surface-expressed SHARP-tag.

### Multipurpose Switches Comprising SHARP-Tags

IL2-dependent NK92 cells were infected with lentiviral constructs that expressed SARs and co-expressed multipurpose switches encoded by SEQ ID NO: 3116, 3115, 3129 and 3130. These switches encode polypeptides comprising different SHARP-tags (e.g., P11 (SEQ ID NO: 674), P13 (SEQ ID NO: 675), CD79b-Iso2-ECD (SEQ ID NO: 655) that are fused to membrane-bound forms of IL2 or IL15. The NK92 cells were withdrawn from IL2 and grown in IL2-free medium to allow the growth of cells that had been transduced with the lentiviral vector and express the multipurpose switch. The survival of NK92 cells transduced with the multipurpose switch encoding lentiviral vectors was observed. The expression of multipurpose switch was detected by staining with Malibu-Glo reagent that binds to SHARP-tags followed by measurement of luminescence. The results showed that luminescence increased from 639 in parental NK92 cells to 780, 1765, 2009 and 4819 in the NK92 cells transduced with constructs represented by SEQ ID NO: 3116, 3115, 3129 and 3130, respectively. Essentially similar results were obtained when JNG cells were transduced with a lentiviral construct (SEQ ID NO: 4938) encoding SAR construct that co-expressed a multipurpose switch. The JNG showed significant binding with the Malibu Glo reagent 110323-BBeT2.

### Expression and Functional Validation of SHARP-Tagged Antibody Constructs

A series of antibody constructs incorporating SHARP-tags (e.g., SEQ ID NO: 674, 557) at the N- or C-termini of the light and/or heavy chains were engineered, with representative sequences provided as SEQ ID NOs: 5526 through 5531. These constructs were transiently transfected into 293FT cells. Culture supernatants were harvested 4-6 days post-transfection, and SHARP-tagged antibodies were purified using column containing immobilized SN8, 2F2, HuMa79b or H2Mab-250. Purified antibodies are found to be functionally active as demonstrated by binding to target cell lines followed by flow cytometry.

### Use of SHARP-Tag in Oncolytic Virus Design

An engineered herpes simplex virus (HSV) was modified to express a SHARP-tagged membrane protein (e.g. SEQ ID NO: 662, 665-666) or SAR during infection. Tumor-bearing mice were injected intratumorally with the virus. Tumors were biopsied and stained with SABR (e.g., SN8 or huMA79b). SABR specifically stains virus-infected cells in the tumor, allowing tracking of viral spread. In a variation, the tag allows depletion using Polatuzumab vedotin.

Malibu-Glo and JNG Assays show specific binding of novel antigen binding domains and Novel SAR designs. The antigen binding properties of the antigen binding domains described in Tables 2-4 were demonstrated by generating Malibu Glo reagents comprising these binding domains. The Malibu-Glo reagents were tested for binding to the target cell lines and were found to show increased binding as compared to the control cell lines. Representative results are shown in Table 7. The binding domains listed in Tables 2-4 are also used to generate SAR and tested in JNG assay after co-culture with the appropriate target cell lines. The JNG cells expressing the SAR showed GFP induction when co-cultured overnight with the target cell lines. The representative results are shown in Table 6. Further, Table 6 also shows GFP induction in JNG cell by a large panel of unispecific, and bispecific SAR constructs, including multi-chain SAR and SHARP-tagged SAR.

### T Cells Expressing NPM1c and Mutant NPM1c Directed SARs Induce Cytotoxicity.

Human T cells isolated using CD3 magnetic beads are infected with lentiviruses expressing the 2^{nd} generation CAR and SIR constructs targeting NPM1c and comprising vL domains represented by SEQ ID NO:836-841 and the complementary vH domains with SEQ ID NO: 959-961. Additionally, T cells are also infected with 2^{nd} generation CAR and SIR constructs targeting mutant NPM1c and comprising vL domains represented by SEQ ID NO:843-845 and the complementary vH domains with SEQ ID NO: 963-965. The CAR-T and SIR-T cells are expanded in vitro for 10-14 days. Target cells (e.g., OCI-AML3 or MOLT13) stably expressing GLuc are cocultured with T cells expressing the different SARs at an E:T ratio of 10:1 for 48 hours. CAR/SIR-T cells mediated lysis of target cells is assayed using the Matador assay. The in vivo activity of the CARs is demonstrated using a xenograft model in NSG mice. Essentially a similar approach is used to demonstrate the in vitro and in vivo activities of SAR targeting Her2 and comprising the vL fragments with SEQ ID NO: 846-7 and vH fragments with SEQ ID NO: 967-970.

### Novel Topanga Reagents for Detecting PSMA SAR-T

Novel constructs (e.g., SEQ ID NO:9148-9153) encoding Topanga reagents (SEQ ID NO:3039-3042)) were generated that recognize PSMA SAR-T. These constructs encode PSMA fusion proteins in which the reporter (e.g., Nluc) is attached to N-terminus to the PSMA extracellular domain. They also comprise deletion mutants of PSMA in fusion with the reporter (e.g., Nluc, Gluc, TurboLuc etc.). These novel Topanga reagents also incorporate a dimerization domain and/or hinge domain (e.g., hIgG1-CH3 domain). The Topanga reagents were expressed in 293FT cells and were found to show superior expression as compared to the PSMA Topanga reagents generated previously. They also recognized JNG cells expressing PSMA SARs (e.g., SEQ ID NO: 5532-5534), including PSMA-SIR-T, with greater sensitivity and specificity as compared to previous Topanga reagents.

### T Cells Expressing CD79b SARs Induce Cytotoxicity in CD79b-Expressing

Human peripheral blood T cells isolated using CD3 magnetic beads were infected with lentiviruses expressing the SAR constructs (e.g., SEQ ID NO: 5060-5071, 5073) targeting CD79b. The constructs with SEQ ID NO: 5070 and 5071 also co-expressed membrane-bound IL2 (SEQ ID NO: 2622). T cells expressing the different SAR constructs show positive staining with CD79b-Fc demonstrating expression of the SAR constructs. T cells expressing the constructs with SEQ ID NO: 5063 (52% positive cells) and 5068 (70% positive cells) show higher staining as compared to others. The cytotoxicity of T cells expressing the different SAR constructs is examined by Matador Glo assay using DAUDI, JEKO-1 and RAJI cells expressing LucPPe-146-1H2 at different E:T ratios and co-culture times of 4 to 48 hours. T cells expressing all constructs show significant cytotoxicity. T cells expressing the constructs with SEQ ID NO: 5063 and 5068 show higher cytotoxicity. The SAR-T cells were expanded in culture and cytokine (IFNy, TNFa, and IL2) production by different SAR-T cells was measured by ELISA in the supernatant after 48 h co-culture with JEKO-1 and Daudi cells. T cells expressing the constructs with SEQ ID NO:5065-5067, 5069 and 5071 show higher IFNγ secretion, those with SEQ ID NO: 5064, 5065, 5066, 5068 and 5069 show high TNFα secretion, and those with SEQ ID NO: 5065, 5066 and 5069 show higher IL2 secretion.

The in vivo activity of the different SAR-T cells was tested using a JEKO-1 xenograft model in NSG mice. Jeko1-Luc-PPE cells were injected via tail vein injection (0.5 million cells/mouse) into mice (n=5 or 4). T cells (5 million cells/mouse) expressing the different SAR constructs were injected via tail vein injection 9 days later. Animals were examined using bioluminescence imaging. All animals given SAR-T cells showed effective tumor control as compared to animals given control T cells, which resulted in improvement in survival. Highest survival is seen in animals given T cell expressing the constructs with SEQ ID NO:5062, 5065 and 5066, while those with SEQ ID NO: 5068, 5070 and 5071 show greater relapse.

Experiment is repeated with T cells expressing the CD79b SAR constructs with SEQ ID NO (DNA):4647, 4648-4655, 4672-4679 and 4720-4727. These constructs encode CD79b SAR with SEQ ID NO (PRT): 2197, 2198-2205, 2222-2229 and 2270-2277, respectively. The SAR constructs with SEQ ID NO: 4647 encodes a vL fragments with SEQ ID NO:2183 and a vH with SEQ ID NO: 2184. The SAR constructs with SEQ ID NO: 4648-4655, 4672-4679 and 4720-4727 encode a vL fragments with SEQ ID NO:2173-2178 and a vH fragments with SEQ ID NO: 2179-2182. The TCRb-ECD-CD3z signaling chains, when present, are represented by SEQ ID NO: 2185-2186. The TCRa-ECD-CD16 signaling chains, when present, are represented by SEQ ID NO: 2187-2188. The TCRα signaling chains, when present, are represented by SEQ ID NO: 2190-2191. The TCRβ signaling chains, when present, are represented by SEQ ID NO: 2189-2190. The TCRb-ECD-CD3z fusion protein, when present, is represented by SEQ ID NO: 2193-2194 and TCRa-CD3z fusion protein, when present is represented by SEQ ID NO:2195-2196. An example of a lentiviral vector comprising a CD79b SAR is provided in SEQ ID NO:6377. The T cells expressing the above constructs would how significant expression on T cells by staining with CD79b-Fc reagent. The T cells expressing the different SAR constructs would be shown to mediate effective cytotoxicity and induce cytokine production as measured by Matador assay and ELISA when co-cultured with JEKO-1 and Daudi cells. Furthermore, T cells expressing the above SAR constructs would show effective control of tumor when tested in vivo using the JEKO-1 xenograft model in NSG mice as detailed above.

Additional CD79b SAR constructs are represented by SEQ ID NO (DNA): 4648-4751, and SEQ ID NO (PRT): 2198-2301. An additional panel of unispecific, bispecific and hybrid chain SAR constructs targeting CD79b is presented in SEQ ID NO: 5551-6365,6367-6375, 6378-6537, 6563-6574, 6589-6660. The constructs would be shown to show significant expression on T cells by staining with CD79b-Fc reagent. The T cells expressing the different SAR constructs would be shown to mediate effective cytotoxicity and induce cytokine production as measured by Matador assay and ELISA when co-cultured with JEKO-1 and Daudi cells. Furthermore, T cells expressing the above SAR constructs would show effective control of tumor when tested in vivo using the JEKO-1 xenograft model in NSG mice as detailed above.

### T Cells Expressing CD19 and CD19×CD22 Bispecific SARs Induce Cytotoxicity and Show Effective Tumor Control In Vivo

Human peripheral blood T cells isolated using CD3 magnetic beads were infected with lentiviruses expressing the SAR constructs (e.g., SEQ ID NO: 5010-5021) targeting CD19 and/or CD22. Constructs with SEQ ID NO:5016, 5018-5020 are TAG-SAR constructs that comprise SHARP-tags with SEQ ID NO: 2 or 675. Expression of the TAG-SAR constructs is confirmed by staining with Malibu-Glo reagent and huMA79b (041924-BBhJ2) and 2F2 (041924-BBhO1) antibody supernatants. In vivo activity of the different SAR-T was demonstrated using a xenograft model of JEKO-1 cells xenografted in NSG mice. The results demonstrated effective tumor clearance by all constructs as compared to the control T cells.

### T Cells Expressing CD19, CD19×CD20 and CD19×BCMA Bispecific SARs Induce Cytotoxicity and Show Effective Tumor Control In Vivo

Human peripheral blood T cells isolated using CD3 magnetic beads were infected with lentiviruses comprising the SAR constructs with SEQ ID NO (DNA): 5039-5043. These constructs encode for SARs with SEQ ID NO (PRT): 2589-2593) targeting CD19, CD19×CD20 and CD19×BCMA. The constructs comprise different SAR designs. The constructs with SEQ ID NO: 5039, 5040 and 5041 encode for unispecific SAR constructs that target CD19 and are double chain SIR, zSIR and hybrid chain SIR (HC-SIR), respectively. The constructs with SEQ ID NO: 5042 and 5043 encode for bispecific SIRs that target CD19 and BCMA, and CD19 and CD20, respectively. The construct with SEQ ID NO:5040 encodes for a TAG-SAR constructs that comprises a SHARP-tag with SEQ ID NO:674. Staining with Protein L shows that 56%, 74%, 59%, 62%, and 40% of T cells shows expression of SAR when transduced with SEQ ID NO: 5039-5043, respectively. Matador cytotoxicity assay confirms specific cytotoxicity of T cells expressing all constructs against RAJI, JEKO-1, and RS;411 cell lines. T cells expressing the SAR with SEQ ID NO: 5042 also show specific cytotoxicity against MM1s cell line that expresses BCMA. T cells expressing the constructs with SEQ ID NO: 5039-5041 show IFNγ and TNFα induction when exposed to RAJI cells but not RAJI cells with knock-out of CD19. T cells expressing the bispecific SAR constructs show IFNγ and TNFα induction when exposed to RAJI cells and RAJI cells with knock-out of CD19 (RAJI-CD19-KO). In vivo activity of the different SAR-T was demonstrated using a xenograft model of JEKO-1 cells xenografted in NSG mice. Mice are injected with 0.5 million JEKO-1 cells expressing LucPPe via tail vein and given SAR-T cells two days later. The results are shown in FIG. 7 and demonstrate effective tumor clearance by all constructs as compared to the control T cells.

T cells expressing PSMA SARs induce cytotoxicity in PSMA-expressing LNCaP cells. Human peripheral blood T cells isolated using CD3 magnetic beads are infected with lentiviruses comprising the PSMA-targeted SAR constructs with SEQ ID NO (DNA): 5532 and 5533, respectively. These constructs encode for SARs with SEQ ID NO (PRT):3037 and 3038. The SAR construct with SEQ ID NO: 5532 encodes for a second-generation CAR, while the construct with SEQ ID NO: 5533 encodes for a zSIR in which the vL and vH domains of a PSMA antibody are separately attached to two CD3z chains via linkers derived from immunoglobulin. The zSIR design is described in PCT/US2019/035096. Staining with Protein L shows that 78% and 41% of T cells show expression of the SAR when transduced with SEQ ID NO: 5532 and 5533, respectively. Matador cytotoxicity assay shows equivalent cytotoxicity of T cells expressing the two SAR on LNCaP and MDAPCa2b cell lines that have been engineered to express LucPPe. T cells expressing SAR with SEQ ID NO: 5532 shows higher TNFα and IFNγ secretion when co-cultured with LNCaP and MDAPCa2b cells as compared to T cells that express SEQ ID NO: 5533. In vivo efficacy is tested using a LNCaP xenograft model in NSG mice. On day 1, one million LNCaP-LucPPE cells are injected subcutaneously with Matrigel (50:50) in the flanks of mice. On Day 13, mice receive 5 million SAR-T cells via tail vein injection. Bioluminescence imaging demonstrates enhanced tumor control in mice treated with SAR-T cells transduced with the construct comprising SEQ ID NO: 5533, compared to mice receiving either control cells or SAR-T cells transduced with the construct comprising SEQ ID NO: 5532 (FIG. 8). These results demonstrate the superior in vivo efficacy of the zSIR construct in controlling tumor growth within a solid tumor model.

In Vivo Treatment with Polatuzumab Vedotin

A patient with prostate cancer is enrolled in a clinical trial for treatment with TAG-SAR-T cells targeting STEAP2. T cells are collected from the subject using leukapheresis, transduced with the appropriate TAG-SAR (SEQ ID NO:5045 and 5046) encoding lentivirus vector and expanded ex vivo using CD3/CD28 beads. The dose of SAR-T product varies from 1×10⁴ SAR+ve CD3 cells/kg to 5×10¹¹ SAR+ve CD3 cells/kg as per the study protocol. Five days after infusion of TAG-SAR-T cells, the patient develops symptoms suggestive of Cytokine release syndrome (e.g., Fever, chills, short of breath, hypotension, oliguria) and ICANS (e.g., confusion, headaches, seizure). The rapid expansion of TAG-SAR-T in peripheral blood is confirmed by staining with FITC-conjugated SN8 antibody and Malibu-Glo reagent (110323-BBeT2). Patient receives Tociluzumab and Dexamethasone per protocol without improvement. Finally, patient is administered Polatuzumab vedotin (Polivy) at dose of 1.8 mg/kg intravenously with improvement.

Virus and virus like particles. A viral envelope protein (e.g., VSV-G or mBAEV glycoprotein) or a transduction particle is engineered to display the SHARP-tag epitope. The viral particles are purified using SABR using affinity chromatography. The invention covers production of host cells (e.g., retroviral packaging lines) engineered with SHARP-tag markers for lot release testing and safe elimination after vector production.

In Vivo Regulation and Termination of SAR-T Cells via SABR and SABR-ADC Scenario: A patient with refractory B-cell lymphoma is treated with TAG-SAR19 T cells (expressing the SHARP-tagged anti-CD19 CAR). However, 1-week post-infusion, the patient develops grade 3 cytokine release syndrome (CRS). To mitigate CRS, the clinical team decides to administer SABR antibody (e.g., 2F2) to partially deplete or dampen the SAR-T cells. The patient receives an intravenous infusion of SABR (human IgG1) at 10 mg/kg. Within 12 hours, the patient's fever and blood pressure begin to improve. One week later, the patient develops neurotoxicity manifesting as confusion and tremors. The patient is given a single dose of Polatuzumab. The dose is 0.1 mg/kg. By day 3 post-ADC, the patient's neurological symptoms improve dramatically, and no SAR-T cells are detectable by flow cytometry or by a sensitive PCR for the SAR transgene in blood.

### Bispecific SABR×CD3 for Selective Killing of SHARP-Tagged Cells

A bispecific antibody SABR×CD3 will be engineered, consisting of the scFv of SABR and an scFv from OKT3 (anti-CD3F), linked in tandem (a BiTE format). SHARP-tagged CAR-T cells (targets) will be mixed with fresh CD3+ T cells (effectors) at a 1:1 ratio. The SABR×CD3 BiTE will be added to the culture at varying concentrations (0, 1, 10, 100 ng/mL). After 16 hours, specific lysis of target cells will be measured.

Co-Expression of SHARP-tag with a CD34 Selection Marker. T cells are transduced with a vector encoding a SAR with SHARP-tag and Truncated CD34 (tCD34). A multiple myeloma patient's T cells are transduced with this vector. Anti-CD34 magnetic beads are used to select the transduced cells. The cells are expanded using standard methods (e.g., anti-CD3/28 beads) or using SABR crosslinking. The patient receives the dual-tagged CAR-T cells. The SHARP-tag on the CAR allows tracking of these cells by flow cytometry. Meanwhile, the co-expressed CD34 is also detectable by flow. If one patient develops a delayed neurotoxicity at week 3, SABR antibody is administered to eliminate the CAR-T cells. Co-expression of SHARP-tag and CD34 does not show any detrimental effects on the CAR functionality.

SHARP-tagged Cytokines: Recombinant cytokines (e.g., IL-2 and IL15) are expressed with a C-terminal or N-terminal SHARP-tag (SEQ ID NO: 674) in HEK293 cells. The representative constructs are shown with SEQ ID NO:3117-3119 and 3121-3123. The cell culture supernatant is passed over a column containing immobilized SN8, 2F2 or HuMa79b. Bound protein is eluted with low-pH buffer. The SHARP-tagged cytokine binds specifically to the SABR (i.e., SN8) column and elutes as a single peak, allowing purification in a single step. Essentially similar results are obtained when the cytokine (IL2) is tagged at N-terminal with a SHARP-tag represented by SEQ ID NO: 557 and purified over a column containing immobilized H2Mab-250. The recombinant cytokines are tested in functional assays and retain their biological activities.

Improved method to determine the titer of a vector. Lentiviral vectors encoding constructs with SEQ ID NO: 5010-5033 are prepared by transfection in 293FT cells. Supernatant containing the viral particles is collected. To determine the titer of the viruses, viral supernatant is used to infect wild-type JNG T cells and JNG cells which have impaired or abolished expression of one or more endogenous TCR constant chains selected from the group consisting of TCRα, TCRβ1 and/or TCRβ2 constant chains. The titer of the lentiviral vector is measured by Protein L staining, Topanga assay, and by JNG-NFAT-GFP assays. Infection of JNG cells with impaired or abolished expression of TCRα and/or TCRβ (e.g., TCRβ1) constant chains results in determination of the titer of the lentiviral vectors that comprise double chain SAR constructs (e.g., SEQ ID NO: 5011-5021) with greater sensitivity and accuracy. These constructs comprise TCRα, TCRβ1 and/or TCRβ2 constant chains. In contrast, the infection of parental JNG cells frequently leads to underestimation of the titer of double-chain SAR constructs comprising TCRα, and/or TCRβ constant chains. Further, the titers of lentiviral vectors encoding SAR constructs comprising hybrid TCR constant chains is also measured with greater sensitivity and accuracy in JNG cells with impaired or abolished expression of TCRα, TCRβ (e.g., TCRβ1) constant chains. Thus, the titer of vector constructs encoding the SAR constructs with the designs of SIR, cTCR, HIT, STAR, HC-SIR and recombinant TCR is measured with greater sensitivity and accuracy in T cells with impaired or abolished expression of an endogenous TCR constant chain. Essentially similar results are obtained when using retroviral vectors, adenoviral vectors, AAV vectors, herpes viral vectors, mRNA vectors, lipid nanoparticles, viral like particles, plasmid vectors, baculoviral vectors, and transposons. Essentially similar results are obtained when the titer of a vector comprising TCRγ and TCRδ chain is determined in T cells with impaired or abolished expression of endogenous TCRγ and/or TCRδ chains. Finally, there is no major advantage of using JNG cells with impaired expression of endogenous TCR chains when measuring the titer of a single chain vector (e.g., a second-generation CAR). Such a vector is represented by SEQ ID NO: 5010.

Novel approach to measure the expression of SAR constructs. JNG and T cells are transduced with lentiviral vectors encoding SARs with SEQ ID NO: 5011-5018, 4946 and 4916-4922, 4951-4955 comprising scFv, vL, vH, vHH and FHVH domains as their antigen binding domains. Expression of the SAR is determined by staining with PE conjugated Goat Anti-Human Ig and PE-conjugated Goat F(ab′)2 Anti-Mouse IgG(H+L). SAR constructs with SEQ ID NO: 5011-5018 show significant staining with PE conjugated Goat Anti-Human Ig and those with SEQ ID NO: 4916-4922, and 4951-4955 show significant staining with Goat F(ab′)2 Anti-Mouse IgG(H+L).

Novel approach to measure the expression of SAR constructs comprising TCR constant chains. A lentiviral vector encoding a SAR (SIR) targeting PSMA (SEQ ID NO: 4957) is used to infect JNG and primary T cells. The expression of the SIR is difficult to detect with the conventional methods, e.g., staining with Protein L due to poor sensitivity and high background. To detect the expression of the SIR in JNG cells with greater sensitivity, the cells are stained with antibodies against human TCRα, TCRβ1 and/or TCRβ2 constant chains. The expression of the TCRβ2 constant chain on JNG cells is used to determine the expression of the SAR. The method is repeated in primary T cells with similar results. Primary T cells express TCRβ2 chain. Therefore, the cells are stained with antibodies against TCRβ1 and TCRβ2 constant chains. The presence of T cells that show staining with both TCRβ1 and TCRβ2 constant chain specific antibodies (i.e., double positive cells) is used as a measure of the expression of the SIR. The method is used to measure the potency of the vector encoding the SIR and as a product release assay. The experiment is repeated using other SAR constructs (e.g., SIR, HC-SIR, Multi-chain SIR, HIT, STAR etc.) comprising human TCR constant chains with similar results.

A Short Method for Manufacturing of a SIR, HC-SIR, zSIR, z16-SIR, CD16-SIR.

A SAR-T cell product is manufactured using a process optionally beginning with administration of a mobilizing agent (e.g., plerixafor) to a healthy donor to mobilize immune effector cells. Immune cells are collected via leukapheresis, optionally cryopreserved and thawed prior to downstream processing. T cells are isolated using CD3 selection or CD4/CD8 enrichment and/or depletion of non-T cells, followed by activation using anti-CD3/CD28 beads or antibodies for 12-18 hours in the presence of IL-2 or IL-15/IL-7. Approximately 300-400 million activated T cells are then transduced with a nucleic acid encoding a Synthetic Antigen Receptor (SAR) (SEQ ID NO:2197-2302, 2304-2514) using a lentiviral vector in the presence of vectofusin-1. Transduced cells are expanded for 12 hours to 7 days with IL-2 or IL-15/IL-7 and CD3/CD28 stimulation, harvested, and formulated. The process is performed in a closed automated system, optionally using the Prodigy® CliniMACS platform and the T Cell Transduction Large Scale (TCT-LS) process.

Combination of a PSMA SAR with anti-androgens. Prostate cancer cell lines (LAPC4, LNCaP, 22Rv1 and C4-2) are treated with 10 μM bicalutamide, 10 μM enzalutamide, 10 μM apalutamide, or 10 μM darolutamide for over 2-4 weeks or left untreated. Subsequently, the cell lines are exposed to PSMA SAR-T cells (SEQ ID NO: 4937-4938). Cell death is measured using Matador cytotoxicity assay and is found to be significantly increased in cell lines treated with the drugs as compared to untreated cells. No increase in toxicity is seen when the experiment is repeated with normal cells from vital tissues, such as lung, kidney, liver, heart, brain etc.

Combined treatment with a SAR-T or SAR-NK cells and antiandrogen. A male patient diagnosed with metastatic castration-resistant prostate cancer (mCRPC) expressing a tumor-associated antigen (e.g., PSMA, PSCA, STEAP2 etc.) targetable by a Synthetic Antigen Receptor (SAR) is treated with autologous CAR-T cells in combination with an anti-androgen agent. The SAR (e.g., CAR, SIR, zSIR, z16SIR etc.) is selected from the group consisting of SEQ ID NO: 4931, 4933, 4937-4943, 5006-5008, 5044, 5045, 5046, or 5059). On Day 0, following lymphodepleting chemotherapy, the patient receives an infusion of 5×10⁶ SAR-T cells/kg body weight. Concomitant to cell infusion, the patient is administered enzalutamide orally at a dose of 160 mg per day, continued daily throughout the observation period. Alternatively, anti-androgens such as bicalutamide (50 mg/day), flutamide (750 mg/day in divided doses), nilutamide (150 mg/day), apalutamide (240 mg/day), darolutamide (1200 mg/day in divided doses), or abiraterone acetate (1000 mg/day with prednisone 5 mg BID) are substituted depending on patient tolerance, prior treatment, and tumor responsiveness. The combination therapy is well tolerated and resulted in a sustained decline in PSA levels and radiographic evidence of tumor regression, supporting synergistic activity between SAR T cells and androgen receptor pathway inhibition. In an alternative case, patient is given SAR-NK targeting PSMA.

Advantages and Conclusions: The SHARP-tag and SABR system represents a comprehensive solution to many challenges in cell therapy and protein drug development—from manufacturing and quality control to real-time monitoring and safety management. The ability to integrate a tiny, non-immunogenic tag into therapeutic cells or proteins and to leverage a clinically deployable antibody for multiple purposes provides a new level of control. This platform can improve the safety of cell and gene therapies, streamline their production, and enhance their clinical efficacy (through adaptive dosing and conditional regulation). The novelty and utility of the present invention are further underscored by the inventor's prior efforts to develop epitope tags derived from FDA-approved antibodies or antibody-drug conjugates, including but not limited to those recognized by rituximab, Obinutuzumab, brentuximab vedotin, and Belantamab mafodotin-blmf. Tags based on epitopes recognized by these clinically validated antibodies were systematically evaluated; however, they either interfered with the expression and/or functional activity of the tagged protein (e.g., a chimeric antigen receptor [CAR]) or exhibited suboptimal sensitivity in detection assays. These limitations highlight the unexpected advantages of the presently disclosed tags and their binding counterparts.

TABLE 2

| | vL Domains and CDRs | | | | |
|---|---|---|---|---|---|
| | | SEQ | CDR1 | CDR2 | CDR3 |
| | | ID NO | (SEQ | (SEQ | (SEQ |
| Target | vL Name | (PRT) | ID NO) | ID NO) | ID NO) |
| EGFRviii | EGFRviii-GC1-Y31F-USC1 | 722 | 6601 | 6970 | 7339 |
| EGFRviii | EGFRviii-GC1 | 723 | 6602 | 6971 | 7340 |
| EGFRviii | EGFRviii-GC1-S51T-USC2 | 724 | 6603 | 6972 | 7341 |
| EGFRviii | EGFRviii-GC1-D29N-USC3 | 725 | 6604 | 6973 | 7342 |
| EGFRviii | EGFRviii-GC2 | 726 | 6605 | 6974 | 7343 |
| EGFRviii | EGFRviii-GC2-S25R | 727 | 6606 | 6975 | 7344 |
| EGFRviii | EGFRviii-GC2-S25N | 728 | 6607 | 6976 | 7345 |
| EGFRviii | EGFRviii-GC2-S31N | 729 | 6608 | 6977 | 7346 |
| GD2 | GD2-hu3F8-USC1 | 730 | 6609 | 6978 | 7347 |
| GD2 | GD2-hu3F8-USC2 | 731 | 6610 | 6979 | 7348 |
| GD2 | GD2-hu-KM666-USC1 | 732 | 6611 | 6980 | 7349 |
| GD2 | GD2-mu-KM666 | 733 | 6612 | 6981 | 7350 |
| CLDN18.2 | huCLD18A2-175D10-USC1 | 734 | 6613 | 6982 | 7351 |
| CLDN18.2 | huCLD18A2-175D10-USC2 | 735 | 6614 | 6983 | 7352 |
| CD19 | hu-mROO5-1-A28T-AT1 | 736 | 6615 | 6984 | 7353 |
| CD19 | hu-mROO5-1-Y32S-AT2 | 737 | 6616 | 6985 | 7354 |
| CD19 | hu-CD19-USC3-AT1 | 738 | 6617 | 6986 | 7355 |
| CD19 | hu-CD19-USC3-AT2 | 739 | 6618 | 6987 | 7356 |
| TAJ/TNFRSF19 | KN5-USC1-R3D | 740 | 6619 | 6988 | 7357 |
| GPRC5D | GPRC5D-Ro-5F11-USC1 | 741 | 6620 | 6989 | 7358 |
| GPRC5D | GPRC5D-Ro-5E11-USC2 | 742 | 6621 | 6990 | 7359 |
| GPRC5D | GPRC5D-Ro-5F11-E11 | 743 | 6622 | 6991 | 7360 |
| GPRC5D | GPRC5D-Ro-5E11-5F11 | 744 | 6623 | 6992 | 7361 |
| GPRC5D | GPRC5D-JJ-5B83-USC3 | 745 | 6624 | 6993 | 7362 |
| FCRH5 | FCRH5-GN-USC2 | 746 | 6625 | 6994 | 7363 |
| FR1 | FR1-huMov19-USC2 | 747 | 6626 | 6995 | 7364 |
| p53-R175H | p53-R175H | 748 | 6627 | 6996 | 7365 |
| p53-R175H | p53-R175H-AT1 | 749 | 6628 | 6997 | 7366 |
| p53-R248Q | p53-R248Q-336-USC1 | 750 | 6629 | 6998 | 7367 |
| Muc16 | huMuc16-3A5 | 751 | 6630 | 6999 | 7368 |
| ROR1 | ROR1-JJ-67 | 752 | 6631 | 7000 | 7369 |
| ROR1 | ROR1-JJ-67-AT1 | 753 | 6632 | 7001 | 7370 |
| DLL3 | DLL3-hSC16-57-USC1 | 754 | 6633 | 7002 | 7371 |
| NPM1c | NPM1c-YG1 | 755 | 6634 | 7003 | 7372 |
| IL2R | IL23R-SANG-11-vL | 756 | 6635 | 7004 | 7373 |
| HLA-A2 | HLA-A2-SANG-76-HL | 757 | 6636 | 7005 | 7374 |
| MOG | MOG01-USC1-vL | 758 | 6637 | 7006 | 7375 |
| MOG | MOG301-Y56N-USC2-vL | 759 | 6638 | 7007 | 7376 |
| MOG | MOG473-USC3-vL | 760 | 6639 | 7008 | 7377 |
| MOG | MOG01-vL | 761 | 6640 | 7009 | 7378 |
| MOG | MOG301-vL | 762 | 6641 | 7010 | 7379 |
| MOG | MOG-AZ17-vL | 763 | 6642 | 7011 | 7380 |
| CSF1R | CSF1R-AXI-vL | 764 | 6643 | 7012 | 7381 |
| CSF1R | CSF1R-AXI-USC1-vL | 765 | 6644 | 7013 | 7382 |
| TAG72 | huTAG72-v59-v15-vL | 766 | 6645 | 7014 | 7383 |
| TAG72 | TAG72-IDEC-HuCC49 | 767 | 6646 | 7015 | 7384 |
| CLDN6 | hu-CLDN6-BNT-USC1 | 768 | 6647 | 7016 | 7385 |
| CLDN6 | hu-CLDN6-BNT-USC3 | 769 | 6648 | 7017 | 7386 |
| CLDN6 | hu-CLDN6-BNT-USC4 | 770 | 6649 | 7018 | 7387 |
| CLDN6 | hu-CLDN6-BNT-USC5 | 771 | 6650 | 7019 | 7388 |
| CLDN6 | CLDN6-USC1-G51S-vL | 772 | 6651 | 7020 | 7389 |
| CLDN6 | CLDN6-129V-N33Y-G51S | 773 | 6652 | 7021 | 7390 |
| CD79b | huMA79b-AT1 | 774 | 6653 | 7022 | 7391 |
| CD79b | huMA79b-AT2 | 775 | 6654 | 7023 | 7392 |
| CD79b | huMA79b-AT3 | 776 | 6655 | 7024 | 7393 |
| CD79b | huCD79b-AT4 | 777 | 6656 | 7025 | 7394 |
| CD79b | huCD79b-AT5 | 778 | 6657 | 7026 | 7395 |
| CD79b | huCD79b-AT6 | 779 | 6658 | 7027 | 7396 |
| CD79b | huMA79b-AT5 | 780 | 6659 | 7028 | 7397 |
| CD79b | huMA79b-AT7 | 781 | 6660 | 7029 | 7398 |
| CD79b | huMA79b-AT8 | 782 | 6661 | 7030 | 7399 |
| CD79b | huCD79b-2F2-AT1 | 783 | 6662 | 7031 | 7400 |
| CD79b | huCD79b-2F2-AT2 | 784 | 6663 | 7032 | 7401 |
| CD79b | huCD79b-2F2-AT3 | 785 | 6664 | 7033 | 7402 |
| CD79b | huCD79b-2F2-AT4 | 786 | 6665 | 7034 | 7403 |
| CD79b | huCD79b-2F2-AT5 | 787 | 6666 | 7035 | 7404 |
| CD79b | huCD79b-2F2-AT6 | 788 | 6667 | 7036 | 7405 |
| CD79b | huCD79b-2F2-AT7 | 789 | 6668 | 7037 | 7406 |
| CD79b | mu-MA79b | 790 | 6669 | 7038 | 7407 |
| CD79b | CD79b-2F2-CY-vL | 791 | 6670 | 7039 | 7408 |
| CD79b | huMA79b-DeltaD | 792 | 6671 | 7040 | 7409 |
| CD79b | huMA79b-E30D-E97D- | 793 | 6672 | 7041 | 7410 |
| | AT5-vL-DelD | | | | |
| CD79b | huMA79b-AT5-vL-DelD | 794 | 6673 | 7042 | 7411 |
| CD79b | CD79b-hAb015-AT1 | 795 | 6674 | 7043 | 7412 |
| CD79b | CD79b-hAb017-AT1 | 796 | 6675 | 7044 | 7413 |
| CD79b-cyno | CynoCD79b-10D10 | 797 | 6676 | 7045 | 7414 |
| GPC3 | GPC3-USC3-HL | 798 | 6677 | 7046 | 7415 |
| GPC3 | GPC3-USC2-vL | 799 | 6678 | 7047 | 7416 |
| GPC3 | GPC3-USC3-vL | 800 | 6679 | 7048 | 7417 |
| GPC3 | GPC3-USC4-vL | 801 | 6680 | 7049 | 7418 |
| BCMA | BCMA-hu72-K30Q-P38K- | 802 | 6681 | 7050 | 7419 |
| | V46L-V84D-vL | | | | |
| BCMA | BCMA-hu72-S30G-P38K- | 803 | 6682 | 7051 | 7420 |
| | V46L-V84D-vL | | | | |
| STEAP2 | STEAP2-REGN-1162- | 804 | 6683 | 7052 | 7421 |
| | USC1-vL | | | | |
| STEAP2 | STEAP2-REGN-1162-USC3 | 805 | 6684 | 7053 | 7422 |
| STEAP2 | STEAP2-REGN-1162-USC4 | 806 | 6685 | 7054 | 7423 |
| STEAP2 | STEAP2-REGN-1162-USC5 | 807 | 6686 | 7055 | 7424 |
| CD79b | huMA79b-vL-AT10 | 808 | 6687 | 7056 | 7425 |
| CD79b | huMA79b-vL-AT11 | 809 | 6688 | 7057 | 7426 |
| CD79b | huMA79b-vL-AT12 | 810 | 6689 | 7058 | 7427 |
| CD79b | huMA79b-vL-AT13 | 811 | 6690 | 7059 | 7428 |
| CD79b | huMA79b-vL-AT14 | 812 | 6691 | 7060 | 7429 |
| CD79b | huMA79b-vL-AT15 | 813 | 6692 | 7061 | 7430 |
| CD79b | huMA79b-vL-AT16 | 814 | 6693 | 7062 | 7431 |
| CD79b | huMA79b-vL-AT17 | 815 | 6694 | 7063 | 7432 |
| CD79b | huMA79b-vL-AT18 | 816 | 6695 | 7064 | 7433 |
| CD79b | huMA79b-vL-AT19 | 817 | 6696 | 7065 | 7434 |
| CD79b | huMA79b-vL-AT20 | 818 | 6697 | 7066 | 7435 |
| CLDN6 | hu-CLDN6-BNT-USC1-6 | 819 | 6698 | 7067 | 7436 |
| CLDN7 | hu-CLDN6-BNT-USC1-7 | 820 | 6699 | 7068 | 7437 |
| CLDN8 | hu-CLDN6-BNT-AT1-vL | 821 | 6700 | 7069 | 7438 |
| CLDN9 | hu-CLDN6-BNT-AT2-vL | 822 | 6701 | 7070 | 7439 |
| CLDN10 | hu-CLDN6-BNT-AT3-vL | 823 | 6702 | 7071 | 7440 |
| CLDN11 | hu-CLDN6-BNT-AT4-vL | 824 | 6703 | 7072 | 7441 |
| CLDN12 | hu-CLDN6-BNT-AT5-vL | 825 | 6704 | 7073 | 7442 |
| CLDN13 | hu-CLDN6-BNT-AT6-vL | 826 | 6705 | 7074 | 7443 |
| CLDN14 | hu-CLDN6-BNT-AT7-vL | 827 | 6706 | 7075 | 7444 |
| STEAP2 | STEAP2-AZ40A3-N30S-vL | 828 | 6707 | 7076 | 7445 |
| STEAP3 | STEAP2-AZ40A3-vL | 829 | 6708 | 7077 | 7446 |
| GCC | GCC-22G5-vL | 830 | 6709 | 7078 | 7447 |
| GCC | GCC-AT22G5-K31N-vL | 831 | 6710 | 7079 | 7448 |
| GCC | GCC-AT22G5-A102S-vL | 832 | 6711 | 7080 | 7449 |
| GCC | GCC-AT22G5-L91Q-vL | 833 | 6712 | 7081 | 7450 |
| GCC | GCC-AT22G5-K31N-L91Q- | 834 | 6713 | 7082 | 7451 |
| | A102S-vL | | | | |
| NPM1c | NPM1c-Ab1-vL | 835 | 6714 | 7083 | 7452 |
| NPM1c | NPM1c-Ab1-AT1-vL | 836 | 6715 | 7084 | 7453 |
| NPM1c | NPM1c-Ab1-AT2-vL | 837 | 6716 | 7085 | 7454 |
| NPM1c | NPM1c-Ab1-AT3-vL | 338 | 6717 | 7086 | 7455 |
| NPM1c | NPM1c-Ab1-AT4-vL | 839 | 6718 | 7087 | 7456 |
| NPM1c | NPM1c-Ab1-AT5-vL | 840 | 6719 | 7088 | 7457 |
| NPM1c | NPM1c-Ab1-AT6-vL | 841 | 6720 | 7089 | 7458 |
| NPM1c-mut | NPM1c-mut-Ab2-vL | 842 | 6721 | 7090 | 7459 |
| NPM1c-mut | NPM1c-Mut-Ab2-AT1-vL | 843 | 6722 | 7091 | 7460 |
| NPM1c-mut | NPM1c-Mut-Ab2-AT2-vL | 844 | 6723 | 7092 | 7461 |
| NPM1c-mut | NPM1c-Mut-Ab2-AT3-vL | 845 | 6724 | 7093 | 7462 |
| Her2 | H2-Mab-250-vL | 846 | 6725 | 7094 | 7463 |
| Her2 | H2-Mab-250-AT1-vL | 847 | 6726 | 7095 | 7464 |

TABLE 3
vH domains and their CDRs

| | | SEQ | CDR1 | CDR2 | CDR3 |
|---|---|---|---|---|---|
| | | ID NO | (SEQ | (SEQ | (SEQ |
| Target | vH Name | (PRT) | ID NO) | ID NO) | ID NO) |
| EGFRviii | EGFRviii-GC1-Y31F-USC1 | 848 | 6727 | 7096 | 7465 |
| EGFRviii | EGFRviii-GC1 | 849 | 6728 | 7097 | 7466 |
| EGFRviii | EGFRviii-GC1-S51T-vL-USC2 | 850 | 6729 | 7098 | 7467 |
| EGFRviii | EGFRviii-GC1-D29N-USC3 | 851 | 6730 | 7099 | 7468 |
| EGFRviii | EGFRviii-GC2 | 852 | 6731 | 7100 | 7469 |
| EGFRviii | EGFRviii-GC2-S25R | 853 | 6732 | 7101 | 7470 |
| EGFRviii | EGFRviii-GC2-S25N | 854 | 6733 | 7102 | 7471 |
| EGFRviii | EGFRviii-GC2-S31N | 855 | 6734 | 7103 | 7472 |
| GD2 | GD2-hu3F8-USC1 | 856 | 6735 | 7104 | 7473 |
| GD2 | GD2-hu3F8-USC2 | 857 | 6736 | 7105 | 7474 |
| GD2 | GD2-hu-KM666-USC1 | 858 | 6737 | 7106 | 7475 |
| GD2 | GD2-mu-KM666 | 859 | 6738 | 7107 | 7476 |
| CLDN18.2 | hu-CLD18A2-175D10-USC1 | 860 | 6739 | 7108 | 7477 |
| CLDN18.2 | hu-CLD18A2-175D10-USC2 | 861 | 6740 | 7109 | 7478 |
| CD19 | hu-mROO5-1-AT1 | 862 | 6741 | 7110 | 7479 |
| CD19 | hu-mROO5-1-AT2 | 863 | 6742 | 7111 | 7480 |
| CD19 | hu-CD19-USC3-AT1 | 864 | 6743 | 7112 | 7481 |
| CD19 | hu-CD19-USC3-AT2 | 865 | 6744 | 7113 | 7482 |
| TAJ | KN5-USC1-R3D | 866 | 6745 | 7114 | 7483 |
| GPRC5D | GPRC5D-Ro-5F11-USC1 | 867 | 6746 | 7115 | 7484 |
| GPRC5D | GPRC5D-Ro-5E11-USC2 | 868 | 6747 | 7116 | 7485 |
| GPRC5D | GPRC5D-Ro-5F11-E11-USC | 869 | 6748 | 7117 | 7486 |
| GPRC5D | GPRC5D-Ro-5E11-5F11-USC | 870 | 6749 | 7118 | 7487 |
| GPRC5D | GPRC5D-JJ-5B83-USC3 | 871 | 6750 | 7119 | 7488 |
| FCRH5 | FCRH5-GN-USC2 | 872 | 6751 | 7120 | 7489 |
| FR1 | FR1-huMov19-USC2 | 873 | 6752 | 7121 | 7490 |
| p53-R175H | p53-R175H | 874 | 6753 | 7122 | 7491 |
| p53-R175H | p53-R175H-AT1 | 875 | 6754 | 7123 | 7492 |
| p53-R248Q | p53-R248Q-336-USC1 | 876 | 6755 | 7124 | 7493 |
| Muc16 | huMuc16-3A5 | 877 | 6756 | 7125 | 7494 |
| ROR1 | ROR1-JJ-67 | 878 | 6757 | 7126 | 7495 |
| ROR1 | ROR1-JJ-67-AT1 | 879 | 6758 | 7127 | 7496 |
| DLL3 | DLL3-hSC16-57-USC1 | 880 | 6759 | 7128 | 7497 |
| NPM1c | NPM1c-YG1 | 881 | 6760 | 7129 | 7498 |
| IL2R | IL23R-SANG-11 | 882 | 6761 | 7130 | 7499 |
| HLA-A2 | HLA-A2-SANG-76-HL | 883 | 6762 | 7131 | 7500 |
| MOG | MOG01-USC1 | 884 | 6763 | 7132 | 7501 |
| MOG | MOG301-Y56N-USC2 | 885 | 6764 | 7133 | 7502 |
| MOG | MOG473-USC3 | 886 | 6765 | 7134 | 7503 |
| MOG | MOG01 | 887 | 6766 | 7135 | 7504 |
| MOG | MOG301 | 888 | 6767 | 7136 | 7505 |
| MOG | MOG-AZ17 | 889 | 6768 | 7137 | 7506 |
| CSF1R | CSF1R-AXI | 890 | 6769 | 7138 | 7507 |
| CSF1R | CSF1R-AXI-USC1 | 891 | 6770 | 7139 | 7508 |
| TAG72 | huTAG72-v59-v15 | 892 | 6771 | 7140 | 7509 |
| TAG72 | TAG72-IDEC-HuCC49 | 893 | 6772 | 7141 | 7510 |
| CLDN6 | hu-CLDN6-BNT-USC1 | 894 | 6773 | 7142 | 7511 |
| CLDN6 | hu-CLDN6-BNT-USC3 | 895 | 6774 | 7143 | 7512 |
| CLDN6 | hu-CLDN6-BNT-USC4 | 896 | 6775 | 7144 | 7513 |
| CLDN6 | CLDN6-USC1 | 897 | 6776 | 7145 | 7514 |
| CLDN6 | CLDN6-USC2-DNA | 898 | 6777 | 7146 | 7515 |
| CD79b | huMA79b-AT1 | 899 | 6778 | 7147 | 7516 |
| CD79b | huMA79b-AT2 | 900 | 6779 | 7148 | 7517 |
| CD79b | huMA79b-AT3 | 901 | 6780 | 7149 | 7518 |
| CD79b | huCD79b-AT4 | 902 | 6781 | 7150 | 7519 |
| CD79b | huCD79b-AT5 | 903 | 6782 | 7151 | 7520 |
| CD79b | huCD79b-AT6 | 904 | 6783 | 7152 | 7521 |
| CD79b | huMA79b-AT5 | 905 | 6784 | 7153 | 7522 |
| CD79b | huMA79b-AT7 | 906 | 6785 | 7154 | 7523 |
| CD79b | huMA79b-AT8 | 907 | 6786 | 7155 | 7524 |
| CD79b | huCD79b-2F2-AT1 | 908 | 6787 | 7156 | 7525 |
| CD79b | huCD79b-2F2-AT2 | 909 | 6788 | 7157 | 7526 |
| CD79b | huCD79b-2F2-AT3 | 910 | 6789 | 7158 | 7527 |
| CD79b | huCD79b-2F2-AT4 | 911 | 6790 | 7159 | 7528 |
| CD79b | huCD79b-2F2-AT5 | 912 | 6791 | 7160 | 7529 |
| CD79b | huCD79b-2F2-AT6 | 913 | 6792 | 7161 | 7530 |
| CD79b | huCD79b-2F2-AT7 | 914 | 6793 | 7162 | 7531 |
| CD79b | mu-MA79b | 915 | 6794 | 7163 | 7532 |
| CD79b | CD79b-2F2-CY | 916 | 6795 | 7164 | 7533 |
| CD79b | huMA79bv28-DeltaD | 917 | 6796 | 7165 | 7534 |
| CD79b | huMA79b-S30T | 918 | 6797 | 7166 | 7535 |
| CD79b | huMA79b | 919 | 6798 | 7167 | 7536 |
| CD79b | CD79b-hAb015-AT1 | 920 | 6799 | 7168 | 7537 |
| CD79b | CD79b-hAb017-AT1 | 921 | 6800 | 7169 | 7538 |
| CD79b-cyno | CynoCD79b-10D10 | 922 | 6801 | 7170 | 7539 |
| GPC3 | GPC3-USC3-HL | 923 | 6802 | 7171 | 7540 |
| GPC3 | GPC3-USC2 | 924 | 6803 | 7172 | 7541 |
| GPC3 | GPC3-USC4 | 925 | 6804 | 7173 | 7542 |
| GPC3 | GPC3-USC5 | 926 | 6805 | 7174 | 7543 |
| BCMA | BCMA-hu72-F35Y-I58S-S64T | 927 | 6806 | 7175 | 7544 |
| BCMA | BCMA-hu72-S32N-F35Y- | 928 | 6807 | 7176 | 7545 |
| | I58S-S64T | | | | |
| STEAP2 | STEAP2-REGN-1162-USC1 | 929 | 6808 | 7177 | 7546 |
| STEAP2 | STEAP2-REGN-1162-USC3 | 930 | 6809 | 7178 | 7547 |
| STEAP2 | STEAP2-REGN-1162-USC4 | 931 | 6810 | 7179 | 7548 |
| STEAP2 | STEAP2-REGN-1162-USC5 | 932 | 6811 | 7180 | 7549 |
| STEAP2 | STEAP2-REGN-1162-USC6 | 933 | 6812 | 7181 | 7550 |
| STEAP2 | STEAP2-REGN-1162-USC7 | 934 | 6813 | 7182 | 7551 |
| STEAP2 | STEAP2-REGN-1162-USC8 | 935 | 6814 | 7183 | 7552 |
| STEAP2 | STEAP2-REGN-1162-USC9 | 936 | 6815 | 7184 | 7553 |
| CD79b | CD79b-huMA79b-AT10 | 937 | 6816 | 7185 | 7554 |
| CD79b | CD79b-huMA79b-AT11 | 938 | 6817 | 7186 | 7555 |
| CD79b | CD79b-huMA79b-AT12 | 939 | 6818 | 7187 | 7556 |
| CD79b | CD79b-huMA79b-AT13 | 940 | 6819 | 7188 | 7557 |
| CD79b | CD79b-huMA79b-AT14 | 941 | 6820 | 7189 | 7558 |
| CLDN6 | hu-CLDN6-BNT-USC1-6 | 942 | 6821 | 7190 | 7559 |
| CLDN6 | hu-CLDN6-BNT-USC1-7 | 943 | 6822 | 7191 | 7560 |
| CLDN6 | hu-CLDN6-BNT-AT1 | 944 | 6823 | 7192 | 7561 |
| CLDN6 | hu-CLDN6-BNT-AT2 | 945 | 6824 | 7193 | 7562 |
| CLDN6 | hu-CLDN6-BNT-AT3 | 946 | 6825 | 7194 | 7563 |
| CLDN6 | hu-CLDN6-BNT-AT4 | 947 | 6826 | 7195 | 7564 |
| CLDN6 | hu-CLDN6-BNT-AT5 | 948 | 6827 | 7196 | 7565 |
| CLDN6 | hu-CLDN6-BNT-AT6 | 949 | 6828 | 7197 | 7566 |
| CLDN6 | hu-CLDN6-BNT-AT8 | 950 | 6829 | 7198 | 7567 |
| STEAP2 | STEAP2-AZ40A3-K58E | 951 | 6830 | 7199 | 7568 |
| STEAP2 | STEAP2-AZ40A3 | 952 | 6831 | 7200 | 7569 |
| GCC | GCC-22G5-LGND | 953 | 6832 | 7201 | 7570 |
| GCC | GCC-AT22G5-S30A | 954 | 6833 | 7202 | 7571 |
| GCC | GCC-AT22G5-V101L | 955 | 6834 | 7203 | 7572 |
| GCC | GCC-AT22G5-S30A-V101L | 956 | 6835 | 7204 | 7573 |
| GCC | GCC-AT22G5-D95E | 957 | 6836 | 7205 | 7574 |
| NPM1c | NPM1c-Ab1 | 958 | 6837 | 7206 | 7575 |
| NPM1c | NPM1c-Ab1-AT1 | 959 | 6838 | 7207 | 7576 |
| NPM1c | NPM1c-Ab1-AT2 | 960 | 6839 | 7208 | 7577 |
| NPM1c | NPM1c-Ab1-AT3 | 961 | 6840 | 7209 | 7578 |
| NPM1c-mut | NPM1c-mut-Ab2-vL | 962 | 6841 | 7210 | 7579 |
| NPM1c-mut | NPM1c-Mut-Ab2-AT1 | 963 | 6842 | 7211 | 7580 |
| NPM1c-mut | NPM1c-Mut-Ab2-AT2 | 964 | 6843 | 7212 | 7581 |
| NPM1c-mut | NPM1c-Mut-Ab2-AT3 | 965 | 6844 | 7213 | 7582 |
| Her2 | H2-Mab-250 | 966 | 6845 | 7214 | 7583 |
| Her2 | H2-Mab-250_AT1 | 967 | 6846 | 7215 | 7584 |
| Her2 | H2-Mab-250_AT2 | 968 | 6847 | 7216 | 7585 |
| Her2 | H2-Mab-250_AT3 | 969 | 6848 | 7217 | 7586 |
| Her2 | H2-Mab-250_AT4 | 970 | 6849 | 7218 | 7587 |

TABLE 4
vHH and FHVH domains and their CDRs

| | | SEQ | CDR1 | CDR2 | CDR3 |
|---|---|---|---|---|---|
| | | ID NO | (SEQ | (SEQ | (SEQ |
| Target | vHH or FHVH Name | (PRT) | ID NO) | ID NO) | ID NO) |
| BCMA | hu-BCMA917-E59D-AT4 | 1081 | 6850 | 7219 | 7588 |
| BCMA | hu-BCMA917-E59D-AT5 | 1082 | 6851 | 7220 | 7589 |
| BCMA | hu-BCMA917-E59D-AT6 | 1083 | 6852 | 7221 | 7590 |
| BCMA | hu-BCMA917-E59D-AT7 | 1084 | 6853 | 7222 | 7591 |
| BCMA | hu-BCMA917-E59D-AT8 | 1085 | 6854 | 7223 | 7592 |
| BCMA | hu-BCMA917-E59D-AT9 | 1086 | 6855 | 7224 | 7593 |
| BCMA | hu-BCMA917-E59D-AT10 | 1087 | 6856 | 7225 | 7594 |
| BCMA | hu-BCMA917-E59D-AT11 | 1088 | 6857 | 7226 | 7595 |
| BCMA | hu-BCMA917-E59D-AT12 | 1089 | 6858 | 7227 | 7596 |
| BCMA | hu-BCMA917-E59D-AT13 | 1090 | 6859 | 7228 | 7597 |
| CD20 | CD20-2HCD27-AT3-vHH | 1091 | 6860 | 7229 | 7598 |
| CD20 | CD20-2HCD28-AT4-vHH | 1092 | 6861 | 7230 | 7599 |
| CD20 | CD20-VHH-AT1-2HCD29 | 1093 | 6862 | 7231 | 7600 |
| CD20 | CD20-VHH-AT1-2HCD30 | 1094 | 6863 | 7232 | 7601 |
| CD22 | CD22-hu-077-AT3-vHH | 1095 | 6864 | 7233 | 7602 |
| CD19 | CD19-huVHH-AT773-N32S-S58T | 1096 | 6865 | 7234 | 7603 |
| CD19 | CD19-huVHH-AT773-N27S-N32S- | 1097 | 6866 | 7235 | 7604 |
| | S58T | | | | |
| CD19 | CD19-huVHH-AT883-N32S-S58T | 1098 | 6867 | 7236 | 7605 |
| CD19 | CD19-huVHH-AT773-N29S-S58T | 1099 | 6868 | 7237 | 7606 |
| CD19 | CD19-VHH-AT83-N29S-S58T | 1100 | 6869 | 7238 | 7607 |
| CD19 | CD19-huVHH-AT773-N27S-S58T | 1101 | 6870 | 7239 | 7608 |
| CD19 | CD19-huVHH-AT773-WT | 1102 | 6871 | 7240 | 7609 |
| CD19 | CD19-VHH-AT83-WT | 1103 | 6872 | 7241 | 7610 |
| CD33 | CD33-vHH-AT264-S30G-V50A | 1104 | 6873 | 7242 | 7611 |
| CD33 | CD33-huvHH-AT264-S30G-V50A | 1105 | 6874 | 7243 | 7612 |
| CD33 | CD33-huvHH-AT264-S30G-V50A- | 1106 | 6875 | 7244 | 7613 |
| | K65R | | | | |
| CD33 | CD33-vHH-AT264-S30G-V50A-K65R | 1107 | 6876 | 7245 | 7614 |
| CD33 | CD33-huvHH-AT873-G30S-A50V | 1108 | 6877 | 7246 | 7615 |
| MSLN | MSLN-vHH-AT2-K65R | 1109 | 6878 | 7247 | 7616 |
| MSLN | MSLN-vHH-AT3-K65R-I109V-R114H | 1110 | 6879 | 7248 | 7617 |
| MSLN | MSLN-vHH-AT4-T61A-S105T-R114H | 1111 | 6880 | 7249 | 7618 |
| MSLN | MSLN-vHH-AT5-T61A-I109V-R114H | 1112 | 6881 | 7250 | 7619 |
| MSLN | MSLN-vHH-AT6-T61A-S105T-I109V- | 1113 | 6882 | 7251 | 7620 |
| | R114H | | | | |
| MSLN | MSLN-vHH-AT7 | 1114 | 6883 | 7252 | 7621 |
| DLL3 | DLL3-vHH-AT6 | 1115 | 6884 | 7253 | 7622 |
| DLL3 | DLL3-vHH-AT7 | 1116 | 6885 | 7254 | 7623 |
| DLL3 | DLL3-vHH-AT8 | 1117 | 6886 | 7255 | 7624 |
| DLL3 | DLL3-vHH-AT9 | 1118 | 6887 | 7256 | 7625 |
| DLL3 | DLL3-vHH-AT10 | 1119 | 6888 | 7257 | 7626 |
| DLL3 | DLL3-vHH-AT11 | 1120 | 6889 | 7258 | 7627 |
| DLL3 | DLL3-vHH-AT12 | 1121 | 6890 | 7259 | 7628 |
| IL13Ra2 | IL13Ra2-huvHH-AT1-C11 | 1122 | 6891 | 7260 | 7629 |
| IL13Ra2 | IL13Ra2-huvHH-AT3-Cl1 | 1123 | 6892 | 7261 | 7630 |
| IL13Ra2 | IL13Ra2-vHH-Cl2 | 1124 | 6893 | 7262 | 7631 |
| IL13Ra2 | IL13Ra2-vHH-AT4-Cl3 | 1125 | 6894 | 7263 | 7632 |
| Muc16 | MUC16-VHH-R3MU29 | 1126 | 6895 | 7264 | 7633 |
| Muc16 | MUC16-vHH-R3MU5-AT1 | 1127 | 6896 | 7265 | 7634 |
| Muc16 | MUC16-vHH-R3MU5-AT3 | 1128 | 6897 | 7266 | 7635 |
| Muc16 | MUC16-AT2-huR3MU30 | 1129 | 6898 | 7267 | 7636 |
| Muc16 | MUC16-VHH-R3MU31-AT4 | 1130 | 6899 | 7268 | 7637 |
| BCMA | BCMA917-V5L-T30S-E59D-AT16 | 1131 | 6900 | 7269 | 7638 |
| BCMA | BCMA-vHH-917-S49N-AT17 | 1132 | 6901 | 7270 | 7639 |
| BCMA | BCMA917-S102A-AT18 | 1133 | 6902 | 7271 | 7640 |
| BCMA | BCMA917-D96E-AT19 | 1134 | 6903 | 7272 | 7641 |
| BCMA | BCMA-vHH-917-AT20 | 1135 | 6904 | 7273 | 7642 |
| BCMA | BCMA-vHH-917-AT21 | 1136 | 6905 | 7274 | 7643 |
| BCMA | BCMA-vHH-917 AT22 | 1137 | 6906 | 7275 | 7644 |
| BCMA | BCMA-vHH-917- AT23 | 1138 | 6907 | 7276 | 7645 |
| BCMA | BCMA-vHH-917-AT24 | 1139 | 6908 | 7277 | 7646 |
| BCMA | BCMA355-F106Y-vHH-AT1 | 1140 | 6909 | 7278 | 7647 |
| BCMA | BCMA355-S57N-A104S-vHH-AT2 | 1141 | 6910 | 7279 | 7648 |
| CD22 | CD22-huVHH-077 | 1142 | 6911 | 7280 | 7649 |
| CD22 | CD22-huVHH-077-F101Y-AT4 | 1143 | 6912 | 7281 | 7650 |
| CD22 | CD22-huVHH-077-V103I-AT5 | 1144 | 6913 | 7282 | 7651 |
| CD22 | CD22-huVHH-077-S58A-AT6 | 1145 | 6914 | 7283 | 7652 |
| CD22 | CD22-huVHH-077F34Y-S58A-AT7 | 1146 | 6915 | 7284 | 7653 |
| CD22 | CD22-huVHH-077-V106I-AT8 | 1147 | 6916 | 7285 | 7654 |
| CD22 | CD22-huVHH-077F34Y-V106I-AT9 | 1148 | 6917 | 7286 | 7655 |
| CD19 | CD19-huVHH-773 | 1149 | 6918 | 7287 | 7656 |
| CD19 | CD19-huVHH-AT773-WT(N32T) | 1150 | 6919 | 7288 | 7657 |
| CD19 | CD19-huVHH-AT773-A106S-AT1 | 1151 | 6920 | 7289 | 7658 |
| CD19 | CD19-huVHH-AT773-A99S-AT2 | 1152 | 6921 | 7290 | 7659 |
| CD19 | CD19-huVHH-AT773-AT3 | 1153 | 6922 | 7291 | 7660 |
| DLL3 | DLL3-vHH-T2 | 1154 | 6923 | 7292 | 7661 |
| DLL3 | DLL3-vHH-T2-N57S-AT12 | 1155 | 6924 | 7293 | 7662 |
| DLL3 | DLL3-vHH-T2-S55G-N57S-AT13 | 1156 | 6925 | 7294 | 7663 |
| DLL3 | DLL3-vHH-T2-D107E-AT14 | 1157 | 6926 | 7295 | 7664 |
| CD20 | CD20-vHH-G100S-AT5-2HCD31 | 1158 | 6927 | 7296 | 7665 |
| CD20 | CD20-vHH-G53A-AT6-2HCD32 | 1159 | 6928 | 7297 | 7666 |
| CD20 | CD20-vHH- AT7-2HCD33 | 1160 | 6929 | 7298 | 7667 |
| CD20 | CD20-vHH-AT8-2HCD34 | 1161 | 6930 | 7299 | 7668 |
| CD20 | CD20-R3CD7-vHH | 1162 | 6931 | 7300 | 7669 |
| BCMA | CD20-R3CD7- AT1 | 1163 | 6932 | 7301 | 7670 |
| BCMA | BCMA-FHVH-93-H32Y-AT1 | 1164 | 6933 | 7302 | 7671 |
| BCMA | BCMA-FHVH-33-Y32H-AT2 | 1165 | 6934 | 7303 | 7672 |
| BCMA | BCMA-FHVH-74-TNSS-AT3 | 1166 | 6935 | 7304 | 7673 |
| CD22 | CD22-FHVH24-D27G-D33S | 1167 | 6936 | 7305 | 7674 |
| CD22 | CD22-FHVH24-V58I | 1168 | 6937 | 7306 | 7675 |
| CD22 | CD22-FHVH24-V58I-T59S | 1169 | 6938 | 7307 | 7676 |
| CD22 | CD22-FHVH24-Y55H-V58I-T59S | 1170 | 6939 | 7308 | 7677 |
| CD22 | CD22-FHVH24-S57N-V58I-T59S | 1171 | 6940 | 7309 | 7678 |
| CD22 | CD22-FHVH24-S57A-V58I-T59S | 1172 | 6941 | 7310 | 7679 |
| CLDN6 | hu-CDLN6-10-LGND | 580 | 6942 | 7311 | 7680 |
| CLDN6 | hu-CDLN6-G51S-AT10-vHH | 581 | 6943 | 7312 | 7681 |
| CLDN6 | hu-CDLN6-D100E-AT11-vHH | 582 | 6944 | 7313 | 7682 |
| CLDN6 | hu-CDLN6-I104V-AT12-vHH | 583 | 6945 | 7314 | 7683 |
| CLDN6 | hu-CDLN6-L97I-AT13-vHH | 584 | 6946 | 7315 | 7684 |
| GPRC5D | GPRC5D-AS300413VH7 | 585 | 6947 | 7316 | 7685 |
| GPRC5D | hu-GPRC5D-AT413-E98D-vHH | 586 | 6948 | 7317 | 7686 |
| GPRC5D | GPRC5D-LGND-AS302399VH4 | 587 | 6949 | 7318 | 7687 |
| GPRC5D | hu-GPRC5D-AT2399-A99S-vHH | 588 | 6950 | 7319 | 7688 |
| GPRC5D | hu-GPRC5D-AT2399-A57S-vHH | 589 | 6951 | 7320 | 7689 |
| GPRC5D | GPRC5D-LGND-AS302399 | 590 | 6952 | 7321 | 7690 |
| GPRC5D | GPRC5D-AT2399-T108S-vHH | 591 | 6953 | 7322 | 7691 |
| GPRC5D | GPRC5D-AT2399-S55N-vHH | 592 | 6954 | 7323 | 7692 |
| GPRC5D | GPRC5D-AS302558VH6 | 593 | 6955 | 7324 | 7693 |
| GPRC5D | hu-GPRC5D-AT2558-E56D-vHH | 594 | 6956 | 7325 | 7694 |
| CD33 | CD33-huvHH-AT873-G30S | 595 | 6957 | 7326 | 7695 |
| CD33 | CD33-huvHH-AT873-G30S-R99K | 596 | 6958 | 7327 | 7696 |
| CD33 | CD33-huvHH-AT873-G30S-T57R- | 597 | 6959 | 7328 | 7697 |
| | R99K | | | | |
| CD33 | CD33-vHH-AT264-K99R | 598 | 6960 | 7329 | 7698 |
| CD33 | CD33-vHH-AT264-V50A-R57T | 599 | 6961 | 7330 | 7699 |
| CD33 | CD33-vHH-AT264-V50A-R57T-K99R | 600 | 6962 | 7331 | 7700 |
| CLL1 | CLL1-IG6-vHH | 601 | 6963 | 7332 | 7701 |
| CLL1 | CLL1-IG6-A30S-AT-vHH | 602 | 6964 | 7333 | 7702 |
| CLL1 | CLL1-2H3-vHH | 603 | 6965 | 7334 | 7703 |
| CLL1 | CLL1-2H3- S27D-AT-vHH | 604 | 6966 | 7335 | 7704 |
| CLL1 | CLL1-2H3-I28T-AT-vHH | 605 | 6967 | 7336 | 7705 |
| CLL1 | CLL1-1H1-WT-vHH | 606 | 6968 | 7337 | 7706 |
| CLL1 | CLL1-1H1-N30S-AT-vHH | 607 | 6969 | 7338 | 7707 |

TABLE 5
Results of JNG activation and ELISA with different tag constructs
TABLE 5 Results of JNG activation and ELISA with different tag construct

| | | | JNG ASSAY (%) | | | | | | |
|---|---|---|---|---|---|---|---|---|---|
| SUPERNATANTS | | | | (SEQ | (SEQ | SEQ | ELISA | | |
| | SEQ | SEQ | | ID NO: | ID NO: | ID NO: | SEQ | SEQ | SEQ |
| | ID NO | ID | JNG-P | 2448 | 2407 | 2448 | ID NO: | ID NO: | ID NO: |
| CLONE ID NO | (DNA) | (PRT) | (%) | (%) | (%) | (%) | 3632) | 3639 | 3633 |
| JNG-P, control | | | 0.08 | 0.09 | 0.27 | 6.70 | | | |
| Supernatant | | | | | | | | | |
| 020224-BBaZ2 | 5075 | 2625 | 0.06 | 25.47 | 13.77 | | | | |
| 020224-BBbA1 | 5076 | 2626 | 0.08 | 15.11 | 15.63 | | 0.121 | 0.436 | 0.071 |
| 020224-BBbB2 | 5077 | 2627 | 0.00 | 25.20 | 14.12 | | 0.195 | 0.617 | 0.085 |
| 020224-BBbC1 | 5078 | 2628 | 0.04 | 10.72 | 8.11 | | 0.194 | 0.993 | 0.068 |
| 020224-BBbD2 | 5079 | 2629 | 0.00 | 6.24 | 4.53 | | 0.082 | 0.215 | 0.061 |
| 020224-BBbE2 | 5080 | 2630 | 0.02 | 8.75 | 4.97 | | 0.079 | 0.179 | 0.070 |
| 020224-BBbF1 | 5081 | 2631 | 0.00 | 40.47 | 23.91 | | 0.124 | 0.352 | 0.072 |
| 020224-BBbH2 | 5082 | 2632 | 0.07 | 36.19 | 21.76 | | 0.595 | 1.307 | 0.173 |
| 020224-BBbK1 | 5083 | 2633 | 0.06 | 9.16 | 7.63 | | 0.084 | 0.146 | 0.062 |
| 020224BBbL2 | 5084 | 2634 | 0.04 | 43.98 | 28.42 | | 0.500 | 0.708 | 0.252 |
| 020224-BBbM1 | 5085 | 2635 | 0.02 | 5.23 | 2.65 | | 0.249 | 0.636 | 0.093 |
| 020224-BBbN1 | 5086 | 2636 | 0.04 | 17.49 | 9.60 | | 0.239 | 0.534 | 0.104 |
| 020224-BBbO2 | 5087 | 2637 | 0.04 | 0.21 | 0.35 | | 0.080 | 0.294 | 0.063 |
| 020224-BBbP1 | 5088 | 2638 | 0.04 | 22.81 | 13.01 | | 0.533 | 1.333 | 0.143 |
| 020224-BBbQ1 | 5089 | 2639 | 0.06 | 25.52 | 11.74 | | 0.091 | 0.179 | 0.061 |
| 020224-BBbS2 | 5090 | 2640 | 0.04 | 31.62 | 23.85 | | 0.720 | 1.334 | 0.258 |
| 020224-BBbT1 | 5091 | 2641 | 0.06 | 12.81 | 11.49 | | 0.967 | 1.271 | 0.272 |
| 021924-BBeA2 | 5092 | 2642 | 0.32 | 22.55 | 23.08 | | 0.436 | 0.950 | 0.385 |
| 031124-BBeR1 | 5093 | 2643 | 0.22 | 36.81 | 16.24 | 57.2 | 0.591 | 0.179 | 0.310 |
| 031124-BBeS1 | 5094 | 2644 | 0.18 | 51.84 | 13.31 | 47.6 | 0.109 | 0.368 | 0.191 |
| 031124-BBeT1 | 5095 | 2645 | 0.24 | 52.12 | 11.18 | 46.1 | 0.102 | 0.092 | 0.131 |
| 031124-BBeW1 | 5096 | 2646 | 0.32 | 22.55 | 9.85 | | | | |
| 031124-BBeW2 | 5097 | 2647 | 0.29 | 18.56 | 9.58 | | | | |
| 031124-BBeW3 | 5098 | 2648 | 0.24 | 23.27 | 8.70 | | | | |
| 031124-BBeW6 | 5099 | 2649 | 0.27 | 15.30 | 6.03 | | | | |
| 031124BBeX1 | 5100 | 2650 | 0.17 | 28.46 | 16.36 | | | | |
| 031124BBeX2 | 5101 | 2651 | 0.29 | 27.37 | 13.48 | | | | |
| 031124BBeX3 | 5102 | 2652 | 0.18 | 38.76 | 22.06 | | | | |
| 031124BBeX4 | 5103 | 2653 | 0.19 | 29.18 | 16.10 | | | | |
| 031124-BBeY1 | 5104 | 2654 | 0.27 | 21.86 | 9.89 | | | | |
| 031124-BBeY2 | 5105 | 2655 | 0.19 | 19.41 | 11.82 | | | | |
| 031124-BBeY3 | 5106 | 2656 | 0.05 | 8.80 | 3.91 | | | | |
| 031124-BBeY4 | 5107 | 2657 | 0.19 | 26.71 | 10.84 | | | | |
| 031124-BBeY5 | 5108 | 2658 | 0.00 | 27.36 | 9.96 | | | | |
| 031124BBeW4 | 5109 | 2659 | 0.29 | 24.33 | 8.17 | | | | |
| 031124-BBeX5 | 5110 | 2660 | 0.07 | 22.35 | 9.47 | | | | |
| 031124BBeW5 | 5111 | 2661 | 0.24 | 23.53 | 8.32 | | | | |
| 031124-BBeX6 | 5112 | 2662 | 0.30 | 24.38 | 9.92 | | | | |
| 031124-BBeU2 | 5113 | 2663 | 0.00 | 39.46 | 17.64 | 0.30 | 1.062 | 1.011 | 0.566 |
| 031124-BBeV2 | 5114 | 2664 | 0.06 | 3.12 | 1.77 | 4.20 | 0.082 | 0.090 | 0.112 |
| 040524-EZeG1 | 5115 | 2665 | 0.08 | 38.24 | 18.45 | | | 0.080 | 0.143 |
| 040524-EZeH1 | 5116 | 2666 | 0.06 | 34.89 | 9.80 | | 0.311 | 0.427 | 0.152 |
| 040524-EZeJ1 | 5117 | 2667 | 0.08 | 0.52 | 0.44 | | | 0.121 | |
| 040524-EZeK1 | 5118 | 2668 | 0.00 | 0.18 | 0.32 | | 0.080 | 0.091 | 0.077 |
| 040524-EZeJ2 | 5119 | 2669 | 0.82 | 0.81 | 0.33 | | 0.116 | 0.106 | 0.082 |
| 040524-EZeJ3 | 5120 | 2670 | 0.71 | 1.27 | 1.61 | | 0.176 | 0.295 | 0.113 |
| 040524-EZeJ4 | 5121 | 2671 | 0.92 | 1.71 | 3.90 | | | | |
| 040524-EZeJ6 | 5122 | 2672 | 0.74 | 0.91 | 0.55 | | 0.165 | 0.132 | 0.105 |
| 040524-EZeJ7 | 5123 | 2673 | 0.89 | 0.77 | 0.79 | | 0.116 | 0.145 | 0.074 |
| 040524-EZeK5 | 5126 | 2676 | 0.68 | 0.64 | 0.20 | | 0.078 | 0.102 | 0.075 |
| 040524-EZeK6 | 5127 | 2677 | 0.72 | 0.83 | 0.24 | | 0.080 | 0.092 | 0.067 |
| 040524-EZeK7 | 5128 | 2678 | 0.78 | 0.86 | 0.15 | | 0.089 | 0.099 | 0.076 |
| 040524-EZeK9 | 5130 | 2680 | 0.68 | 0.79 | 0.09 | | 0.085 | 0.174 | 0.070 |
| 040524-EZeK10 | 5131 | 2681 | 0.91 | 0.69 | 0.24 | | 0.079 | 0.124 | 0.069 |
| 041024-EZeJ1 | 5134 | 2684 | 0.23 | 7.72 | 2.48 | | 0.275 | 0.173 | 0.114 |
| 041024-EZeJ3 | 5135 | 2685 | 0.26 | 0.33 | | | 0.097 | 0.108 | 0.084 |
| 041024-EZeJ4 | 5136 | 2686 | 0.18 | 1.54 | 0.30 | | 0.188 | 0.182 | 0.111 |
| 041024-EZeJ7 | 5138 | 2688 | 0.36 | 2.23 | 0.48 | | 0.199 | 0.214 | 0.110 |
| 041024-EZeJ9 | 5139 | 2689 | 0.25 | 0.22 | | | 0.104 | 0.106 | 0.089 |
| 041024-EZeJ10 | 5140 | 2690 | 0.42 | 34.8 | 8.56 | 36.1 | | | |
| 041024-EZeK4 | 5143 | 2693 | 0.25 | | 0.38 | | 0.094 | 0.102 | 0.082 |
| 041024-EZeK8 | 5147 | 2697 | 0.34 | | 0.37 | | 0.092 | 0.102 | 0.078 |
| 041024-EZeK9 | 5148 | 2698 | 0.28 | | 0.15 | | 0.094 | 0.106 | 0.075 |
| 041024-EZeK10 | 5149 | 2699 | 0.29 | | 0.36 | | 0.092 | 0.092 | 0.076 |
| 041924-BBhA1 | 5150 | 2700 | 0.26 | | 1.05 | | | | |
| 041924-BBhA2 | 5151 | 2701 | 0.25 | | 0.19 | | 0.089 | 0.093 | 0.075 |
| 041924-BBhA3 | 5152 | 2702 | 0.28 | | 0.46 | | 0.122 | 0.108 | 0.083 |
| 041924-BBhA4 | 5153 | 2703 | 0.20 | | 0.29 | | 0.095 | 0.102 | 0.082 |
| 041924-BBhA5 | 5154 | 2704 | 0.30 | | 0.37 | | 0.092 | 0.107 | 0.082 |
| 041924BBhA10 | 5155 | 2705 | 0.33 | | 0.34 | | 0.085 | 0.100 | 0.078 |
| 041924BBhA22 | 5160 | 2710 | 0.17 | | 0.28 | | 0.101 | 0.102 | 0.096 |
| 050524-EZqA1 | 5188 | 2738 | | | | 0.10 | 0.070 | 0.132 | 0.071 |
| 050524-EZqE2 | 5190 | 2740 | | | | 0.21 | 0.085 | 0.103 | 0.076 |
| 050524-EZqF1 | 5192 | 2742 | | | | 53.8 | 0.706 | 0.105 | 0.428 |
| 050524-EZqF3 | 5193 | 2743 | | | | 44.3 | 0.522 | 0.083 | 0.178 |
| 050524-EZqG1 | 5194 | 2744 | | | | 7.65 | 0.174 | 0.109 | 0.073 |
| 050524-EZqH2 | 5195 | 2745 | | | | 0.13 | 0.093 | 0.184 | 0.075 |
| 050524-EZqK1 | 5199 | 2749 | 0.00 | 41.97 | 49.11 | 25.4 | 0.641 | 0.433 | 0.379 |
| 050524-EZqN2 | 5200 | 2750 | | | | 0.09 | 0.088 | 0.099 | 0.080 |
| 050524-EZqB3 | 5203 | 2753 | 0.11 | 0.08 | 0.01 | | 0.081 | 0.099 | 0.084 |
| 050524-EZqB6 | 5205 | 2755 | 0.15 | 0.13 | 0.1 | | 0.075 | 0.134 | 0.073 |
| 050524-EZqB7 | 5206 | 2756 | 0.16 | 0.11 | 0.04 | | 0.086 | 0.100 | 0.102 |
| 050524-EZqB9 | 5208 | 2758 | 0.2 | 0.11 | 0.07 | | 0.085 | 0.105 | 0.085 |
| 050524-EZqB1 | 5209 | 2759 | 0.3 | 0.11 | 0.02 | | 0.089 | 0.117 | 0.075 |
| 050524-EZqB1 | 5210 | 2760 | 0.19 | 0.1 | 0.12 | | 0.085 | 0.100 | 0.077 |
| 050524-EZqF8 | 5223 | 2773 | 0.13 | 26.6 | 16.71 | | 0.271 | 0.108 | 0.173 |
| 050524-EZqF9 | 5224 | 2774 | 0.16 | 0.15 | 0.08 | | 0.131 | 0.093 | 0.084 |
| 050524-EZqJ8 | 5236 | 2786 | 0.25 | 0.09 | 0.05 | | 0.077 | 0.100 | 0.075 |
| 050524-EZqK2 | 5237 | 2787 | 0.04 | 0.09 | 0.05 | | 0.102 | 0.094 | 0.078 |
| 050524-EZqK5 | 5240 | 2790 | 0.14 | 0.19 | 0.08 | | 0.110 | 0.093 | 0.077 |
| 050524-EZqK7 | 5242 | 2792 | 0.25 | 0.13 | 0.05 | | 0.119 | 0.138 | 0.082 |
| 050524-EZqK8 | 5243 | 2793 | 0.17 | 0.18 | 0.07 | | 0.082 | 0.133 | 0.067 |
| 050524-EZqN9 | 5251 | 2801 | 0.14 | 0.12 | 0.06 | | 0.085 | 0.107 | 0.085 |
| 050524-EZqB17 | 5253 | 2803 | 0 | 0.06 | 0.03 | | 0.079 | 0.134 | 0.072 |
| 050524-EZqF13 | 5266 | 2816 | 0.07 | 1.74 | 1.27 | | 0.156 | 0.080 | 0.095 |
| 050524-EZqH13 | 5273 | 2823 | 0 | 4.86 | 6.93 | | | | |
| 050524-EZqH15 | 5274 | 2824 | 0 | 0.21 | 0.1 | | 0.089 | 0.105 | 0.085 |
| 050524-EZqH16 | 5275 | 2825 | 0 | 0.13 | 0.03 | | 0.095 | 0.326 | 0.071 |
| 050524-EZqN11 | 5289 | 2839 | 0.04 | 0.08 | 0.1 | | 0.084 | 0.163 | 0.076 |
| 050524-EZqN17 | 5292 | 2842 | 0.11 | 0.03 | 0.03 | | 0.087 | 0.102 | 0.083 |
| 051524-EZqK6 | 5314 | 2864 | | 0.18 | | | 0.081 | 0.099 | 0.060 |
| 051524-EZqK7 | 5315 | 2865 | | 14.15 | | | 0.131 | 0.495 | 0.064 |
| 051524-EZqK8 | 5316 | 2866 | | 25.07 | | | 0.145 | 0.106 | 0.073 |
| 051524-EZqN1 | 5317 | 2867 | | 0.05 | | | 0.100 | 0.083 | 0.076 |
| 051524-EZqF3 | 5338 | 2888 | | 0.13 | | | 0.086 | 0.126 | 0.078 |
| 051524-EZqH2 | 5343 | 2893 | | 0.19 | | | 0.085 | 0.115 | 0.082 |
| 051524-EZqJ8 | 5355 | 2905 | | 0.08 | | | 0.099 | 0.083 | 0.075 |
| 052924-EZqB2 | 5356 | 2906 | 0.05 | 0.00 | | | 0.084 | 0.181 | 0.072 |
| 052924-EZqB4 | 5357 | 2907 | 0.05 | 17.00 | | | 0.290 | 0.085 | 0.184 |
| 052924-EZqB5 | 5358 | 2908 | 0.03 | 0.09 | | | 0.087 | 0.096 | 0.074 |
| 052924-EZqB7 | 5359 | 2909 | 0.07 | 0.13 | | | 0.080 | 0.098 | 0.069 |
| 052924-EZqF2 | 5368 | 2918 | 0.03 | 26.36 | | | 0.239 | 0.096 | 0.174 |
| 052924-EZqF6 | 5372 | 2922 | 0.08 | 0.26 | | | 0.129 | 0.097 | 0.103 |
| 052924-EZqF7 | 5373 | 2923 | 0.00 | 0.20 | | | 0.131 | 0.089 | 0.094 |
| 052924-EZqF10 | 5374 | 2924 | 0.03 | 0.23 | | | 0.169 | 0.095 | 0.111 |
| 052924-EZqH1 | 5375 | 2925 | 0.03 | 0.06 | | | 0.075 | 0.161 | 0.077 |
| 052924-EZqH2 | 5376 | 2926 | 0.03 | 0.03 | | | 0.081 | 0.525 | 0.075 |
| 052924-EZqH3 | 5377 | 2927 | 0.03 | 25.23 | | | 0.391 | 0.402 | 0.284 |
| 052924-EZqH4 | 5378 | 2928 | 0.05 | 0.09 | | | 0.087 | 0.584 | 0.077 |
| 052924-EZqH5 | 5379 | 2929 | 0.05 | 0.13 | | | 0.079 | 0.149 | 0.073 |
| 052924-EZqH6 | 5380 | 2930 | 0.03 | 0.00 | | | 0.089 | 0.132 | 0.076 |
| 052924-EZqH7 | 5381 | 2931 | 0.03 | 0.00 | | | 0.079 | 0.438 | 0.076 |
| 052924-EZqH8 | 5382 | 2932 | 0.03 | 0.13 | | | 0.081 | 0.111 | 0.071 |
| 052924-EZqH9 | 5383 | 2933 | 0.05 | 0.03 | | | 0.087 | 0.144 | 0.070 |
| 052924-EZqK1 | 5391 | 2941 | 0.03 | 0.18 | | | 0.120 | 0.236 | 0.076 |
| 052924-EZqN1 | 5397 | 2947 | 0.03 | 0.03 | | | 0.082 | 0.225 | 0.076 |
| 052924-EZqN2 | 5398 | 2948 | 0.03 | 0.03 | | | 0.080 | 0.134 | 0.078 |
| 052924-EZqN4 | 5399 | 2949 | 0.00 | 0.07 | | | 0.096 | 0.224 | 0.082 |
| 052924-EZqN9 | 5400 | 2950 | 0.03 | 0.10 | | | 0.081 | 0.213 | 0.085 |
| 052924-EZqK6 | 5402 | 2952 | 0.05 | 0.10 | | | 0.083 | 0.171 | 0.070 |
| 052924-EZqN6 | 5406 | 2956 | 0.06 | 0.06 | | | 0.075 | 0.110 | 0.065 |
| 052924-EZqN7 | 5407 | 2957 | 0.03 | 0.10 | | | 0.069 | 0.103 | 0.065 |
| 052924-EZqN8 | 5408 | 2958 | 0.03 | 0.10 | | | 0.080 | 0.118 | 0.077 |

TABLE 6
Jurkat NFA GFP (JNG) ASSAY

| | | | | SEQ | |
|---|---|---|---|---|---|
| | | | | ID NO | |
| Target | Type | Tag | Clone Name | (DNA) | JNG Assay |
| CD19 | | (−) | 012424-EZqJ1 | 4754 | RAJI 0.5+; Jeko+/− |
| TAJ | | (−) | 011624-EZkV1 | 4755 | Hutu-80 0.5+; |
| CD79b | | (−) | 011624-EZkX1 | 4756 | Jeko 0.5+; Daudi 0.5+ |
| CD19 | | (−) | 012424-EZqB1 | 4757 | RAJI 2+; Jeko 1.5+; Daudi |
| | | | | 2+ | |
| CD19 | | (+) | 012424-EZqC1 | 4758 | RAJI 1.5+; Daudi 2+ |
| CD19 | | (−) | 012424-EZqD1 | 4759 | RAJI 3+; Daudi 4.5++ |
| CD19-BCMA | | (+) | 012424-EZqE1 | 4760 | RAJI 3+; Daudi 4+ |
| CD19 | | (+) | 011924-BBaO1 | 4761 | RAJI 2.5+; Daudi 2+ |
| CD19-BCMA | | (−) | 011924-BBaP1 | 4762 | RAJI 2.5+; Daudi 2+ |
| BCMA | | (+) | 011924-BBaE1 | 4763 | H929 1.5+; MM.1S 2.5+; |
| CD79b | | (−) | 011624-EZmE1 | 4764 | RAJI+/−; Daudi+/− |
| CD19 | | (−) | 012424-EZoA1 | 4765 | RAJI+; Jeko+; Daudi 2+ |
| CD19 | | (+) | 012424-EZoB1 | 4766 | RAJI+/−; Daudi+/− |
| CD19 | | (+) | 012424-EZoC1 | 4767 | RAJI 0.5+; Daudi+/− |
| CD19 | | (+) | 012424-EZoD1 | 4768 | RAJI 1.5+; Jeko+; Daudi+ |
| CD19 | | (+) | 012424-EZoF1 | 4769 | RAJI+/−; Daudi 0.5+ |
| CD19 | | (+) | 012424-EZoG1 | 4770 | RAJI 0.5+; Daudi+ |
| CD19 | | (+) | 012424-EZoH1 | 4771 | RAJI+/−; ; Daudi+/− |
| CD19 | | (+) | 012424-EZoM1 | 4774 | RAJI 0.5+; Daudi+ |
| BCMA | | (+) | 012424-EZoP1 | 4777 | RAJI 0.5+; Daudi+ |
| BCMA | | (+) | 012424-EZoQ1 | 4778 | Jeko+/−; Daudi+ |
| CD19 | | (+) | 012424-EZoR1 | 4779 | Jeko+/−; Daudi+/− |
| CD19 | | (+) | 012424-EZoS1 | 4780 | RAJI+/− |
| CD19 | | (+) | 012424-EZoT1 | 4781 | RAJI 0.5+; Daudi 0.5+ |
| CD19 | | (+) | 012424-EZoU1 | 4782 | RAJI 1.5+; Daudi 1.5+ |
| CD19 | | (+) | 012424-EZoW1 | 4783 | RAJI+/− |
| CD19 | | (+) | 012424-EZoX1 | 4784 | RAJI+; Daudi+ |
| CD19 | | (+) | 012424-EZoZ1 | 4785 | RAJI+; Daudi 1.5+ |
| CD19 | | (+) | 012424-EZpA1 | 4786 | Jeko+/−; Daudi+/− |
| CD19 | | (+) | 012424-EZpB1 | 4787 | RAJI 1.5+; Daudi 2+ |
| CD19 | | (+) | 012424-EZpC1 | 4788 | RAJI 1.5+; J Daudi 2+ |
| BCMA | | (+) | 012424-EZpF1 | 4789 | H929 1.5+; L363+; |
| CD19 | | (+) | 012424-EZpH1 | 4790 | RAJI 0.5+; Daudi 0.5+ |
| CD19 | | (+) | 012424-EZqH1 | 4791 | RAJI 0.5+; Daudi+/− |
| BCMA | | (+) | 011924-BBaJ1 | 4792 | RPMI 8226 2+ |
| CD79b | | (−) | 011924-BBaL1 | 4793 | RAJI+; Jeko+; Daudi 1.5+ |
| CD19 | | (−) | 020224-BBaU1 | 4794 | RAJI 4+; Daudi 1.5+ |
| CD19 | | (+) | 020224-BBaV1 | 4795 | RAJI 4+; Jeko+ |
| CD79b | | (+) | 012424-JFL2 | 4796 | RAJI+; Jeko 0.5+; |
| CD19 | | C(+) | 020824-BBcH1 | 4797 | RAJI 3.5+; Daudi 2+ |
| CD19 | | C(+) | 020824-BBci2 | 4798 | RAJI 4+; Daudi 1.5+ |
| CD19 | | C(+) | 020824-BBcJ2 | 4799 | RAJI 3.5+; Daudi 2+ |
| BCMA | | (+) | 020824-JScE1 | 4801 | RAJI 1.5+; MM.1S 2+ |
| CD19 | | (+) | 020824-JSbX2 | 4802 | RAJI 4.5+; Daudi 2+ |
| CD19-BCMA | | (+) | 022124-EZuE1 | 4803 | RAJI+; L363 0.5+; |
| CD19 | | (+) | 022124-EZuB1 | 4804 | RAJI+; Jeko 0.5+; Daudi+ |
| CD19 | | (+) | 022124-EZuF1 | 4805 | RAJI 2+; Jeko+; Daudi 2+ |
| CD19-CD22 | | (+) | 022724-EZwA1 | 4806 | RAJI 2.5+; Daudi 2.5+ |
| CD19 | | (−) | 022724-EZwK1 | 4807 | RAJI 0.5+; Daudi+ |
| CD19 | | (+) | 022724-EZwM1 | 4808 | RAJI 2.5+; Daudi 2.5+ |
| CD19 | | (+) | 022724-EZwN1 | 4809 | RAJI 0.5+; Daudi+ |
| CD19 | | (+) | 022724-EZwO1 | 4810 | RAJI+; Jeko+; Daudi 1.5+ |
| CD19 | | (+) | 022724-EZwS1 | 4811 | RAJI+; Jeko 0.5+; Daudi+ |
| CD19 | | (+) | 022724-EZwT1 | 4812 | RAJI 0.5+; Jeko+; Daudi+ |
| CD19 | | (+) | 022724-EZwU1 | 4813 | RAJI+/− |
| CD19 | | (−) | 022724-EZxF1 | 4814 | RAJI 0.5+; |
| BCMA | | (−) | 022824-EZyC1 | 4815 | MM.1S 1.5+; |
| BCMA | | (−) | 022824-EZyF1 | 4816 | RAJI 0.5+; L363 0.5+; MM.1S 1.5+; |
| BCMA | | (−) | 022824-EZyH1 | 4817 | L363+; MM.1S 2+; |
| CD19 | | (−) | 022924-JSdC1 | 4818 | RAJI+ |
| CD19 | | (+) | 022924-JSdB2 | 4819 | RAJI 3+ |
| CD19 | | (+) | 022924-JSdA4 | 4820 | RAJI 0.5+; Daudi 0.5+ |
| CD19 | | (+) | 022924-JSdA5 | 4821 | RAJI 0.5+; Daudi+ |
| CD19 | | (+) | 022924-JSdA8 | 4822 | RAJI+/−Daudi+/− |
| CD19 | | (+) | 022924-JSdA10 | 4823 | RAJI+/−; Daudi+/− |
| CD19 | | (+) | 022924-JSdA11 | 4824 | RAJI 1.5+; Daudi 1.5+ |
| CD19 | | (+) | 022924-JSdA12 | 4825 | RAJI 2+; Jeko 2+; |
| CD19 | | (+) | 022924-JSdA13 | 4826 | RAJI+/−; Daudi+/− |
| CD19 | | (+) | 022924-JSdA14 | 4827 | RAJI+/−; Daudi+/− |
| CD79b | | (+) | 011624-EZgE1 | 4828 | RAJI 1.5+; Daudi 1.5+ |
| CD79b | | (−) | 011624-EZgA1 | 4829 | RAJI 2+; Daudi 2+ |
| CD79b | | (−) | 112523-EZsD1 | 4830 | RAJI 2+; Daudi 2.5+ |
| CD79b | | (−) | 120323-EZwA1 | 4831 | RAJI+/−; Jeko 0.5+ |
| STEAP2 | | (−) | 022124-BBdC1 | 4832 | LnCaP+ |
| IL13Ra2 and Her2 | | (+) | 031124-EZzG1 | 4833 | U87MG 0.5+ |
| CD19 | | (−) | 031124-EZzH1 | 4834 | RAJI+ |
| CLDN18.2 | | (+) | 031124-EZzM1 | 4835 | NUGC4+/−; KATOIII+/− |
| CLDN18.2 | | (+) | 031124-EZzN1 | 4836 | NUGC4+/−; KATOIII+/− |
| CLDN18.2 | | (+) | 031124-EZzO1 | 4837 | NUGC4+/−; KATOIII+/− |
| STEAP2 | | (+) | 031124-EUM1 | 4838 | LnCaP+ |
| CLDN18.2 | | (+) | 031124-EUN1 | 4839 | NUGC4 0.5+; KATOIII+ |
| CLDN6 | | (+) | 031124-EUQ1 | 4840 | OVCAR3+/−; OV90+/− |
| PSCA | | (−) | 031124-EUS1 | 4842 | H1993+/−; N2126+/− |
| PSMA | | (−) | 031124-EUU1 | 4843 | LnCaP 0.5+ |
| STEAP2 | | (−) | 031124-EUaB1 | 4844 | LnCaP+ |
| CLDN18.2 | | (+) | 031124-EUaC1 | 4845 | NUGC4+/−; KATOIII+/− |
| CLDN18.2 | | (+) | 031124-EUaD1 | 4846 | NUGC4 0.5+; KATOIII |
| | | | | | 0.5+ |
| CD19 | | (+) | 042122-SLbM4 | 4847 | RAJI 2+; RAJI CD19KO |
| | | | | | 1+ |
| STEAP2 | | | 031124-BBeH1 | 4848 | LnCaP 1.5+ |
| MSLN | | (+) | 031124-BBeM1 | 4849 | OVCAR3 1.5+; Skov3+ |
| BCMA | | (+) | 031124-BBeQ1 | 4850 | L363+; MM.1S 2+ |
| STEAP2 | | (+) | 022824-EZyN1 | 4851 | LnCaP 2+ |
| STEAP2 | | (+) | 022824-EZyO1 | 4852 | LnCaP 3+ |
| CLD18A2 | | (+) | 022824-EZyQ1 | 4853 | NUGC4 1.5+ |
| CLD18A2 | | (+) | 022824-EZyR1 | 4854 | NUGC4 0.5+; KATOIII+/− |
| CD19 | | | 031724-BBfF1 | 4855 | RAJI 2+ |
| CD79b | | | 012424-JFI3 | 4856 | Jeko+/−; Daudi+/− |
| CD79b | | | 020824-JScG2 | 4857 | RAJI 2+; Daudi 2.5+ |
| PSMA | | | 010824-PBB3 | 4858 | LnCaP 1.5+ |
| CD19 | | | 021924-BBdY2 | 4859 | RAJI 3.5+ |
| STEAP2 | | | 022124-BBdC1 | 4860 | LnCaP 1.5+ |
| MPL | | | 102023-PBU3 | 4861 | HEL 2+ |
| CD19 | | | 021924-BBdT1 | 4862 | RAJI+/− |
| CD19 | | | 021924-BBdX1 | 4863 | RAJI 3+ |
| CD19 | | | 032124-EZbD1 | 4864 | RAJI 2+ |
| CD19 | | | 032124-EZbE1 | 4865 | RAJI 2.5+ |
| CD19 | | | 032124-EZbH1 | 4866 | RAJI 0.5+ |
| CD19-BCMA | | | 032124-EZbJ1 | 4867 | RAJI 4.5+ |
| CD19-CD20 | | | 032124-EZbK1 | 4868 | RAJI 4+ |
| CD19-BCMA | | | 032124-EZbN1 | 4869 | RAJI 1.5+ |
| CD19 | | | 032724-JSfN1 | 4870 | RAJI+; Jeko 0.5+ |
| CLDN6 | | | 022824-EZyP1 | 4871 | OVCAR3 3+; OV90 1.5+ |
| CLDN18.2 | | | 022824-EZyS1 | 4872 | NUGC4 2+; KATOIII+ |
| CLDN18.2 | | | 032724-EZbP1 | 4873 | NUGC4 2.5+; KATOIII+ |
| CD19 | | | 032724-EZbQ1 | 4874 | RAJI+/−; MM.1S+/− |
| PSMA | | | 032724-EZbS1 | 4875 | LnCaP 5+ |
| CD19-BCMA | | | 032724-EZbU1 | 4876 | RAJI 2+; L363 1.5+; |
| BCMA | | | 031824-EZaA1 | 4877 | MM.1S 0.5+; U266 0.5+ |
| CD19 | | | 031824-EZaB1 | 4878 | RAJI 2.5+ |
| CD19-CD20 | | | 031824-EZaH1 | 4879 | RAJI 2.5+ |
| BCMA | | | 031824-EZaJ1 | 4880 | MM.1S 2.5+; U266+ |
| CD19 | | | 031824-EZaK1 | 4881 | RAJI+ |
| CD19 | | | 031824-EZaM1 | 4882 | RAJI 2.5+ |
| CD19 | | | 031824-EZaN1 | 4883 | RAJI+ |
| CD19 | | | 031824-EZaO1 | 4884 | RAJI+ |
| CD19 | | | 031824-EZaP1 | 4885 | RAJI 2.5+ |
| CD19 | | | 031824-EZaQ1 | 4886 | RAJI 0.5+ |
| CD19-CD20 | | | 031824-EZaR1 | 4887 | RAJI 5+ |
| CD19 | | | 031824-EZaS1 | 4888 | RAJI 0.5+ |
| CD19 | | | 031824-EZaT1 | 4889 | RAJI+ |
| CD19 | | | 031824-EZaU1 | 4890 | RAJI+ |
| CD19 | | | 031824-EZaV1 | 4891 | RAJI+/− |
| STEAP2 | | | 031124-BBeH1 | 4892 | LnCaP 4+ |
| CLDN18.2 | | | 031124-BBeJ2 | 4893 | NUGC4 1.5+ |
| MSLN | | | 031124-BBeM1 | 4894 | Skov3+; OVCAR3+ |
| CD79b | | | 121723-BBjC1 | 4895 | RAJI 1.5+; Daudi 2+ |
| CD79b | | | 011624-EZfE1 | 4896 | RAJI+; Jeko 0.5+ |
| CD19 | | | 040524-EZcE1 | 4900 | RAJI 2.5+; Jeko 0.5+ |
| CD19-CD22 | | | 040524-EZdJ1 | 4901 | RAJI 2+; Jeko+ |
| CD19 | | | 040524-EZcM1 | 4902 | RAJI 1.5+ |
| PSMA | | | 040524-EZcQ1 | 4903 | LnCaP 0.5+ |
| PSMA | | | 040524-EZcS1 | 4904 | LnCaP 2+ |
| CD19 | | | 040524-EZdK1 | 4905 | RAJI 3+ |
| PSMA | | | 040524-EZdO1 | 4906 | LnCaP+ |
| PSMA | | | 040524-EZdP1 | 4907 | LnCaP+/− |
| BCMA | | | 032724-JSfS3 | 4908 | RAJI+; L363 2+ |
| CD19 | | | 022124-EZuC1 | 4909 | RAJI 0.5+; Jeko 0.5+ |
| CD19 | | | 022124-EZuH1 | 4910 | RAJI 5+ |
| CD19 | | | 022724-EZwH1 | 4911 | RAJI 0.5+ |
| BCMA | | | 022724-EZwJ1 | 4912 | RAJI 1.5+; L363 3+; |
| CLDN18.2 | | | 022724-EZwR1 | 4913 | NUGC4 0.5+ |
| BCMA | | | 022824-EZyD1 | 4914 | L363 2+; MM.1S 2+ |
| BCMA | | | 022824-EZyE1 | 4915 | MM.1S 0.5+ |
| CD19 | | | 022924-JSdA1 | 4916 | RAJI 2+ |
| CD19 | | | 030124-EUA2 | 4917 | RAJI+ |
| BCMA | | | 022924-PPB2 | 4918 | L363 0.5+; MM.1S 1.5+ |
| CD79b | | | 112923-EZuH2 | 4919 | Jeko 1.5+; Daudi 2.5+ |
| CD19 | | | 040524-EZcF1 | 4920 | RAJI+/− |
| CD19 | | | 040524-EZcH1 | 4921 | RAJI+/− |
| CD19 | | | 040524-EZcJ1 | 4922 | RAJI 1.5+ |
| PSMA | | | 040524-EZcO1 | 4923 | LnCaP+ |
| PSMA | | | 040524-EZcP1 | 4925 | LnCaP 0.5+ |
| PSMA | | | 040524-EZcT1 | 4927 | LnCaP+/− |
| CD19 | | | 040524-EZdA1 | 4929 | RAJI+/− |
| CD19 | | | 040524-EZdB 1 | 4930 | RAJI+/− |
| PSMA | | | 040524-EZdC1 | 4931 | LnCaP 0.5+ |
| PSMA | | | 040524-EZdD1 | 4933 | LnCaP 0.5+ |
| PSMA | | | 040524-EZdE1 | 4935 | LnCaP+/− |
| PSMA | | | 040524-EZdF1 | 4937 | LnCaP 0.5+ |
| PSMA | | | 040524-EZdX1 | 4938 | LnCaP+/− |
| PSMA | | | 040524-EZdY1 | 4939 | LnCaP+/− |
| CD19 | | | 041124-EZgJ1 | 4946 | RAJI+ |
| CD79b | | | 122523-EZdV1 | 4947 | Jeko 1.5+; Daudi 2.5+ |
| CD19 | | | 031124-BBeA1 | 4948 | RAJI CD19 KO 6.5+ |
| CD19 | | | 041624-JSgM1 | 4951 | RAJI+/− |
| CD19 | | | 041624-JSgS2 | 4955 | RAJI+/− |
| PSMA | | | 032724-EZbR5 | 4957 | LnCaP+/− |
| CD19 | | | 040524-EZcD4 | 4960 | RAJI+/− |
| | | | 042524-EZpA1 | 4966 | RAJI 0.5+; Daudi 0.5+ |
| PSMA | | | 040524-EZdN1 | 4967 | LnCaP 4+ |
| CD19 | | | 040524-EZcN2 | 4968 | Daudi 1+; RAJI+/− |
| PSMA | | | 040524-EZdW1 | 4980 | LnCaP 3+ |
| PSMA | | | 040524-EZcU2 | 4981 | LnCaP 4.5+ |
| CD19 | | | 050124-EZpR1 | 4993 | RAJI+/−; Daudi+/− |
| CD19 | | | 050124-EZpS1 | 4994 | RAJI+ |
| CD19-BCMA | | | 050124-EZpQ1 | 4995 | RAJI 1+, MM1s 1.5+ |
| CD19 | | | 050224-EZrB1 | 4996 | RAJI 0.5+ |
| CD19 | | | 042524-EZpA2 | 5004 | RAJI+/− |
| BCMA-CD19 | | | 050124-EZpO1 | 5005 | RAJI 0.5+; MM.1S+ |
| PSMA | | | 051824-EZyF1 | 5006 | LnCaP 6+ |
| PSMA | | | 051824-EZyG1 | 5007 | LnCaP 2.5+ |
| PSMA | | | 051824-EZyH1 | 5008 | LnCaP 3.5+ |
| STEAP2 | CAR | (−) | 110824-EZnN1 | 8704 | LnCaP+ |
| STEAP1 | CAR | (−) | 020725-EZgE1 | 8705 | LnCaP-WT 3+ |
| STEAP1 | CAR | P11 | 020725-EZgF1 | 8706 | LnCaP-WT 1.5+ |
| STEAP1 | CAR | (−) | 020725-EZgG1 | 8707 | LnCaP-WT 0.5+ |
| STEAP1 | CAR | P11 | 020725-EZgH1 | 8708 | LnCaP-WT+/− |
| STEAP1 | CAR | (−) | 041725-TCdD2 | 8709 | LnCaP+/− |
| CD19 | SIR | (−) | 012225-EZeR2 | 8710 | RAJI 0.5+ |
| CD19 | SIR | (−) | 032818-B01-PS | 8711 | RAJI+/− |
| CD19 | SIR | (−) | 032818-C01-PS | 8712 | RAJI+/− |
| CD19 | SIR | (−) | 021025-BBcV1 | 8713 | RAJI+ |
| CD19 | SIR | (−) | 021025-BBcW1 | 8714 | RAJI 0.5+ |
| CD19 | SIR | P11 | 032725-EZsP1 | 8715 | RAJI 2.5+; L363 3.5+; |
| | | | | | U266 5+; MM.1S 4.5+; |
| | | | | | NALM6 3+ |
| CD19 | SIR | P11 | 032725-EZsO1 | 8716 | RAJI 0.5+ |
| CLDN6 | SIR | (−) | 040725-BBfA2 | 8717 | OVCAR3+/−; PA-1 2.5+ |
| GPC3 | SIR | P11 | 052125-AKmD2 | 8718 | HepG2+/− |
| CD20 | zSIR | P8 | 112624-EZqK3 | 8719 | RAJI 0.5+; Jeko+/− |
| CD19 | zSIR | (−) | 030725-BBdY2 | 8720 | RAJI+; NALM6+/−; |
| CD19 | zSIR | (−) | 030725-BBdY2 | 8721 | RAJI 2.5+; NALM6 0.5+ |
| CD19 | zSIR | (−) | 031825-BBeE2 | 8722 | RAJI 2+; NALM6+/− |
| CD19 | zSIR | (−) | 041025-JGH1 | 8723 | RAJI+/−; Jeko 0.5+ |
| BCMA | zSIR | (−) | 050625-EZwF1 | 8724 | MM.1S 0.5+; L363 0.5+ |
| CD19 | z16SAR | (−) | 022725-BBdN2 | 8725 | RAJI 4.5+; Jeko 2.5+ |
| CD19 | z16SAR | (−) | 041025-JGB1 | 8726 | RAJI 3+; Jeko 2+ |
| CD19 | z16SAR | (−) | 050625-EZwA1 | 8727 | RAJI 2+ |
| CD19 | z16SAR | (−) | 050625-EZwB1 | 8728 | RAJI 3+ |
| CD19 | z16SAR | (−) | 050625-EZwD1 | 8729 | RAJI 1.5+ |
| CD79b | z16SAR | (−) | 031825-BBeF2 | 8730 | RAJI+; Jeko+/− |
| CD19 | z16SAR | (−) | 031825-BBeH1 | 8731 | RAJI 2+; Jeko+/− |
| CD19 | z16SAR | (−) | 041025-JGD1 | 8732 | RAJI 2.5+; Jeko 1.5+ |
| CD19 | z16SAR | (−) | 050825-EZxB1 | 8733 | RAJI 1.5+; Jeko 0.5+ |
| CD19 | z16SAR | (−) | 050825-EZxC1 | 8734 | RAJI 1.5+; Jeko 0.5+ |
| CD19 | z16SAR | (−) | 050825-EZxD1 | 8735 | RAJI 2.5+; NALM6 2.5+; |
| | | | | | MM.1S 3.5+; L363 3+ |
| CD19 | z16SAR | (−) | 050825-EZxG1 | 8736 | RAJI 0.5+; Jeko+/− |
| CD19 | z16SAR | (−) | 050825-EZxH1 | 8737 | RAJI 0.5+ |
| CD19 | z16SAR | (−) | 050825-EZxJ1 | 8738 | RAJI 0.5+; NALM6 0.5+; |
| BCMA | z16SAR | P11 | 051625-EZyS1 | 8739 | MM.1S 0.5+; L363+/− |
| CD20 | MC-SAR | (−) | 011225-EZyH1 | 8740 | RAJI+/− |
| ROR1-CD20 | MC-SAR | (−) | 011225-EZzC1 | 8741 | RAJI+/− |
| ROR1-CD20 | MC-SAR | (−) | 011225-EZzE1 | 8742 | RAJI+/− |
| Her2 | MC-SAR | (−) | 011725-EZbO1 | 8743 | Skov3+/− |
| Her2-CDH19 | MC-SAR | (−) | 011725-EZbP1 | 8744 | SK-MEL-31+/− |
| NKG2D-Her2 | MC-SAR | (−) | 011725-EZbV1 | 8745 | Skov3+/− |
| MSLN | MC-SAR | (−) | 012225-EZcO1 | 8746 | OVCAR3+/− |
| CS1 | MC-SAR | (−) | 012225-EZcP1 | 8747 | MM.1S+/− |
| MSLN | MC-SAR | (−) | 012225-EZdW1 | 8748 | OVCAR3+/− |
| CS1 | MC-SAR | (−) | 012225-EZdX1 | 8749 | MM.1S+/− |
| CD123- | MC-SAR | (−) | 011725-AKbR1 | 8750 | L428 0.5+ |
| TGFBR2 | | | | | |
| CD20-CD123 | MC-SAR | (−) | 012825-BBaA1 | 8751 | RAJI+; Jeko+ |
| CD20 | MC-SAR | (−) | 012825-BBaB1 | 8752 | RAJI+; Jeko+ |
| CD20-ROR1 | MC-SAR | (−) | 012825-BBaC1 | 8753 | RAJI+; Jeko 0.5+ |
| ROR1- | MC-SAR | (−) | 012825-BBaD1 | 8754 | Jeko+ |
| TGFBR2 | | | | | |
| CD20-CD123 | MC-SAR | (−) | 012825-BBaE1 | 8755 | RAJI+; Jeko 0.5+; |
| CD20 | MC-SAR | (−) | 012825-BBaF1 | 8756 | RAJI 0.5+; Jeko 0.5+ |
| ROR1-CD20- | MC-SAR | (−) | 012825-BBaG1 | 8757 | RAJI 0.5+; Jeko 0.5+ |
| TGFRB2 | | | | | |
| CD20- | MC-SAR | (−) | 012825-BBaH1 | 8758 | RAJI 0.5+; Jeko+ |
| TGFBR2 | | | | | |
| CD20-CD123 | MC-SAR | (−) | 012825-BBaJ1 | 8759 | RAJI 2+; Jeko 1.5+ |
| CD20 | MC-SAR | (−) | 012825-BBaK1 | 8760 | RAJI+; Jeko+ |
| CD20-ROR1 | MC-SAR | (−) | 012825-BBaL1 | 8761 | RAJI 1.5+ |
| CD19-CS1 | MC-SAR | (−) | 012825-BBaQ1 | 8762 | RAJI 0.5+ |
| CD19-CS1 | MC-SAR | P11 | 012825-BBaR1 | 8763 | RAJI 0.5+; Jeko+/− |
| CD20-CD123 | MC-SAR | (−) | 012825-BBaU1 | 8764 | RAJI+; Jeko+ |
| CD20 | MC-SAR | (−) | 012825-BBaV1 | 8765 | RAJI+; Jeko 0.5+ |
| CD20-ROR1 | MC-SAR | (−) | 012825-BBaW1 | 8766 | RAJI 2+; Jeko 1.5+ |
| CD20- | MC-SAR | (−) | 012825-BBaX1 | 8767 | RAJI 1.5+; Jeko+ |
| TGFBR2 | | | | | |
| CD20-CD123 | MC-SAR | (−) | 012825-BBaY1 | 8768 | RAJI 2+; Jeko+ |
| CD20-ROR1 | MC-SAR | (−) | 012825-BBaZ1 | 8769 | RAJI+; Jeko+ |
| CD20- | MC-SAR | (−) | 012825-BBbA1 | 8770 | RAJI 2+; Jeko+ |
| TGFBR2 | | | | | |
| CD20-CD123 | MC-SAR | (−) | 012825-BBbB1 | 8771 | RAJI+; Jeko+ |
| CD20-ROR1 | MC-SAR | (−) | 012825-BBbC1 | 8772 | RAJI 0.5+; Jeko 0.5+ |
| CD20- | MC-SAR | (−) | 012825-BBbD1 | 8773 | RAJI 1.5+; Jeko+; |
| TGFBR2 | | | | | L428+/−; KG1 0.5+ |
| CD20-CD123 | MC-SAR | (−) | 012825-BBaE1 | 8774 | RAJI+/−; Jeko+/− |
| CD19-CS1 | MC-SAR | (−) | 012825-BBaQ1 | 8775 | RAJI 0.5+ |
| CD19-CS1 | MC-SAR | P11 | 012825-BBaR1 | 8776 | RAJI 0.5+ |
| CD20-CD123 | MC-SAR | (−) | 012825-BBaU1 | 8777 | RAJI+ |
| CD20 | MC-SAR | (−) | 012825-BBaV1 | 8778 | RAJI 0.5+ |
| CD20-ROR1 | MC-SAR | (−) | 012825-BBaW1 | 8779 | RAJI+; Jeko 0.5+ |
| TGFBR2- | MC-SAR | (−) | 020125-BBbK1 | 8780 | RAJI+ |
| CD20 | | | | | |
| CD19-CS1 | MC-SAR | (−) | 021825-EZmN1 | 8781 | RAJI+/− |
| CD19-CS1 | MC-SAR | (−) | 021825-EZmP1 | 8782 | RAJI 0.5+; L363+/− |
| Her2-CDH19 | MC-SAR | (−) | 022025-EZnJ1 | 8783 | Skov3 2+ |
| Her2-NKG2D | MC-SAR | (−) | 022025-EZnK1 | 8784 | Skov3 4+; BT-474 2.5+ |
| CD22 | MC-SAR | (−) | 022025-EZnM1 | 8785 | RAJI+/− |
| CD22-MSLN | MC-SAR | (−) | 022025-EZnN1 | 8786 | RAJI 2+; Jeko 3.5+ |
| CD22-Her2 | MC-SAR | (−) | 031325-EZpO1 | 8787 | BT-474 0.5+; Skov3+ |
| CD19 | MC-SAR | (−) | 022425-EZmF1 | 8788 | RAJI 2.5+ |
| CD19-CS1 | MC-SAR | (−) | 022425-EZmJ1 | 8789 | RAJI 1.5+; U266+ |
| CD19-CS1 | MC-SAR | (−) | 022425-EZmN1 | 8790 | RAJI+; L363 0.5+; |
| CD19-CS1 | MC-SAR | (−) | 022425-EZmO1 | 8791 | RAJI 0.5+; L363+/− |
| CD19 | MC-SAR | (−) | 022425-EZmA2 | 8792 | RAJI 2.5+ |
| CD19 | MC-SAR | (−) | 022425-EZmB2 | 8793 | RAJI 2.5+ |
| CD19 | MC-SAR | (−) | 022425-EZmH2 | 8794 | RAJI 1.5+ |
| CS1 | MC-SAR | (−) | 022425-EZmK2 | 8795 | RAJI 2+; L363+/− |
| CD22-MSLN | MC-SAR | (−) | 022025-EZnW2 | 8796 | RAJI 1.5+; Jeko 0.5+ |
| CD19-CS1 | MC-SAR | (−) | 031825-EZqE1 | 8797 | RAJI 0.5+; MM.1S+ |
| CD19 | MC-SAR | (−) | 051325-BBgT1 | 8798 | RAJI+ |
| CD19 | MC-SAR | (−) | 051325-BBhC1 | 8799 | RAJI 2+; L363 0.5+ |
| CD79b | MC-SAR | (−) | 051525-HWG2 | 8800 | Jeko+/− |
| CD79b | MC-SAR | (−) | 051525-HWH2 | 8801 | RAJI+/− |
| CD79b | MC- | (−) | 011725-EZaF1 | 8802 | RAJI+/−; Jeko+/− |
| | zSAR | | | | |
| CD79b | MC- | (−) | 021825-EZjC1 | 8803 | RAJI 0.5+ |
| | zSAR | | | | |
| CD19 | CAR | (−) | 052224-JSmS2 | 8804 | RAJI+ |
| STEAP2 | CAR | P11- | 051524-EZwA1 | 8805 | LnCaP+/− |
| | | P14(+) | | | |
| STEAP2 | CAR | P11- | 051524-EZwB1 | 8806 | LnCaP+/− |
| | | P14(+) | | | |
| STEAP2 | CAR | P11- | 051524-EZwD1 | 8807 | LnCaP+/− |
| | | P11(+) | | | |
| STEAP2 | CAR | P11- | 051524-EZwF1 | 8808 | LnCaP+/− |
| | | P11(+) | | | |
| STEAP2 | CAR | P11(+) | 051524-EZwG1 | 8809 | LnCaP 3.5+ |
| BCMA | CAR | (−) | 070924-EZkN1 | 8810 | MM.1S 1.5+; RAJI+ |
| BCMA | CAR | (−) | 070924-EZkP1 | 8811 | MM.1S 4+; RAJI 2.5+ |
| BCMA | CAR | (−) | 070924-EZkQ1 | 8812 | MM.1S 1.5+; RAJI+ |
| BCMA | CAR | (−) | 070924-EZkR1 | 8813 | RAJI+; NALM6 0.5+; |
| | | | | | MM.1S 1.5+ |
| BCMA | CAR | (−) | 071224-EZoQ1 | 8814 | MM.1S+; RAJI+/− |
| BCMA | CAR | (−) | 071224-EZoR1 | 8815 | MM.1S 2.5+; RAJI+/− |
| BCMA | CAR | (−) | 071224-EZoS1 | 8816 | MM.1S+/− |
| BCMA | CAR | (−) | 071224-EZoT1 | 8817 | MM.1S+/− |
| BCMA | CAR | (−) | 071224-EZoU1 | 8818 | MM.1S+/− |
| BCMA | CAR | (−) | 071224-EZoY1 | 8819 | MM.1S 2.5+; RAJI+ |
| CD19 | CAR | (−) | 071224-EZnO1 | 8820 | RAJI 3+; Jeko-1 2.5+ |
| CD19 | CAR | (−) | 071224-EZnP1 | 8821 | RAJI 3.5+; Jeko-1 2+ |
| CD19 | CAR | (−) | 071224-EZnS1 | 8823 | RAJI 3.5+; Jeko-1 3+ |
| CD19 | CAR | (−) | 071224-EZnQ1 | 8824 | RAJI+; Jeko-1+ |
| CD20 | CAR | (−) | 071224-EZoV1 | 8825 | RAJI+; Jeko-1+ |
| CD20 | CAR | (−) | 071224-EZoW1 | 8826 | RAJI 1.5+; Jeko-1+ |
| CD20 | CAR | (−) | 070924-EZkS1 | 8827 | RAJI+; Jeko-1+ |
| CD20 | CAR | (−) | 070924-EZkT1 | 8828 | RAJI 1.5+; Jeko-1+ |
| CD22 | CAR | (−) | 071224-EZoX1 | 8829 | RAJI 2+; Jeko-1+/− |
| CD33 | CAR | (−) | 071224-EZnU1 | 8830 | HL60 1.5+ |
| CD33 | CAR | (−) | 071224-EZnX1 | 8831 | HL60 1.5+ |
| CD33 | CAR | (−) | 071224-EZnY1 | 8832 | HL60 2+ |
| MSLN | CAR | (−) | 071224-EZoA1 | 8834 | SKOV-3+/− |
| MSLN | CAR | (−) | 071224-EZoB1 | 8835 | SKOV-3+/− |
| MSLN | CAR | (−) | 071224-EZoC1 | 8836 | SKOV-3+/− |
| MSLN | CAR | (−) | 071224-EZoD1 | 8837 | SKOV-3+/− |
| DLL3 | CAR | (−) | 071224-EZoJ1 | 8838 | SK-MEL-5+/− |
| DLL3 | CAR | (−) | 071224-EZoM2 | 8840 | SK-MEL-5 0.5+ |
| STEAP2 | CAR | (−) | 080224-BBnP1 | 8841 | LnCaP 4.5+ |
| CLDN6 | CAR | (−) | 080224-BBnU1 | 8842 | H1197+/− |
| CLDN6 | CAR | (−) | 080224-BBnW1 | 8843 | OVCAR3 1.5+; OV90 2+; |
| CD19 | SIR | (−) | 091018-E02-AJ | 8844 | Daudi+; Jeko-1 0.5+; Jeko- |
| | | | | | 1 CD22KO 0.5+; Raji+; |
| CD19-CD22 | SIR | (−) | 051824-EZxA1 | 8845 | Raji 1.5+; Raji CD19KO+; |
| CD19-CD22 | SIR | (−) | 051824-EZxC1 | 8846 | Raji 1.5+; Raji CD19KO+; |
| CD19-CD22 | SIR | P13(+) | 051824-EZxD1 | 8847 | Raji+; Raji CD19KO+; |
| CD19-CD22 | SIR | (−) | 051824-EZxB1 | 8848 | Raji+; Raji CD19KO+; |
| | | | | | Raji CD22KO+ |
| CD19-CD22 | SIR | P13(+) | 051824-EZxF1 | 8849 | Raji 1.5+; Raji CD19KO+; |
| | | | | | Raji CD22KO+ |
| CD19-CD22 | SIR | P7(+) | 051824-EZxG1 | 8850 | Raji+; Raji CD19KO 0.5+; |
| | | | | | Raji CD22KO+ |
| CD19-CD22 | SIR | P7(+) | 051824-EZxJ1 | 8851 | Raji 1.5+; Raji CD19KO+; |
| | | | | | Raji CD22KO+ |
| CD19-CD22 | SIR | P7(+) | 051824-EZxK1 | 8852 | Raji 1.5+; Raji CD19KO+; |
| | | | | | Raji CD22KO+ |
| CD19 | SIR | (−) | 052224-JSmB1 | 8853 | RAJI+/− |
| CD19-CD22 | SIR | (−) | 052224-JSmC1 | 8854 | RAJI+/− |
| CD19-CD22 | SIR | P13(+) | 052224-JSmD1 | 8855 | RAJI+/− |
| PSMA | SIR | P8(+) | 051824-EZyH1 | 8856 | LnCaP 1.5+ |
| CD19-CD22 | SIR | P13(+) | 042524-EZnV1 | 8857 | Daudi 1.5+; Jeko-1+; Jeko- |
| | | | | | 1 CD19KO 0.5+ |
| CD19-CD22 | SIR | P13(+) | 042524-EZnX1 | 8858 | Raji 0.5+; Raji CD19KO |
| | | | | | 0.5+; Raji CD22KO+/− |
| CD19-BCMA | SIR | P13(+) | 042524-EZnY1 | 8859 | RAJI+; NALM6 2.5+; |
| | | | | | L363 2+; MM.1S 3+ |
| CD19-CD22 | SIR | P13(+) | 042524-EZoA1 | 8860 | Raji 1.5+; Raji CD19KO |
| | | | | | 1.5+; Raji CD22KO+ |
| CD19-CD22 | SIR | P13(+) | 042524-EZoF1 | 8861 | Raji 0.5+; Raji CD19KO |
| | | | | | 1.5+; Raji CD22KO+ |
| CD19-CD22 | SIR | P13(+) | 042524-EZoH1 | 8862 | Raji+; Raji CD19KO 0.5+; |
| | | | | | Raji CD22KO+ |
| CD19-CD22 | SIR | P13(+) | 042524-EZoC2 | 8863 | Raji 0.5+; Raji CD19KO |
| | | | | | 0.5+; Raji CD22KO 0.5+; |
| CD19 | SIR | (−) | 052324-EZzG1 | 8864 | RAJI 0.5+ |
| CD19-CD22 | SIR | P7(+) | 051824-EZxH1 | 8865 | Raji 3.5+; Raji CD19KO |
| | | | | | 3+; Raji CD22KO 2.5+ |
| CD19-CD22 | SIR | P13(+) | 051824-EZxE1 | 8866 | Raji 3+; Raji CD19KO 3+; |
| | | | | | Raji CD22KO 2.5+; |
| CLD18A2 | SIR | P11- | 051524-EZwT3 | 8867 | KATOIII+/−; NUGC4 0.5+ |
| | | P11(+) | | | |
| CD79b- | SIR | (−) | 060424-EZbA1 | 8868 | RAJI 0.5+; NALM6+/−; |
| BCMA | | | | | L363 0.5+; MM.1S+ |
| CD79b-CD20 | SIR | (−) | 060424-EZbB1 | 8869 | RAJI 0.5+; Jeko+ |
| CD79b-CD22 | SIR | (−) | 060424-EZbC1 | 8870 | RAJI 0.5+; Jeko 0.5+; |
| | | | | | Daudi 0.5+ |
| CD79b | SIR | (−) | 061924-EZdO1 | 8871 | RAJI 2+; Jeko 0.5+; |
| CD79b-CD22 | SIR | (−) | 061924-EZcQ1 | 8872 | RAJI 3.5+; RAJI CD22KO |
| | | | | | 3+; Jeko 0.5+; Daudi 0.5+ |
| BCMA | SIR | P8-C(+) | 061924-EZdA1 | 8873 | L363 1.5+; MM.1S 3.5+ |
| CD19 | SIR | P8-C(+) | 061924-EZdB1 | 8874 | RAJI 3+ |
| BCMA | SIR | P11- | 061924-EZdC1 | 8875 | RAJI+; MM.1S 2.5+ |
| | | C(+) | | | |
| BCMA | SIR | C(+) | 061924-EZdE1 | 8876 | RAJI 0.5+; MM.1S 3+ |
| CD19 | SIR | P11- | 061924-EZdG1 | 8878 | RAJI+ |
| | | P13- | | | |
| | | C(+) | | | |
| CD19 | SIR | P13- | 061924-EZdH1 | 8879 | RAJI 2+; RAJI |
| | | P13- | | | CD19KO+/− |
| | | C(+) | | | |
| CD19 | SIR | P13- | 061924-EZdJ1 | 8880 | RAJI 1.5+; RAJI |
| | | C(+) | | | CD19KO+/− |
| CD19 | SIR | P13- | 061924-EZdK1 | 8881 | RAJI 2+ |
| | | C(+) | | | |
| CD19-CD22 | SIR | P13(+) | 042524-EZoG1 | 8882 | RAJI 2.5+; RAJI CD19 KO |
| | | | | | 3+; RAJI CD22 KO 3+; |
| CD79b | SIR | (−) | 070924-BBjV1 | 8883 | RAJI 0.5+; Jeko+; Daudi+ |
| CD79b | SIR | (−) | 070924-BBkA1 | 8884 | RAJI+; Jeko 1.5+; |
| CD79b | SIR | (−) | 070924-BBkG1 | 8885 | RAJI 2.5+; Daudi 2+ |
| CD20-CD79b | SIR | (−) | 070924-BBjQ1 | 8886 | RAJI 3.5+; Jeko 2+; |
| CD20-BCMA | SIR | C(+) | 070924-BBjS1 | 8887 | RAJI+; Jeko+; Daudi 2.5+ |
| CD20-CD79b | SIR | (−) | 070924-BBjU1 | 8888 | RAJI 3.5+; Daudi 2.5+ |
| BCMA- | SIR | (−) | 070924-BBjX1 | 8889 | RAJI 1.5+; Jeko 2+ |
| CD79b | | | | | |
| CD20-CD79b | SIR | (−) | 070924-BBjY1 | 8890 | RAJI 4+; Daudi 3+ |
| CD79b | SIR | (−) | 070924-BBjZ1 | 8891 | RAJI+; Jeko+; Daudi+ |
| CD79b- | SIR | (−) | 070924-BBkD1 | 8892 | RAJI 1.5+; NALM6+; |
| BCMA | | | | | L363+; MM.1S 2.5+ |
| CD79b-CD20 | SIR | (−) | 070924-BBkE1 | 8893 | RAJI 3.5+; Daudi 3.5+ |
| CD79b | SIR | (−) | 070924-BBkF1 | 8894 | RAJI 2+; Daudi 3.5+ |
| CD19 | SIR | P11(+) | 071024-BBkQ1 | 8895 | RAJI 2.5+ |
| CD19 | SIR | P11(+) | 071024-BBkR1 | 8896 | RAJI 3+ |
| CD19 | SIR | P11(+) | 071024-BBkS1 | 8897 | RAJI 3+ |
| CD19 | SIR | P11(+) | 040524-EZcY2 | 8898 | RAJI 2.5+ |
| CD79b | SIR | (−) | 070924-BBkB2 | 8899 | RAJI+; Daudi 1.5+ |
| BCMA | zSIR | (−) | 061824-CLC1 | 8901 | L363+/−; MM.1S 0.5+ |
| PSMA | zSIR | P11(+) | 061824-CLH1 | 8902 | LnCaP 2+ |
| BCMA | zSIR | (−) | 061824-CLJ1 | 8903 | RAJI 0.5+; MM.1S+ |
| CD19 | zSIR | (−) | 061824-CLN1 | 8904 | RAJI 2.5+ |
| CD19-CD20 | zSIR | (−) | 061824-CLG2 | 8905 | RAJI 2+ |
| CD19 | zSIR | C(+) | 061924-EZcM1 | 8906 | RAJI 0.5+ |
| CD19 | zSIR | (−) | 061924-EZcN1 | 8907 | RAJI 3.5+ |
| BCMA | zSIR | (−) | 062124-CLM2 | 8908 | RAJI+; MM.1S 1.5+ |
| CD19 | zSIR | C(+) | 061924-EZcP2 | 8909 | RAJI 2+ |
| CD19 | zSIR | (−) | 061924-EZcJ2 | 8910 | RAJI+/− |
| CD19 | zSIR | (−) | 061824-CLF5 | 8911 | RAJI+/− |
| CD19 | zSIR | C(+) | 071524-MGH2 | 8913 | RAJI 2+ |
| CD19 | zSIR | C(+) | 071524-MGI1 | 8914 | RAJI+ |
| CD19 | zSIR | C(+) | 071524-MGJ2 | 8915 | RAJI 0.5+ |
| CD19-CD22 | CD16- | P11- | 060424-EZbD1 | 8916 | LnCaP+/− |
| | SAR | C(+) | | | |
| PSMA | CD16- | P11-(+) | 060424-EZbD1 | 8917 | LnCaP 4+ |
| | SAR | | | | |
| PSMA | CD16- | P8-C(+) | 060424-EZbH2 | 8918 | LnCaP 1.5+ |
| | SAR | | | | |
| PSMA | z16SAR | P11(+) | 051824-EZxW1 | 8919 | LnCaP 0.5+ |
| PSMA | z16SAR | P11(+) | 051824-EZxY1 | 8920 | LnCaP 0.5+ |
| PSMA | z16SAR | P11(+) | 051824-EZyA1 | 8921 | LnCaP 0.5+ |
| CD19 | z16SAR | (−) | 061924-EZcO1 | 8922 | RAJI 1.5+ |
| CD19 | z16SAR | (−) | 110323-BBeK2 | 8923 | RAJI 1.5+ |
| CD19 | z16SAR | (−) | 061924-EZcK2 | 8924 | RAJI+ |
| CD79b | HC-SIR | (−) | 061924-EZdN1 | 8925 | RAJI+; Jeko+/− |
| CD79b | HC-SIR | (−) | 061924-EZdP2 | 8926 | RAJI 2+; Jeko+/− |
| CD79b | HC-SIR | (−) | 070924-BBjW1 | 8927 | RAJI+; Jeko+; Daudi 1.5+ |
| BCMA | HC-SIR | C(+) | 070924-EZdD1 | 8928 | L363 2+; MM.1S 3+ |
| CD20-CD79b | HC-SIR | (−) | 070924-BBjR1 | 8929 | RAJI 2+; Jeko+; Daudi 2+ |
| CD79b | HC-SIR | (−) | 070924-BBkC2 | 8930 | RAJI 1.5+; Daudi 1.5+ |
| CD79b | MC-SAR | (−) | 080124-EZtE1 | 8931 | RAJI 0.5+; Daudi+ |
| CD79b | MC-SAR | (−) | 080124-EZtM1 | 8932 | RAJI 0.5+; Daudi 2+ |
| CD79b | MC-SAR | (−) | 080124-EZtO1 | 8933 | RAJI 0.5+; Daudi 2+ |
| CD19-PSMA | MC-SAR | (−) | 092624-JSuA2 | 8934 | LnCaP+/− |
| CD19-BCMA | MC-SAR | (−) | 092624-JSuD1 | 8935 | RAJI 0.5+; MM.1S 0.5+ |
| CD19-BCMA | MC-SAR | (−) | 092624-JSuB1 | 8936 | RAJI 0.5+; MM.1S 0.5+ |
| CD19-BCMA | MC-SAR | (−) | 092624-JSuC2 | 8937 | RAJI 0.5+; MM.1S 0.5+ |
| DLL3-BCMA | MC-SAR | (−) | 100424-EZeT1 | 8938 | MM.1S 0.5+; H82+/−; |
| CD19-BCMA | MC-SAR | (−) | 100324-BBtQ1 | 8939 | RAJI 0.5+; MM.1S 0.5+ |
| TAJ | MC-SAR | P11(+) | 100424-EZeK1 | 8940 | HepG2+ |
| MPL | MC-SAR | P11(+) | 100424-EZeE1 | 8941 | HEL 0.5+ |
| MPL | MC-SAR | (−) | 100424-EZeF1 | 8942 | HEL 0.5+ |
| TAJ | MC-SAR | P11(+) | 100424-EZeG1 | 8943 | A2058+/−; Hutu-80 0.5+; |
| MPL | MC-SAR | (−) | 100424-EZeH1 | 8944 | HEL+/− |
| MPL | MC-SAR | (−) | 100424-EZeJ1 | 8945 | HEL 0.5+ |
| CD19 | MC-SAR | (−) | 100324-BBtP2 | 8946 | RAJI 0.5+ |
| CD19-BCMA | MC-SAR | (−) | 100324-BBtR2 | 8947 | RAJI 0.5+ |
| DLL3 | MC-SAR | (−) | 100424-XWA1 | 8948 | H82+/−; SK-MEL-5+/−; |
| DLL3 | MC-SAR | (−) | 100424-XWB1 | 8949 | H82+/−; SK-MEL-5+/−; |
| DLL3 | MC-SAR | (−) | 100424-XWC1 | 8950 | H82+/−; SK-MEL-5+/−; |
| CD79b | MC-SAR | (−) | 101124-EZfA1 | 8951 | RAJI+; Jeko+ |
| CD79b | MC-SAR | (−) | 101124-EZfB1 | 8952 | RAJI+; Jeko 1.5+ |
| CD79b | MC-SAR | (−) | 101124-EZfC1 | 8953 | RAJI+; Jeko 1.5+ |
| CD79b | MC-SAR | (−) | 101124-EZfD1 | 8954 | RAJI+; Jeko 1.5+ |
| CD79b | MC-SAR | (−) | 101124-EZfE1 | 8955 | RAJI+; Jeko 1.5+ |
| CD79b | MC-SAR | (−) | 101124-EZfF1 | 8956 | RAJI+; Jeko 1.5+ |
| CD79b | MC-SAR | (−) | 101124-EZfG1 | 8957 | RAJI+; Jeko 1.5+ |
| CD79b | MC-SAR | (−) | 101124-EZfH1 | 8958 | RAJI+; Jeko+ |
| CD79b | MC-SAR | (−) | 101124-EZfJ1 | 8959 | RAJI+; Jeko 1.5+ |
| DLL3-BCMA | MC-SAR | (−) | 100424-EZeU2 | 8960 | H1436+/−; COLO679+/− |
| CD19-BCMA | MC-SAR | (−) | 102524-EZeQ1 | 8961 | RAJI 0.5+; MM.1S 0.5+ |
| CD20 | MC-SAR | (−) | 010425-EZyN1 | 8962 | Jeko+/−; RAJI+/− |
| CD20 | MC-SAR | (−) | 010425-EZyQ1 | 8963 | Jeko+/−; RAJI 0.5+ |
| CD20 | MC-SAR | (−) | 010425-EZyT1 | 8964 | Jeko 0.5+; RAJI+ |
| CD20 | MC-SAR | (−) | 010425-EZyW1 | 8965 | Jeko 0.5+; RAJI+ |
| TGFBR2 | MC-SAR | (−) | 010425-EZyY1 | 8966 | Jeko+/− |
| CD20 | MC-SAR | (−) | 010425-EZzA1 | 8967 | Jeko+/− |
| CD20 | MC-SAR | (−) | 010425-EZzB1 | 8968 | Jeko 0.5+ |
| CD20 | MC-SAR | (−) | 010425-EZzD1 | 8969 | Jeko+/− |
| DLL3-BCMA | MC- | (−) | 100424-EZeS1 | 8970 | MM.1S+; H82+/ |
| | 16SAR | | | | |
| CD19-BCMA | MC- | (−) | 102524-EZeP1 | 8971 | RAJI 0.5+; MM.1S+ |
| | 16SAR | | | | |
| TAJ | MC- | P11(+) | 080124-EZtQ1 | 8972 | Hutu-80+/− |
| | zSAR | | | | |
| TAJ | MC- | P11(+) | 080124-EZtT1 | 8973 | Hutu-80+/− |
| | zSAR | | | | |
| TAJ | MC- | P11(+) | 100424-EZeA1 | 8974 | A2058 0.5+; HepG2+/−; |
| | zSAR | | | | Hutu-80 0.5+; SW1573+ |
| MPL | MC- | (−) | 100424-EZeB1 | 8975 | HEL 1.5+ |
| | zSAR | | | | |
| MPL | MC- | (−) | 100424-EZeC1 | 8976 | HEL 1.5+ |
| | zSAR | | | | |
| TAJ | MC- | P11(+) | 100424-EZeD1 | 8977 | A2058+/−; HepG2+/−; |
| | zSAR | | | | |

The presence or absence of Tag and the type of Tag is shown as (+) and (−). C(+) indicates that the SHARP-tagged protein is co-expressed with the SAR. Abbreviations used are MC-SAR, multichain SAR; MC-zSAR, multichain zSAR; MC-16SAR, multi-chain CD16 SAR; HC-SAR, Hybrid Chain SAR; zSIR, CD3z synthetic immune receptor; SIR, Synthetic Immune Receptor; CAR, chimeric antigen receptor.

TABLE 7
Table 7: Malibu-Glo Assay

| SEQ ID | Cell line | Fold Increase |
|---|---|---|
| 8979 | RAJI | 117.02 |
| 8980 | RAJI | 144.08 |
| 8981 | RAJI | 114.69 |
| 8982 | RAJI | 119.35 |
| 8983 | RAJI | 160.99 |
| 8984 | RAJI | 50.82 |
| 8985 | RAJI | 121.15 |
| 8986 | RAJI | 47.57 |
| 8987 | RAJI | 1.13 |
| 8988 | RAJI | 39.22 |
| 8989 | RAJI | 38.28 |
| 8990 | RAJI | 270.34 |
| 8991 | RAJI | 39.11 |
| 8992 | RAJI | 26.86 |
| 8993 | RAJI | 21.17 |
| 8994 | RAJI | 17.30 |
| 9001 | RAJI | 0.48 |
| 9057 | RAJI | 481.39 |
| 9058 | RAJI | 125.51 |
| 9059 | RAJI | 64.94 |
| 9060 | RAJI | 26.92 |
| 9061 | RAJI | 2.47 |
| 9062 | RAJI | 628.83 |
| 9067 | RAJI | 68.28 |
| 9068 | RAJI | 68.73 |
| 9069 | RAJI | 66.56 |
| 9070 | RAJI | 33.56 |
| 9071 | RAJI | 2.19 |
| 9072 | RAJI | 1.93 |
| 9080 | RAJI | 7.40 |
| 9081 | RAJI | 2.05 |
| 9082 | RAJI | 6.97 |
| 9083 | RAJI | 8.37 |
| 9084 | RAJI | 9.36 |
| 9085 | RAJI | 17.40 |
| 9086 | RAJI | 96.80 |
| 8995 | HL60 | 6.13 |
| 8997 | HL60 | 7.19 |
| 9006 | HL60 | 5.02 |
| 9007 | HL60 | 1.31 |
| 9008 | HL60 | 3.70 |
| 9009 | PA-1 | 18.63 |
| 9010 | PA-1 | 16.56 |
| 9011 | PA-1 | 6.65 |
| 9012 | PA-1 | 2.47 |
| 9013 | PA-1 | 9.96 |
| 9014 | PA-1 | 4.13 |
| 9015 | PA-1 | 9.03 |
| 9016 | PA-1 | 152.05 |
| 9017 | PA-1 | 685.82 |
| 9019 | MM1S | 2.40 |
| 9020 | MM1S | 2.37 |
| 9021 | MM1S | 5.94 |
| 9022 | MM1S | 3.42 |
| 9023 | MM1S | 1.59 |
| 9024 | MM1S | 1.29 |
| 9025 | MM1S | 1.16 |
| 9027 | MM1S | 1.12 |
| 9028 | MM1S | 1.06 |
| 9034 | MM1S | 7.19 |
| 9037 | MM1S | 12.23 |
| 9038 | MM1S | 9.38 |
| 9048 | MM1S | 4.37 |
| 9049 | MM1S | 6.30 |
| 9050 | MM1S | 3.56 |
| 9051 | MM1S | 4.76 |
| 9052 | MM1S | 15.81 |
| 9053 | MM1S | 1.53 |
| 9055 | MM1S | 10.88 |
| 9056 | MM1S | 7.43 |
| 9087 | SKOV3 | 9.12 |
| 9088 | SKOV3 | 11.52 |
| 9089 | SKOV3 | 2.04 |
| 9090 | SKOV3 | 1.49 |
| 9091 | SKOV3 | 1.31 |
| 9092 | SKOV3 | 1.22 |
| 9093 | SKOV3 | 1.24 |
| 9099 | SK-MEL-5 | 2.61 |
| 9100 | SK-MEL-5 | 0.88 |
| 9101 | SK-MEL-5 | 29.65 |
| 9102 | SK-MEL-5 | 14.67 |
| 9103 | NCI-H82 | 23.80 |
| 9104 | 786-O | 15.38 |
| 9105 | SKOV3 | 671.60 |
| 9106 | LNCaP | 27.37 |
| 9107 | HepG2 | 278.48 |
| 9108 | HepG2 | 87.82 |
| 9109 | HepG2 | 85.78 |
| 9110 | MM1S | 1.92 |
| 9111 | LoVo | 1.25 |
| 9112 | MM1S | 4.28 |
| 9116 | MM1S | 2.46 |
| 9122 | MM1S | 5.74 |
| 9123 | MM1S | 4.49 |
| 9130 | LoVo | 1.84 |
| 9131 | LoVo | 1.44 |
| 9132 | LoVo | 2.02 |
| 9133 | LoVo | 10.00 |
| 9140 | HL60 | 11.80 |
| 9141 | HL60 | 8.29 |
| 9142 | HL60 | 10.11 |
| 9143 | HL60 | 5.11 |
| 9144 | HL60 | 3.94 |
| 9145 | HL60 | 2.95 |
| 9000 | HL60 | 5.40 |
| 9002 | HL60 | 5.34 |
| 9003 | HL60 | 7.51 |
| 9004 | HL60 | 10.00 |
| 8998 | HL60 | 17.02 |
| 8999 | HL60 | 28.67 |
| 9094 | SK-MEL-5 | 1.84 |
| 9095 | SK-MEL-5 | 3.87 |
| 9096 | SK-MEL-5 | 3.14 |
| 9097 | SK-MEL-5 | 3.82 |
| 9098 | SK-MEL-5 | 2.77 |

### Definitions

The invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the disclosure are generally performed according to conventional methods well-known in the art and as described in various general and more specific references that are cited and discussed throughout the present specification unless otherwise indicated. See, e.g., Sambrook et al., Molecular Cloning: A Laboratory Manual (4th ed., Cold Spring Harbor Laboratory Press, Cold Spring Harbor, N.Y. (2013)). The nomenclatures used in connection with, and the laboratory procedures and techniques of, immunology, molecular biology, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “autonomous antigen binding domain” or “AABD” as used herein refers to an antigen binding domain that can bind to an antigen autonomously, i.e., in the absence of another antigen binding domain. In an embodiment, the AABD is a non-scFv antigen binding domain. An exemplary non-scFV based autonomous antigen binding domain includes but is not limited to a vHH domain, a humanized vHH domain, a single variable domain-TCR (svd-TCR), and non-immunoglobulin antigen binding scaffold such as a DARPIN, an affibody, a ZIP domain (e.g., RZIP, EZIP, E4, R4 etc.), an affilin, an adnectin, an affitin, an obody, a repebody, a fynomer, an alphabody, an avimer, an atrimer, a centyrin, a pronectin, an anticalin, a kunitz domain, an Armadillo repeat protein or a fragment thereof. Additional examples of non-scFV based autonomous antigen binding domains include the ligand binding domain of a receptor (e.g., CD16-V158A, NKG2D) or a fragment thereof, the receptor binding domain of a ligand (e.g., APRIL, Thrombopoietin etc.) or a fragment thereof, an adaptor (e.g., RZIP, EZIP, NKG2D-AF etc.) or a fragment thereof, an adaptor binding protein (e.g. ULBP2R, ULBP2-S3 etc.) or a fragment thereof, an epitope or a tag (e.g., SHARP-tag etc.), an autoantigen or a fragment thereof and the like.

The disclosure described the use of AABD, such as human V_{H} (or vH) domains, such as multiple human V_{H} domains, as building blocks to make unispecific, bispecific and multi-specific SARs. In an embodiment, the disclosure describes the use of AABD, such as human V_{H} domains, such as multiple human V_{H} domains, as building blocks to make unispecific, bispecific and multispecific novel SARs.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods or describe the compositions herein. Moreover, any value or range (e.g., less than 20 or similar terminology) explicitly includes any integer between such values or up to the value. Thus, for example, “one to five mutations” explicitly includes 1, 2, 3, 4, and/or 5 mutations.

The term “Ab-TCR” or “AbTCR” refers to a next generation CAR platform as described in WO 2017/070608 A1 which is incorporated herein by reference. In an embodiment, an Ab-TCR comprises an antibody moiety that specifically binds to a target antigen fused to a TCR module capable of recruiting at least one TCR signaling module. Exemplary TCR modules that can be used in the construction of Ab-TCR are provided in SEQ ID NO:6009-6014 and in WO 2017/070608 A1 which is incorporated herein by reference.

The term “accessory module” refers to any one or more of PDL1, PDL2, CD80, CD86, 41BBL, CD40L, vFLIP-K13, MC159, cFLIP-L/MRITa, cFLIP-p22, a multi-purpose switch (e.g., IL2-tBCMA, IL15-tBCMA, IL2-RQR, IL15-RQR etc.), NKG2C, CD94, DAP10, DAP12, CD3ε, CD3γ, CD3δ, CD3ζ, FcRγ, and combination thereof that is expressed in an immune cell (e.g., NK cell or T cell, e.g., SAR-NK cell, SAR-T cell or TCR-T cell) to decrease, regulate or modify the activity of the immune cell. In an embodiment, an accessory module is a therapeutic control (e.g., iCaspase 9).

As used herein “affinity” is meant to describe a measure of binding strength. Affinity generally refers to the “ability” of the binding agent to bind its target. There are numerous ways used in the art to measure “affinity”. In an embodiment, the antibodies, antibody fragments (e.g., scFV), antibody-conjugates (e.g., antibody drug conjugates) that bind with 1.5-fold, 2-fold, 5-fold, 10-fold, 20-fold or 50-fold lower affinity to a variant epitope tag described herein as compared to the wild-type epitope tag represented by SEQ ID NO; 1-11. In an embodiment, the antibodies, antibody fragments (e.g., scFV), antibody-conjugates (e.g., antibody drug conjugates) that bind with 1.5-fold, 2-fold, 5-fold, 10-fold, 20-fold or 50-fold higher affinity to a variant epitope tag described herein as compared to the wild-type epitope tag represented by SEQ ID NO; 1-11.

The term “antibody,” as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. The antibody may be ‘humanized’, ‘chimeric’, fully human or non-human. An antibody may have a single domain (e.g., a single vH domain).

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies (sdAb) such as either vL or vH, camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide mini-bodies).

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (K) and lambda (k) light chains refer to the two major antibody light chain isotypes. [00463]“Anticancer agent” refers to agents that inhibit aberrant cellular division and growth, inhibit migration of neoplastic cells, inhibit invasiveness or prevent cancer growth and metastasis. The term includes chemotherapeutic agents, biological agent.

The term “anticancer effect” or “anti-tumor effect” refers to a biological effect which can be manifested by various means, including but not limited to, a decrease in tumor volume, a decrease in the number of cancer cells.

The term “antigen” or “Ag” refers to a molecule that provokes an immune response. This immune response may involve either antibody production, or the activation of specific immunologically-competent cells, or both. Non-limiting examples of antigen or antigens that can be specifically bound by any of the antigen-binding domains are described in Table 1.

An “antigen binding domain” or “antigen binding module” or “antigen binding segment” or “antigen specific domain” (ASD) or “binding domain” refers to a polypeptide or peptide that due to its primary, secondary or tertiary sequence, post-translational modifications and/or charge binds to an antigen with a high degree of specificity. In exemplary embodiments, the target antigens and SEQ ID Nos of various antigen binding domains are set forth herein in Tables 2-4 of this disclosure. In further exemplary embodiments, the target antigen and SEQ ID NOs of vL, vH, scFVs, and their CDR regions are set forth herein in Tables 6A-C of patent application PCT/US18/53247 and in Tables 3-4 of patent application PCT/US19/035096, which are incorporated in their entirety by reference herein.

The term “Association constant (Ka)” is defined as the equilibrium constant of the association of a receptor and ligand.

The term “autoantigen” refers to an endogenous antigen that stimulates production of an autoimmune response, such as production of autoantibodies. Examples of autoantigens include, but are not limited to, desmoglein 1, desmoglein 3, and fragments thereof.

As used herein, the term “backbone” or “architecture” or “design” refers to the configuration of the different components (e.g., antigen binding domains, hinge domains, transmembrane domains, signaling domains) that comprise different SAR and any accessory module which is generally optional. The expression of nucleic acids encoding two or more components of the SAR and accessory modules may be driven by separate promoters. Exemplary promoters include EF1α, EFS, RSV, MNDU3 (SEQ ID NO: 3526-7), Hsp70 and Hsp90.

As referred to here, the term “binds” or “binding” or “recognized by” refers to a non-covalent binding, in particular ionic bonds, Van der Wall forces, hydrogen bonds and/or hydrophobic interactions.

The term “cTCR” refers to a wild-type TCR nucleic acid coding sequence and the corresponding wild-type TCR protein linked to an antigen binding domain that is not derived from a TCR. cTCR have been described in (Gross, Waks, & Eshhar, 1989). cTCRs are used in some embodiments and as reference controls.

As used herein, an “epitope” is defined to be the portion of an antigen capable of eliciting an immune response, or the portion of an antigen that binds to an antibody or antibody fragment. Epitopes can be a protein sequence or subsequence.

The term “expression vector” refers to a vector comprising a recombinant polynucleotide comprising expression control sequences operatively linked to a nucleotide sequence to be expressed.

A “functional portion” (“biologically active portion”) of a protein refers to a portion of a protein that retains one or more functions of full length or mature protein.

“Functional variant” or “Variant” of a given entity (tag, antibody, protein, etc.) means a version that may not be exactly identical in sequence but performs substantially the same function. For example, a functional variant of the SHARP-tag may have one or two amino acids substituted yet still be bound by SABR. A functional variant of SABR may be a variant with slightly different sequence but still binds is SHARP tag with high affinity. Functional variants of a SAR or protein retain the intended activity (antigen recognition, signaling, etc.).

The term “FcRγ” or “FCER1G” or “FCRG” or “FcRy” as used herein refers to gene represented by Gene ID: 2207.

The term “functional portion” when used in reference to a SAR refers to any part or fragment of the SAR, which part or fragment retains the biological activity of the SAR of which it is a part (the parent SAR). Functional portions encompass, for example, those parts of a SAR that retain the ability to recognize target cells, or detect, treat, or prevent a disease, to a similar extent, the same extent, or to a higher extent, as the parent SAR. In reference to the parent SAR, the functional portion can comprise, for instance, about 10%, 25%, 30%, 50%, 68%, 80%, 90%, 95%, or more, of the parent SAR.

The term “flexible polypeptide linker” as used herein refers to a peptide linker that consists of amino acids such as glycine and/or serine residues used alone or in combination, to link polypeptide chains together (e.g., variable heavy and variable light chain regions together).

“Host cell” or “engineered cell” or “Genetically modified cells,” or “redirected cells,” or “genetically engineered cells” refers to a cell that has been genetically modified to express a recombinant polypeptide (here, typically a SAR or a SHARP-tagged construct). “Transduced,” “transfected,” or “transformed” are used interchangeably to denote introduction of genetic material into a cell (via viral vector, plasmid, mRNA electroporation, etc.).

An “HLA-independent TCR” or an “MHC-independent TCR” as defined herein is a TCR that can recognize an antigen independent of MHC restriction.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.).

The term “heterologous” when used in context of protein domains refer to domains that is not in its natural environment. For example, a heterologous protein domain is not part of a single naturally occurring polypeptide or protein. Stated in another way, two domains are heterologous if they are derived from two different polypeptides or proteins found in nature. For example, TCRα constant domain and TCRβ transmembrane domains are heterologous to each other as they are derived from two different polypeptide or proteins.

The two domains are considered “interspecies heterologous domains” if they are derived from homologs of the same protein but are derived from two different species and if there is <80% amino acid sequence identity between the two domains. For example, human TCRα constant domain and mouse TCRα transmembrane domains are heterologous to each other if the human TCRα constant domain is less than 80% identical to mouse TCRα constant domain at the amino acid level or if the human TCRα transmembrane domain is less than 80% identical to mouse TCRα transmembrane at the amino acid level. For the purpose of this disclosure, two domains are not considered interspecies heterologous domains if <20% of amino acid residues of a protein domain belonging to one species are replaced by the corresponding residues found in a different species. For example, human TCRα transmembrane domain and a variant of human TCR constant domain are not considered interspecies heterologous if <20% amino acid residues of the human TCRα constant domain are replaced by the corresponding residues found in the mouse TCRα constant domain.

“Hinge region” (HR) as used herein refers to the hydrophilic region which is between the antigen binding domain and the transmembrane domain of a SAR.

“Hybrid TCR Chain” or “Hybrid Chain” as the term is used herein, refers to a chain that comprises at least one domain selected from the group of TCR constant domain (CD or C), TCR connecting peptide (ConnP), TCR transmembrane (TM) domain and TCR intracytoplasmic domain (CP or IC) that is heterologous. In an example embodiment, a hybrid TCR chain refers to a TCR chain in which the TM domain is derived from one TCR chain and at least one of the domains selected from CD, ConnP, and IC is derived from a different TCR chain. For example, if TM is derived from human TCRα, then at least one of the domains selected from CD, ConnP, and IC is derived from TCRβ1/β2, TCRγ, TCRS or pre-TCRα. A hybrid chain may also comprise of domains derived from two different species. Thus, a hybrid chain may comprise of human TCRα constant domain and a non-human TCRα transmembrane domain. A hybrid TCR chain may have 1, 2, 3 or more domains that are heterologous. Hybrid chains are also described in PCT/US2024/10592 (incorporated by reference herein).

“Hybrid Chain SIR” or (HC-SIR) or a Hybrid Chain SAR (HC-SAR) as the term is used herein, refers to a heterodimeric synthetic immune receptor (SIR) or synthetic antigen receptor (SAR) in which at least one TCR constant chain is a hybrid chain. A hybrid chain SIR or a hybrid chain SAR may have both TCR chains that are hybrid. HC-SIR and HC-SAR are also described in PCT/US2024/10592.

As used herein, the term “hydrophilic” refers to a molecular property or characteristic of a compound, moiety, residue, or domain that exhibits an affinity for water or aqueous environments. Examples of hydrophilic residues include, but are not limited to, serine (Ser), threonine (Thr), asparagine (Asn), glutamine (Gln), arginine (Arg), lysine (Lys), aspartic acid (Asp), and glutamic acid (Glu).

As used herein, the term “hydrophobic” refers to a molecular property or characteristic of a compound, moiety, residue, or domain that repels or is insoluble in water and tends to associate with non-polar solvents or lipid environments.

As used herein, the term “intracellular protein” refers to a protein or polypeptide that is normally localized within the interior of a cell, including but not limited to the cytoplasm, nucleus, mitochondria, endoplasmic reticulum, Golgi, lysosomes, or other subcellular compartments.

As used herein, the term “polar” refers to a molecule, group, or residue that exhibits an unequal distribution of electric charge, resulting in a permanent electric dipole moment. Polar amino acid residues include, but are not limited to, serine (Ser), threonine (Thr), tyrosine (Tyr), asparagine (Asn), and glutamine (Gln). Charged residues (e.g., Lys, Arg, Asp, Glu) are also considered polar.

A “hydrophobic portion,” as used herein, means any amino acid sequence having a three-dimensional structure that is thermodynamically stable in a cell membrane, and generally ranges in length from about 15 amino acids to about 30 amino acids.

As used herein, the term “hypoimmunogenic” refers to a molecule, polypeptide, domain, or composition that elicits a reduced or attenuated immune response compared to a reference immunogenic molecule when administered to a subject. A hypoimmunogenic entity may still be recognized by components of the immune system (e.g., T cells, B cells, antibodies), but to a significantly lesser extent than a naturally occurring, wild-type, or unmodified counterpart. Hypoimmunogenicity may be achieved, for example, through sequence modification, removal of known T-cell epitopes, PEGylation, glycosylation, or other engineering strategies that minimize antigen presentation or immune recognition.

As used herein, the term “non-immunogenic” refers to a molecule, polypeptide, domain, or composition that does not elicit a detectable immune response when administered to a subject under conditions in which a comparable immunogenic control would produce such a response. A non-immunogenic entity lacks epitopes capable of stimulating B cell or T cell activation and is not subject to antigen-specific immune surveillance or memory response. For the purposes of the present disclosure, a “non-immunogenic” sequence may include naturally occurring human-derived sequences or engineered sequences that exhibit no detectable antibody generation or T cell activation in a validated in vivo or in vitro immune assay.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, monocytes/macrophages, and myeloid-derived phagocytes.

“Immune effector function” or “immune effector response,” “effector function” refers to the specialized function of a differentiated cell. Effector function of a T-cell or NK-cells, for example, may be cytolytic activity or helper activity including the secretion of cytokines. For example, an immune effector function or response refers a property of a T or NK cell that promotes killing or the inhibition of growth or proliferation, of a target cell.

An “intracellular signaling domain,” (ISD) or “activation domain” “effector domain” as the term is used herein, refers to an intracellular signaling portion of a molecule.

The term “isolated” as used herein refers to molecules or biologicals or cellular materials being substantially free from other materials. An “isolated SHAPR-tagged cell” is one that has been separated from unmodified cells, typically by use of the SABR in a selection process.

“Junction amino acids” or “junction amino acid residues” refer to one or more (e.g., about 2-20) amino acid residues between two adjacent motifs, regions or domains of a polypeptide.

A “long linker” or “long linker domain” is a linker that is between 25 to 500 amino acids in length. In an embodiment, a long linker is about 25-500 amino acids and any number in between in length.

As used herein, the term “linker” (also “linker domain” or “linker region”) refers to an oligo or a polypeptide (or an oligo encoding the polypeptide) that joins together two or more domains or regions of a polynucleotide or polypeptide, respectively, disclosed herein. In an embodiment, the polypeptide can be a SAR (e.g., a CAR, SIR, zSIR, Ab-TCR, z16SAR, TCR etc.), an antibody, an antibody fragment, a non-immunoglobulin antigen binding scaffold, a cytokine, a chemokine or any recombinant protein. The linker may join two or more domains or regions of a SAR (e.g., a CAR, SIR, zSIR, Ab-TCR, z16SAR, TCR etc.). The linker may join the epitope tag described herein to a region of a SAR (e.g., a CAR, SIR, zSIR, Ab-TCR, z16SAR, TCR etc.). The linker may join two or more epitope tags. The linker can be anywhere from 1 to 500 amino acids in length or 3 to 1500 nucleotide in length. In some embodiments the “linker” is cleavable or non-cleavable. The linker may be 5-10 amino acids in length, 5-15 amino acids in length, 6-120 amino acids in length. The linker may be 2, 3, 4, 5, 6, 7, 8, 9, 10, 11, 12, 13, 14, 15, 16, 17, 19, 20, 21, 22, 23 24, 25, 26, 27, 28, 29, 30 amin acids in length. The linker may be about 5-15 amino acids in length, about 6-20 amino acids, about 7-30 amino acids in length. In some embodiment, the one or more epitope tags may serve as the linker. For example, the epitope tag may join the antigen binding domain of a CAR (i.e., scFv region of a CAR) to the hinge domain of the CAR. Alternatively, the epitope tag may join the vL and/or vH regions of a SAR (e.g., SIR, Ab-TCR, zSIR, z16SAR etc.) to the signaling chains (e.g., TCRα/TCRβ, TCRγ/TCRδ or CD3ζ/CD3ζ etc.). In an embodiment, the linker joins the one or more epitope tags to an antibody (e.g., monoclonal antibody, a camelid vHH domain, a single vH domain etc.), an antibody fragment (e.g., scFv, Fab etc.), a non-immunoglobulin antigen binding scaffold (e.g., a DARPIN, Centyrin etc.), a cytokine, a chemokine or any recombinant protein. In an embodiment, the linker is a flexible linker, Exemplary flexible linkers are provided in SEQ ID NO: 1330-1331 and 1453. Additional flexible linkers are known in the art.

The term “lentivirus” refers to a genus of the Retroviridae family. The term “lentiviral vector” refers to a vector derived from at least a portion of a lentivirus genome, including especially a self-inactivating lentiviral vector. Example of a vector is provided in SEQ ID NO: 3530.

A “multipurpose switch” or “multipurpose gene” encodes for a protein that provide suicide, survival and marker functions. In an embodiment, all the above functions are provided by a single polypeptide chain. Example multipurpose switches include SEQ ID NO: 679-684, 688, 699.

The term “multi-chain synthetic antigen receptor” “multi-chain SAR” means a synthetic antigen receptor comprising two or more polypeptide chains. A multi-chain SAR can be a double chain SAR or a three chain SAR. A double chain SAR comprises two membrane associated domain (e.g., transmembrane or membrane anchoring domains).

“Native” or “Naturally occurring” or “endogenous” or “Natural” as used herein refers to an amino acid, nucleotide, gene, protein, peptide, nucleic acid (e.g., DNA, RNA etc.) or fragment thereof that is native to a cell or is naturally expressed in a cell. An “endogenous peptide”, “Native peptide” or “Naturally occurring peptide” or “Natural peptide” refers to a peptide or polypeptide that is naturally produced or expressed within a particular organism, tissue, or cell type, without the introduction of exogenous genetic material or recombinant expression systems. An endogenous or a natural peptide may be a fragment of an endogenous protein or polypeptide and/or derived from an endogenous protein or polypeptide. An endogenous or a natural peptide is identical in amino acid sequence to a peptide found in nature. An endogenous peptide or a natural peptide may be isolated directly from a biological source or produced synthetically or recombinantly, provided its sequence match those occurring in a naturally derived counterpart. Endogenous or natural peptides are distinguished from synthetic, engineered, or mutated peptides that contain non-naturally occurring sequences, modifications, or amino acid analogs. In an example embodiment, SEQ ID NO: 1 to 10 represent endogenous peptides that are derived from human CD79b. In the context of a TCR chain, a native or endogenous TCRα chain polypeptide of a T cell consists of a variable domain (Vα) joined to a TCRα constant chain. Examples of a naturally occurring amino acids or natural amino acids include Glycine, Serine, Alanine etc. Examples of non-naturally occurring amino acids include beta-amino acids, N-methyl amino acids, alpha-methyl amino acids, homo-amino acids, D-amino acids, norleucin, citrulline, ornithine, peptoid derivatives etc. The epitope tag and polypeptide described herein may comprise naturally occurring or non-naturally occurring amin acids.

“Native receptor” or “Naturally occurring receptor” “natural receptor” or “endogenous receptor” or “native receptor” as used herein refers to any receptor that occurs in nature and comprises an antigen binding or a ligand binding domain. The term includes functional variants, isoforms, and homologs from other mammalian species. A native receptor can be “native signaling receptor” or a “naturally occurring signaling receptor” if it is capable of transmitting a cell signal upon binding to its target. A naturally occurring receptor or native receptor is native to a cell or is naturally expressed in a cell. Examples of naturally occurring signaling receptors or native receptors include, but are not limited to, CD16A, CD16B, NKp30, NKp44, NKp46, KIR2DS4, NKG2D etc. For the purpose of this disclosure, the CD3 signaling chains (CD3E, CD37, CD36 and CD3ζ) are not included within the definition of a “naturally occurring receptor” and are instead classified as a signaling adaptor.

As used herein, the term “non-TCR naturally occurring receptor” or “non-TCR naturally occurring signaling receptor” or “non-TCR receptor” or ‘non-TCR signaling receptor” refers to a receptor that is not a T cell receptor (TCR). A non-TCR receptor can be expressed in cells other than a T cell. A non-TCR receptor can be expressed in cells that lack the expression of CD3ζ, CD3R, CD36 and/or CD37 chains.

As used herein, the term “non-T cell receptor module” or ““non-TCR module” or “non-TCR signaling module” or “NTCRM” refers to a module that lacks sequences comprised of the T cell receptor transmembrane domains and may further lack all or a portion of T cell receptor connecting peptides and/or intracellular domains. An NTCRM lacks sequences comprised of the transmembrane domains of TCRα, TCRβ, TCRγ, TCRδ or pre-TCRα. An NTCRM may further lack all or a portion of the connecting peptides and/or intracellular domains of TCRα, TCRβ, TCRγ, TCRδ or pre-TCRα. An example non-TCR module (NTCRM) comprises of two CD3z transmembrane domains. Another example NTCRM comprises of a CD3z transmembrane domain and a CD16 transmembrane domain.

As used herein, the term “non-CD3 adaptor module” or “non-CD3 adaptor” or “non-TCR/CD3 adaptor” or ““non-TCR/CD3 signaling adaptor” or ““NCAM” refers to a signaling adaptor that is not a component of the T cell receptor/CD3 receptor complex. In an embodiment, a “non-TCR/CD3 adaptor” does not comprise the transmembrane and/or cytosolic regions of CD3ε, CD3ζ, CD3γ or CD3δ chains or variants thereof.

The term “near the N-terminus” as used herein means within the N-terminal 30 amino acids. For example, the term “an AABD operably linked to the N-terminus or near the N-terminus of a vL and/or vH domain”, mean an AABD that is operably linked at the N-terminus of a vL or a vH fragment or operably linked to the N-terminal 2, 3, 4, 5, 6, 7, 8, 9, 10, 11, 12, 13, 14, 15, 16, 17, 18, 19, 25 or 30 amino acid comprising the vL or the vH domain. Similarly, the term “an AABD operably linked to the N-terminus or near the N-terminus of a Va and/or Vb domain”, mean an AABD that is operably linked at the N-terminus of a Va or a Vb fragment or operably linked to the N-terminal 2, 3, 4, 5, 6, 7, 8, 9, 10, 11, 12, 13, 14, 15, 16, 17, 18, 19, 20, 21, 22, 23, 24, 25 or 30 amino acid comprising the Va or the Vb domain. An AABD of the disclosure may be operably linked to or near the N-terminus of another domain either directly or via an intervening linker sequence.

As used herein a “non-naturally occurring agent” or “non-native” or “exogenous”, “heterologous” or “non-endogenous” or “non-natural” refers to an agent (e.g., a gene, protein, compound, molecule) that is not naturally expressed in a cell or a subject. Stated another way, the non-naturally occurring agent or heterologous agent is “engineered” to be expressed in a cell.

As used herein a “non-naturally occurring immune receptor” or “exogenous immune receptor” “non-naturally occurring receptor” refers to an immune receptor that is not naturally expressed in an immune cell. Stated another way, the non-naturally occurring immune receptor is “engineered” to be expressed in an immune cell. An example non-naturally occurring immune receptors is a SAR (e.g., 2^{nd} generation CAR, SIR, cTCR, STAR, zSIR, Ab-TCRs, TFPs and recombinant TCR).

As used herein a “non-naturally occurring TCR antigen binding domain” or “exogenous TCR antigen binding domain” refers to a binding domain operably linked to a TCR constant region that is chimeric and non-naturally occurring with respect to a TCR present in nature. Stated another way, the non-naturally occurring TCR antigen binding domain is “engineered” using recombinant molecular biology techniques to be operably linked to a TCR and moreover, that the antigen binding domain is obtain or derived from a molecule that is distinct from a TCR found in nature. An antigen binding domain that is distinct from a TCR in nature includes antibody, antibody fragments, vH and vL fragments, scFv, humanized antibody fragments, chimeric antibody fragments, adaptors, non-immunoglobulin antigen binding scaffold, receptor, ligands, and the like.

As used herein a “non-naturally occurring antigen binding domain” or “non-naturally occurring extracellular antigen binding domain” or “heterologous antigen binding domain” refers to an antigen binding domain that is not part of a naturally occurring receptor. Example heterologous antigen binding domains include antibodies, antibody fragments (e.g., vL, vH, scFv, Fab, F(ab)2 etc.), single domain antibodies (e.g., sVH, FHVH, vHH etc.), non-immunoglobulin antigen binding domains, single variable domain-TCR (svd-TCR), recombinant TCRs, HLA-independent TCR, scTCR, epitopes, adaptors, ligands, and receptors.

The term “non-TCR antigen binding domain” refers to an antigen binding domain that is not a TCR antigen binding domain. A non-TCR antigen binding domain is structurally distinct from the variable domains (i.e., Vα, Vβ, Vγ and Vδ) found in a TCR. Example non-TCR antigen binding domains include antibody, antibody fragments (e.g., vL, vH, scFv, Fab, F(ab)2 etc.), single domain antibodies (e.g., sVH, FHVH, vHH etc.), chimeric antibody fragments, adaptors, non-immunoglobulin antigen binding scaffold (e.g., DARPIN, D domains etc.), adaptors, extracellular Fc binding domains of receptors (e.g., CD16, CD64 etc.), ligands, cytokines and the like.

The term “operably linked” or “functionally linked” or “operationally linked” refers to functional linkage or association between a first component and a second component such that each component can be functional.

“Percent identity” in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more typically over a region that is 100 to 500 or 1000 or more nucleotides (or 20, 50, 200 or more amino acids) in length. Two examples of algorithms that can be used for determining percent sequence identity and sequence similarity are the BLAST and BLAST 2.0 algorithms.

The term “peptide” as used herein refers to a linear series of amino acids connected one to the other preferably by peptide bonds between the alpha-amino and carboxy groups of adjacent residues. As used herein the term “amino acid” refers to either natural and/or unnatural or synthetic amino acids, including glycine and both the D or L optical isomers, and amino acid analogs and peptidomimetics, with proteogenic amino acids being preferred. A “proteinogenic amino acid” is an amino acid that can be incorporated biosynthetically into proteins during translation. Currently, there are 22 known genetically encoded (proteinogenic) amino acids, 20 in the standard genetic code and an additional 2 that can be incorporated by special translation mechanisms. The “peptide” as used herein preferably comprises no more than about 50 amino acids.

The term “polypeptide” as used herein usually refers to a peptide having at least about 30, at least about 40, or at least about 50 amino acids. The term “protein” as used herein comprises one or more polypeptides.

The term “fusion protein” as used herein refers to a polypeptide or protein comprising two or more subunits. At least one of the subunits is preferably a protein or polypeptide, and at least one of the subunits is preferably a peptide. Within the fusion protein, these subunits may be linked by covalent or non-covalent linkage. Preferably, the fusion protein is a translational fusion between the two or more subunits. The translational fusion may be generated by genetically engineering the coding nucleotide sequence for one subunit in a reading frame with the coding nucleotide sequence of a further subunit. Subunits may be interspersed by a linker.

As used here “Polatuzumab vedotin”, “Polatuzumab” or “Pola” or “DCDS4501A”, “RG7596”, “RG-7596” “polatuzumab vedotin-piiq” “ACD79B-VCMMAE”, “R05541077-000” sold under the brand name Polivy®, is a CD79b-directed antibody-drug conjugate medication used for the treatment of diffuse large B-cell lymphoma. Polatuzumab vedotin-piiq contains an anti-CD79b monoclonal antibody linked to the toxin monomethyl auristatin E. The drug has CAS Number 1313206-42-6, IUPHAR/BPS number 8404, DrugBank number DB12240 and INN number 9714. The peptide sequence of the antibody is described in U.S. Pat. No. 8,088,378 as the claiming patent. The sequence of the heavy chain and light chain of Polatuzumab vedotin are presented in SEQ ID NO (PRT): 1194 and 1195, respectively.

As used herein, the term “receptor” refers to a polypeptide, or portion thereof, present on a cell membrane that selectively binds one or more ligand.

As used herein, the terms “region” or “portion” when used in reference to a nucleic acid molecule refers to a set of linked nucleotides that is less than the entire length of the molecule, such as a CD3ζ signaling region described herein.

The term “retrovirus vector” refers to a vector derived from at least a portion of a retrovirus genome. Examples of retrovirus vector include MSCVneo or MSCVpac.

The term “SABR” or “SHARP Antigen Binding Reagent” as used herein, refers to any reagent that binds to a SHARP-tag or an epitope tag described herein. Examples of SABR include antibodies, antibody fragments, antibody conjugates and non-immunoglobulin antigen binding scaffolds. Polatuzumab vedotin, SN8, 2F2, huMA79b, H2Mab-250 are some examples of SABRs described in this disclosure.

The term “SAR” or “Synthetic Antigen Receptor,” as used herein, refers to any non-native antigen binding receptor that is expressed on the surface of a cell (e.g., immune cell). The “Synthetic Antigen Receptor” or “SAR” is a non-naturally occurring receptor or a synthetic receptor that can be expressed on the surface of a cell and comprises at least one heterologous antigen binding domain and at least one membrane associated domain, wherein the membrane associated domain can be a transmembrane domain or a membrane anchoring domain (i.e., a GPI linked domain). The antigen binding domain of the SAR is heterologous to its membrane associated domain, i.e., the antigen binding domain is derived from a different source than the membrane associated domain. A SAR may further comprise a hinge domain, an extracellular ligand binding domain and/or an optional cytosolic domain. In an embodiment, a SAR comprises a polypeptide or a set of polypeptides, which when expressed in an effector cell, provides the cell with specificity for a target cell, typically a cancer cell, and with intracellular signal generation. A SAR can be single chain, two chains or more than two chains. A SAR can be unispecific, bispecific, or multi-specific. A SAR may have one or more heterologous antigen binding domains. The term SAR comprises conventional CARs (e.g., 2^{nd} generation CARs comprising 41BB or CD28 costimulatory domains and CD3z activation domain) and also encompasses newer approaches to conferring antigen specificity onto cells, such as Antibody-TCR chimeric molecules or Ab-TCR (WO 2017/070608 A1 incorporated herein by reference), TCR receptor fusion proteins or TFP (WO 2016/187349 A1 incorporated herein by reference), Synthetic Immune Receptors (SIRs) (see, WO 2018/102795 A1, incorporated herein by reference), STAR (see, WO 2020/029774), HLA-independent TCR (see, WO2019157454A1), Tri-functional T cell antigen coupler (Tri-TAC or TAC) (see, WO 2015/117229 A1, incorporated herein by reference) and zSIR (see, PCT/US2019/035096, incorporated herein by reference). Bispecific and multi-specific SARs have been described in PCT/US2021/022641. The term “SAR” covers CAR as well as other antigen binding receptors, including but not limited to recombinant TCR. The SAR also comprises compositions comprising one or more regions derived from CD16A, CD16B, CD3ζ, DAP10, DAP12, FcRγ, TCRαβ and TCRγδ etc. and variants and fragments thereof.

The term “single-chain synthetic antigen receptor” or “single chain SAR” means a synthetic antigen receptor comprising a single polypeptide chain.

The term “double-chain synthetic antigen receptor” or “double chain SAR” means a synthetic antigen receptor comprising two polypeptide chains wherein each chain comprises at least one antigen binding domain and a signaling chain. Example double chain SARs include Synthetic Immune Receptors (SIRs) (see, WO 2018/102795 A1, incorporated herein by reference), and zSIR (see, PCT/US2019/035096, incorporated herein by reference). Bispecific and multi-specific SARs have been described in PCT/US2021/022641.

The term “One and half-chain synthetic antigen receptor” means a synthetic antigen receptor comprising two polypeptide chains wherein one chain comprises at least one antigen binding domain and a signaling chain (e.g., TCRα constant chain) and the other chain comprises a signaling chain (e.g., TCRβ constant chain) but lacks an antigen binding domain. Example one and a half chain SARs include Synthetic Immune Receptors (SIRs) (see, WO 2018/102795 A1, incorporated herein by reference).

Typically, the term “SAR-T” is used, to refer to T-cells that have been engineered to express a Synthetic antigen receptor. The term “SAR-NK” refers to an NK cell that has been engineered to express a SAR.

The term “Synthetic Immune Receptor” or alternatively an “SIR” refers to a set of polypeptides, typically two in some embodiments, which when expressed in an effector cell, provides the cell with specificity for a target cell, typically a cancer cell, and with intracellular signal generation. SIRs represent next generation CAR platforms that are described in WO 2018/102795 A1 which is incorporated herein by reference. In a typical embodiment, a SIR comprises one or more antigen binding domains (e.g., antibody or antibody fragment, a ligand, or a receptor) that bind to antigens as described herein and are joined to one or more T cell receptor constant chains or regions via an optional linker. In some embodiments, the set of polypeptides are contiguous with each other. In some embodiments, a SIR comprises two or more sets of two or more polypeptides. The polypeptides of each set of SIRs are contiguous with each other (functional polypeptide unit 1) but are not contiguous with the polypeptides of the other set (functional polypeptide unit 2). In some embodiments, the T cell receptor constant chains (or regions) of the SIR is chosen from the constant chain of human T cell receptor-alpha (TCR-alpha or TCRα or TCRa or hTCR-alpha or hTCRα or hTCRa or Cα), human T cell receptor-beta1 (TCR-beta1 or TCRβ1 or TCRb1 or hTCR-beta1 or hTCRβ1 or hTCRb1 or Cβ1), human T cell receptor-beta 2 (TCR-beta2 or TCRβ2 or TCRb2 or hTCR-beta2 or hTCRβ2 or hTCRb2 or Cβ2 also designated TCR-beta, TCRβ or TCRb or Cβ), human Pre-T cell receptor alpha ((preTCR-alpha or preTCRα or preTCRa or preCα), human T cell receptor-gamma (TCR-gamma or TCRγ or TCRg or hTCRgamma or hTCRγ or hTCRg or hTCRγ1 or hTCRgamma1, or Cγ), or human T cell receptor-delta (TCR-delta or TCRd or TCRδ or hTCR-delta or hTCRd or hTCRδ or Cδ). In some embodiments, the TCR constant chains of SIR are encoded by their wild-type nucleotide sequences while in other aspects the TCR constant chains of SIR are encoded by the nucleotide sequences that are not wild-type. In some embodiments, the TCR constant chains of SIR are encoded by their human codon optimized sequences. In some embodiments, the TCR constant chains of SIR encode for the wild-type polypeptide sequences while in other embodiments the TCR constant chains of SIR encoded for polypeptides that carry one or more mutations. In some embodiments, the TCR constant chains of SIR are encoded by their codon optimized sequences that carry one or more mutations.

As use herein, the term “specifically binds” or “is specific for” refers to measurable and reproducible interactions, such as binding between a target and an antibody or antibody moiety, that is determinative of the presence of the target in the presence of a heterogeneous population of molecules, including biological molecules.

The term “signaling domain” refers to the functional region of a protein which transmits information within the cell to regulate cellular activity via defined signaling pathways by generating second messengers or functioning as effectors by responding to such messengers.

The term “signaling module” refers to a molecule or molecular complex comprising one or more signaling mediators or signaling adaptors that is capable of initiating a cell signal.

The term “signaling mediator” or “signaling adaptor” refers to molecule that is capable of initiating or inhibiting a cell signal when recruited by a natural or a non-natural signaling receptor.

The term “signaling chain” or “signaling fragment” refers to a polypeptide comprising the transmembrane and/or intracellular region and optionally the extracellular hinge/connecting peptide regions of a cell signaling receptor. Example signaling chains include the constant chains of TCRα, TCRβ, TCRγ and TCRδ. Additional example signaling chains include chains comprising the transmembrane and/or intracellular regions of CD16, NKp30, NKp44, NKp46, DAP10, DAP12, DNAM-1, NKG2D, CD32, CD64, KIR3DL1, KIR2DS4, FcRγ and CD3z.

The term SVH domain as used herein refers to a single human V_{H} domain antibody (V_{H} sdAb). These terms are thus used interchangeably. The term SVH is also used interchangeably with independent vH domains.

The term “subject” is intended to include living organisms in which an immune response can be elicited (e.g., any domesticated mammals or a human).

As used herein, “tag cassette”, “tag”, “epitope tag”, “mAb specific-epitope”, “SHARP-tag”. or “SHARP-tag” refers to a unique peptide sequence affixed to, fused to, or that is part of a protein of interest, to which a heterologous or non-endogenous cognate binding molecule (e.g., receptor, ligand, antibody, or other binding partner) is capable of specifically binding where the binding property can be used to detect, identify, isolate or purify, track, enrich for, or target a tagged protein or cells expressing a tagged protein, particularly when a tagged protein is part of a heterogeneous population of proteins or other material, or when cells expressing a tagged protein are part of a heterogeneous population of cells (e.g., a biological sample like peripheral blood). In certain embodiments, a cell expressing a tagged protein can be contacted with a heterologous or non-endogenous cognate binding molecule and induce a biological response, such as promote cell activation, cell proliferation or cell death. In the provided fusion proteins, the ability of the tag cassette(s) to be specifically bound by the cognate binding molecule(s) is distinct from or in addition to the ability of the binding domain(s) to specifically bind to the target molecule(s). The tag cassette generally is not an antigen-binding molecule, for example, is not an antibody or TCR or an antigen-binding portion thereof. Examples of tags (e.g., SHARP-tags) are provided in SEQ ID NO: 1-123, 150-167, 251-440, 550-579, 631-650, 651-654, 656, 674-676, 686, 689, and 692-720. Tags may be present in multiple copies in fusion proteins of this disclosure. For example, a fusion protein of this disclosure can have one, two, three, four or five tag cassettes. In certain embodiments, a connector region of a TAG-SAR includes one tag cassette, two tag cassettes, three tag cassettes, four tag cassettes, or five tag cassettes. Each of the plurality of tag cassettes may be the same or different composition (e.g., P8, P9, P10, P11 etc.). Each of the plurality of tag cassettes may be recognized by the same or different binding agents (e.g., monoclonal antibody). For example, a TAG-SAR may comprise P11 (SEQ ID NO: 674), P13 (SEQ ID NO: 675) or P16 (SEQ ID NO: 675) tags that are all recognized by Polatuzumab vedotin, SN8 or 2F2 antibodies. Alternatively, a TAG-SAR may comprise a P11 tag (SEQ ID NO: 674) and a Her2 tag (SEQ ID NO: 557) that are recognized by Polatuzumab and H2Mab-250 antibodies, respectively. Alternatively, a TAG-SAR will have multiple tag cassettes of the same type or same amino acid sequence, such as two, three, four or five tag cassettes (e.g., P8, P9, P10, P11 etc.).

Unless otherwise indicated, “SHARP-tag” encompasses the sequences represented by SEQ ID NO: 1-123, 150-167, 251-440, 550-579, 631-650, 651-654, 656, 674-676, 686, 689, and 692-720 as well as variants that maintain function (i.e., recognition by SABR). Such variants may include insertions, deletions, or substitutions of one or more residues (for example, up to 1-3 conservative substitutions) or longer versions (e.g., 9-15 amino acids) that retain the core epitope conformation. A SHARP-tag that is recognized by Polatuzumab is referred as a “Pola-tag” or “Pola tag”. The P8, P9, P10, P11, P12, P13 tags refer to SEQ ID NO:47-55, respectively. A SHARP-tag that is recognized by H2Mab-250 is referred as a “Her2-tag” or “H-tag”. The examples of Her2 or H-tags include SEQ ID NO:557-566 and functional variants that specifically bind to H2Mab-250.

A SHARP-tagged polypeptide, such as a TAG-SAR, may have two different epitope tags or tag cassettes. In some embodiments, a first tag cassette can provide a stimulation signal and a distinct second tag cassette might be used to associate with a detection reagent or associate with an antibody-toxin conjugate or with an antibody-imaging agent conjugate. In further embodiments, the two or more first tag cassettes may be located in different areas of a TAG-SAR. In certain embodiments, a first tag cassette in the connector region and a second tag cassette is located at the amino-terminus or carboxy terminus or both of a TAG-SAR.

In the fusion protein of the invention, the peptide, i.e. the epitope tag, may be located at any position of the fusion protein. The peptide may be fused to the N-terminus or the C-terminus of the polypeptide. Alternatively, the peptide may be fused to the polypeptide at a position between the N-terminus and the C-terminus of the polypeptide. As an illustrative example, the peptide may be fused in between two domains of the polypeptide.

The polypeptide comprised in the fusion protein may have a stable fold that is independent from the presence or absence of the peptide. This means that the peptide preferably does not alter or interfere with the native structure of the polypeptide.

In the fusion protein of the invention, the peptide may be fused to the polypeptide either by direct fusion or through a linker. A “linker” as used herein joins together two or more subunits of a fusion protein as described herein. The linkage can be covalent. A preferred covalent linkage is via a peptide bond, such as a peptide bond between amino acids. A preferred linker is a peptide linker. Said linker preferably comprises one or more amino acids, such as 1-20 or more amino acids. Preferred peptide linkers include glycine-serine (GS) linkers, glycosylated GS linkers, and proline-alanine-serine polymer (PAS) linkers.

The polypeptide comprised in the fusion protein of the invention may comprise at least one protein domain. A “protein domain” as used herein refers to a part of a given protein sequence and (tertiary) structure that can function and/or exist independently of the rest of the protein chain. The protein domain preferably forms a compact three-dimensional structure and often can be independently stable and folded. A protein domain may further form a functional unit. A preferred location for the peptide may be outside of the protein domain. This can be N-terminal or C-terminal of the at least one protein domain of the polypeptide, or in between two protein domains of the polypeptide. The polypeptide comprised in the fusion protein of the invention may be a globular protein, and membrane protein, a fibrous protein, or a natively unfolded protein, or a subunit or domain of the globular protein, membrane protein, fibrous protein, or natively unfolded protein.

This sequence defines the core structure of the peptide and may further comprise up to two additional amino acids at the N terminus and up to two additional amino acids at the C terminus. Such additional amino acids at the ends of the core structure of the peptide usually do not necessarily influence the secondary structure of the of the peptide or specific binding of the peptide to an antibody specific for the peptide but may serve as linker structures in the fusion protein. Accordingly, type and number of the additional amino acids may depend on the location of the peptide in the fusion protein and may vary depending on whether the peptide is located N-terminal or C-terminal or somewhere in between of the polypeptide.

In certain embodiments, a tag is located within a connector region of a fusion protein of this disclosure. For example, a connector region may further comprise a linker module adjacent to a tag cassette, wherein the linker module comprises a flexible linker. Exemplary flexible linkers are provided in SEQ ID NO: 1330-1331 and 1453. Additional flexible linkers are known in the art.

As used herein, the term “TCR” or “T cell receptor” refers to a dimeric heterologous cell surface signaling protein forming an alpha-beta or gamma-delta receptor typically involved in recognizing an antigen presented by an MHC molecule.

As used herein, the term “TCR constant chain” refers to the constant chain TCRα, TCRβ1, TCRβ2, TCRγ, TCRS and pre-TCRα and functional variants, mutants, alternative spliced isoforms, and homologs from non-human species.

As used herein, the term “T lymphocyte” or “T cell” refers to a cell expressing CD3 (CD3+) and a T Cell Receptor (TCR+). The term “non-T cell” refers to a cell that is not a T cell. In an embodiment, a non-T cell lacks the cell surface expression of CD3 and a T cell receptor.

The term “T cell receptor module,” or “TCRM,” refers to a heterodimer comprising sequences derived from a T cell receptor. The TCRM comprises T cell receptor transmembrane domains and may further comprise all or a portion of T cell receptor connecting peptides and/or intracellular domains.

The term “canonical TCRM” refers to a TCRM that is formed by heterodimerization between canonical TCR chains, i.e., TCRα and TCRβ1 or TCRβ2 chains, TCRγ and TCRδ chains, and pre-TCRα and TCRβ1 or P2 chains. Further, a canonical TCRM refers to a TCRM that is formed between two TCR chains that belong to the same species (e.g., human, mouse etc.).

The term “non-canonical TCRM” refers to a TCRM that is not formed by the heterodimerization between canonical TCR chains, i.e., TCRα and TCRβ1 or TCRβ2 chains, TCRγ and TCRδ chains, and pre-TCRα and TCRβ1 or P2 chains. A non-canonical TCRM can be formed by heterodimerization of TCRα and TCRγ chains, TCRβ and TCRδ chains. A non-canonical TCRM is also formed between variants of TCRα, β, γ and δ chains, including their deletion mutants and hybrid chains.

The term “interspecies non-canonical TCRM” refers to a TCRM that is formed by heterodimerization of two TCR chains that belong to different species (e.g., between human TCRα and mouse TCRβ chain).

The term “Fv” as used here refers to an antigen binding module that is formed by the variable domains of an antibody. A Fv can be formed by the vL and vH domains.

As used herein a “transmembrane module” or “TMM” refers to a molecule or a molecular complex comprising a transmembrane protein (e.g., TCRα, TCRβ, CD16A or CD3z).

The term “membrane associated module” or “MAM” refers to a molecule or a molecular complex comprising a transmembrane protein (e.g., CD16A, CD3ζ) or a membrane anchored protein (e.g., CD16B). The term encompasses transmembrane proteins, such as CD16A, CD3z (or CD3ζ) and GPI-linked proteins, such as CD16B. A MAM may further comprise all or portions of hinge domains and/or cytosolic domains.

The term “TAG-SAR” refers to a SAR comprising at least (i.e., one or more) one tag cassette, e.g., a tag described herein, e.g., SEQ ID NO: 1-123, 150-167, 251-440, 550-579, 631-650, 651-654, 656, 674-676, 686, 689, and 692-720. The tag can be present at any location in a TAG-SAR. The TAG-SAR may comprise one or more than one copy of the tag. The SAR may be a single chain SAR (e.g., a second-generation CAR) or a double chain SAR (e.g., SIR, cTCR, Ab-TCR, STAR, zSIR, z16-SAR, TCR, uTCR-SAR etc.).

The term “TAG-cell” refers to a cell that expresses a recombinant polypeptide (e.g., a TAG-polypeptide) comprising at least one SHARP-tag or a tag cassette, e.g., a tag described herein, e.g., SEQ ID NO: 1-123, 150-167, 251-440, 550-579, 631-650, 651-654, 656, 674-676, 686, 689, and 692-720. In an embodiment, the recombinant polypeptide comprising a tag cassette can encode for CD79b, including its isoforms. In another embodiment, the recombinant polypeptide comprising a tag cassette can encode for a protein other than CD79b (e.g., a SAR, a multipurpose switch, a cytokine etc.). A TAG-cell can be derived from any cell, e.g., immune effector cell, hematopoietic stem cell, lung cell, breast cell, skin cell, brain cell, muscle cell, iPSC, embryonic stem cell etc. In an embodiment, the TAG-cell is an immune effector cell, e.g., a T cell, NK cell, NKT, macrophage, monocyte, B cell etc. In an embodiment, the TAG-cell is not a B cell. In an embodiment, a TAG-cell is used for the purpose of cell therapy.

The term “TAG-polypeptide” refers to a recombinant polypeptide comprising at least one SHARP-tag or tag cassette, e.g., a tag described herein, e.g., SEQ ID NO: 1-123, 150-167, 251-440, 550-579, 631-650, 651-654, 656, 674-676, 686, 689, and 692-720.

The term “TAG-polynucleotide” refers to a recombinant polynucleotide that encodes for at least one SHARP-tag or tag cassette, e.g., a tag described herein, e.g., SEQ ID NO: 1-123, 150-167, 251-440, 550-579, 631-650, 651-654, 656, 674-676, 686, 689, and 692-720. In an embodiment, the recombinant polynucleotide comprising a tag cassette can encode for CD79b, including its isoforms. In another embodiment, the recombinant polynucleotide comprising a tag cassette can encode for a protein other than CD79b (e.g., a SAR, a multipurpose switch, a cytokine etc.). Examples of TAG-polynucleotides are presented in SEQ ID NO: 3105-3123, 3129-3135, 3137-3138, 3140-41, 4757 and 5075-5457. In an embodiment, a TAG-polynucleotide is ectopically introduced in a cell for the purpose of cell therapy. [00561]“Therapeutic agents” as used herein refers to agents that are used to, for example, treat, inhibit, prevent, mitigate the effects of, reduce the severity of, reduce the likelihood of developing, slow the progression of and/or cure, a disease. “Therapeutic Controls” as used herein refers to an element used for controlling the activity of a SAR expressing cell. Examples of therapeutic controls are provided in Table 24 of the provisional application. The term “therapeutic effect” refers to a biological effect which can be manifested by various means, including but not limited to, e.g., decrease in tumor volume, a decrease in the number of cancer cells etc. The phrase “therapeutically effective amount” as used herein means a sufficient amount of the composition to treat a disorder, at a reasonable benefit/risk ratio applicable to any medical treatment.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic compounds, plasmids, and viruses. [00563]“Transmembrane domain” (or TM domain) as used herein refers to the region of a receptor, (e.g., a SAR) which crosses the plasma membrane.

The term “variant” or “functional variant” or “biological variant” refers to a molecule (e.g., DNA, RNA, or protein) that is derived from a reference sequence through one or more alterations, such as substitutions, deletions, insertions, or combinations thereof, and retains at least one biological function or binding specificity of the reference molecule. Unless specified otherwise, the term variant refers to a functional variant or a biological variant. [00565]“Vector,” “cloning vector” and “expression vector” as used herein refer to the vehicle by which a polynucleotide sequence (e.g., a foreign gene) can be introduced into a host cell, so as to transform the host and promote expression (e.g., transcription and translation) of the introduced sequence. Vectors include plasmids, phages, viruses, etc.

The term “viral vector” refers to a vector obtained or derived from a virus.

The term “zeta” or alternatively “zeta chain”, “CD3-zeta” or “TCR-zeta” “CD3ζ” is defined as the protein provided as GenBank Ace. No. BAG36664.1, or the equivalent residues from a non-human species, e.g., mouse, rodent, monkey, ape and the like.

### Embodiments

EMBODIMENT 1. A fusion protein comprising: (a) a peptide tag that is at least 60% identical in composition to a peptide present in the extracellular region of an endogenous protein, wherein the peptide tag has the following characteristics:
a. is between 5-50 amino acids in length;
b. lacks a disulfide bond;
c. is hydrophilic;
   (b) and a polypeptide.
   EMBODIMENT 2. The fusion protein of embodiment 1, wherein the peptide tag has one or more of features selected from the group consisting of:
a) is hypoimmunogenic, nonimmunogenic or minimally immunogenic;
b) has more than 60% sequence identity to a peptide located in the N-terminal, C-terminal, the juxta membrane region, hinge and/or stalk regions of the extracellular region of an endogenous protein;
c) has more than 60% sequence identity to a peptide located in the unfolded region of an endogenous protein;
d) has more than 60% sequence identity to a peptide located in the random coil or unstructured region of an endogenous protein;
e) lacks a cysteine residue
f) lacks an Asn-X-Ser/Thr motif, wherein X can be any amino acid residue;
g) lacks an N-linked glycosylation site;
h) has an α-helical secondary structure;
i) is recognized by a drug that is approved by a regulatory agency;
j) is recognized by a drug that is approved by a regulatory agency for in vivo administration to a subject, optionally wherein the subject is a human subject;
k) has less than 50% sequence identity to a region of an endogenous protein that is a mutational hot-spot and/or is associated with a congenital or acquired disease;
l) is not an autoantigen;
m) has less than 50% sequence identity to a peptide present in the human La protein;
n) is not a nuclear antigen;
o) comprises a linear epitope; and/or
p) does not comprise, consist of, or contain any sequence represented by SEQ ID NOs: 450-455 or a variant with at least 80% sequence identity thereto.
   EMBODIMENT 3. The fusion protein of embodiment 1, wherein the peptide tag comprises a sequence represented by SEQ ID NO: 1-123, 150-167, 251-440, 550-579, 631-650, 651-654, 656, 674-676, 686, 689, and 692-720, or functional variants thereof.
   EMBODIMENT 4. The fusion protein of embodiment 1, wherein the peptide tag comprises or consists of a sequence selected from the group consisting of one or more of the following:
a) X₁X₂EX₃X₄X₅X₆ wherein X₁ is selected form R, E, or H; X₂ is selected form S, T or A; X₃ is selected form D, S, N, G, A, T, K, L; X₄ is selected form R or H; X₅ is selected form Y or M; and X₆ is any amino acid or any naturally occurring amino acid;
b) X₁X₂EX₃X₄X₅X₆ wherein X₁ is selected form R, E, or H; X₂ is selected form S, T or A; X₃ is selected form D, S, N, G, A, T, K, L; X₄ is selected form R or H; X₅ is selected form Y or M; and X₆ is selected form R, K, E, D, G, H, L, M, Q, V, W, S, F, I, Y, P, N, T or A;
c) X₁X₂EX₃X₄X₅X₆ wherein X₁ is selected from R, E, S, N, D, F or H; X₂ is S, T, I, L, M, V, F, H, G, or A; X₃ is D, S, N, G, A, T, K, Q, L, Y, W, R, or V; X₄ is R, H, P, L, T or C; X₅ is Y, M, F, H, D, A, G, I or V; and X₆ is any amino acid or any naturally occurring amino acid;
d) X₁X₂EX₃X₄X₅X₆ wherein X₁ is selected from R, E, S, N, D, F or H; X₂ is selected from S, T, I, L, M, V, F, H, G, or A; X₃ is selected from D, S, N, G, A, T, K, Q, L, Y, W, R, or V; X₄ is R, H, P, L, T or C; X₅ is selected from Y, M, F, H, D, A, G, I or V; and X₆ is selected from R, K, E, D, G, H, L, M, Q, V, W, S, F, I, Y, P, N, T or A;
e) X₁X₂EX₃X₄X₅X₆ wherein X₁ is selected from K or R; X₂ is selected from S, T, I, L, M, V, F, H, G, or A; X₃ is selected from D, S, N, G, A, T, K, Q, L, Y, W, R, or V; X₄ is selected from L, R, H, P, T or C; X₅ is selected from Y, M, F, H, D, A, G, I or V; and X₆ is any amino acid or any naturally occurring amino acid;
f) X₁EX₂ X₃X₄X₅X₆ wherein X₁ is selected from S, T, I, L, M, V, F, H, G, or A; X₂ is selected from D, S, N, G, A, T, K, Q, L, Y, W, R, or V; X₃ is selected from L, R, H, P, T or C; X₄ is selected from Y, M, F, H, D, A, G, I or V; X₅ is P; and X₆ is any amino acid or any naturally occurring amino acid; and/or
g) X₁X₂X₃EX₄X₅X₆ wherein X₁ is selected from A, G or S; X₂ is selected from K or R; X₃ is selected from S, T, I, L, M, V, F, H, G, or A; X₄ is selected from D, S, N, G, A, T, K, Q, L, Y, W, R, or V; X₅ is selected from L, R, H, P, T or C; and X₆ is selected from Y, M, F, H, D, A, G, I or V.
   EMBODIMENT 5. The fusion protein of embodiment 1, wherein the peptide specifically binds to an antibody, an antibody fragment or an antibody conjugate, wherein the antibody, the antibody fragment or the antibody conjugate comprises
a) a variable light chain (vL) region represented by SEQ ID NO: 774-794, 808-818 and 2173-2178, and 2183 or variants thereof with up to 20 amino acid substitutions in the framework regions and a complementary variable heavy chain (vH) region represented by SEQ ID NO: 899-919, 937-941, 2179-2182 and 2184 or variants thereof with up to 20 amino acid substitutions in the framework regions; or
b) a variable light chain (vL) region represented by SEQ ID NO: 783-789 and 791 or variants thereof with up to 20 amino acid substitutions in the framework regions and a complementary variable heavy chain (vH) region represented by SEQ ID NO: 908-914 and 916 or variants thereof with up to 20 amino acid substitutions in the framework regions; or
c) a variable light chain (vL) region represented by SEQ ID NO: 797 or variants thereof with up to 20 amino acid substitutions in the framework regions and a complementary variable heavy chain (vH) region represented by SEQ ID NO: 922 or variants thereof with up to 20 amino acid substitutions in the framework regions; or
d) a variable light chain (vL) region represented by SEQ ID NO: 846-847 or variants thereof with up to 20 amino acid substitutions in the framework regions and a complementary variable heavy chain (vH) region represented by SEQ ID NO: 966-970 or variants thereof with up to 20 amino acid substitutions in the framework regions;
f) a variable light chain (vL) region comprising the light chain CDR1, CDR2 and CDR3 represented by SEQ ID NO: 6653-6673, 7022-7042, 7391-7411, respectively, and a complementary variable heavy chain (vH) region comprising the heavy chain CDR1, CDR2 and CDR3 represented by SEQ ID NO: 6778-6798, 7147-7167, and 7516-7536, respectively.
   EMBODIMENT 6. The antibody, the antibody fragment or the antibody conjugate of embodiment 5, wherein the antibody, the antibody fragment or the antibody conjugate has one or more features selected from the group consisting of:
a) is approved by the US Food and Drug administration (FDA), optionally wherein the antibody, the antibody fragment or the antibody conjugate is approved for in vivo administration to a human subject;
b) the antibody fragment or the antibody conjugate is chimeric, partially humanized, or fully human;
c) is an antibody drug conjugate or a radiolabeled antibody;
d) is Polatuzumab vedotin, 2F2, SN8, 10D10 or H2Mab-250 or a variant thereof, optionally wherein in the variant is a generic variant.
   EMBODIMENT 7. A fusion protein comprising a peptide tag that the antibody of any one of embodiment 5 binds to
   EMBODIMENT 8. A nucleic acid encoding a fusion protein of embodiment 1.
   EMBODIMENT 0.9. A vector comprising a nucleic acid of embodiment 8.
   EMBODIMENT 10. A host cell comprising the nucleic acid of embodiment 8 or the vector of embodiment 9 or expressing the fusion protein of embodiment 1 or the antibody of any one of embodiment 5.
   EMBODIMENT 11. Use of a peptide tag as defined in embodiment 1 as an epitope tag or for the detection, immobilization, isolation, regulation, control or purification of the fusion protein of embodiment 1 or for the detection, immobilization, isolation, control, depletion, elimination or purification of a host cell of embodiment 10.
   EMBODIMENT 12. A method of detecting, isolating, regulating, controlling or purifying the fusion protein of embodiment 1 or the host cell of embodiment 11, comprising contacting the fusion protein with an antibody of embodiment 5.
   EMBODIMENT 13. A method of enrichment, selection or depletion of a host cell of embodiment 11 by use of an antibody that recognizes the peptide tag of embodiment 1, wherein optionally the antibody is Polatuzumab vedotin.
   EMBODIMENT 14. A kit comprising a nucleic acid or a nucleic acid expression construct encoding a peptide as defined in embodiment 1, or a vector of embodiment 9 and optionally an antibody of embodiment 5.

The foregoing detailed description and examples are intended to illustrate the breadth and versatility of the invention, not to limit it. It will be evident to those skilled in the art that various modifications and adaptations can be made without departing from the scope of the invention as defined in the claims below.

What is claimed is:

1. A fusion protein comprising:
(a) a peptide tag that is at least 60% identical in composition to a peptide present in the extracellular region of an endogenous protein, wherein the peptide tag has the following characteristics:
a. is between 5-50 amino acids in length;
b. lacks a disulfide bond;
c. is hydrophilic;
(b) and a polypeptide.

2. The fusion protein of claim 1, wherein the peptide tag has one or more of features selected from the group consisting of:
a) is hypoimmunogenic, nonimmunogenic or minimally immunogenic;
b) has more than 60% sequence identity to a peptide located in the N-terminal, C-terminal, the juxta membrane region, hinge and/or stalk regions of the extracellular region of an endogenous protein;
c) has more than 60% sequence identity to a peptide located in the unfolded region of an endogenous protein;
d) has more than 60% sequence identity to a peptide located in the random coil or unstructured region of an endogenous protein;
e) lacks a cysteine residue
f) lacks an Asn-X-Ser/Thr motif, wherein X can be any amino acid residue;
g) lacks an N-linked glycosylation site;
h) has an α-helical secondary structure;
i) is recognized by a drug that is approved by a regulatory agency;
j) is recognized by a drug that is approved by a regulatory agency for in vivo administration to a subject, optionally wherein the subject is a human subject;
k) has less than 50% sequence identity to a region of an endogenous protein that is a mutational hot-spot and/or is associated with a congenital or acquired disease;
l) is not an autoantigen;
m) has less than 50% sequence identity to a peptide present in the human La protein;
n) is not a nuclear antigen;
o) comprises a linear epitope; and/or
p) does not comprise, consist of, or contain any sequence represented by SEQ ID NOs: 450-455 or a variant with at least 80% sequence identity thereto.

3. The fusion protein of claim 1, wherein the peptide tag comprises a sequence represented by SEQ ID NO: 1-123, 150-167, 251-440, 550-579, 631-650, 651-654, 656, 674-676, 686, 689, and 692-720, or functional variants thereof.

4. The fusion protein of claim 1, wherein the peptide tag comprises or consists of a sequence selected from the group consisting of one or more of the following:
a) X₁X₂EX₃X₄X₅X₆ wherein X₁ is selected form R, E, or H; X₂ is selected form S, T or A; X₃ is selected form D, S, N, G, A, T, K, L; X₄ is selected form R or H; X₅ is selected form Y or M; and X₆ is any amino acid or any naturally occurring amino acid;
b) X₁X₂EX₃X₄X₅X₆ wherein X₁ is selected form R, E, or H; X₂ is selected form S, T or A; X₃ is selected form D, S, N, G, A, T, K, L; X₄ is selected form R or H; X₅ is selected form Y or M; and X₆ is selected form R, K, E, D, G, H, L, M, Q, V, W, S, F, I, Y, P, N, T or A;
c) X₁X₂EX₃X₄X₅X₆ wherein X₁ is selected from R, E, S, N, D, F or H; X₂ is S, T, I, L, M, V, F, H, G, or A; X₃ is D, S, N, G, A, T, K, Q, L, Y, W, R, or V; X₄ is R, H, P, L, T or C; X₅ is Y, M, F, H, D, A, G, I or V; and X₆ is any amino acid or any naturally occurring amino acid;
d) X₁X₂EX₃X₄X₅X₆ wherein X₁ is selected from R, E, S, N, D, F or H; X₂ is selected from S, T, I, L, M, V, F, H, G, or A; X₃ is selected from D, S, N, G, A, T, K, Q, L, Y, W, R, or V; X₄ is R, H, P, L, T or C; X₅ is selected from Y, M, F, H, D, A, G, I or V; and X₆ is selected from R, K, E, D, G, H, L, M, Q, V, W, S, F, I, Y, P, N, T or A;
e) X₁X₂EX₃X₄X₅X₆ wherein X₁ is selected from K or R; X₂ is selected from S, T, I, L, M, V, F, H, G, or A; X₃ is selected from D, S, N, G, A, T, K, Q, L, Y, W, R, or V; X₄ is selected from L, R, H, P, T or C; X₅ is selected from Y, M, F, H, D, A, G, I or V; and X₆ is any amino acid or any naturally occurring amino acid;
f) X₁EX₂ X₃X₄X₅X₆ wherein X₁ is selected from S, T, I, L, M, V, F, H, G, or A; X₂ is selected from D, S, N, G, A, T, K, Q, L, Y, W, R, or V; X₃ is selected from L, R, H, P, T or C; X₄ is selected from Y, M, F, H, D, A, G, I or V; X₅ is P; and X₆ is any amino acid or any naturally occurring amino acid; and/or
g) X₁X₂X₃EX₄X₅X₆ wherein X₁ is selected from A, G or S; X₂ is selected from K or R; X₃ is selected from S, T, I, L, M, V, F, H, G, or A; X₄ is selected from D, S, N, G, A, T, K, Q, L, Y, W, R, or V; X₅ is selected from L, R, H, P, T or C; and X₆ is selected from Y, M, F, H, D, A, G, I or V.

5. The fusion protein of claim 1, wherein the peptide specifically binds to an antibody, an antibody fragment or an antibody conjugate, wherein the antibody, the antibody fragment or the antibody conjugate comprises
a) a variable light chain (vL) region represented by SEQ ID NO: 774-794, 808-818 and 2173-2178, and 2183 or variants thereof with up to 20 amino acid substitutions in the framework regions and a complementary variable heavy chain (vH) region represented by SEQ ID NO: 899-919, 937-941, 2179-2182 and 2184 or variants thereof with up to 20 amino acid substitutions in the framework regions; or
b) a variable light chain (vL) region represented by SEQ ID NO: 783-789 and 791 or variants thereof with up to 20 amino acid substitutions in the framework regions and a complementary variable heavy chain (vH) region represented by SEQ ID NO: 908-914 and 916 or variants thereof with up to 20 amino acid substitutions in the framework regions; or
c) a variable light chain (vL) region represented by SEQ ID NO: 797 or variants thereof with up to 20 amino acid substitutions in the framework regions and a complementary variable heavy chain (vH) region represented by SEQ ID NO: 922 or variants thereof with up to 20 amino acid substitutions in the framework regions; or
d) a variable light chain (vL) region represented by SEQ ID NO: 846-847 or variants thereof with up to 20 amino acid substitutions in the framework regions and a complementary variable heavy chain (vH) region represented by SEQ ID NO: 966-970 or variants thereof with up to 20 amino acid substitutions in the framework regions;
f) a variable light chain (vL) region comprising the light chain CDR1, CDR2 and CDR3 represented by SEQ ID NO: 6653-6673, 7022-7042, 7391-7411, respectively, and a complementary variable heavy chain (vH) region comprising the heavy chain CDR1, CDR2 and CDR3 represented by SEQ ID NO: 6778-6798, 7147-7167, and 7516-7536, respectively.

6. The antibody, the antibody fragment or the antibody conjugate of claim 5, wherein the antibody, the antibody fragment or the antibody conjugate has one or more features selected from the group consisting of:
a) is approved by the US Food and Drug administration (FDA), optionally wherein the antibody, the antibody fragment or the antibody conjugate is approved for in vivo administration to a human subject;
b) the antibody fragment or the antibody conjugate is chimeric, partially humanized, or fully human;
c) is an antibody drug conjugate or a radiolabeled antibody;
d) is Polatuzumab vedotin, 2F2, SN8, 10D10 or H2Mab-250 or a variant thereof, optionally wherein in the variant is a generic variant.

7. A fusion protein comprising a peptide tag that the antibody of any one of claim 5 binds to.

8. A nucleic acid encoding a fusion protein of claim 1.

9. A vector comprising a nucleic acid of claim 8.

10. A host cell comprising the nucleic acid of claim 8 or the vector of claim 9 or expressing the fusion protein of claim 1 or the antibody of any one of claim 5.

11. Use of a peptide tag as defined in claim 1 as an epitope tag or for the detection, immobilization, isolation, regulation, control or purification of the fusion protein of claim 1 or for the detection, immobilization, isolation, control, depletion, elimination or purification of a host cell of claim 10.

12. A method of detecting, isolating, regulating, controlling or purifying the fusion protein of claim 1 or the host cell of claim 11, comprising contacting the fusion protein with an antibody of claim 5.

13. A method of enrichment, selection or depletion of a host cell of claim 11 by use of an antibody that recognizes the peptide tag of claim 1, wherein optionally the antibody is Polatuzumab vedotin, 2F2 or H2Mab-250.

14. A kit comprising a nucleic acid or a nucleic acid expression construct encoding a peptide as defined in claim 1, or a vector of claim 9 and optionally an antibody of claim 5.

15. An isolated peptide tag that is at least 60% identical in composition to a peptide present in the extracellular region of an endogenous protein, wherein the peptide tag has the following characteristics:
a. is between 5-50 amino acids in length;
b. lacks a disulfide bond; and
c. is hydrophilic.

16. The peptide tag of claim 15, wherein the peptide tag has one or more of features selected from the group consisting of:
a) is hypoimmunogenic, nonimmunogenic or minimally immunogenic;
b) has more than 60% sequence identity to a peptide located in the N-terminal, C-terminal, the juxta membrane region, hinge and/or stalk regions of the extracellular region of an endogenous protein;
c) has more than 60% sequence identity to a peptide located in the unfolded region of an endogenous protein;
d) has more than 60% sequence identity to a peptide located in the random coil or unstructured region of an endogenous protein;
e) lacks a cysteine residue
f) lacks an Asn-X-Ser/Thr motif, wherein X can be any amino acid residue;
g) lacks an N-linked glycosylation site;
h) has an α-helical secondary structure;
i) is recognized by a drug that is approved by a regulatory agency;
j) is recognized by a drug that is approved by a regulatory agency for in vivo administration to a subject, optionally wherein the subject is a human subject;
k) has less than 50% sequence identity to a region of an endogenous protein that is a mutational hot-spot and/or is associated with a congenital or acquired disease;
l) is not an autoantigen;
m) has less than 50% sequence identity to a peptide present in the human La protein;
n) is not a nuclear antigen;
o) comprises a linear epitope; and/or
p) does not comprise, consist of, or contain any sequence represented by SEQ ID NOs: 450-455 or a variant with at least 80% sequence identity thereto.

17. The peptide tag of claim 1, wherein the peptide tag comprises a sequence represented by SEQ ID NO: 1-123, 150-167, 251-440, 550-579, 631-650, 651-654, 656, 674-676, 686, 689, and 692-720, or functional variants thereof.

18. The peptide tag of claim 1, wherein the peptide tag comprises or consists of a sequence selected from the group consisting of one or more of the following:
a) X₁X₂EX₃X₄X₅X₆ wherein X₁ is selected form R, E, or H; X₂ is selected form S, T or A; X₃ is selected form D, S, N, G, A, T, K, L; X₄ is selected form R or H; X₅ is selected form Y or M; and X₆ is any amino acid or any naturally occurring amino acid;
b) X₁X₂EX₃X₄X₅X₆ wherein X₁ is selected form R, E, or H; X₂ is selected form S, T or A; X₃ is selected form D, S, N, G, A, T, K, L; X₄ is selected form R or H; X₅ is selected form Y or M; and X₆ is selected form R, K, E, D, G, H, L, M, Q, V, W, S, F, I, Y, P, N, T or A;
c) X₁X₂EX₃X₄X₅X₆ wherein X₁ is selected from R, E, S, N, D, F or H; X₂ is S, T, I, L, M, V, F, H, G, or A; X₃ is D, S, N, G, A, T, K, Q, L, Y, W, R, or V; X₄ is R, H, P, L, T or C; X₅ is Y, M, F, H, D, A, G, I or V; and X₆ is any amino acid or any naturally occurring amino acid;
d) X₁X₂EX₃X₄X₅X₆ wherein X₁ is selected from R, E, S, N, D, F or H; X₂ is selected from S, T, I, L, M, V, F, H, G, or A; X₃ is selected from D, S, N, G, A, T, K, Q, L, Y, W, R, or V; X₄ is R, H, P, L, T or C; X₅ is selected from Y, M, F, H, D, A, G, I or V; and X₆ is selected from R, K, E, D, G, H, L, M, Q, V, W, S, F, I, Y, P, N, T or A;
e) X₁X₂EX₃X₄X₅X₆ wherein X₁ is selected from K or R; X₂ is selected from S, T, I, L, M, V, F, H, G, or A; X₃ is selected from D, S, N, G, A, T, K, Q, L, Y, W, R, or V; X₄ is selected from L, R, H, P, T or C; X₅ is selected from Y, M, F, H, D, A, G, I or V; and X₆ is any amino acid or any naturally occurring amino acid;
f) X₁EX₂ X₃X₄X₅X₆ wherein X₁ is selected from S, T, I, L, M, V, F, H, G, or A; X₂ is selected from D, S, N, G, A, T, K, Q, L, Y, W, R, or V; X₃ is selected from L, R, H, P, T or C; X₄ is selected from Y, M, F, H, D, A, G, I or V; X₅ is P; and X₆ is any amino acid or any naturally occurring amino acid; and/or
g) X₁X₂X₃EX₄X₅X₆ wherein X₁ is selected from A, G or S; X₂ is selected from K or R; X₃ is selected from S, T, I, L, M, V, F, H, G, or A; X₄ is selected from D, S, N, G, A, T, K, Q, L, Y, W, R, or V; X₅ is selected from L, R, H, P, T or C; and X₆ is selected from Y, M, F, H, D, A, G, I or V.

19. The peptide tag of claim 1, wherein the peptide specifically binds to an antibody, an antibody fragment or an antibody conjugate, wherein the antibody, the antibody fragment or the antibody conjugate comprises
a) a variable light chain (vL) region represented by SEQ ID NO: 774-794, 808-818 and 2173-2178, and 2183 or variants thereof with up to 20 amino acid substitutions in the framework regions and a complementary variable heavy chain (vH) region represented by SEQ ID NO: 899-919, 937-941, 2179-2182 and 2184 or variants thereof with up to 20 amino acid substitutions in the framework regions; or
b) a variable light chain (vL) region represented by SEQ ID NO: 783-789 and 791 or variants thereof with up to 20 amino acid substitutions in the framework regions and a complementary variable heavy chain (vH) region represented by SEQ ID NO: 908-914 and 916 or variants thereof with up to 20 amino acid substitutions in the framework regions; or
c) a variable light chain (vL) region represented by SEQ ID NO: 797 or variants thereof with up to 20 amino acid substitutions in the framework regions and a complementary variable heavy chain (vH) region represented by SEQ ID NO: 922 or variants thereof with up to 20 amino acid substitutions in the framework regions; or
d) a variable light chain (vL) region represented by SEQ ID NO: 846-847 or variants thereof with up to 20 amino acid substitutions in the framework regions and a complementary variable heavy chain (vH) region represented by SEQ ID NO: 966-970 or variants thereof with up to 20 amino acid substitutions in the framework regions;
f) a variable light chain (vL) region comprising the light chain CDR1, CDR2 and CDR3 represented by SEQ ID NO: 6653-6673, 7022-7042, 7391-7411, respectively, and a complementary variable heavy chain (vH) region comprising the heavy chain CDR1, CDR2 and CDR3 represented by SEQ ID NO: 6778-6798, 7147-7167, and 7516-7536, respectively.

20. The antibody, the antibody fragment or the antibody conjugate of claim 19 wherein the antibody, the antibody fragment or the antibody conjugate has one or more features selected from the group consisting of:
a) is approved by the US Food and Drug administration (FDA), optionally wherein the antibody, the antibody fragment or the antibody conjugate is approved for in vivo administration to a human subject;
b) the antibody fragment or the antibody conjugate is chimeric, partially humanized, or fully human;
c) is an antibody drug conjugate or a radiolabeled antibody;
d) is Polatuzumab vedotin, 2F2, SN8, 10D10 or H2Mab-250 or a variant thereof, optionally wherein in the variant is a generic variant.
